(19) United States
(12) Patent Application Publication
(10) Pub. No.: US 2004/0157866 A1
(43) Pub. Date: Aug. 12, 2004

(54) Amide compounds

(76) Inventors: Hisashi Takasugi, Osaka-shi (JP); Takeshi Terasawa, Osaka-shi (JP); Yoshikazu Inoue, Osaka (JP); Hideko Nakamura, Osaka (JP); Akira Nagayoshi, Osaka (JP); Yoshiro Furukawa, Osaka (JP); Masafumi Mikami, Osaka (JP); Kasumasa Hinoue, Osaka (JP); Makoto Ohtsubo, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/476,386

(22) PCT Filed: Apr. 9, 2002
(86) PCT No.: PCT/JP02/03529

(30) Foreign Application Priority Data
Apr. 30, 2001 (AU) PR4722
Jan. 11, 2002 (AU) PR9937

(51) Int. Cl. C 07D 4 9/02; C 07D 4 3/02; A61K 31/517

(52) U.S. Cl. 514/266.2; 514/310; 514/314; 544/284; 546/165; 546/148

(57) ABSTRACT

The present invention relates to compounds of the formula (I) wherein X¹ is wherein R¹, R² and R¹⁰ are independently hydrogen or a suitable substituent; R¹¹ and R¹² are independently hydrogen or a suitable substituent; R is unsaturated 5 to 6-membered heteromonocyclic group; A is direct bond or —NH—; X² is monocyclic arylene, unsaturated 5 to 6-membered heteromonocyclic group or cycloalkenylene; Y is bivalent group selected from ethylene, trimethylene and vinylene, wherein CH² is optionally replaced by NH or O, and CH is optionally replaced by N; and Z is —(CH₂)ₙ—, —CO—(CH₂)ₘ—, —CH═CH— or —CO—NH—, wherein n is 1, 2 or 3 and m is 1 or 2, or a salt thereof. The compounds of the present invention inhibit apolipoprotein B (Apo B) secretion and are useful as a medicament for prophylactic and treatment of diseases or conditions resulting from elevated circulating levels of Apo B.

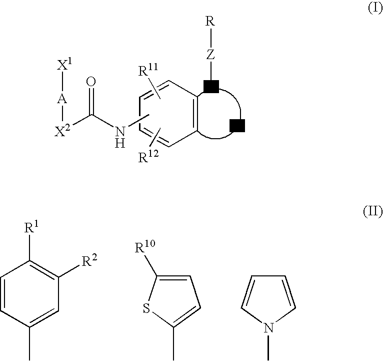

### TECHNICAL FIELD

This invention relates to new amide compounds and salts thereof which inhibit apolipoprotein B (Apo B) secretion and are useful as a medicament.

### BACKGROUND ART

Apo B is the main component of lipoprotein such as VLDL (very low density lipoprotein), IDL (intermediate density lipoprotein) and LDL (low density lipoprotein). Compounds that inhibit Apo B secretion are useful for the treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity and coronary heart diseases. Compounds that inhibit Apo B secretion have been described in WO96/40640, WO98/23593, WO98/56790 and WO00/32582. Compounds that inhibit Apo B secretion are also useful in reducing intestinal fat absorption, reducing food intake and treating obesity in combination with a known anti-obesity agent (EP 1 099 438, EP 1 099 439 and EP 1 099 441).

### DISCLOSURE OF INVENTION

This invention relates to new amide compounds.

One object of this invention is to provide the new and useful amide compounds and salts thereof that inhibit Apo B secretion.

A further object of this invention is to provide a pharmaceutical composition comprising said amide compound or a pharmaceutically acceptable salt thereof.

Still further object of this invention is to provide a use of said amide compounds or pharmaceutically acceptable salts thereof as a medicament for prophylactic and therapeutic treatment of diseases or conditions resulting from elevated circulating levels of Apo B, such as hyperlipemia, hyperlipidemia, hyperlipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

Another object of this invention is to provide a method for inhibiting or decreasing Apo B secretion in a mammal, which comprises administering an Apo B secretion inhibiting or decreasing amount of said amide compound or a pharmaceutically acceptable salt thereof to the mammal.

Still a further object of this invention is to provide a method for preventing or treating a disease or condition resulting from elevated circulating levels of Apo B in a mammal, such as hyperlipemia, hyperlipidemia, hyperlipoproteinemia, hypercholesterolemia, hypertriglyceridemia, atherosclerosis, pancreatitis, NIDDM, obesity, coronary heart diseases, myocardial infarction, stroke, restenosis and Syndrome X, which method comprises administering an effective amount of said amide compound or a pharmaceutically acceptable salt thereof to the mammal.

The object amide compounds of the present invention are novel and can be represented by the following general formula (I):

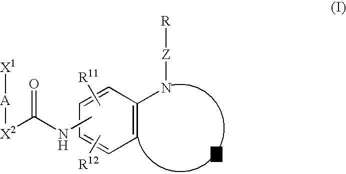

wherein

X¹ is

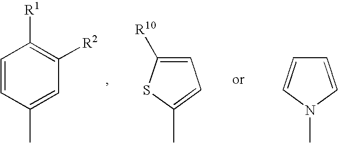

wherein R¹, R² and R¹⁰ are independently hydrogen or a suitable substituent;

R¹¹ and R¹² are independently hydrogen or a suitable substituent;

R is unsaturated 5 to 6-membered heteromonocyclic group, which is optionally substituted by one or more suitable substituent(s);

A is direct bond or —NH—;

X² is monocyclic arylene, unsaturated 5 to 6-membered heteromonocyclic group or cycloalkenylene, each of which is optionally substituted by one or more suitable substituent(s);

Y is bivalent group selected from the group consisting of ethylene, trimethylene and vinylene, wherein CH₂ is optionally replaced by NH or O, and CH is optionally replaced by N, and said bivalent group is optionally substituted by one or more suitable substituent(s);

and

Z is —(CH₂)ₙ—, —CO— (CH₂)ₘ, —CH═CH— or —CO—NH—, wherein n is 1, 2 or 3 and m is 1 or 2,

or a salt thereof.

The preferred embodiments of the amide compound of the present invention represented by the general formula (I) are as follows.

(1) The compound of the formula (I) wherein

R¹ is hydrogen, lower alkyl, lower alkenyl, lower alkoxy, aryl, aryloxy, halogen, trihalo(lower)alkyl, trihalo(lower)alkoxy, nitro, optionally protected amino, lower alkylamino, di(lower)alkylamino, cyclic amino group, lower alkylthio, lower alkylsulfonyl, lower alkylsulfonyloxy, hydroxy(lower)alkyl, optionally protected amino(lower)alkyl, lower alkanoyl, optionally protected carboxy or N,N-di(lower)alkylcarbamoyl;

R² is hydrogen, lower alkyl, lower alkoxy, halogen or trihalo(lower)alkyl;

R¹⁰ is hydrogen or halogen;

R¹¹ and R¹² are independently hydrogen or lower alkyl;

R is unsaturated 5-membered heteromonocyclic group containing 1 or 2 nitrogen atom(s) and a sulfur atom, unsaturated 5-membered heteromonocyclic group containing 1 or 3 nitrogen atom(s), or unsaturated 6-membered heteromonocyclic group containing 1 or 2 nitrogen atom(s),

each of said heteromonocyclic groups is optionally substituted by one or more substituent(s) selected from the group consisting of lower alkyl, optionally protected amino, lower alkylamino, aryl(lower)alkyl, guanidino and oxido;

X² is bivalent group selected from the group consisting of phenylene,

cycloalkenylene,

unsaturated 5-membered heteromonocyclic group containing 1 or 2 hetero atom(s) selected from the group consisting of nitrogen, oxygen and sulfur atoms, and

unsaturated 6-membered heteromonocyclic group containing 1 or 2 nitrogen atom(s),

said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl; and

Y is bivalent group selected from the group consisting of ethylene, trimethylene and vinylene, wherein CH₂ is optionally replaced by NH or O, and CH is optionally replaced by N, and said bivalent group is optionally substituted by one or more substituent(s) selected from the group consisting of lower alkyl, oxo and amino,

or a salt thereof.

(2) The compound of (1) above wherein

R is pyridinyl, pyrimidinyl, pyrazinyl, thiazolyl, thiadiazolyl or triazolyl, each of which is optionally substituted by lower alkyl, optionally protected amino, lower alkylamino, aryl(lower)alkyl, guanidino or oxido; and X² is bivalent group selected from

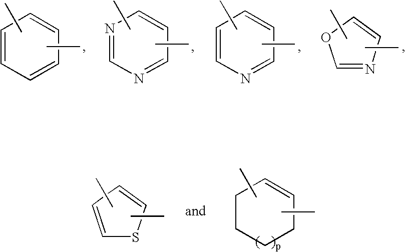

wherein p is 0, 1 or 2;

said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl,

or a salt thereof.

(3) The compound of (2) above wherein

R is

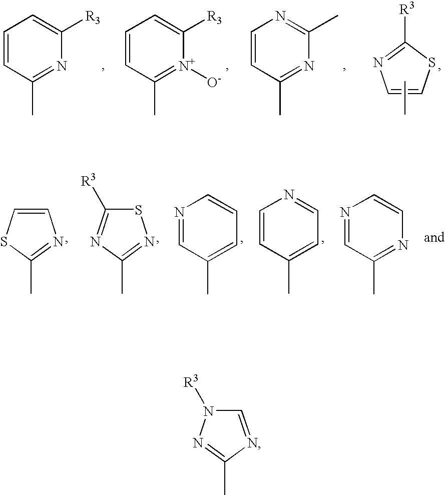

wherein R³ is hydrogen, lower alkyl, optionally protected amino, lower alkylamino, trityl or guanidino;

X² is

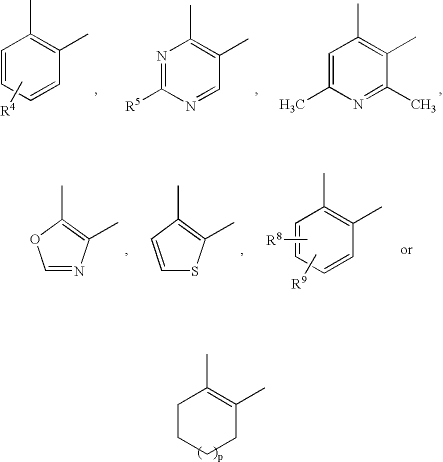

wherein R⁴ is hydrogen, lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl or lower alkanoyloxy(lower)alkyl;

R⁵ is hydrogen or lower alkyl;

R⁸ and R⁹ are independently lower alkyl or lower alkoxy; and

p is 0, 1 or 2; and

Y is

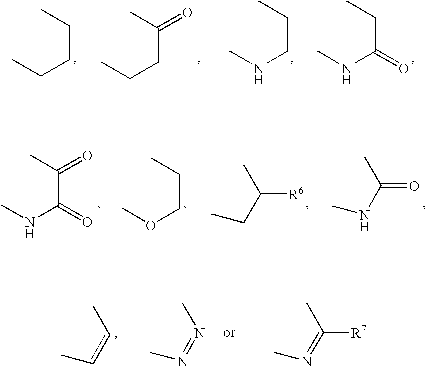

wherein R⁶ is hydrogen or lower alkyl; and

R⁷ is hydrogen, lower alkyl or amino,

or a salt thereof.

(4) The compound of (3) above wherein

R¹ is hydrogen, methyl, ethyl, isopropyl, isopropenyl, methoxy, ethoxy, phenyl, phenoxy, chloro, fluoro, trifluoromethyl, trifluoromethoxy, nitro, amino, dimethylamino, piperidino, 4-morpholinyl, 4-thiomorpholinyl, 1,1-dioxothiomorpholin-4-yl, methylthio, isopropylthio, methylsulfonyl, methylsulfonyloxy, 1-hydroxyethyl, 1-hydroxy-1-methylethyl, 1-aminoethyl, 1-(benzylamino)ethyl, acetyl, acetylamino, carboxy, methoxycarbonyl, isopropoxycarbonyl, pivaloylmethoxycarbonyl or N,N-diethylcarbamoyl;

R² is hydrogen, methyl, methoxy, chloro or trifluoromethyl;

R¹⁰ is chloro;

R¹¹ and R¹² are independently hydrogen or methyl;

A is direct bond;

Z is —CH₂CH₂—, —CO—CH₂—, —CH═CH— or —CO—NH—;

R³ is hydrogen, methyl, amino, methylamino, formylamino, tert-butoxycarbonylamino,

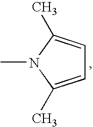

trityl or guanidino;

R⁴ is hydrogen, methyl, methoxy, chloro, nitro, amino, dimethylamino, hydroxymethyl, methoxymethyl, N,N-dimethylaminomethyl or acetyloxymethyl;

R⁵ is hydrogen, methyl or isopropyl;

R⁶ is hydrogen or methyl;

R⁷ is hydrogen, methyl or amino; and

R⁸ and R⁹ are independently methyl or methoxy,

or a salt thereof.

(5) A compound of formula (II):

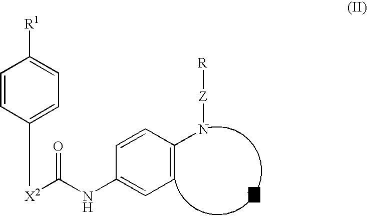

wherein

R¹ is hydrogen, methyl, ethyl, isopropyl, isopropenyl, methoxy, ethoxy, phenyl, phenoxy, chloro, fluoro, trifluoromethyl, trifluoromethoxy, nitro, amino, dimethylamino, piperidino, 4-morpholinyl, 4-thiomorpholinyl, 1,1-dioxothiomorpholin-4-yl, methylthio, isopropylthio, methylsulfonyl, methylsulfonyloxy, 1-hydroxyethyl, 1-hydroxy-1-methylethyl, 1-aminoethyl, 1-(benzylamino)ethyl, acetyl, acetylamino, carboxy, methoxycarbonyl, isopropoxycarbonyl, pivaloyloxymethoxycarbonyl or N,N-diethylcarbamoyl;

R is

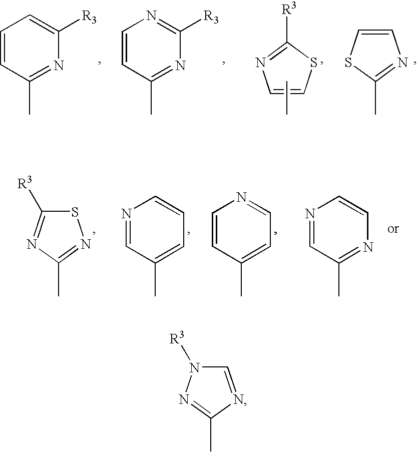

wherein R³ is hydrogen, methyl, amino, methylamino, formylamino, tert-butoxycarbonylamino,

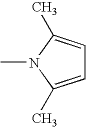

or trityl;

X² is

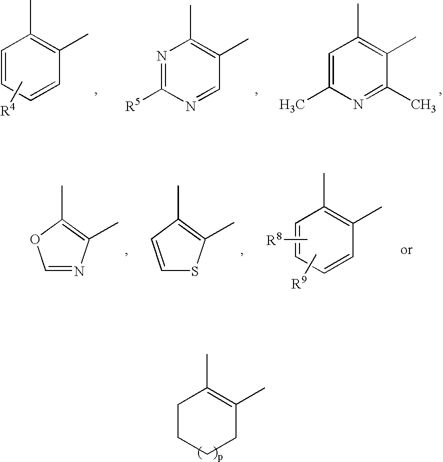

wherein R⁴ is hydrogen, methyl, methoxy, chloro, nitro, amino, dimethylamino, hydroxymethyl, methoxymethyl, N,N-dimethylaminomethyl or acetyloxymethyl;

R⁵ is hydrogen, methyl or isopropyl;

R⁸ and R⁹ are independently methyl or methoxy; and

p is 0, 1 or 2;

Y is

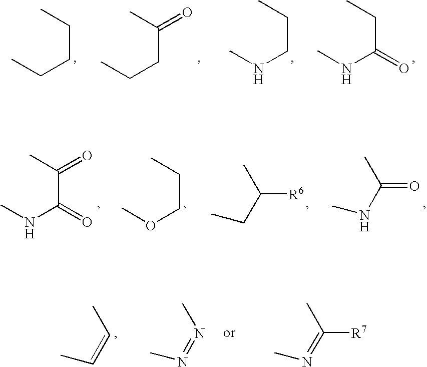

wherein R⁶ is hydrogen or methyl; and

R⁷ is hydrogen, methyl or amino; and

Z is —CH₂CH₂—, —CO—CH₂— or —CH═CH—,

or a salt thereof.

(6) A compound of formula (III):

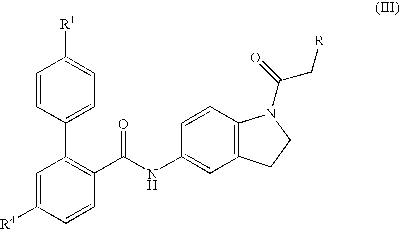

wherein

R¹ is hydrogen, lower alkyl, halogen, trihalo(lower)alkyl or di(lower)alkylamino;

R is

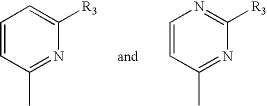

wherein R³ is hydrogen or amino; and

R⁴ is hydrogen or lower alkyl;

or a salt thereof.

(7) The compound of (6) above, which is selected from the group consisting of

N-[1-(2-pyridinylacetyl)-2,3-dihydro-1H-indol-5-yl]-4′-(trifluoromethyl)-1,1′-biphenyl-2-carboxamide (Example 4),

4′-ethyl-N-[1-(2-pyridinylacetyl)-2,3-dihydro-1H-indol-5-yl]-1,1′-biphenyl-2-carboxamide (Example 7),

N-{1-[(6-amino-2-pyridinyl)acetyl]-2,3-dihydro-1H-indol-5-yl}-4′-(trifluoromethyl)-1,1′-biphenyl-2-carboxamide (Example 9),

4′,5-dimethyl-N-[1-(2-pyridinylacetyl)-2,3-dihydro-1H-indol-5-yl]-1,1′-biphenyl-2-carboxamide (Example 11), 4′-chloro-5-methyl-N-[1-(2-pyridinylacetyl)-2,3-dihydro-1H-indol-5-yl]-1,1′-biphenyl-2-carboxamide (Example 16),

4′-(dimethylamino)-N-[1-(2-pyridinylacetyl)-2,3-dihydro-1H-indol-5-yl]-1,1′-biphenyl-2-carboxamide (Example 30),

N-{1-[(6-amino-2-pyridinyl)acetyl]-2,3-dihydro-1H-indol-5-yl}-4′-methyl-1,1′-biphenyl-2-carboxamide (Example 117),

N-{1-[(6-amino-2-pyridinyl)acetyl]-2,3-dihydro-1H-indol-5-yl}-4′-ethyl-1,1′-biphenyl-2-carboxamide (Example 156),

N-{1-[(2-amino-4-pyrimidinyl)acetyl]-2,3-dihydro-1H-indol-5-yl}-4′-ethyl-1,1′-biphenyl-2-carboxamide (Example 179), and

N-{1-[(2-amino-4-pyrimidinyl)acetyl]-2,3-dihydro-1H-indol-5-yl}-4′-methyl-1,1′-biphenyl-2-carboxamide (Example 234),

or a salt thereof.

Other preferred embodiments of the amide compound of the present invention represented by the general formula (I) are as follows.

(8) The compound of the formula (I) wherein

X¹ is

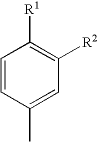

R¹ and R² are independently hydrogen or a suitable substituent;

X² is monocyclic arylene or unsaturated 5 or 6-membered heteromonocyclic group, each of which is optionally substituted by one or more suitable substituent(s);

and

Z is —(CH₂)ₙ—, —CO—(CH₂)ₘ— or —CH═CH—, wherein n is 1, 2 or 3 and m is 1 or 2,

or a salt thereof.

(9) The compound of (8) above wherein

R¹ is hydrogen, lower alkyl, lower alkoxy, aryloxy, halogen, trihalo(lower)alkyl, trihalo(lower)alkoxy, nitro, optionally protected amino, lower alkylamino or di(lower)alkylamino;

R² is hydrogen, lower alkyl, lower alkoxy, halogen or trihalo(lower)alkyl;

R is unsaturated 5-membered heteromonocyclic group containing 1 or 2 nitrogen atom(s) and a sulfur atom, or

unsaturated 6-membered heteromonocyclic group containing 1 or 2 nitrogen atom(s),

each of said heteromonocyclic groups is optionally substituted by one or more substituent(s) selected from the group consisting of lower alkyl, optionally protected amino and lower alkylamino;

X² is bivalent group selected from the group consisting of phenylene,

unsaturated 5-membered heteromonocyclic group containing 1 or 2 hetero atom(s) selected from the group consisting of nitrogen, oxygen and sulfur atoms, or

unsaturated 6-membered heteromonocyclic group containing 1 or 2 nitrogen atom(s),

said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl; and

Y is bivalent group selected from the group consisting of ethylene, trimethylene and vinylene, wherein CH₂ is optionally replaced by NH or O, and CH is optionally replaced by N, and said bivalent group is optionally substituted by one or more substituent(s) selected from the group consisting of lower alkyl, oxo and amino,

or a salt thereof.

(10) The compound of (9) above wherein

R is pyridinyl, pyrimidinyl, pyrazinyl, thiazolyl or thiadiazolyl, each of which is optionally substituted by lower alkyl, optionally protected amino or lower alkylamino; and

X² is bivalent group selected from

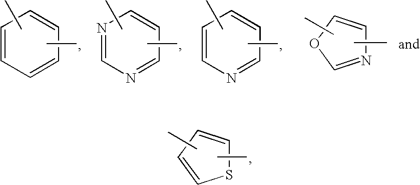

said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl,

or a salt thereof.

(11) The compound of (10) above wherein

R is

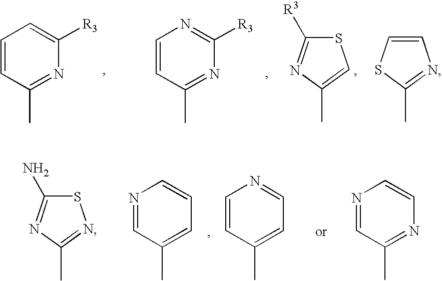

wherein R³ is hydrogen, lower alkyl, optionally protected amino or lower alkylamino;

X² is

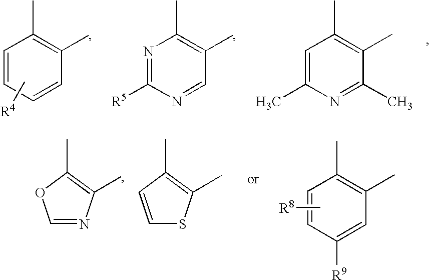

wherein R⁴ is hydrogen, lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl or lower alkanoyloxy(lower)alkyl;

R⁵ is hydrogen or lower alkyl; and

R⁸ and R⁹ are independently lower alkyl or lower alkoxy; and

Y is

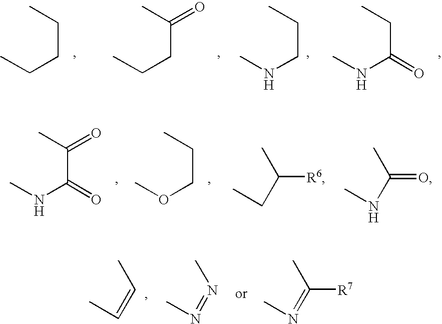

wherein R⁶ is hydrogen or lower alkyl; and

R⁷ is hydrogen or amino,

or a salt thereof.

(12) The compound of (11) above wherein

R¹ is hydrogen, methyl, ethyl, methoxy, ethoxy, phenoxy, chloro, fluoro, trifluoromethyl, trifluoromethoxy, nitro, amino or dimethylamino;

R² is hydrogen, methyl, methoxy, chloro or trifluoromethyl;

A is direct bond;

Z is —CH₂CH₂—, —CO—CH₂— or —CH═CH—;

R³ is hydrogen, methyl, amino, methylamino, formylamino, tert-butoxycarbonylamino or

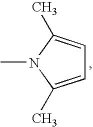

R⁴ is hydrogen, methyl, methoxy, chloro, nitro, amino, dimethylamino, hydroxymethyl, methoxymethyl, N,N-dimethylaminomethyl or acetyloxymethyl;

R⁵ is hydrogen, methyl or isopropyl;

R⁶ is hydrogen or methyl; and

R⁸ and R⁹ are independently methyl or methoxy,

or a salt thereof.

(13) The compound of the formula (I) wherein

X¹ is

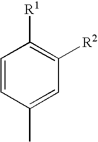

R¹ and R² are independently hydrogen or a suitable substituent;

X² is monocyclic arylene or unsaturated 5 or 6-membered heteromonocyclic group, each of which is optionally substituted by one or more suitable substituent(s); and

Z is —(CH₂)ₙ— or —CO—(CH₂)ₘ, wherein n is 1, 2 or 3 and m is 1 or 2,

or a salt thereof.

(14) The compound of (13) above wherein

R¹ is hydrogen, lower alkyl, lower alkoxy, aryloxy, halogen, trihalo(lower)alkyl, trihalo(lower)alkoxy, nitro, optionally protected amino, lower alkylamino or di(lower)alkylamino;

R² is hydrogen, lower alkyl, lower alkoxy, halogen or trihalo(lower)alkyl;

R is

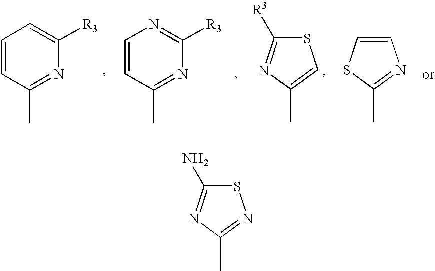

wherein R³ is hydrogen, lower alkyl, optionally protected amino, or lower alkylamino;

X² is

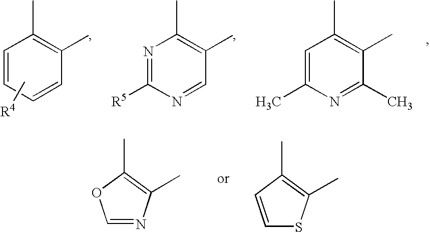

wherein R⁴ is hydrogen, lower alkyl, lower alkoxy, halogen, nitro, optionally protected amino, (lower)alkylamino, di(lower)alkylamino, hydroxy(lower)alkyl, lower alkoxy(lower)alkyl, amino(lower)alkyl, N-lower alkylamino(lower)alkyl, or N,N-di(lower)alkylamino(lower)alkyl; and

R⁵ is hydrogen or lower alkyl; and

Y is

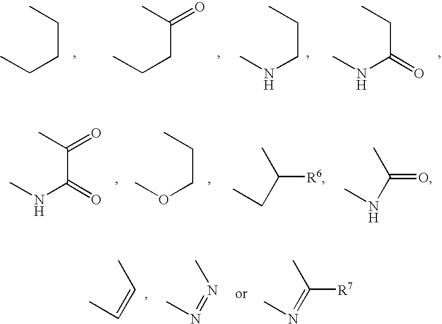

wherein R⁶ is hydrogen or lower alkyl; and

R⁷ is hydrogen or amino,

or a salt thereof.

(15) The compound of (14) above wherein

R¹ is hydrogen, methyl, ethyl, methoxy, ethoxy, phenoxy, chloro, fluoro, trifluoromethyl, trifluoromethoxy, nitro, amino or dimethylamino;

R² is hydrogen, methyl, methoxy, chloro or trifluoromethyl;

R³ is hydrogen, methyl, amino, methylamino, formylamino, tert-butoxycarbonylamino or

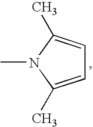

R⁴ is hydrogen, methyl, methoxy, chloro, nitro, amino, dimethylamino, hydroxymethyl, methoxymethyl or N,N-dimethylaminomethyl;

R⁵ is hydrogen, methyl or isopropyl; and

R⁶ is hydrogen or methyl,

or a salt thereof.

(16) The compound of (15) above wherein A is direct bond and Z is —CH₂CH₂— or —CO—CH₂—, or a salt thereof.

Suitable salts of the object compound (I) may be pharmaceutically acceptable salts such as conventional nontoxic salts and include, for example, a salt with a base or an acid addition salt such as a salt with an inorganic base, for example, an alkali metal salt (e.g., sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.); an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, citrate, fumarate, methanesulfonate, benzenesulfonate, toluenesulfonate, etc.); and a salt with a basic or acidic amino acid (e.g., arginine, aspartic acid, glutamic acid, etc.).

In the above and subsequent descriptions of the present specification, suitable examples and illustration of the various definitions which the present invention intends to include within the scope thereof are explained in detail as follows.

The term “lower” is used to intend a group having 1 to 6, preferably 1 to 4, carbon atom(s), unless otherwise provided.

Suitable “lower alkyl” and “lower alkyl” moiety in the terms “trihalo(lower)alkyl”, “lower alkylamino”, “di(lower)alkylamino”, “hydroxy(lower)alkyl”, “lower alkoxy(lower)alkyl”, “amino(lower)alkyl”, “N-lower alkylamino(lower)alkyl”, “N,N-di(lower)alkylamino(lower)alkyl”, “lower alkanoyl(lower)alkyl”, “lower alkylthio”, “lower alkylsulfonyl”, “lower alkylsulfonyloxy”, “N,N-di(lower)alkylcarbamoyl” and “aryl(lower)alkyl” include straight or branched one having 1 to 6 carbon atom(s), such as methyl, ethyl, propyl, isopropyl, butyl, isobutyl, sec-butyl, tert-butyl, pentyl, tert-pentyl and hexyl, in which more preferred one is C₁-C₄ alkyl.

Suitable “lower alkenyl” includes straight or branched alkenyl having 2 to 6 carbon atom(s), such as vinyl, 1-propenyl, allyl, isopropenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl and 5-hexenyl, in which more preferred one is C₂-C₄ alkenyl.

Suitable “lower alkoxy” and “lower alkoxy” moiety in the terms “trihalo(lower)alkoxy” and “lower alkoxy(lower)alkyl” include straight or branched alkoxy having 1 to 6 carbon atom(s), such as methoxy, ethoxy, propoxy, isopropoxy, butoxy, isobutoxy, sec-butoxy, tert-butoxy, pentyloxy, tert-pentyloxy and hexyloxy, in which more preferred one is C₁-C₄ alkoxy.

Suitable “aryl” and “aryl” moiety in the term “aryl(lower)alkyl” include C₆-C₁₀ aryl such as phenyl and naphthyl (e.g., 1-naphthyl and 2-naphthyl), in which more preferred one is phenyl.

Suitable “aryl(lower)alkyl” include mono-, di- or tri(C₆-C₁₀)aryl(C₁-C₆)alkyl such as benzyl, benzhydryl, trityl, phenethyl, 1-phenylethyl, 1-naphthylmethyl and 2-naphthylmethyl, in which more preferred one is benzyl and trityl.

Suitable “lower alkanoyl” moiety in the term “lower alkanoyloxy(lower)alkyl” includes C₁-C₆ alkanoyl such as formyl, acetyl, propanoyl, butanoyl, 2-methylpropanyl, pentanoyl, 3-methylbutanoyl, 2,2-dimethylpropanoyl and hexanoyl, in which more preferred one is C₁-C₄ alkanoyl.

Suitable “aryloxy” include C₆-C₁₀ aryloxy such as phenoxy and naphthyloxy, in which more preferred one is phenoxy.

Suitable “halogen” and “halogen” moiety in the terms “trihalo(lower)alkyl” and “trihalo(lower)alkoxy” include, for example, fluorine, bromine, chlorine and iodine.

Suitable “trihalo(lower)alkyl” includes trihalo(C₁-C₆)alkyl such as trifluoromethyl, trichloromethyl and tribromomethyl, in which more preferred one is trifluoromethyl.

Suitable “trihalo(lower)alkoxy” includes trihalo(C₁-C₆)alkoxy such as trifluoromethoxy, trichloromethoxy and tribromomethoxy, in which more preferred one is trifluoromethoxy.

Suitable “lower alkylamino” includes C₁-C₆ alkylamino such as methylamino, ethylamino, propylamino, isopropylamino, butylamino, pentylamino, and hexylamino, in which more preferred one is methylamino.

Suitable “di(lower)alkylamino” includes di(C₁-C₆)alkylamino such as dimethylamino, diethylamino, dipropylamino, diisopropylamino, dibutylamino, dipentylamino, dihexylamino, ethylmethylamino, methylpropylamino, and ethylpropylamino, in which more preferred one is dimethylamino.

Suitable “cyclic amino group” includes 6-membered cyclic amino group such as piperidino and 6-membered heterocyclic amino group such as 4-morpholinyl, 4-thiomorpholinyl and 1,1-dioxothiomorpholin-4-yl.

Suitable “lower alkylthio” includes C₁-C₆ alkylthio such as methylthio, ethylthio, propylthio, isopropylthio, butylthio, isobutylthio, sec-butylthio, tert-butylthio, pentylthio, tert-pentylthio and hexylthio, in which more preferred ones are methylthio and isopropylthio.

Suitable “lower alkylsulfonyl” includes C₁-C₆ alkylsulfonyl such as methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, tert-pentylsulfonyl and hexylsulfonyl, in which more preferred one is methylsulfonyl.

Suitable “lower alkylsulfonyloxy” includes C₁-C₆ alkylsulfonyloxy such as methylsulfonyloxy, ethylsulfonyloxy, propylsulfonyloxy, isopropylsulfonyloxy, butylsulfonyloxy, isobutylsulfonyloxy, sec-butylsulfonyloxy, tert-butylsulfonyloxy, pentylsulfonyloxy, tert-pentylsulfonyloxy and hexylsulfonyloxy, in which more preferred one is methylsulfonyloxy.

Suitable “hydroxy(lower)alkyl” includes hydroxy(C₁-C₆)alkyl such as hydroxymethyl, 2-hydroxyethyl, 1-hydroxyethyl, 3-hydroxypropyl, 2-hydroxypropyl, 1-hydroxypropyl, 1-hydroxy-1-methylethyl, 4-hydroxybutyl, 5-hydroxypentyl and 6-hydroxyhexyl, in which more preferred ones are 1-hydroxyethyl and 1-hydroxy-1-methylethyl.

Suitable “N,N-di(lower)alkylcarbamoyl” includes N,N-dimethylcarbamoyl, N,N-diethylcarbamoyl, N,N-dipropylcarbamoyl, N,N-diisopropylcarbamoyl, N,N-dibutylcarbamoyl, N-ethyl-N-methylcarbamoyl, N-methyl-N-propylcarbamoyl, N-ethyl-N-propylcarbamoyl, in which more preferred one is N,N-diethylcarbamoyl.

Suitable “lower alkoxy(lower)alkyl” includes C₁-C₆ alkoxy(C₁-C₆)alkyl such as methoxymethyl, 2-methoxyethyl, 1-methoxyethyl, 2-ethyoxyethyl, 3-methoxypropyl, 3-ethoxypropyl, 4-methoxybutyl, 5-methoxypentyl and 6-methoxyhexyl, in which more preferred one is methoxymethyl.

Suitable “N-lower alkylamino(lower)alkyl” includes N-(C₁-C₆)alkylamino(C₁-C₆)alkyl such as N-methylaminomethyl, N-ethylaminomethyl, N-propylaminomethyl, N-isopropylaminomethyl, N-butylaminomethyl, N-pentylaminomethyl, N-hexylaminomethyl, 2-(N-methylamino)ethyl and 2-(N-ethylamino)ethyl, in which more preferred one is N-methylaminomethyl.

Suitable “N,N-di(lower)alkylamino(lower)alkyl” includes N,N-di(C₁-C₆)alkylamino(C₁-C₆)alkyl such as N,N-dimethylaminomethyl, N,N-diethylaminomethyl, N,N-dipropylaminomethyl, N,N-diisopropylaminomethyl, N,N-dibutylaminomethyl, N-ethyl-N-methylaminomethyl, N-methyl-N-propylaminomethyl, N-ethyl-N-propylaminomethyl, 2-(N,N-dimethylamino)ethyl, 2-(N,N-diethylamino)ethyl, 2-(N,N-dipropylamino)ethyl and 2-(N,N-ethylmethylamino)ethyl, in which more preferred one is N,N-dimethylaminomethyl.

Suitable “lower alkanoyloxy(lower)alkyl” includes C₁-C₆ alkanoyloxy(C₁-C₆)alkyl such as formyloxymethyl, acetyloxymethyl, propanoyloxymethyl, butanoyloxymethyl, 2-methylpropanyloxymethyl, pentanoyloxymethyl, 3-methylbutanoyloxymethyl, 2,2-dimethylpropanoyloxymethyl, hexanoyloxymethyl, 2-acetyloxyethyl and 3-acetyloxypropyl, in which more preferred one is acetyloxymethyl.

Suitable “monocyclic arylene” includes phenylene (e.g., 1,2-phenylene, 1,3-phenylene and 1,4-phenylene), in which the more preferred one is 1,2-phenylene.

Suitable “cycloalkenylene” includes C₅-C₇ cycloalkenylene such as cyclopentenylene (e.g., 1,2-cyclopentenylene, 1,3-cyclopentenylene and 1,4-cyclopentenylene), cyclohexenylene (e.g., 1,2-cyclohexenylene, 1,3-cyclohexenylene and 1,4-cyclohexenylene), cycloheptenylene (e.g., 1,2-cycloheptenylene, 1,3-cycloheptenylene, 1,4-cycloheptenylene and 1,5-cycloheptenylene), in which the more preferred one is cyclohexenylene and cycloheptenylene, and the most preferred ones are

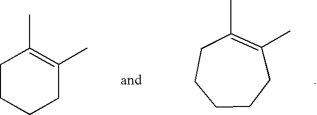

Suitable “unsaturated 5 or 6-membered heteromonocyclic group” includes 5 or 6-membered aromatic heteromonocyclic group containing 1 to 3 hetero atom(s) selected from nitrogen, oxygen and sulfur atoms. Suitable examples of unsaturated 5 or 6-membered heteromonocyclic group include

unsaturated 5-membered heteromonocyclic group containing 1 or 2 hetero atom(s) selected from the group consisting of nitrogen, oxygen and sulfur atoms such as thiazolyl, isothiazolyl, oxazolyl, isoxazolyl, imidazolyl, pyrazolyl, furanyl, thienyl and pyrrolyl;

unsaturated 5-membered heteromonocyclic group containing 1 or 2 nitrogen atom(s) and a sulfur atom such as thiazolyl, isothiazolyl and thiadiazolyl;

unsaturated 5-membered heteromonocyclic group containing 1 or 3 nitrogen atom(s) such as triazolyl; and

unsaturated 6-membered heteromonocyclic group containing 1 or 2 nitrogen atom(s) such as pyridinyl (also referred to as pyridyl), pyrimidinyl, pyrazinyl and pyridazinyl.

Suitable examples of “unsaturated 5 or 6-membered heteromonocyclic group” for R include unsaturated 5-membered heteromonocyclic group containing 1 or 2 nitrogen atom(s) and a sulfur atom, unsaturated 5-membered heteromonocyclic group containing 1 or 3 nitrogen atom(s), and unsaturated 6-membered heteromonocyclic group containing 1 or 2 nitrogen atom(s). More preferred examples include pyridinyl, pyrimidinyl, pyrazinyl, thiazolyl, thiadiazolyl and triazolyl.

“Unsaturated 5 or 6-membered heteromonocyclic group” at R is optionally substituted by one or more suitable substituent(s), preferably by 1 to 3 substituent(s). Suitable examples of such substituent include lower alkyl, optionally protected amino, lower alkylamino, aryl(lower)alkyl, guanidino and oxido.

Suitable examples of “unsaturated 5 or 6-membered heteromonocyclic group” for X² include

unsaturated 5-membered heteromonocyclic group containing 1 or 2 hetero atom(s) selected from the group consisting of nitrogen, oxygen and sulfur atoms, and

unsaturated 6-membered heteromonocyclic group containing 1 or 2 nitrogen atom(s). More preferred examples include

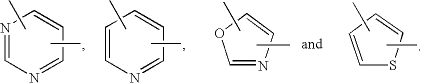

Each of “monocyclic arylene” and “unsaturated 5 or 6-membered heteromonocyclic group” at X is optionally substituted by suitable substituent(s), preferably by 1 to 3 substituent(s). Suitable examples of such substituent include lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyl(lower)alkyl.

Suitable examples of “amino protective group” include acyl such as lower alkanoyl (e.g., formyl, acetyl, etc.), lower alkoxycarbonyl (e.g., tert-butoxycarbonyl, etc.), mono(or di or tri)phenyl(lower)alkoxy carbonyl (e.g., benzyloxycarbonyl, etc.), and a conventional protective group such as mono(or di or tri)aryl(lower)alkyl, for example, mono(or di or tri)phenyl(lower)alkyl (e.g., benzyl, trityl, etc.) and

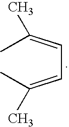

“Optionally protected amino” includes amino and protected amino. Suitable examples of “amino protective group” in the term “protected amino” include the same amino protective groups as mentioned above. Suitable examples of “protected amino” include formylamino, acetylamino, tert-butoxycarbonylamino, benzylamino or

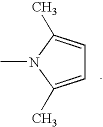

“Optionally protected amino(lower)alkyl” includes amino(lower)alkyl and protected amino(lower)alkyl.

Suitable “amino(lower)alkyl” includes amino(C₁-C₆)alkyl such as aminomethyl, 1-aminoethyl, 2-aminoethyl, 1-aminopropyl, 2-aminopropyl, 3-aminopropyl, 4-aminobutyl, 5-aminopentyl and 6-aminohexyl.

Suitable examples of “amino protective group” in the term “protected amino(lower)alkyl” include the same amino protective groups as mentioned above. Suitable examples of “protected amino(lower)alkyl” include protected amino(C₁-C₆)alkyl, such as formylaminomethyl, acetylaminomethyl, tert-butoxycarbonylaminomethyl, benzylaminomethyl, 1-(formylamino)ethyl, 1-(acetylamino)ethyl, 1-(benzylamino)ethyl, 2-(formylamino)ethyl, 2-(acetylamino)ethyl, 2-(benzylamino)ethyl or

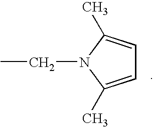

Suitable examples of “carboxy protective group” include lower alkyl (e.g., methyl, ethyl, tert-butyl, etc.), mono(or di or tri)phenyl(lower)alkyl optionally substituted by nitro (e.g., benzyl, 4-nitrobenzyl, benzhydryl, trityl, etc.) and lower alkylcarbonyloxy(lower)alkyl (e.g., pivaloyloxymethyl).

“Optionally protected carboxy” includes carboxy and protected carboxy. Suitable examples of “carboxy protective group” in the term “protected carboxy” include the same carboxy protective groups as mentioned above. Suitable examples of “protected carboxy” include lower alkoxycarbonyl, lower alkylcarbonyloxy(lower)alkoxycarbonyl. Suitable “lower alkoxycarbonyl” includes C₁-C₆ alkoxycarbonyl such as methoxycarbonyl, ethoxycarbonyl, propoxycarbonyl, isopropoxycarbonyl and tert-butoxycarbonyl. Suitable “lower alkylcarbonyloxy(lower)alkoxycarbonyl” includes C₁-C₆ alkylcarbonyloxy(C₁-C₆)alkoxycarbonyl such as pivaloyloxymethoxycarbonyl.

Preferably, R¹ is hydrogen, lower alkyl, lower alkenyl, lower alkoxy, aryl, aryloxy, halogen, trihalo(lower)alkyl, trihalo(lower)alkoxy, nitro, optionally protected amino, lower alkylamino, di(lower)alkylamino, lower alkylthio, lower alkylsulfonyl, lower alkylsufonyloxy, hydroxy(lower)alkyl, optionally protected amino(lower)alkyl, lower alkanoyl, optionally protected carboxy or N,N-di(lower)alkylcarbamoyl, and more preferably, R¹ is hydrogen, methyl, ethyl, isopropyl, isopropenyl, methoxy, ethoxy, phenyl, phenoxy, chloro, fluoro, trifluoromethyl, trifluoromethoxy, nitro, amino, dimethylamino, piperidino, 4-morpholinyl, 4-thiomorpholinyl, 1,1-dioxothiomorpholin-4-yl, methylthio, isopropylthio, methylsulfonyl, methylsulfonyloxy, 1-hydroxyethyl, 1-hydroxy-1-methylethyl, 1-aminoethyl, 1-(benzylamino)ethyl, acetyl, acetylamino, carboxy, methoxycarbonyl, isopropoxycarbonyl, pivaloyloxymethoxycarbonyl or N,N-diethylcarbamoyl.

Preferably, R² is hydrogen, lower alkyl, lower alkoxy, halogen or trihalo(lower)alkyl, and more preferably, R² is hydrogen, methyl, methoxy, chloro or trifluoromethyl;

Preferably, R¹⁰ is chloro.

Preferably, R¹¹ and R¹² are independently hydrogen or methyl.

Preferably, A is direct bond.

Examples of a preferable group represented by R include

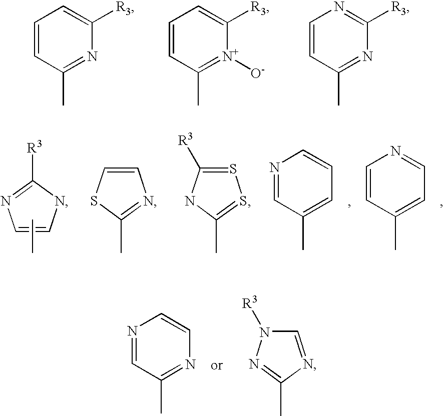

wherein R³ is hydrogen, lower alkyl, optionally protected amino, lower alkylamino, trityl or guanidino, and more preferable examples include

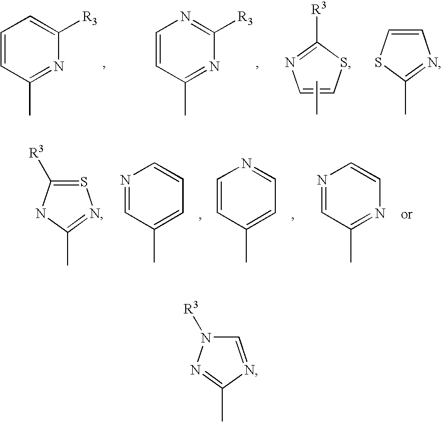

wherein R³ is hydrogen, methyl, amino, methylamino, formylamino, tert-butoxycarbonylamino,

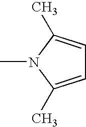

or trityl.

Examples of a preferable group represented by X² include

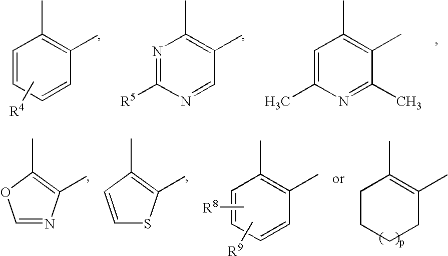

wherein R⁴ is hydrogen, lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl or lower alkanoyloxy(lower)alkyl;

R⁵ is hydrogen or lower alkyl;

R⁸ and R⁹ are independently lower alkyl or lower alkoxy; and

p is 0, 1 or 2,

and more preferable examples include

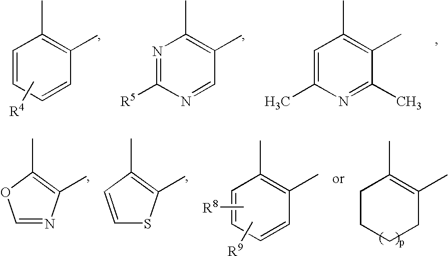

wherein R⁴ is hydrogen, methyl, methoxy, chloro, nitro, amino, dimethylamino, hydroxymethyl, methoxymethyl, N,N-dimethylaminomethyl or acetyloxymethyl;

R⁵ is hydrogen, methyl or isopropyl;

R⁸ and R⁹ are independently methyl or methoxy; and

p is 0, 1 or 2.

Preferable examples of the fused ring moiety represented by the formula

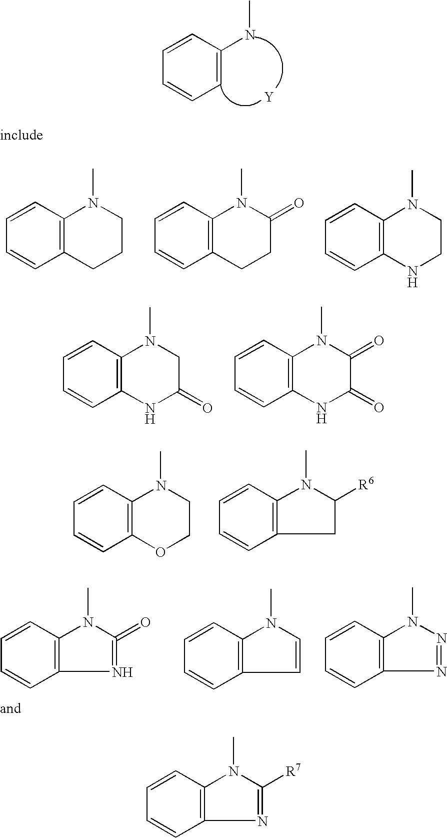

wherein R⁶ is hydrogen or lower alkyl; and R⁷ is hydrogen, methyl or amino.

Preferably, Z is —CH₂CH₂—, —CO—CH₂— or —CH═CH—.

When Z is —CO—(CH₂)ₘ— or —CO—NH—, the carbonyl group in —CO—(CH₂)ₘ or —CO—NH— is bonded to the nitrogen atom on the fused ring moiety.

The object compound (I) of the present invention can be prepared by the following processes.

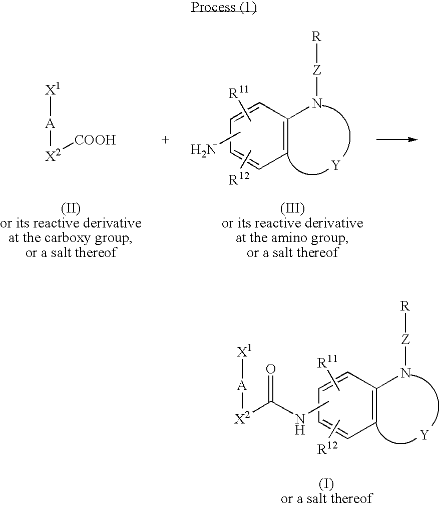

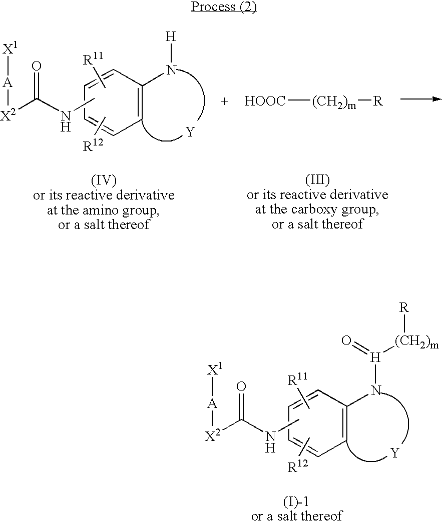

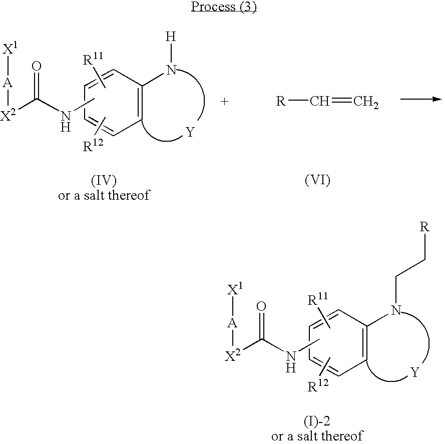

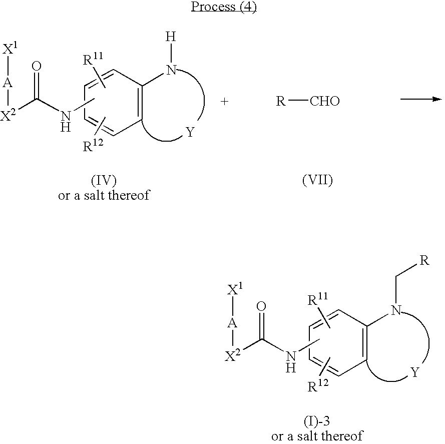

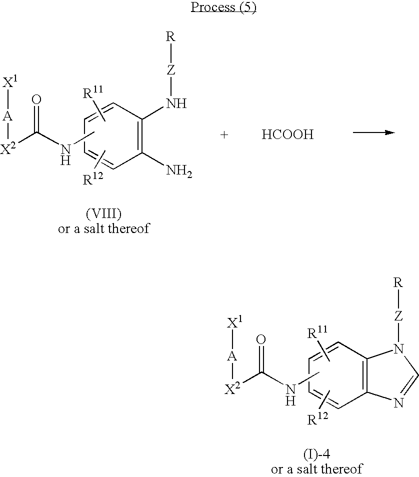

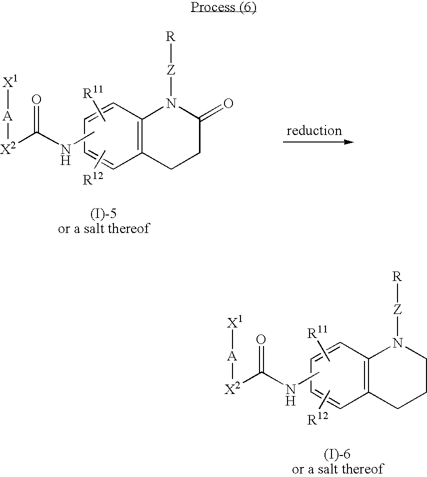

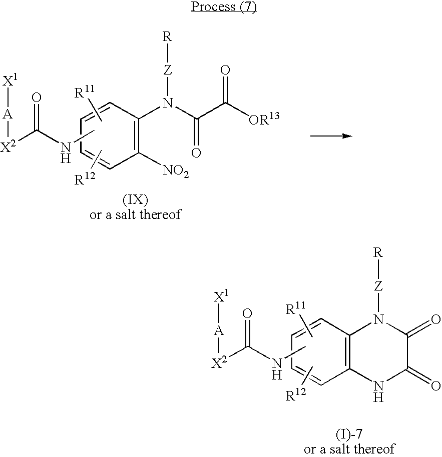

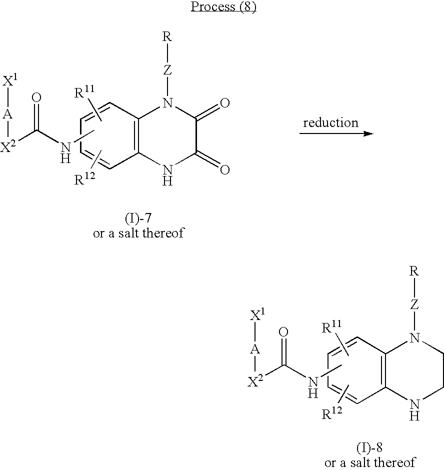

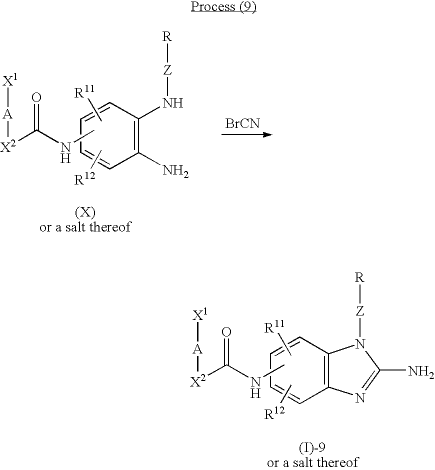

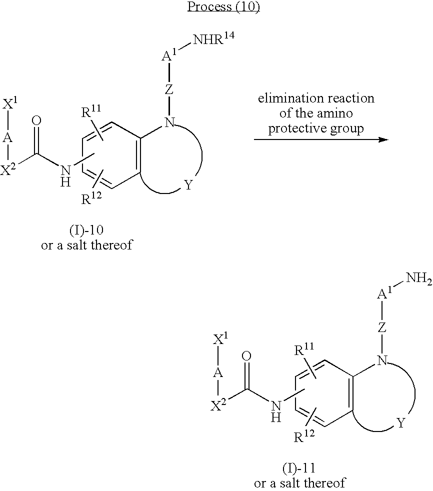

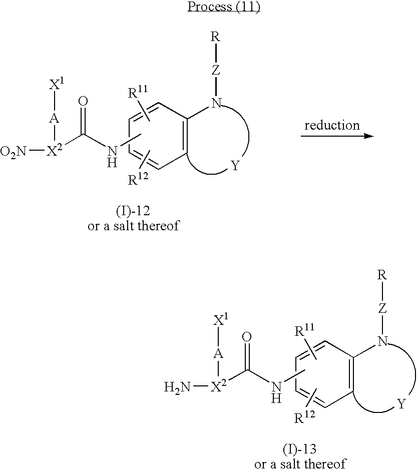

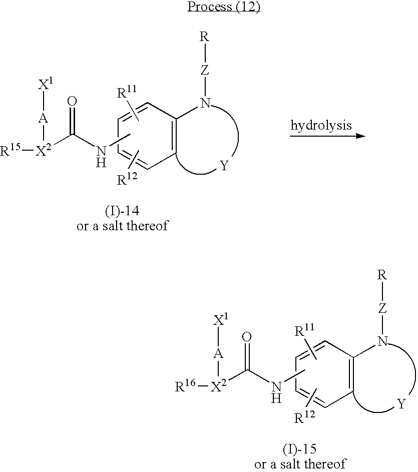

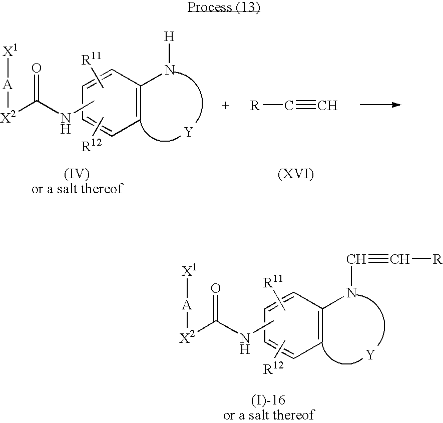

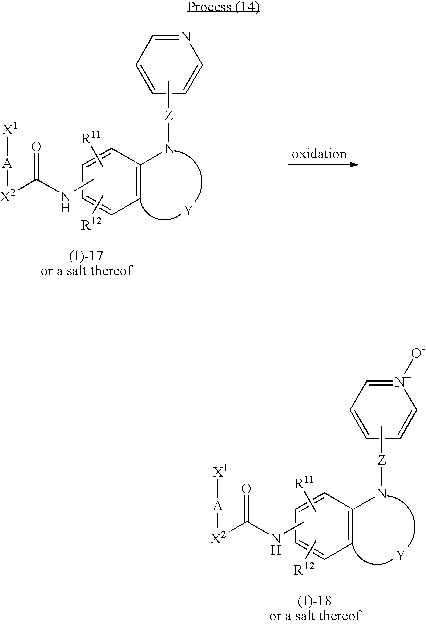

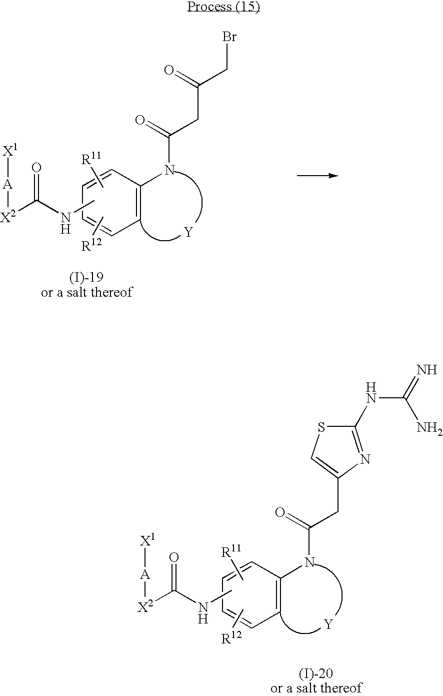

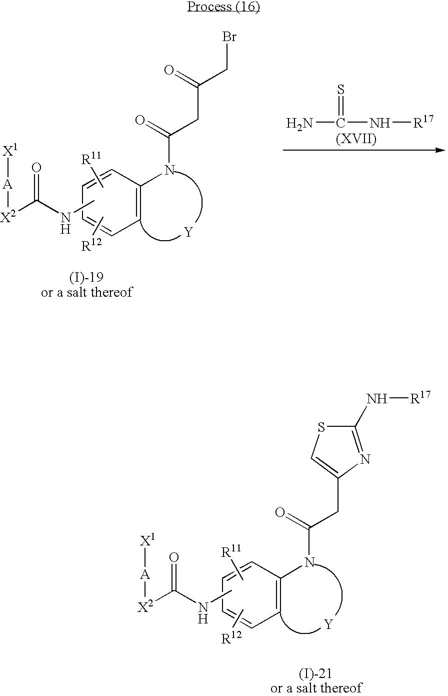

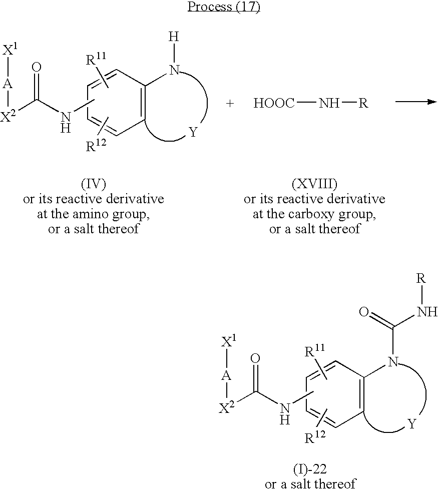

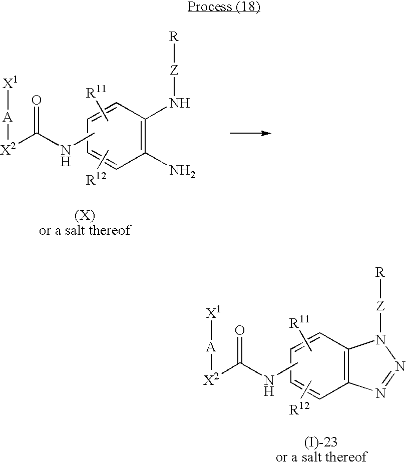

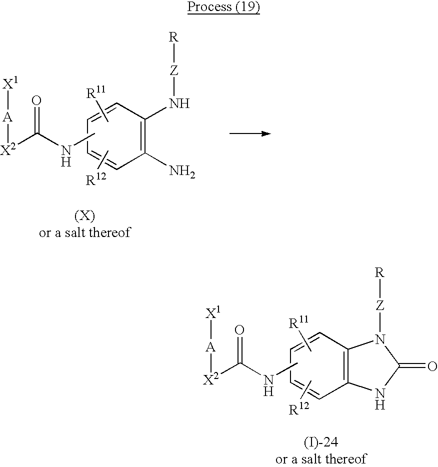

wherein R¹¹, R¹², R, A, X¹, X², y, Z and m are as defined above,

R¹³ is lower alkyl,

R¹⁴ is amino protective group,

R¹⁵ is lower alkanoyloxy(lower)alkyl,

R¹⁶ is hydroxy(lower)alkyl,

R¹⁷ is lower alkyl, and

A¹ is unsaturated 5 or 6-membered heteromonocyclic group.

The starting compounds can be prepared by the following processes or by the method of Preparation mentioned below or by a process known in the art for preparing their structurally analogous compounds.

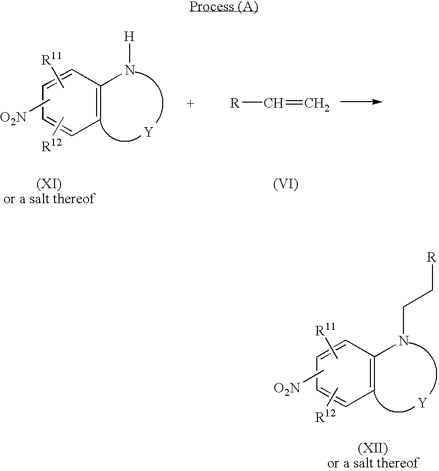

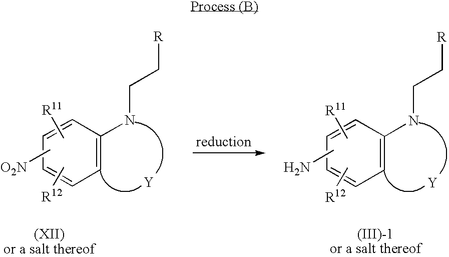

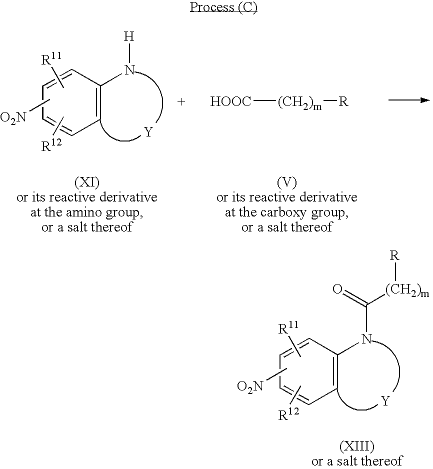

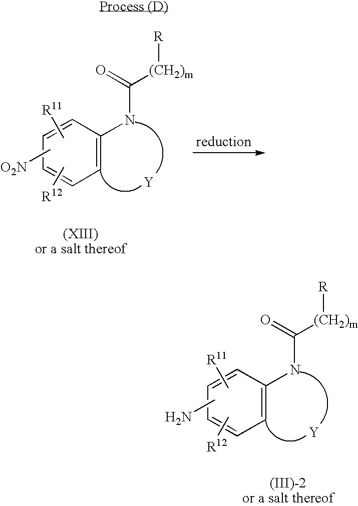

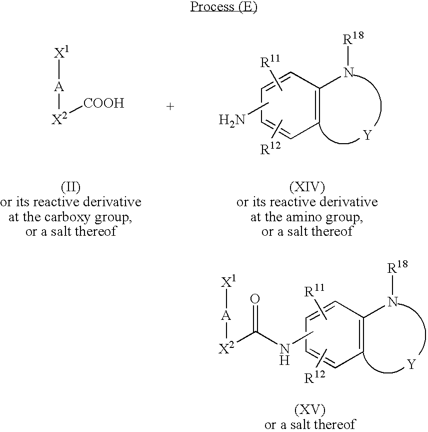

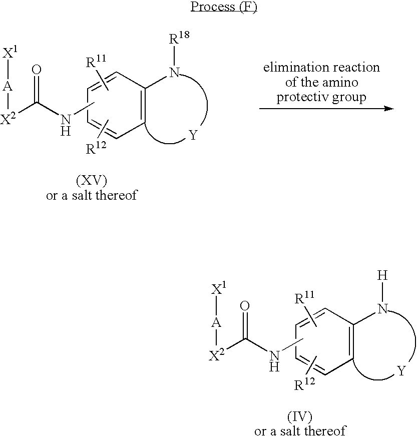

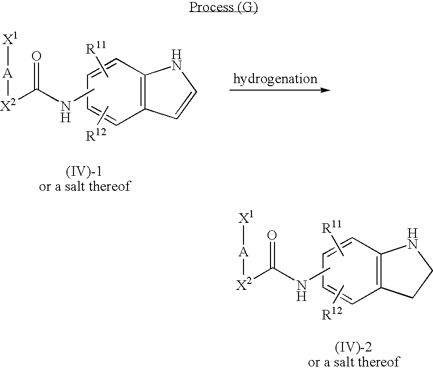

wherein R¹¹, R¹², R, A, X¹, X², Y and m are as defined above, and

R¹⁸ is amino protective group.

The processes for preparing the object compounds and starting compounds are explained in detail in the following.

Process (1)

The compound (I) or a salt thereof can be prepared by reacting the compound (II) or its reactive derivative at the carboxy group, or a salt thereof with the compound (III) or its reactive derivative at the amino group, or a salt thereof.

Suitable reactive derivative of the compound (III) includes Schiff's base type imino or its tautomeric enamine type isomer formed by the reaction of the compound (III) with a carbonyl compound such as aldehyde, ketone or the like; a silyl derivative formed by the reaction of the compound (III) with a silyl compound such as N,O-bis(trimethylsilyl)acetamide, N-trimethylsilylacetamide or the like; a derivative formed by the reaction of the compound (III) with phosphorus trichloride or phosgene.

Suitable reactive derivative of the compound (II) includes an acid halide, an acid anhydride and an activated ester. The suitable example may be an acid chloride; an acid azide; a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid, etc.), dialkylphosphorous acid, sulfurous acid, thiosulfuric acid, alkanesulfonic acid (e.g., methanesulfonic acid, ethanesulfonic acid, etc.), sulfuric acid, alkylcarbonic acid, aliphatic carboxylic acid (e.g., pivalic acid, pentanoic acid, isopentanoic acid, 2-ethylbutyric acid, trichloroacetic acid, etc.); aromatic carboxylic acid (e.g., benzoic acid, etc.); a symmetrical acid anhydride; an activated amide with imidazole, 4-substituted imidazole, dimethylpyrazole, triazole or tetrazole; an activated ester (e.g., cyanomethyl ester, methoxymethyl ester, dimethyliminomethyl [(CH₃)₂N⁺═CH—]ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenyl thioester, p-nitrophenyl thioester, p-cresyl thioester, carboxymethyl thioester, pyranyl ester, pyridinyl ester, piperidyl ester, 8-quinolyl thioester, etc.); or an ester with an N-hydroxy compound (e.g., N,N-dimethylhydroxylamine, 1-hydroxy-2-(1H)-pyridone, N-hydroxysuccinimide, N-hydroxybenzotriazole, N-hydroxyphthalimide, 1-hydroxy-6-chloro-1H-benzotriazole, etc.). These reactive derivatives can optionally be selected from them according to the kind of the compound (II) to be used.

The reaction is usually carried out in a conventional solvent such as water, acetone, dioxane, acetonitrile, chloroform, methylene chloride, ethylene dichloride, tetrahydrofuran, ethyl acetate, N,N-dimethylformamide, pyridine or any other organic solvents which do not adversely affect the reaction, or a mixture thereof.

When the compound (II) is used in free acid form or its salt form in the reaction, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diisopropylcarbodiimide; N-ethyl-N′-(3-dimethylaminopropyl)carbodiimide; N,N-carbonyl-bis-(2-methylimidazole); pentamethyleneketene-N-cyclohexylimine; diphenylketene-N-cyclohexylimine; ethoxyacetylene; 1-alkoxy-1-chloroethylene; trialkyl phosphite; isopropyl polyphosphate; phosphorus oxychloride (phosphoryl chloride); phosphorus trichloride; thionyl chloride; oxalyl chloride; triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, phosphorus oxychloride, etc.; or the like.

The reaction may also be carried out in the presence of an organic or inorganic base such as an alkali metal bicarbonate, tri(lower)alkylamine, pyridine, N-(lower)alkylmorpholine, N,N-di(lower)alkylbenzylamine, or the like.

The reaction temperature is not critical, and the reaction is usually carried out under cooling to heating.

Process (2)

The compound (I)-1 or a salt thereof can be prepared by reacting the compound (IV) or its reactive derivative at the amino group, or a salt thereof with the compound (V) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (3)

The compound (I)-2 or a salt thereof can be prepared by reacting the compound (IV) or a salt thereof with the compound (VI) in the presence of an acid.

This reaction can be carried out in a solvent such as alcohol (e.g., methanol, ethanol, isopropyl alcohol, 2-methoxyethanol, etc.) or any other organic solvents which do not adversely affect the reaction. A preferable example of an acid is acetic acid. The reaction temperature is not critical, and the reaction is usually carried out under cooling to heating.

This reaction can be carried out in a similar manner as in Example 38 mentioned below.

Process (4)

The compound (I)-3 or a salt thereof can be prepared by reacting the compound (IV) or a salt thereof with the compound (VII) in the presence of a reducing agent.

This reaction can be carried out in a solvent such as methylene chloride, ethylene dichloride or any other organic solvents which do not adversely affect the reaction. Preferable examples of a reducing agent are sodium triacetoxyborohydride and sodium cyanoborohydride. The reaction temperature is not critical, and the reaction is usually carried out under cooling to heating.

This reaction can be carried out in a similar manner as in Example 77 mentioned below.

Process (5)

The compound (I)-4 or a salt thereof can be prepared by reacting the compound (VIII) or a salt thereof with formic acid.

This reaction can be carried out in formic acid or an aqueous solution of formic acid. The reaction temperature is not critical, and the reaction is usually carried out under cooling to heating.

This reaction can be carried out in a similar manner as in Example 72 mentioned below.

Process (6)

The compound (I)-6 or a salt thereof can be prepared by subjecting the compound (I)-5 or a salt thereof to reduction using a suitable reducing agent.

Suitable reducing agents to be used in the reduction are hydrides (e.g., sodium borohydride, sodium cyanoborohydride, lithium aluminum hydride, etc.).

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to heating.

Process (7)

The compound (I)-7 or a salt thereof can be prepared by subjecting the compound (IX) or a salt thereof to ring closure reaction in the presence of iron and ammonium chloride.

This reaction can be carried out in a solvent such as alcohol (e.g., methanol, ethanol, isopropyl alcohol, etc.) or any other organic solvents which do not adversely affect the reaction. The reaction temperature is not critical, and the reaction is usually carried out under cooling to heating.

This reaction can be carried out in a similar manner as in Example 80 mentioned below.

Process (8)

The compound (I)-8 or a salt thereof can be prepared by subjecting the compound (I)-7 or a salt thereof to reduction using a suitable reducing agent.

Suitable reducing agents to be used in the reduction are hydrides (e.g., sodium borohydride, sodium cyanoborohydride, lithium aluminum hydride, etc.).

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to heating.

Process (9)

The compound (I)-9 or a salt thereof can be prepared by reacting the compound (X) or a salt thereof with cyanogen bromide.

This reaction can be carried out in a solvent such as alcohol (e.g., methanol, ethanol, isopropyl alcohol, etc.), tetrahydrofuran or any other organic solvents which do not adversely affect the reaction, or a mixture thereof. The reaction temperature is not critical, and the reaction is usually carried out under cooling to heating.

This reaction can be carried out in a similar manner as in Example 84 mentioned below.

Process (10)

The compound (I)-11 or a salt thereof can be prepared by subjecting the compound (I)-10 or a salt thereof to elimination reaction of the amino protective group.

Suitable method of this elimination reaction includes conventional one such as hydrolysis, reduction and the like.

(i) For Hydrolysis:

The hydrolysis is preferably carried out in the presence of a base or an acid including Lewis acid.

Suitable base includes an inorganic base and an organic base such as an alkali metal [e.g., sodium, potassium, etc.), an alkaline earth metal [e.g., magnesium, calcium, etc.], hydroxide or carbonate or hydrogencarbonate thereof, trialkylamine [e.g., trimethylamine, triethylamine, etc.], picoline, 1,5-diazabicyclo[4.3.0]non-5-ene, or the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], or the like is preferably carried out in the presence of a cation trapping agent [e.g., anisole, phenol, etc.]. This reaction is usually carried out without solvent.

The reaction may be carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical and the reaction is usually carried out under cooling to warming.

(ii) For Reduction:

Reduction is carried out in a conventional manner, including chemical reduction and catalytic reduction.

Suitable reducing reagent to be used in chemical reduction are hydrides (e.g., hydrogen iodide, hydrogen sulfide, lithium aluminum hydride, sodium borohydride, sodium cyanoborohydride, etc.), or a combination of a metal (e.g., tin, zinc, iron, etc.) or metallic compound (e.g., chromium chloride, chromium acetate, etc.) and an organic acid or inorganic acid (e.g., formic acid, acetic acid, propionic acid, trifluoroacetic acid, p-toluenesulfonic acid, hydrochloric acid, hydrobromic acid, etc.).

Suitable catalysts to be used in catalytic reduction are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), nickel catalysts (e.g., reduced nickel, nickel oxide, Raney nickel, etc.), cobalt catalysts (e.g., reduced cobalt, Raney cobalt, etc.), iron catalysts (e.g., reduced iron, Raney iron, Ullman iron, etc.), and the like.

The reduction is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

Additionally, in case that the above-mentioned acids to be used in chemical reduction are in a liquid state, they can also be used as a solvent.

The reaction temperature of this reduction is not critical and the reaction is usually carried out under cooling to warming.

Process (11)

The compound (I)-13 or a salt thereof can be prepared by subjecting the compound (I)-12 or a salt thereof to reduction.

Suitable method of the reduction is catalytic hydrogenation.

Suitable catalysts to be used in the catalytic hydrogenation are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, palladium hydroxide on carbon, colloidal palladium, palladium on barium sulfate, palladium on barium carbonate, etc.), and the like.

The hydrogenation is usually carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical, and the reaction is usually carried out under cooling to warming.

Process (12)

The compound (I)-15 or a salt thereof can be prepared by subjecting the compound (I)-14 or a salt thereof to hydrolysis.

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], hydroxide or carbonate or hydrogencarbonate thereof, trialkylamine (e.g., trimethylamine, triethylamine, etc.], picoline, 1,5-diazabicyclo[4.3.0]non-5-ene, or the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], or the like is preferably carried out in the presence of a cation trapping agent [e.g., anisole, phenol, etc.]. This reaction is usually carried out without solvent.

The reaction may be carried out in a conventional solvent such as water, alcohol (e.g., methanol, ethanol, isopropyl alcohol, etc.), tetrahydrofuran, dioxane, toluene, methylene chloride, ethylene dichloride, chloroform, N,N-dimethylformamide, N,N-dimethylacetamide or any other organic solvents which do not adversely affect the reaction, or a mixture thereof.

The reaction temperature is not critical and the reaction is usually carried out under cooling to warming.

Process (13)

The compound (I)-16 or a salt thereof can be prepared by reacting the compound (IV) or a salt thereof with the compound (XVI).

This reaction can be carried out in a solvent such as alcohol (e.g., methanol, ethanol, isopropyl alcohol, 2-methoxyethanol, etc.) or any other organic solvents which do not adversely affect the reaction. The reaction temperature is not critical, and the reaction is usually carried out under cooling to heating.

This reaction can be carried out in a similar manner as in Example 112 mentioned below.

Process (14)

The compound (I)-18 or a salt thereof can be prepared by subjecting the compound (I)-17 or a salt thereof to oxidation using a suitable oxidizing agent.

Suitable oxidizing agents to be used in the oxidation are potassium peroxymonosulfate (OXONE), hydrogenperoxide, m-chloroperbenzoic acid, monopermaleic acid and trifluoroperacetic acid.

The oxidation can be carried out in a solvent such as alcohol (e.g., methanol, ethanol, isopropyl alcohol, 2-methoxyethanol, etc.), acetonitrile, acetone, acetic acid, trifluoroacetic acid, dichloromethane, water, a mixture thereof, or any other organic solvents which do not adversely affect the reaction. The reaction temperature is not critical, and the reaction is usually carried out under cooling to heating.

This reaction can be carried out in a similar manner as in Example 149 mentioned below.

Process (15)

The compound (I)-20 or a salt thereof can be prepared by reacting the compound (I)-19 or a salt thereof with amino((amino(imino))methyl)amino)thioxomethane.

This reaction can be carried out in a solvent such as alcohol (e.g., methanol, ethanol, isopropyl alcohol, 2-methoxyethanol, etc.) or any other organic solvents which do not adversely affect the reaction. The reaction temperature is not critical, and the reaction is usually carried out under cooling to heating.

This reaction can be carried out in a similar manner as in Example 144 mentioned below.

Process (16)

The compound (I)-21 or a salt thereof can be prepared by reacting the compound (I)-19 or a salt thereof with the compound (XVII).

This reaction can be carried out in a solvent such as alcohol (e.g., methanol, ethanol, isopropyl alcohol, 2-methoxyethanol, etc.) or any other organic solvents which do not adversely affect the reaction. The reaction temperature is not critical, and the reaction is usually carried out under cooling to heating.

This reaction can be carried out in a similar manner as in Example 145 mentioned below.

Process (17)

The compound (I)-22 or a salt thereof can be prepared by reacting the compound (IV) or its reactive derivative at the amino group, or a salt thereof with the compound (XVIII) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (18)

The compound (I)-23 or a salt thereof can be prepared by reacting the compound (X) or a salt thereof with tert-butyl nitrite.

This reaction can be carried out in a solvent such as tetrahydrofuran or any other organic solvents which do not adversely affect the reaction, or a mixture thereof. The reaction temperature is not critical, and the reaction is usually carried out under cooling to heating.

This reaction can be carried out in a similar manner as in Example 192 mentioned below.

Process (19)

The compound (I)-24 or a salt thereof can be prepared by reacting the compound (X) or a salt thereof with 1-(1H-imidazol-1-ylcarbonyl)-1H-imidazole.

This reaction can be carried out in a solvent such as tetrahydrofuran, dichloromethane, chloroform or any other organic solvents which do not adversely affect the reaction, or a mixture thereof. The reaction temperature is not critical, and the reaction is usually carried out under cooling to heating.

This reaction can be carried out in a similar manner as in Example 193 mentioned below.

Process (A)

The compound (XII) or a salt thereof can be prepared by reacting the compound (XI) or a salt thereof with the compound (VI) in the presence of an acid.

This reaction can be carried out in a solvent such as alcohol (e.g., methanol, ethanol, isopropyl alcohol, etc.), methoxyethanol or any other organic solvents which do not adversely affect the reaction. A preferable example of an acid is acetic acid. The reaction temperature is not critical, and the reaction is usually carried out under cooling to heating.

This reaction can be carried out in a similar manner as in Preparation 1 mentioned below.

Process (B)

The compound (III)-1 or a salt thereof can be prepared by subjecting the compound (XII) or a salt thereof to reduction using a suitable reducing agent.

This reaction can be carried out in a conventional solvent such as alcohol (e.g., methanol, ethanol, isopropyl alcohol, etc.) or any other organic solvents which do not adversely affect the reaction. A preferable example of a reducing agent is hydrazine or hydrazine hydrate. Suitable example of a catalyst to be used in the reduction is iron(III) chloride.

The reaction temperature is not critical, and the reaction is usually carried out under cooling to heating.

This reaction can be carried out in a similar manner as in Preparation 2 mentioned below.

Process (C)

The compound (XIII) or a salt thereof can be prepared by reacting the compound (XI) or its reactive derivative at the amino group, or a salt thereof with the compound (V) or its reactive derivative at the carboxy group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (D)

The compound (III)-2 or a salt thereof can be prepared by subjecting the compound (XIII) or a salt thereof to reduction.

This reaction can be carried out in the same manner as in the aforementioned Process (11), and therefore the reagents to be used and the reaction conditions (e.g., solvent, reaction temperature, etc.) can be referred to those of Process (11).

Process (E)

The compound (XV) or a salt thereof can be prepared by reacting the compound (II) or its reactive derivative at the carboxy group, or a salt thereof with the compound (XIV) or its reactive derivative at the amino group, or a salt thereof.

This reaction can be carried out in the same manner as in the aforementioned Process (1), and therefore the reagents to be used and the reaction conditions (e.g., solvent, reaction temperature, etc.) can be referred to those of Process (1).

Process (F)

The compound (IV) or a salt thereof can be prepared by subjecting the compound (XV) or a salt thereof to elimination reaction of the amino protective group.

This reaction can be carried out in the same manner as in the aforementioned Process (10), and therefore the reagents to be used and the reaction conditions (e.g., solvent, reaction temperature, etc.) can be referred to those of Process (10).

Process (G)

The compound (IV)-2 or a salt thereof can be prepared by subjecting the compound (IV)-1 or a salt thereof to hydrogenation.

This reaction can be carried out in a solvent such as acetic acid in the presence of a hydride such as sodium cyanoborohydride. The reaction temperature is not critical, and the reaction is usually carried out under cooling to heating.

This reaction can be carried out in a similar manner as in Preparation 10 mentioned below.

Suitable salts of the starting compounds and their reactive derivatives in Processes (1) to (19) and (A) to (G) can be referred to the ones as exemplified for the compound (I).

The compounds obtained by the above processes can be isolated and purified by a conventional method such as pulverization, recrystallization, column chromatography, reprecipitation, or the like.

It is to be noted that the compound (I) and the other compounds may include one or more stereoisomer(s) such as optical isomer(s) and geometrical isomer(s) due to asymmetric carbon atom(s) and double bond(s), and all of such isomers and mixtures thereof are included within the scope of this invention.

The object compounds (I) and pharmaceutically acceptable salts thereof include solvates [e.g., enclosure compounds (e.g., hydrate, etc.)].

The object compounds (I) and pharmaceutically acceptable salts thereof possess a strong inhibitory activity on the secretion of Apo B.

Accordingly, the object compounds (I) and pharmaceutically acceptable salts thereof are useful as an Apo B secretion inhibitor.

The object compounds (I) and pharmaceutically acceptable salts thereof are useful as a medicament for the prophylaxis or treatment of diseases or conditions resulting from elevated circulating levels of Apo B such as hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

The present invention therefore provides a method for inhibiting or decreasing Apo B secretion in a mammal, in particular in human, which comprises administering an Apo B secretion inhibiting or decreasing amount of a compound of formula (I) or a pharmaceutically acceptable salt thereof to the mammal.

The present invention also provides a method for preventing or treating diseases or conditions resulting from elevated circulating levels of Apo B in a mammal, in particular in human, which comprises administering an effective amount of a compound of formula (I) or a pharmaceutically acceptable salt thereof to the mammal.

The object compounds (I) and pharmaceutical acceptable salts thereof are also useful in reducing intestinal fat absorption and reducing food intake for the prophylaxis or treatment of obesity. Furthermore, the object compounds (I) and pharmaceutical acceptable salts thereof possess an inhibitory activity on the lipid transfer of microsomal triglyceride transfer protein (MTP).

In order to illustrate the usefulness of the object compound (I), the pharmacological test result of the compound (I) is shown in the following.

Test Compounds:

N-{1-[2-(2-Pyridinyl)ethyl]-2,3-dihydro-1H-indol-5-yl}-4′-(trifluoromethyl)-1,1′-biphenyl-2-carboxamide (Example 1, more polar compound)

N-[1-(2-Pyridinylacetyl)-2,3-dihydro-1H-indol-5-yl]-4′-(trifluoromethyl)-1,1′-biphenyl-2-carboxamide (Example 4)

4′,5-Dimethyl-N-[1-(2-pyridinylacetyl)-2,3-dihydro-1H-indol-5-yl]-1,1′-biphenyl-2-carboxamide (Example 11)

N-{1-[(2-Amino-1,3-thiazol-4-yl)acetyl]-2,3-dihydro-1H-indol-5-yl}-4′-(trifluoromethyl)-1,1′-biphenyl-2-carboxamide (Example 58)

Test 1: Measurement of Inhibition of Apo B Secretion

HepG2 cells were seeded in Eagles medium containing 10% fetal calf serum (FCS) at a density of 30000 cells/well in 96-well plates and allowed to grow for 3 days before treatment. At this time, the medium was replaced with fresh medium containing 0.1% dimethyl sulfoxide (DMSO) and the indicated concentrations of a test compound. After 15-hour incubation, the amount of Apo B and Apo AI accumulated in the media was determined by ELISA.

The assay was carried out at room temperature. A flat bottomed micro ELISA plate (manufactured by Nunc) was coated with an anti Apo B monoclonal antibody solution (5 mg/ml in 0.05% carbonate buffer, pH 9.6) by adding the antibody solution at a volume of 100 μl per well. After 1-hour incubation on a plate mixer, the unbound materials were removed by washing the well 3 times with a washing buffer (phosphate buffered saline, pH 7.2 containing 0.1% bovine serum albumin and 0.05% Tween-20). Then 20 μl of a solution of the test compound (dissolved in the culture medium) and 100 μl of a solution of peroxidase coupled anti Apo B antibody were added. After 1-hour incubation on a plate mixer, washing was performed 3 times to remove the unbound materials. A freshly prepared substrate solution (2.5 mg/ml ortho-phenylene diamine and 0.018% H₂O₂ in 0.11M Na₂HPO₄-0.044 M sodium citrate buffer, pH 5.4) at a volume of 200 μl was then added to each well. After 20-minute incubation, the enzyme reaction was terminated by adding 50 μl of 0.5 M sulfuric acid. Absorbance of each well was determined at 490 nm using a microplate reader. Apo B concentration was calculated from a standard curve generated from purified Apo B standard that was run in parallel in the same plate. Inhibition of Apo B secretion by the test compound is calculated taking 0.1% DMSO treated cells as controls.

Measurement of Apo AI was performed similar to that of Apo B, except for diluting the sample 11-fold with a dilution buffer (phosphate buffered saline, pH 7.2 containing 0.5% bovine serum albumin and 0.05% Tween-20).

Apo B secretion inhibitors are identified as compounds that decrease Apo B secretion without affecting the secretion of Apo AI.

Test Results:

TABLE 1

| Test | |
|---|---|
| compound | Inhibition of Apo B |
| (Example | secretion at 1 × 10⁻⁸ M |
| No.) | (%) |
| 1 | 92.2 |
| 4 | 92.0 |
| 11 | 97.2 |
| 58 | 90.7 |

Test 2: Lipids Lowering Effect on ddY-Mice

Male ddY-mice were housed in temperature- and humidity-controlled rooms and fed with laboratory chow. The animals were randomized according to their body weight and deprived of food just before the experiment. A blood sample (baseline blood sample) was collected from the retro orbital venous plexus before administration of the test drug, and then the animals were orally dosed with the test drug in a vehicle (aqueous solution of 0.5% methylcellulose). Blood samples were drawn at 2 hours after drug administration for the measurement of cholesterol and triglyceride.

Plasma total-cholesterol and plasma triglyceride were determined by conventional enzyme methods using commercially available kits. The cholesterol CII-Test Wako (Wako Pure Chemical Industries, Ltd.) was used for the measurement of cholesterol, and the triglyceride E-test Wako (Wako Pure Chemical Industries, Ltd.) was used for the measurement of triglyceride.

Lipids lowering effects were shown in percent relative to the baseline level (level at 0 hr).

Test Results:

TABLE 2

| Test | | | |
|---|---|---|---|
| compound | | Cholesterol | Triglyceride |
| (Example | Dose | (% of 0 hr) | (% of 0 hr) |
| No.) | (mg/kg) | 2 hr | 2 hr |
| 1 | 10 | 90 | 13 |
| 4 | 10 | 75 | 13 |

For therapeutic administration, the object compound (I) of the present invention and pharmaceutically acceptable salts thereof are used in the form of a conventional pharmaceutical preparation in admixture with a conventional pharmaceutically acceptable carrier such as an organic or inorganic solid or liquid excipient which is suitable for oral, parenteral or external administration. The pharmaceutical preparation may be compounded in a solid form such as granule, capsule, tablet, dragee, suppository or ointment, or in a liquid form such as solution, suspension or emulsion for injection, intravenous drip, ingestion, eye drop, etc. If needed, there may be included in the above preparation auxiliary substance such as stabilizing agent, wetting or emulsifying agent, buffer or any other commonly used additives.

The effective ingredient may usually be administered in a unit dose of 0.01 mg/kg to 100 mg/kg, preferably 0.1 mg/kg to 10 mg/kg, 1 to 4 times a day. However, the above dosage may be increased or decreased according to age, body weight and conditions of the patient or administering method.

The following Preparations and Examples are given for the purpose of illustrating the present invention in detail.

Preparation 1

To a solution of 5-nitroindoline (4.93 g) in 2-methoxyethanol (15 ml) were added 2-vinylpyridine (4.1 g) and acetic acid (1.8 g) at ambient temperature and the mixture was refluxed for 16 hours. The mixture was cooled to ambient temperature and evaporated in vacuo. The residue was purified by column chromatography on silica gel eluting with ethyl acetate to give 5-nitro-1-[2-(2-pyridinyl)ethyl]indoline (5.58 g) as pale brown crystals.

¹H-NMR (DMSO-d₆):δ 3.0-3.15 (4H, m), 3.65-3.8 (4H, m), 6.38 (1H, d, J=8.9 Hz), 7.21 (1H, dd, J=6.2 Hz, 4.8 Hz), 7.34 (1H, ddd, J=7.6 Hz, 7.4 Hz, 1.8 Hz), 7.78 (1H, d, J=2.3 Hz), 7.90 (1H, dd, J=8.7 Hz, 2.3 Hz), 8.50 (1H, d, J=4.7 Hz)

APCI-MS (m/z): 270 (M+H)⁺

Preparation 2

To a solution of 5-nitro-1-[2-(2-pyridinyl)ethyl]indoline (5.52 g) in ethanol (40 ml) were added iron(III) chloride (anhydrous) (166 mg) and active-charcoal (5.5 g) and the mixture was heated to 80° C. To the mixture was added dropwise hydrazine hydrate (4.1 g) and stirred at 80° C. for 3 hours. The active-charcoal was filtered off by celite and washed with ethanol. The filtrate was evaporated in vacuo and the residue was purified by column chromatography on silica gel eluting with ethyl acetate:methanol (10:1 v/v) to give 1-[2-(2-pyridinyl)ethyl]-5-indolinamine (3.45 g) as a pale yellow solid.

¹H-NMR (DMSO-d₆):δ 2.27 (2H, t, J=8.2 Hz), 2.94 (2H, t, J=6.8 Hz), 3.16 (2H, t, J=8.2 Hz), 3.25 (2H, t, J=6.8 Hz), 4.32 (2H, br s), 6.29 (2H, s), 6.40 (1H, s), 7.2-7.4 (3H, m), 7.19 (1H, dd, J=7.6 Hz, 5.8 Hz), 7.34 (1H, d, J=6.4 Hz), 7.69 (1H, ddd, J=7.6 Hz, 6.4 Hz, 1.8 Hz), 8.50 (1H, d, J=4.0 Hz)

APCI-MS (m/z): 240 (M+H)⁺

### EXAMPLE 1

To a solution of 1-[2-(2-pyridinyl)ethyl]-5-indolinamine (1.13 g) in dichloromethane (20 ml) was added triethylamine (573 mg), followed by dropwise addition of a solution of 4′-(trifluoromethyl)-1,1′-biphenyl-2-carbonyl chloride (1.48 g) in dichloromethane (10 ml) at ambient temperature. The mixture was stirred for 16 hours and poured into water. The separated organic layer was washed with brine, dried over magnesium sulfate and dried in vacuo. The residue was purified by column chromatography on silica gel eluting with dichloromethane:ethyl acetate (2:1 v/v) to give two compounds. The more polar compound was the target and crystallized from ethyl acetate to give N-{1-[2-(2-pyridinyl)ethyl]-2,3-dihydro-1H-indol-5-yl}-4′-(trifluoromethyl)-1,1′-biphenyl-2-carboxamide (697 mg) as white crystals. The less polar compound has the indole nucleus and was crystallized from ethyl acetate to give N{1-[2-(2-pyridinyl)ethyl]-1H-indol-5-yl}-4′-(trifluoromethyl)-1,1′-biphenyl-2-carboxamide (378 mg) as white crystals.

N-{1-[2-(2-Pyridinyl)ethyl]-1H-indol-5-yl}-4′-(trifluoromethyl)-1,1′-biphenyl-2-carboxamide (less polar compound):

¹H-NMR (DMSO-d₆):δ 3.19 (2H, t, J=7.2 Hz), 4.53 (2H, t, J=7.2 Hz), 6.28 (1H, d, J=3.0 Hz), 7.14 (1H, d, J=3.0 Hz), 7.24 (1H, s), 7.33 (1H, d, J=8.8 Hz), 7.55-7.8 (8H, m), 8.52 (1H, d, J=4.7 Hz), 10.13 (1H, s)

APCI-MS (m/z): 486 (M+H)⁺

N-{1-[2-(2-Pyridinyl)ethyl]-2,3-dihydro-1H-indol-5-yl}-4′-(trifluoromethyl)-1,1′-biphenyl-2-carboxamide (more polar compound):

¹H-NMR (DMSO-d₆):δ 2.85 (2H, t, J=8.0 Hz), 2.96 (2H, t, J=6.8 Hz), 3.31 (2H, t, J=8.0 Hz), 3.38 (2H, t, J=6.8 Hz), 6.41 (1H, d, J=8.4 Hz), 7.08 (1H, d, J=8.4 Hz), 7.15-7.2.5 (2H, m), 7.34 (1H, d, J=7.8 Hz), 7.45-7.8 (9H, m), 8.50 (1H, d, J=4.6 Hz), 9.95 (1H, s)

APCI-MS (m/z): 488 (M+H)⁺

### EXAMPLE 2

4′-Methoxy-N-{1-[2-(2-pyridinyl)ethyl]-2,3-dihydro-1H-indol-5-yl}-1,1′-biphenyl-2-carboxamide

The title compound was obtained in the same manner as in Example 1 as a light yellow solid.

¹H-NMR (DMSO-d₆):δ 2.82 (2H, t, J=8.4 Hz), 2.96 (2H, t, J=6.8 Hz), 3.31 (2H, t, J=8.4 Hz), 3.38 (2H, t, J=6.4 Hz), 3.75 (3H, s), 6.41 (1H, d, J=8.4 Hz), 9.94 (2H, d, J=8.1 Hz), 7.11 (1H, d, J=8.4 Hz), 7.2-7.3 (2H, m), 7.35-7.55 (8H, m), 7.70 (1H, ddd, J=7.8 Hz, 7.6 Hz, 1.8 Hz), 8.51 (1H, d, J=4.5 Hz), 9.82 (1H, s)

APCI-MS (m/z): 450 (M+H)⁺

Preparation 3

6-Nitro-1-[2-(2-pyridinyl)ethyl]indoline

The title compound was obtained in the same manner as in Preparation 1 as a light yellow solid.

¹H-NMR (DMSO-d₆):δ 2.95-3.1 (4H, m), 3.45-3.6 (4H, m), 7.1-7.8 (6H, m), 8.5-8.6 (1H, m)

APCI-MS (m/z): 270 (M+H)⁺

Preparation 4

1-[2-(2-Pyridinyl)ethyl]-6-indolinamine

The title compound was obtained in the same manner as in Preparation 2 as a light yellow solid.

¹H-NMR (DMSO-d₆):δ 2.68 (2H, t, J=8.1 Hz), 2.95 (2H, t, J=6.8 Hz), 3.2-3.4 (4H, m), 4.68 (2H, br s), 5.80 (1H, d, J=7.7 Hz), 5.83 (1H, s), 6.65 (1H, d, J=7.7 Hz), 7.15-7.3 (1H, m), 7.33 (1H, d, J=7.8 Hz), 7.71 (1H, ddd, J=7.8 Hz, 7.6 Hz, 1.9 Hz), 8.51 (1H, d, J=4.9 Hz)

APCI-MS (m/z): 240 (M+H)⁺

### EXAMPLE 3

N-{1-[2-(2-Pyridinyl)ethyl]-2,3-dihydro-1H-indol-6-yl}-4′-(trifluoromethyl)-1,1′-biphenyl-2-carboxamide

The title compound was obtained in the same manner as in Example 1 as a light yellow solid.

¹H-NMR (DMSO-d₆):δ 2.80 (2H, t, J=8.2 Hz), 2.96 (2H, t, J=6.8 Hz), 3.3-3.5 (4H, m), 6.85-7.0 (3H, m), 7.2-7.4 (3H, m), 7.5-7.8 (8H, m), 8.51 (1H, d, J=4.8 Hz), 10.09 (1H, s)

ESI-MS(m/z): 488 (M+H)⁺

Preparation 5

To a suspension of 5-nitroindoline (3.28 g), 2-pyridylacetic acid hydrochloride (3.82 g), 1-[3-(dimethylamino)propyl]-3-ethylcarbodiimide hydrochloride (4.22 g) and 1-hydroxybenzotriazole hydrate (3.37 g) in dichloromethane (100 ml) was added dropwise triethylamine (4.45 g) at ambient temperature and the resultant solution was stirred at ambient temperature for 18 hours. The mixture was poured into water and the separated organic layer was washed with water and brine, dried over magnesium sulfate and evaporated in vacuo. The residue was purified by column chromatography on silica gel eluting with ethyl acetate to give 5-nitro-1-(2-pyridinylacetyl)indoline (3.58 g) as a yellow solid.

¹H-NMR (DMSO-d₆):δ 3.26 (2H, t, J=8.5 Hz), 4.10 (2H, s), 4.33 (2H, t, J=8.5 Hz), 7.25-7.35 (1H, m), 7.38 (1H, d, J=7.8 Hz), 7.75-7.9 (1H, m), 8.1-8.2 (3H, m), 8.50-8.55 (1H, m)

APCI-MS (m/z): 284 (M+H)⁺

Preparation 6

To a solution of 5-nitro-1-(2-pyridinylacetyl)indoline (3.54 g) in methanol (50 ml) and tetrahydrofuran (THF) (50 ml) was added 10% palladium on carbon (50% wet, 3.5 g) and the mixture was hydrogenated under hydrogen at atmospheric pressure for 5 hours. After removing the palladium on carbon by filtration, the filtrate was evaporated in vacuo. The residue was purified by column chromatography on silica gel eluting with ethyl acetate:methanol (10:1 v/v) to give 1-(2-pyridinylacetyl)-5-indolinamine (2.16 g) as pale brown crystals.

¹H-NMR (DMSO-d₆):δ 3.01 (2H, t, J=8.4 Hz), 3.92 (2H, s), 4.11 (2H, t, J=8.4 Hz), 4.84 (2H, br s), 6.32 (1H, d, J=8.4 Hz), 6.45 (1H, s), 7.1-7.2 (1H, m), 7.33 (1H, d, J=7.8 Hz), 7.7-7.85 (2H, m), 8.48 (1H, d, J=4.0 Hz)

APCI-MS (m/z): 254 (M+H)⁺

### EXAMPLE 4

N-[1-(2-Pyridinylacetyl)-2,3-dihydro-1H-indol-5-yl]-4′-(trifluoromethyl)-1,1′-biphenyl-2-carboxamide

The title compound was obtained in the same manner as in Example 1 as white crystals.

¹H-NMR (DMSO-d₆):δ 3.11 (2H, t, J=8.3 Hz), 3.99 (2H, s), 4.19 (2H, t, J=8.3 Hz), 7.15-7.4 (3H, m), 7.45-7.8 (10H, m), 7.91 (1H, d, J=8.7 Hz), 8.49 (1H, d, J=4.8 Hz), 10.26 (1H, s)

APCI-MS (m/z): 502 (M+H)⁺

Preparation 7

A mixture of 6-nitroindoline (3.28 g) and methyl 2-pyridinylacetate (3.63 g) was stirred at 150° C. for 18 hours. The mixture was cooled to ambient temperature and purified by column chromatography on silica gel eluting with ethyl acetate to give 6-nitro-1-(2-pyridinylacetyl)indoline (3.13 g) as a brown solid.

¹H-NMR (DMSO-d₆):δ 3.29 (2H, t, J=8.7 Hz), 4.08 (2H, s), 4.33 (2H, t, J=8.7 Hz), 7.31 (1H, dd, J=10.5 Hz, 5.1 Hz), 7.38 (1H, d, J=7.8 Hz), 7.50 (1H, d, J=8.2 Hz), 7.78 (1H, ddd, J=8.2 Hz, 7.8 Hz, 1.7 Hz), 7.92 (1H, dd, J=8.2 Hz, 2.3 Hz), 8.51 (1H, d, J=4.9H), 8.80 (1H, d, J=2.0 Hz)

APCI-MS (m/z): 284 (M+H)⁺

Preparation 8

1-(2-Pyridinylacetyl)-6-indolinamine

The title compound was obtained in the same manner as in Preparation 6 as white crystals.

¹H-NMR (DMSO-d₆):δ 2.94 (2H, t, J=8.4 Hz), 3.97 (2H, s), 4.12 (2H, t, J=8.4 Hz), 4.93 (2H, s), 6.21 (1H, dd, J=7.9 Hz, 2.0 Hz), 6.83 (1H, d, J=7.9 Hz), 7.25 (1H, dd, J=7.8 Hz, 4.9 Hz), 7.31 (1H, dd, J=7.8 Hz), 7.41 (1H, d, J=2.0 Hz), 7.75 (1H, ddd, J=7.8 Hz, 7.7 Hz, 1.8 Hz), 8.49 (1H, d, J=4.9 Hz)

APCI-MS (m/z): 254 (M+H)⁺

### EXAMPLE 5

N-[1-(2-Pyridinylacetyl)-2,3-dihydro-1H-indol-6-yl]-41-(trifluoromethyl)-1,1′-biphenyl-2-carboxamide

The title compound was obtained in the same manner as in Example 1 as white crystals.

¹H-NMR (DMSO-d₆):δ 3.07 (2H, t, J=8.3 Hz), 4.05 (2H, s), 4.19 (2H, t, J=8.3 Hz), 7.09 (2H, d, J=8.1 Hz), 7.18 (2H, d, J=8.1 Hz), 7.25 (1H, dd, J=7.7 Hz, 6.6 Hz), 7.35 (1H, d, J=7.7 Hz), 7.45-7.8 (9H, m), 8.31 (1H, s), 8.48 (1H, d, J=4.8 Hz), 10.29 (1H, s)

APCI-MS (m/z): 502 (M+H)⁺

### EXAMPLE 6

To a suspension of 1-(2-pyridinylacetyl)-5-indolinamine (506 mg), 2-[3-(trifluoromethyl)anilino]-benzoic acid (562 mg), 1-[3-(dimethylamino)propyl]-3-ethylcarbodiimide hydrochloride (421 mg) and 1-hydroxybenzotriazole hydrate (337 mg) in dichloromethane (40 ml) was added dropwise triethylamine (445 mg) at ambient temperature and the resultant solution was stirred at ambient temperature for 18 hours. The mixture was poured into water and the separated organic layer was washed with water and brine, dried over magnesium sulfate and evaporated in vacuo. The residue was purified by column chromatography on silica gel eluting with ethyl acetate to give N-[1-(2-pyridinylacetyl)-2,3-dihydro-1H-indol-5-yl]-2-{[3-(trifluoromethyl)phenyl]amino}benzamide (716 mg) as pale yellow crystals.

¹H-NMR (DMSO-d₆):δ 3.15 (2H, t, J=8.3 Hz), 4.01 (2H, s), 4.22 (2H, t, J=8.3 Hz), 7.0-7.5 (10H, m), 7.64 (2H, m), 7.97 (1H, d, J=8.7 Hz), 8.50 (1H, d, J=4.9 Hz), 9.09 (1H, s), 10.31 (1H, s)

APCI-MS (m/z): 517 (M+H)⁺

### EXAMPLE 7

4′-Ethyl-N-[1-(2-pyridinylacetyl)-2,3-dihydro-1H-indol-5-yl]-1,1′-biphenyl-2-carboxamide

The title compound was obtained in the same manner as in Example 6 as white crystals.

¹H-NMR (DMSO-d₆):δ 1.17 (3H, t, J=7.6 Hz), 2.59 (2H, t, J=7.6 Hz), 3.11 (2H, d, J=8.4 Hz), 3.99 (2H, s), 4.19 (2H, t, J=8.4 Hz), 7.2-7.6 (1H, m), 7.7-7.8 (1H, m), 7.91 (1H, d, J=8.6 Hz), 8.49 (1H, d, J=4.1 Hz), 10.15 (1H, s)

APCI-MS (m/z): 462 (M+H)⁺

Preparation 9

N-(1H-Indol-5-yl)-4′-(trifluoromethyl)-1,1′-biphenyl-2-carboxamide

The title compound was obtained from 5-indolamine and 4′-(trifluoromethyl)-1,1′-biphenyl-2-carbonyl chloride in the same manner as in Example 1 as brown crystals.

¹H-NMR (DMSO-d₆):δ 6.35-6.4 (1H, m), 7.1-7.2 (1H, m), 7.25-7.35 (2H, m), 7.3-7.8 (9H, m), 10.11 (1H, s), 10.99 (1H, br s)

APCI-MS (m/z): 381 (M+H)⁺

Preparation 10

To a solution of N-(1H-indol-5-yl)-4′-(trifluoromethyl)-1,1′-biphenyl-2-carboxamide (7.61 g) in acetic acid (80 ml) was added portionwise sodium cyanoborohydride (1.26 g) at 10° C. The mixture was gradually warmed to ambient temperature and stirred at ambient temperature for 18 hours. After removing the acetic acid by evaporation in vacuo, the residue was poured into a mixture of ethyl acetate and water. The separated organic layer was washed with brine, dried over magnesium sulfate and evaporated in vacuo. The residue was purified by column chromatography on silica gel eluting with n-hexane:ethyl acetate (1:3 v/v) to give N-(2,3-dihydro-1H-indol-5-yl)-4′-(trifluoromethyl)-1,1′-biphenyl-2-carboxamide (5.38 g) as a pale brown solid.

¹H-NMR (DMSO-d₆):δ 2.84 (2H, t, J=8.4 Hz), 3.37 (2H, t, J=8.4 Hz), 5.33 (1H, br s), 6.38 (1H, d, J=8.3 Hz), 6.98 (1H, dd, J=8.3 Hz, 2.2 Hz), 7.19 (1H, d, J=2.2 Hz), 9.90 (1H, s)

APCI-MS (m/z): 383 (M+H)⁺

Preparation 11

To a solution of 6-methyl-2-pyridinamine (25.0 g) and 2,5-hexanedione (29.0 g) in toluene (150 ml) was added p-toluenesulfonic acid hydrate (4.4 g) at ambient temperature and the mixture was refluxed for 18 hours. The mixture was evaporated in vacuo and the residue was purified by column chromatography on silica gel eluting with n-hexane:ethyl acetate (4:1 v/v) to give 2-(2,5-dimethyl-1H-pyrrol-1-yl)-6-methylpyridine (35.8 g) as a yellow oil.

¹H-NMR (DMSO-d₆):δ 2.04 (6H, s), 2.51 (3H, s), 5.78 (2H, s), 7.18 (1H, d, J=7.8 Hz), 7.29 (1H, d, J=7.6 Hz), 7.86 (1H, dd, J=7.8 Hz, 7.6 Hz)

APCI-MS (m/z): 187 (M+H)⁺

Preparation 12

To a solution of diisopropylamine (11.1 g) in THF (80 ml) was added dropwise n-butyllithium (1.59M solution in hexane, 69.1 ml) at −60° C. under a nitrogen atmosphere and the mixture was stirred at −60° C. for 30 minutes. To the mixture was added dropwise a solution of 2-(2,5-dimethyl-1H-pyrrol-1-yl)-6-methylpyridine (18.63 g) in THF (200 ml) at −60° C. over 50 minutes and the reaction mixture was stirred for 30 minutes. Powdered Dry Ice was added carefully and the mixture was gradually warmed to ambient temperature. The mixture was quenched by addition of a saturated aqueous solution of ammonium chloride and poured into a mixture of ethyl acetate and water. The mixture was adjusted to pH 2 with 6N HCl. The separated organic layer was washed with water and brine, dried over magnesium sulfate and evaporated in vacuo. The residue was purified by column chromatography on silica gel to give [6-(2,5-dimethyl-1H-pyrrol-1-yl)-2-pyridinyl]acetic acid (9.69 g) as pale brown crystals.

¹H-NMR (DMSO-d₆):δ 2.04 (6H, s), 3.79 (2H, s), 5.79 (2H, s), 7.28 (2H, d, J=7.9 Hz), 7.38 (2H, d, J=7.9 Hz), 7.93 (1H, dd, J=7.9 Hz, 7.9 Hz), 12.30 (1H, br)

ESI-MS(m/z): 253 (M+Na)⁺, 231 (M+H)+

### EXAMPLE 8

To a solution of [6-(2,5-dimethyl-1H-pyrrol-1-yl)-2-pyridinyl]acetic acid (1.15 g), N-(2,3-dihydro-1H-indol-5-yl)-4′-(trifluoromethyl)-1,1′-biphenyl-2-carboxamide (2.08 g) and PyBOP (benzotriazol-1-yloxytripyrrolidinophosphonium hexafluorophosphate) (3.12 g) in N,N-dimethylformamide (30 ml) was added dropwise diisopropylethylamine (1.94 g) at 5° C. The mixture was gradually warmed to ambient temperature and stirred for 18 hours. The mixture was poured into a mixture of ethyl acetate and water and the separated organic layer was washed with water and brine, dried over magnesium sulfate and evaporated in vacuo. The residue was purified by column chromatography on silica gel eluting with ethyl acetate to give N-(1-{[6-(2,5-dimethyl-1H-pyrrol-1-yl)-2-pyridinyl]acetyl}-2,3-dihydro-1H-indol-5-yl)-4′-(trifluoromethyl)-1,1′-biphenyl-2-carboxamide (1.82 g) as a pale brown solid.

¹H-NMR (DMSO-d₆):δ 2.02 (6H, s), 3.09 (2H, t, J=8.5 Hz), 4.05 (2H, s), 4.16 (2H, t, J=8.5 Hz), 5.77 (2H, s), 7.2-8.0 (14H, m), 0.26 (1H, s)

APCI-MS (m/z): 595 (M+H)⁺

### EXAMPLE 9

To a suspension of N-(1-{[6-(2,5-dimethyl-1H-pyrrol-1-yl)-2-pyridinyl]acetyl}-2,3-dihydro-1H-indol-5-yl)-4′-(trifluoromethyl)-1,1′-biphenyl-2-carboxamide (1.79 g) in a mixture of ethanol (40 ml) and water (10 ml) were added hydroxylamine hydrochloride (2.09 g) and triethylamine (609 mg) at ambient temperature. The mixture was refluxed for 20 hours and evaporated to dryness. The residue was extracted from ethyl acetate and the organic layer was washed with brine, dried over magnesium sulfate and evaporated in vacuo. The residue was purified by column chromatography on silica gel eluting with ethyl acetate:methanol (10:1 v/v) to give N-{1-[(6-amino-2-pyridinyl)acetyl]-2,3-dihydro-1H-indol-5-yl}-4′-(trifluoromethyl)-1,1′-biphenyl-2-carboxamide (945 mg) as white crystals.

¹H-NMR (DMSO-d₆):δ 3.09 (2H, t, J=8.5 Hz), 3.69 (2H, s), 4.17 (2H, t, J=8.5 Hz), 5.85 (2H, br s), 6.30 (1H, d, J=7.9 Hz), 6.42 (1H, d, J=6.7 Hz), 7.19 (1H, d, J=8.1 Hz), 7.45-7.65 (8H, m), 7.75 (2H, d, J=8.3 Hz), 7.92 (2H, d, J=8.3 Hz), 10.25 (1H, s)

APCI-MS (m/z): 517 (M+H)⁺

Preparation 13

1-[3-(Dimethylamino)propyl]-3-ethylcarbodiimide (23.4 g) was added to a solution of indoline (15.4 ml), 2-pyridylacetic acid hydrochloride (25.0 g), 1-hydroxybenzotriazole (23.1 g) and 4-dimethylaminopyridine (0.34 g) in dichloromethane (231 ml) under ice-cooling and the mixture was stirred at ambient temperature for 20 hours. The reaction mixture was poured into water and the mixture was adjusted to pH 9 with 20% aqueous potassium carbonate. The separated organic layer was washed with water, dried over magnesium sulfate and evaporated in vacuo. The residue was triturated with diisopropyl ether to give 1-(2-pyridinylacetyl)indoline (25.14 g).

¹H-NMR (DMSO-d₆):δ 3.15 (2H, t, J=8.5 Hz), 4.01 (2H, s), 4.20 (2H, t, J=8.5 Hz), 7.02 (1H, t, J=7.4 Hz), 7.14 (1H, t, J=7.6 Hz), 7.26-7.31 (2H, m), 7.36 (1H, d, J=7.6 Hz), 7.76 (1H, t, J=7.6 Hz), 8.04 (1H, d, J=7.8 Hz), 8.49 (1H, d, J=4.0 Hz)

Preparation 14

Fuming nitric acid, (25.0 ml, d=1.50) was dropwise added to a mixture of 1-(2-pyridinylacetyl)indoline (25.0 g) in acetic acid (250 ml) at 15-20° C. and the mixture was stirred at 15-20° C. for 3 hours. The reaction mixture was poured into ice-cold water (750 ml) and the mixture was adjusted to pH 8 with 20% aqueous potassium carbonate. The mixture was extracted with a mixture of ethyl acetate and tetrahydrofuran. The extract was washed with water, dried over magnesium sulfate and evaporated in vacuo. The residue was recrystallized from ethanol to give 5-nitro-1-(2-pyridinylacetyl)indoline (18.5 g).

¹H-NMR (DMSO-d₆):δ 3.26 (2H, t, J=8.5 Hz), 4.10 (2H, s), 4.33 (2H, t, J=8.5 Hz), 7.25-7.33 (1H, m), 7.38 (1H, d, J=7.8 Hz), 7.78 (1H, dt, J=1.9 Hz, 7.8 Hz), 8.10-8.20 (3H, m), 8.48-8.53 (1H, m)

Preparation 15

To a mixture of methyl 4-methyl-2-(trifluoromethanesulfonyloxy)benzoate (42.0 g), lithium chloride (17.9 g) and tetrakis(triphenylphosphine)palladium(0) (8.1 g) in toluene (630 ml) was added a solution of sodium carbonate (38.8 g) in water (170 ml) under stirring, followed by addition of 4-(trifluoromethyl)phenylboronic acid (29.4 g). The mixture was stirred at 100° C. for 6 hours. The reaction mixture was poured into a mixture of ethyl acetate and water. The separated organic layer was washed with water and evaporated in vacuo. To the residue was added a mixture of sodium hydroxide (14.0 g) in water (140 ml) and ethanol (207 ml), and the mixture was stirred under reflux for 4 hours. The solvent was removed by concentration in vacuo. To the residue was added a mixture of ethyl acetate and water, and the mixture was adjusted to pH 2 with 6N hydrochloric acid. The separated organic layer was washed with water, dried over magnesium sulfate and evaporated in vacuo. The residue was purified by column chromatography on silica gel eluting with ethyl acetate:n-hexane (2:3 v/v). The eluted fractions containing the desired product were collected and evaporated in vacuo to give 5-methyl-4′-(trifluoromethyl)-1,1′-biphenyl-2-carboxylic acid (25.5 g).

¹H-NMR (DMSO-d₆):δ 2.39 (3H, s), 7.21 (1H, d, J=0.9 Hz), 7.33 (1H, dd, J=0.9 Hz, 7.9 Hz), 7.52 (2H, d, J=8.0 Hz), 7.75 (2H, d, J=8.0 Hz), 7.77 (1H, d, J=7.9 Hz), 12.70 (1H, s)

### EXAMPLE 10

5-Methyl-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 2.42 (3H, s), 3.10 (2H, t, J=8.2 Hz), 3.99 (2H, s), 4.18 (2H, t, J=8.2 Hz), 7.17-7.38 (5H, m), 7.47-7.63 (4H, m), 7.71-7.95 (3H, m), 7.92 (1H, d, J=8.6 Hz), 8.49 (1H, d, J=4.2 Hz), 10.19 (1H, s)

APCI-MS (m/z): 516 (M+H)⁺

Preparation 16

4′,5-Dimethyl-1,1′-biphenyl-2-carboxylic acid

The title compound was obtained in the same manner as in Preparation 15.

¹H-NMR (DMSO-d₆):δ 2.34 (3H, s), 2.37 (3H, s), 7.15-7.30 (6H, m), 7.62 (1H, d, J=7.8 Hz), 12.54 (1H, s)

### EXAMPLE 11

4′,5-Dimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.28 (3H, s), 2.39 (3H, s), 3.11 (2H, t, J=8.5 Hz), 3.99 (2H, s), 4.19 (2H, t, J=8.5 Hz), 7.16 (2H, d, J=8.1 Hz), 7.22-7.44 (8H, m), 7.50 (1H, s), 7.76 (1H, dt, J=1.8 Hz, 7.7 Hz), 7.90 (1H, d, J=8.7 Hz), 8.49 (1H, d, J=4.0 Hz), 10.06 (1H, s)

APCI-MS (m/z): 462 (M+H)⁺

### EXAMPLE 12

4′,5-Dimethyl-N-[1-(2-pyridinylacetyl)-2,3-dihydro-1H-indol-5-yl]-1,1′-biphenyl-2-carboxamide (1.0 g) was dissolved in a mixture of methanol (7 ml) and tetrahydrofuran (7 ml) and to the solution was added 4N HCl in dioxane (0.7 ml) under stirring. To the mixture was added diisopropyl ether (12 ml). The precipitated crystals were collected by filtration and recrystallized from a mixture of methanol, tetrahydrofuran and diisopropyl ether to give 41,5-dimethyl-N-[1-(2-pyridinylacetyl)-2,3-dihydro-1H-indol-5-yl]-1,1′-biphenyl-2-carboxamide hydrochloride (0.47 g).

¹H-NMR (DMSO-d₆):δ 2.28 (3H, s), 2.39 (3H, s), 3.18 (2H, t, J=8.2 Hz), 4.24 (2H, t, J=8.2 Hz), 4.46 (2H, s), 7.16 (2H, d, J=8.0 Hz), 7.23-7.37 (5H, m), 7.42 (1H, d, J=8.1 Hz), 7.55 (1H, s), 7.81-7.99 (3H, m), 8.50 (1H, dt, J=1.1 Hz, 7.8 Hz), 8.88 (1H, d, J=5.6 Hz), 10.14 (1H, s)

Anal. calcd for C₃₀H₂₇N₃O₂.HCl—H₂O: C; 69.83%, H; 5.86%, N; 8.14%, found: C; 69.90%, H; 6.02%, N; 7.95%

### EXAMPLE 13

4′,5-Dimethyl-N-[1-(2-pyridinylacetyl)-2,3-dihydro-1H-indol-5-yl]-1,1′-biphenyl-2-carboxamide (1.0 g) was dissolved in a mixture of methanol (7 ml) and tetrahydrofuran (7 ml) and to the solution was added methanesulfonic acid (0.17 ml) under stirring. To the mixture was added diisopropyl ether (10 ml). The precipitated crystals were collected by filtration and recrystallized from a mixture of methanol and diisopropyl ether to give 4′,5-dimethyl-N-[1-(2-pyridinylacetyl)-2,3-dihydro-1H-indol-5-yl]-1,1′-biphenyl-2-carboxamide methanesulfonate (0.75 g).

¹H-NMR (DMSO-d₆):δ 2.28 (3H, s), 2.34 (3H, s), 2.40 (3H, s), 3.20 (2H, t, J=8.3 Hz), 4.23 (2H, t, J=8.3 Hz), 4.39 (2H, s), 7.16 (2H, d, J=8.0 Hz), 7.23-7.36 (5H, m), 7.42 (1H, d, J=8.2 Hz), 7.54 (1H, s), 7.86 (1H, d, J=8.7 Hz), 7.90-8.01 (2H, m), 8.52 (1H, dt, J=1.4 Hz, 7.8 Hz), 8.92 (1H, d, J=5.2 Hz), 10.11 (1H, s)

Anal. calcd for C₃₀H₂₇N₃O₂.CH₄O₃S.⅓H₂O: C; 66.06%, H; 5.66%, N; 7.45%, found: C; 66.15%, H; 5.53%, N; 7.61%

Preparation 17

4′-Methoxy-5-methyl-1,1′-biphenyl-2-carboxylic acid

The title compound was obtained in the same manner as in Preparation 15.

¹H-NMR (DMSO-d₆):δ 2.37 (3H, s), 3.78 (3H, s), 6.95 (2H, d, J=8.7 Hz), 7.16 (1H, s), 7.18-7.27 (1H, m), 7.24 (2H, d, J=8.7 Hz), 7.61 (1H, d, J=7.8 Hz), 12.54 (1H, s)

### EXAMPLE 14

4′-Methoxy-5-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.39 (3H, s), 3.11 (2H, t, J=8.3 Hz), 3.74 (3H, s), 3.98 (2H, s), 4.18 (2H, t, J=8.3 Hz), 6.92 (2H, d, J=8.7 Hz), 7.20-7.43 (8H, m), 7.50 (1H, s), 7.76 (1H, dt, J=1.8 Hz, 7.6 Hz), 7.90 (1H, d, J=8.7 Hz), 8.47-8.51 (1H, m), 10.03 (1H, s)

APCI-MS (m/z): 478 (M+H)⁺

Prepration 19

4′-Fluoro-5-methyl-1,1′-biphenyl-2-carboxylic acid

The title compound was obtained in the same manner as in Preparation 15.

¹H-NMR (DMSO-d₆):δ 2.38 (3H, s), 7.15-7.37 (5H, m), 7.55-7.72 (2H, m), 12.62 (1H, s)

### EXAMPLE 15

4′-Fluoro-5-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.40 (3H, s), 3.11 (2H, t, J=8.3 Hz), 3.99 (2H, s), 4;18 (2H, t, J=8.3 Hz), 7.11-7.50 (11H, m), 7.75 (1H, dt, J=1.9 Hz, 7.7 Hz), 7.90 (1H, d, J=8.7 Hz), 8.48-8.51 (1H, m), 10.07 (1H, s)

APCI-MS (m/z): 466 (M+H)⁺

Prepration 19

4′-Chloro-5-methyl-1,1′-biphenyl-2-carboxylic acid

The title compound was obtained in the same manner as in Preparation 15.

¹H-NMR (DMSO-d₆):δ 2.38 (3H, s), 7.18 (1H, s) 7.25-7.35 (3H, m), 7.39-7.48 (2H, m), 7.70 (1H, d, J=7.9 Hz), 12.66 (1H, s)

### EXAMPLE 16

4′-Chloro-5-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.40 (3H, s), 3.11 (2H, t, J=8.3 Hz), 3.99 (2H, s), 4.18 (2H, t, J=8.3 Hz), 7.20-7.52 (11H, m), 7.75 (1H, dt, J=1.9 Hz, 7.8 Hz), 7.91 (1H, d, J=8.7 Hz), 8.47-8.51 (1H, m), 10.12 (1H, s)

APCI-MS (m/z): 482 (M+H)⁺

Preparation 20

6-Methoxy-4′-(trifluoromethyl)-1,1′-biphenyl-2-carboxylic acid

The title compound was obtained in the same manner as in Preparation 15.

¹H-NMR (DMSO-d₆):δ 3.71 (3H, s), 7.30 (1H, dd, J=1.3 Hz, 8.1 Hz), 7.34-7.45 (3H, m), 7.49 (1H, t, J=8.1 Hz), 7.71 (2H, d, J=8.0 Hz), 12.70 (1H, s)

### EXAMPLE 17

6-Methoxy-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 3.07 (2H, t, J=8.3 Hz), 3.74 (3H, s), 3.98 (2H, s), 4.17 (2H, t, J=8.3 Hz), 7.07-7.40 (6H, m), 7.45-7.56 (1H, m), 7.49 (2H, d, J=8.3 Hz), 7.69 (2H, d, J=8.3 Hz), 7.75 (1H, dt, J=1.7 Hz, 7.6 Hz), 7.87 (1H, d, J=8.7 Hz), 8.49 (1H, d, J=4.1 Hz), 10.08 (1H, s)

APCI-MS (m/z): 532 (M+H)⁺

Preparation 21

6-Methoxy-4′-methyl-1,1′-biphenyl-2-carboxylic acid

The title compound was obtained in the same manner as in Preparation 15.

¹H-NMR (DMSO-d₆):δ 2.32 (3H, s), 3-0.68 (3H, s), 7.04-7.26 (2H, m), 7.06 (2H, d, J=8.2 Hz), 7.16 (2H, d, J=8.2 Hz), 7.39 (1H, d, J=7.9 Hz), 12.52 (1H, s)

### EXAMPLE 18

6-Methoxy-4′-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.26 (3H, s), 3.08 (2H, t, J=8.3 Hz), 3.71 (3H, s), 3.94 (2H, s), 4.17 (2H, t, J=8.3 Hz), 7.06-7.47 (7H, m), 7.09 (2H, d, J=8.4 Hz), 7.18 (2H, d, J=8.4 Hz), 7.75 (1H, dt, J=1.9 Hz, 7.6 Hz), 7.87 (1H, d, J=8.7 Hz), 8.48 (1H, d, J=4.0 Hz), 9.95 (1H, s)

ESI-MS(m/z): 478 (M+H)⁺

Preparation 22

4′,6-Dimethoxy-1,1′-biphenyl-2-carboxylic acid

The title compound was obtained in the same manner as in Preparation 15.

¹H-NMR (DMSO-d₆):δ 3.69 (3H, s), 3.77 (3H, s), 6.91 (2H, d, J=8.8 Hz), 7.11 (2H, d, J=8.8 Hz), 7.13-7.24 (2H, m), 7.38 (1H, t, J=7.9 Hz), 12.53 (1H, s)

### EXAMPLE 19

4′,6-Dimethoxy-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.08 (2H, t, J=8.3 Hz), 3.71 (6H, s), 3.97 (2H, s), 4.17 (2H, t, J=8.3 Hz), 6.86 (2H, d, J=8.8 Hz), 7.06-7.45 (7H, m), 7.21 (2H, d, J=8.8 Hz), 7.75 (1H, dt, J=1.9 Hz, 7.7 Hz), 7.87 (1H, d, J=8.7 Hz), 8.48 (1H, dd, J=0.9 Hz, 4.8 Hz), 9.93 (1H, s)

ESI-MS(m/z): 494 (M+H)⁺

Preparation 23

4′-Chloro-6-methoxy-1,1′-biphenyl-2-carboxylic acid

The title compound was obtained in the same manner as in Preparation 15.

¹H-NMR (DMSO-d₆):δ 3.75 (3H, s), 7.15-7.34 (4H, m), 7.37-7.64 (3H, m), 12.66 (1H, s)

### EXAMPLE 20

4′-Chloro-6-methoxy-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.09 (2H, t, J=8.3 Hz), 3.73 (3H, s), 3.98 (2H, s), 4.17 (2H, t, J=8.3 Hz), 7.14 (2H, d, J=7.5 Hz), 7.18-7.51 (9H, m), 7.75 (1H, dt, J=1.8 Hz, 7.7 Hz), 7.88 (1H, d, J=8.7 Hz), 8.49 (1H, d, J=4.1 Hz), 10.04 (1H, s)

ESI-MS(m/z): 498 (M+H)⁺

Preparation 24

6-Methyl-4′-(trifluoromethyl)-1,1′-biphenyl-2-carboxylic acid

The title compound was obtained in the same manner as in Preparation 15.

¹H-NMR (DMSO-d₆):δ 2.02 (3H, s), 7.36-7.52 (2H, m), 7.39 (2H, d, J=8.0 Hz), 7.67 (1H, dd, J=1.2 Hz, 7.5 Hz), 7.76 (2H, d, J=8.0 Hz), 12.59 (1H, s)

### EXAMPLE 21

6-Methyl-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 2.09 (3H, s), 3.07 (2H, t, J=8.3 Hz), 3.97 (2H, s), 4.17 (2H, t, J=8.3 Hz), 7.10 (1H, d, J=8.6 Hz), 7.26 (1H, dd, J=5.0 Hz, 7.4 Hz), 7.31-7.50 (7H, m), 7.70-7.80 (3H, m), 7.87 (1H, d, J=8.7 Hz), 8.49 (1H, d, J=4.0 Hz), 10.73 (1H, s)

APCI-MS (m/z): 516 (M+H)⁺

Preparation 25

6-Methyl-3′-(trifluoromethyl)-1,1′-biphenyl-2-carboxylic acid

The title compound was obtained in the same manner as in Preparation 15.

¹H-NMR (DMSO-d₆):δ 2.02 (3H, s), 7.36-7.53 (4H, m), 7.59-7.74 (3H, m), 12.61 (1H, s)

### EXAMPLE 22

6-Methyl-N-[1-(2-pyridinylacetyl)-2,3-dihydro-1H indol-5-yl]-3′-(trifluoromethyl)-1,1-biphenyl-2-carboxamide

The title compound was obtained in the same manner as in Example 6.

¹H-NMR (DMSO-d₆):δ 2.10 (3H, s), 3.06 (2H, t, J=8.4 Hz), 3.97 (2H, s), 4.17 (2H, t, J=8.4 Hz), 7.08 (1H, d, J=8.7 Hz), 7.26 (1H, dd, J=5.0 Hz, 7.3 Hz), 7.31-7.46 (5H, m), 7.52-7.65 (4H, m), 7.75 (1H, dt, J=1.8 Hz, 7.7 Hz), 7.86 (1H, d, J=8.7 Hz), 8.49 (1H, d, J=4.0 Hz), 10.05 (1H, s)

ESI-MS(m/z): 516 (M+H)⁺

Preparation 26

4′,6-Dimethyl-1,1′-biphenyl-2-carboxylic acid

The title compound was obtained in the same manner as in Preparation 15.

¹H-NMR (DMSO-d₆):δ 2.03 (3H, s), 2.34 (3H, s), 7.03 (2H, d, J=8.0 Hz), 7.19 (2H, d, J=8.0 Hz), 7.32 (1H, t, J=7.4 Hz), 7.42 (1H, dd, J=1.2 Hz, 7.4 Hz), 7.52 (1H, dd, J=1.2 Hz, 7.4 Hz), 12.41 (1H, s)

### EXAMPLE 23

4′,6-Dimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.08 (3H, s), 2.28 (3H, s), 3.08 (2H, t, J=8.4 Hz), 3.98 (2H, s), 4.17 (2H, t, J=8.4 Hz), 7.10-7.16 (5H, m), 7.23-7.41 (6H, m), 7.75 (1H, dt, J=1.7 Hz, 7.6 Hz), 7.86 (1H, d, J=8.7 Hz), 8.49 (1H, d, J=4.9 Hz), 9.94 (1H, s)

ESI-MS(m/z): 462 (M+H)⁺

Preparation 27

4′-Methoxy-6-methyl-1,1′-biphenyl-2-carboxylic acid

The title compound was obtained in the same manner as in Preparation 15.

¹H-NMR (DMSO-d₆):δ 2.05 (3H, s), 3.79 (3H, s), 6.95 (2H, d, J=8.8 Hz), 7.07 (2H, d, J=8.8 Hz), 7.31 (1H, t, J=7.5 Hz), 7.41 (1H, d, J=7.5 Hz), 7.51 (1H, d, J=7.5 Hz), 12.41 (1H, s)

### EXAMPLE 24

4′-Methoxy-6-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.09 (3H, s), 3.07 (2H, t, J=8.3 Hz), 3.73 (3H, s), 3.97 (2H, s), 4.17 (2H, t, J=8.3 Hz), 6.90 (2H, d, J=8.5 Hz), 7.10-7.43 (7H, m), 7.18 (2H, d, J=8.5 Hz), 7.75 (1H, t, J=7.6 Hz), 7.86 (1H, d, J=8.6 Hz), 8.49 (1H, d, J=4.3 Hz), 9.92 (1H, s)

ESI-MS(m/z): 478 (M+H)⁺

Preparation 28

4′-Chloro-6-methyl-1,1′-biphenyl-2-carboxylic acid

The title compound was obtained in the same manner as in Preparation 15.

¹H-NMR (DMSO-d₆):δ 2.03 (3H, s), 7.17 (2H, d, J=8.5 Hz), 7.32-7.48 (4H, m), 7.61 (1H, dd, J=1.2 Hz, 7.4 Hz), 12.53 (1H, s)

negative ESI-MS(m/z): 245, 247 (M−H)⁻

### EXAMPLE 25

4′-Chloro-6-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.09 (3H, s), 3.09 (2H, t, J=8.4 Hz), 3.98 (2H, s), 4.17 (2H, t, J=8.4 Hz), 7.14 (1H, d, J=8.7 Hz), 7.24-7.44 (10H, m), 7.75 (1H, dt, J=1.8 Hz, 7.6 Hz), 7.87 (1H, d, J=8.7 Hz), 8.49 (1H, d, J=4.0 Hz), 10.03 (1H, s)

ESI-MS(m/z): 482 (M+H)⁺

Preparation 29

5-Chloro-4′-(trifluoromethyl)-1,1′-biphenyl-2-carboxylic acid

The title compound was obtained in the same manner as in Preparation 15.

¹H-NMR (DMSO-d₆):δ 7.51 (1H, s), 7.52-7.67 (3H, m), 7.74-7.95 (3H, m), 13.05 (1H, s)

### EXAMPLE 26

5-Chloro-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 3.11 (2H, t, J=8.4 Hz), 3.99 (2H, s), 4.19 (2H, t, J=8.4 Hz), 7.19-7.38 (3H, m), 7.45 (1H, s), 7.58-7.82 (8H, m), 7.92 (1H, d, J=8.7 Hz), 8.46-8.51 (1H, m), 10.29 (1H, s)

APCI-MS (m/z): 536, 537 (M+H)⁺

Preparation 30

5-Chloro-4′-methyl-1,1′-biphenyl-2-carboxylic acid

The title compound was obtained in the same manner as in Preparation 15.

¹H-NMR (DMSO-d₆):δ 2.35 (3H, s), 7.23 (4H, s), 7.41 (1H, d, J=2.1 Hz), 7.50 (1H, dd, J=2.1 Hz, 8.3 Hz), 7.73 (1H, d, J=8.3 Hz), 12.92 (1H, s)

### EXAMPLE 27

5-Chloro-4′-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.29 (3H, s), 3.16 (2H, t, J=8.3 Hz), 3.99 (2H, s), 4.18 (2H, t, J=8.3 Hz), 7.16-7.38 (7H, m), 7.48-7.59 (4H, m), 7.75 (1H, dt, J=1.8 Hz, 8.7 Hz), 7.92 (1H, d, J=8.7 Hz), 8.49 (1H, dd, J=0.7 Hz, 4.8 Hz), 10.20 (1H, s)

APCI-MS (m/z): 482, 484 (M+H)⁺

Preparation 31

4′,5-Dichloro-1,1′-biphenyl-2-carboxylic acid

The title compound was obtained in the same manner as in Preparation 15.

¹H-NMR (DMSO-d₆):δ 7.36 (2H, d, J=8.6 Hz), 7.44-7.62 (2H, m), 7.48 (2H, d, J=8.6 Hz), 7.80 (1H, d, J=8.3 Hz), 13.04 (1H, s)

### EXAMPLE 28

4′,5-Dichloro-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.11 (2H, t, J=8.4 Hz), 3.98 (2H, s), 4.19 (2H, t, J=8.4 Hz), 7.20-7.28 (2H, m), 7.35 (1H, d, J=7.8 Hz), 7.44-7.50 (5H, m), 7.52-7.66 (3H, m), 7.76 (1H, dt, J=1.8 Hz, 7.6 Hz), 7.92 (1H, d, J=8.7 Hz), 8.49 (1H, dd, J=0.8 Hz, 4.8 Hz), 10.24 (1H, s)

APCI-MS (m/z): 502, 504 (M+H)⁺

### EXAMPLE 29

4′-Nitro-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.11 (2H, t, J=8.2 Hz), 3.99 (2H, s), 4.18 (2H, t, J=8.2 Hz), 7.20-7.30 (2H, m)., 7.35 (1H, d, J=7.8 Hz), 7.53-7.80 (8H, m), 7.92 (1H, d, J=8.7 Hz), 8.25 (2H, d, J=8.8 Hz), 8.49 (1H, d, J=4.8 Hz), 10.33 (1H, s)

APCI-MS (m/z): 479 (M+H)⁺

Preparation 32

To a mixture of 4′-nitro-1,1′-biphenyl-2-carboxylic acid (1.0 g) and 37% aqueous formaldehyde (6.2 ml) in methanol (10 ml) and tetrahydrofuran (10 ml) was added 10% palladium on carbon (1.0 g, 50% wet). The reaction mixture was stirred at ambient temperature for 8 hours under hydrogen atmosphere. The catalyst was filtered off and the solvent was removed by concentration in vacuo and the residue was triturated with a mixture of diethyl ether and diisopropyl ether to give 4′-(dimethylamino)-1,1′-biphenyl-2-carboxylic acid (0.68 g).

¹H-NMR (DMSO-d₆):δ 2.92 (6H, s), 6.75 (2H, d, J=8.7 Hz), 7.18 (2H, d, J=8.7 Hz), 7.29-7.38 (2H, m), 7.45-7.54 (1H, m), 7.61 (1H, dt, J=1.5 Hz, 8.4 Hz), 12.63 (1H, s)

APCI-MS (m/z): 242 (M+H)⁺

### EXAMPLE 30

To a suspension of 4′-(dimethylamino)-1,1′-biphenyl-2-carboxylic acid (0.34 g) in dichloromethane (5 ml) were added thionyl chloride (0.17 ml) and N,N-dimethylformamide (2 drops) and the mixture was stirred under reflux for 2 hours. The mixture was evaporated in vacuo and the residue was dissolved in dichloromethane (2.0 ml). The acid chloride solution was added to a solution of 1-(2-pyridinylacetyl)-5-indolinamine (0.3 g) and triethylamine (0.41 ml) in dichloromethane (5 ml) under ice-cooling and the mixture was stirred at ambient temperature for 3 hours. The reaction mixture was poured into water and the mixture was adjusted to pH 1 with 6N hydrochloric acid. The separated aqueous layer was adjusted to pH 9 with 20% aqueous potassium carbonate. The aqueous layer was extracted with a mixture of ethyl acetate and tetrahydrofuran. The extract was washed with water, dried over magnesium sulfate and evaporated in vacuo. The residue was recrystallized from a mixture of tetrahydrofuran and diisopropyl ether to give 4′-(dimethylamino)-N-[1-(2-pyridinylacetyl)-2,3-dihydro-1H-indol-5-yl]-1,1′-biphenyl-2-carboxamide (0.16 g).

¹H-NMR (DMSO-d₆):δ 2.88 (6H, s), 3.11 (2H, t, J=8.3 Hz), 3.99 (2H, s), 4.19 (2H, t, J=8.3 Hz), 6.70 (2H, d, J=8.8 Hz), 7.23-7.55 (10H, m), 7.75 (1H, dt, J=1.8 Hz, 7.7 Hz), 7.92 (1H, d, J=8.7 Hz), 8.49 (1H, d, J=4.0 Hz), 10.13 (1H, s)

APCI-MS (m/z): 477 (M+H)⁺

Preparation 33

To a mixture of methyl 4-(dimethylamino)-2-(trifluoromethanesulfonyloxy)benzoate (6.5 g), lithium chloride (2.5 g) and tetrakis(triphenylphosphine)palladium(0) (1.1 g) in toluene (100 ml) was added a solution of sodium carbonate (5.5 g) in water (28 ml) under stirring, followed by addition of 4-(trifluoromethyl)phenylboronic acid (3.4 g). The mixture was stirred at 100° C. for 6 hours. The reaction mixture was poured into a mixture of ethyl acetate and water. The separated organic layer was washed with water, dried over magnesium sulfate and evaporated in vacuo. The residue was purified by column chromatography on silica gel eluting with ethyl acetate:n-hexane (1:9 v/v). The eluted fractions containing the desired product were collected and evaporated in vacuo to give methyl 5-(dimethylamino)-4′-(trifluoromethyl)-1,1′-biphenyl-2-carboxylate (3.26 g).

¹H-NMR (DMSO-d₆):δ 3.01 (6H, s), 3.53 (3H, s), 6.50 (1H, d, J=2.7 Hz), 6.77 (1H, dd, J=2.7 Hz, 8.9 Hz), 7.46 (2H, d, J=8.0 Hz), 7.61 (2H, d, J=8.0 Hz), 7.81 (1H; d, J=8.9 Hz)

Preparation 34

A mixture of methyl 5-(dimethylamino)-4′-(trifluoromethyl)-1,1′-biphenyl-2-carboxylate (3.2 g) and sodium hydroxide (1.0 g) in water (10 ml) and ethanol (16 ml) was stirred under reflux for 6 hours. The solvent was removed by concentration in vacuo. The residue was dissolved in a mixture of ethyl acetate and water, and the mixture was adjusted to pH 5 with 6N hydrochloric acid. The separated organic layer was washed with water, dried over magnesium sulfate and evaporated in vacuo to give 5-(dimethylamino)-4′-(trifluoromethyl)-1,1′-biphenyl-2-carboxylic acid (2.38 g).

¹H-NMR (DMSO-d₆):δ 3.00 (6H, s), 6.46 (1H, d, J=2.6 Hz), 6.76 (1H, dd, J=2.6 Hz, 8.9 Hz), 7.48 (2H, d, J=8.1 Hz), 7.70 (2H, d, J=8.1 Hz), 7.81 (1H, d, J=8.9 Hz), 11.93 (1H, s)

### EXAMPLE 31

5-(Dimethylamino)-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 3.00 (6H, s), 3.09 (2H, t, J=7.5 Hz), 3.98 (2H, s), 4.18 (2H, t, J=7.5 Hz), 6.65 (1H, d, J=2.4 Hz), 6.80 (1H, dd, J=2.4 Hz, 8.7 Hz), 7.13-7.31 (2H, m), 7.35 (1H, d, J=7.8 Hz), 7.46-7.60 (4H, m), 7.67-7.81 (3H, m), 7.89 (1H, d, J=8.7 Hz), 8.49 (1H, dd, J=0.7 Hz, 4.0 Hz), 9.92 (1H, s)

APCI-MS (m/z): 545 (M+H)⁺

Preparation 35

Methyl 5-(dimethylamino)-4′-methyl-1,1′-biphenyl-2-carboxylate

The title compound was obtained in the same manner as in Preparation 33.

¹H-NMR (DMSO-d₆):δ 2.33 (3H, s), 2.99 (6H, s), 3.51 (3H, s), 6.46 (1H, d, J=2.6 Hz), 6.71 (1H, dd, J=2.6 Hz, 8.8 Hz), 7.12 (2H, d, J=8.4 Hz), 7.17 (2H, d, J=8.4 Hz), 7.70 (1H, d, J=8.8 Hz)

Preparation 36

5-(Dimethylamino)-4′-methyl-1,1′-biphenyl-2-carboxylic acid

The title compound was obtained in the same manner as in Preparation 34.

¹H-NMR (DMSO-d₆):δ 2.33 (3H, s), 2.98 (6H, s), 6.43 (1H, d, J=2.6 Hz), 6.79 (1H, dd, J=2.6 Hz, 8.8 Hz), 7.15 (4H, s), 7.71 (1H, d, J=8.8 Hz), 11.80 (1H, s)

### EXAMPLE 32

5-(Dimethylamino)-4′-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.29 (3H, s), 2.98 (6H, s), 3.09 (2H, t, J=8.4 Hz), 3.98 (2H, s), 4.18 (2H, t, J=8.4 Hz), 6.60 (1H, d, J=1.9 Hz), 6.72 (1H, dd, J=1.9 Hz, 8.7 Hz), 7.10-7.50 (9H, m), 7.75 (1H, t, J=7.8 Hz), 7.88 (1H, d, J=8.7 Hz), 8.49 (1H, d, J=4.1 Hz), 9.74 (1H, s)

APCI-MS (m/z): 491 (M+H)⁺

Preparation 37

Methyl 4′-chloro-5-(dimethylamino)-1,1′-biphenyl-2-carboxylate

The title compound was obtained in the same manner as in Preparation 33.

¹H-NMR (DMSO-d₆):δ 3.00 (6H, s), 3.52 (3H, s), 6.47 (1H, d, J=2.6 Hz), 6.76 (1H, dd, J=2.6 Hz, 8.8 Hz), 7.26 (2H, d, J=8.4 Hz), 7.41 (2H, d, J=8.4 Hz), 7.76 (1H, d, J=8.8 Hz)

Preparation 38

4′-Chloro-5-(dimethylamino)-1,1′-biphenyl-2-carboxylic acid

The title compound was obtained in the same manner as in Preparation 34.

¹H-NMR (DMSO-d₆):δ 2.99 (6H, s), 6.44 (1H, d, J=2.6 Hz), 6.72 (1H, dd, J=2.6 Hz, 8.8 Hz), 7.27 (2H, d, J=8.5 Hz), 7.40 (2H, d, J=8.5 Hz), 7.76 (1H, d, J=8.8 Hz), 11.90 (1H, s)

### EXAMPLE 33

4′-Chloro-5-(dimethylamino)-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.99 (6H, s), 3.06 (2H, t, J=8.3 Hz), 3.98 (2H, s), 4.19 (2H, t, J=8.3 Hz), 6.61 (1H, d, J=2.4 Hz), 6.77 (1H, dd, J=2.4 Hz, 8.7 Hz), 7.16-7.52 (9H, m), 7.76 (1H, dt, J=1.9 Hz, 7.6 Hz), 7.89 (1H, d, J=8.7 Hz), 8.49 (1H, d, J=4.2 Hz), 9.85 (1H, s)

APCI-MS (m/z): 511, 513 (M+H)⁺

Preparation 39

Methyl 6-nitro-4′-(trifluoromethyl)-1,1′-biphenyl-2-carboxylate

The title compound was obtained in the same manner as in Preparation 33.

¹H-NMR (DMSO-d₆):δ 3.56 (3H, s), 7.48 (2H, d, J=8.0 Hz), 7.79 (2H, d, J=8.0 Hz), 7.83 (1H, t, J=8.0 Hz), 8.17 (1H, dd, J=1.3 Hz, 8.0 Hz), 8.25 (1H, dd, J=1.3 Hz, 8.0 Hz)

Preparation 40

6-Nitro-4′-(trifluoromethyl)-1,1′-biphenyl-2-carboxylic acid

The title compound was obtained in the same manner as in Preparation 34.

¹H-NMR (DMSO-d₆):δ 7.49 (2H, d, J=8.0 Hz), 7.51-7.85 (3H, m), 8.12 (1H, dd, J=1.3 Hz, 8.0 Hz), 8.18 (1H, dd, J=1.3 Hz, 8.0 Hz)

### EXAMPLE 34

6-Nitro-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 30.

¹H-NMR (DMSO-d₆):δ 3.08 (2H, t, J=8.4 Hz), 3.98 (2H, s), 4.18 (2H, t, J=8.4 Hz), 7.07 (1H, d, J=8.7 Hz), 7.22-7.38 (3H, m), 7.52 (2H, d, J=8.0 Hz), 7.70-7.99 (6H, m), 8.16 (1H, dd, J=1.3 Hz, 8.0 Hz), 8.49 (1H, d, J=4.0 Hz), 10.31 (1H, s)

APCI-MS (m/z): 547 (M+H)⁺

### EXAMPLE 35

To a solution of 6-nitro-N-[1-(2-pyridinylacetyl)-2,3-dihydro-1H-indol-5-yl]-4′-(trifluoromethyl)-1,1′-biphenyl-2-carboxamide (1.2 g) in a mixture of methanol (12 ml) and tetrahydrofuran (12 ml) was added 10% palladium on carbon (0.4 g, 50% wet). The reaction mixture was stirred at ambient temperature for 5 hours under hydrogen atmosphere. The catalyst was filtered off and the solvent was removed by concentration in vacuo and the residue was triturated with ethyl acetate to give 6-amino-N-[1-(2-pyridinylacetyl)-2,3-dihydro-1H-indol-5-yl]-41-(trifluoromethyl)-1,1′-biphenyl-2-carboxamide (0.97 g).

¹H-NMR (DMSO-d₆):δ 3.06 (2H, t, J=8.3 Hz), 3.97 (2H, s), 4.16 (2H, t, J=8.3 Hz), 4.82 (2H, s), 6.76 (1H, d, J=7.3 Hz), 6.88 (1H, d, J=7.3 Hz), 7.06-7.37 (5H, m), 7.48 (2H, d, J=8.0 Hz), 7.69-7.80 (3H, m), 7.85 (1H, d, J=8.7 Hz), 8.48 (1H, dd, J=0.9 Hz, 4.0 Hz), 9.94 (1H, s)

negative APCI-MS (m/z): 515 (M−H)⁻

Preparation 41

6-(Dimethylamino)-4′-(trifluoromethyl)-1,1′-biphenyl-2-carboxylic acid

The title compound was obtained in the same manner as in Preparation 32.

¹H-NMR (DMSO-d₆):δ 2.38 (6H, s), 7.27-7.44 (3H, m), 7.48 (2H, d, J=8.1 Hz), 7.74 (2H, d, J=8.1 Hz), 12.64 (1H, s)

### EXAMPLE 36

6-(Dimethylamino)-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 2.41 (6H, s), 3.07 (2H, t, J=8.3 Hz), 3.98 (2H, s), 4.17 (2H, t, J=8.3 Hz), 7.09 (1H, d, J=8.7 Hz), 7.16 (1H, d, J=7.0 Hz), 7.22-7.37 (4H, m), 7.44 (1H, t, J=7.8 Hz), 7.56 (2H, d, J=8.1 Hz), 7.70 (2H, d, J=8.1 Hz), 7.75 (1H, dt, J=1.8 Hz, 7.8 Hz), 7.87 (1H, d, J=8.7 Hz), 8.48 (1H, d, J=4.8 Hz), 10.04 (1H, s)

APCI-MS (m/z): 545 (M+H)⁺

Preparation 42

Methyl 4′-methyl-6-nitro-1,1′-biphenyl-2-carboxylate

The title compound was obtained in the same manner as in Preparation 33.

¹H-NMR (DMSO-d₆):δ 2.35 (3H, s), 3.54 (3H, s), 7.07 (2H, d, J=7.9 Hz), 7.22 (2H, d, J=7.9 Hz), 7.73 (1H, t, J=8.0 Hz), 8.01 (1H, dd, J=1.3 Hz, 8.0 Hz), 8.11 (1H, dd, J=1.3 Hz, 8.0 Hz)

Preparation 43

4′-Methyl-6-nitro-1,1′-biphenyl-2-carboxylic acid

The title compound was obtained in the same manner as in Preparation 34.

¹H-NMR (DMSO-d₆):δ 2.34 (3H, s), 7.11 (2H, d, J=8.0 Hz), 7.21 (2H, d, J=8.0 Hz), 7.69 (1H, t, J=8.0 Hz), 7.98 (1H, dd, J=1.3 Hz, 8.0 Hz), 8.04 (1H, dd, J=1.3 Hz, 8.0 Hz), 12.90 (1H, br s)

Preparation 44

6-(Dimethylamino)-4′-methyl-1,1′-biphenyl-2-carboxylic acid

The title compound was obtained in the same manner as in Preparation 32.

¹H-NMR (DMSO-d₆):δ 2.33 (3H, s), 2.38 (6H, s), 7.10-7.22 (6H, m), 7.32 (1H, t, J=7.3 Hz), 12.43 (1H, s)

APCI-MS (m/z): 256 (M+H)⁺

### EXAMPLE 37

6-(Dimethylamino)-4′-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.26 (3H, s), 2.40 (6H, s), 3.07 (2H, t, J=8.2 Hz), 3.97 (2H, s), 4.16 (2H, t, J=8.2 Hz), 7.03-7.40 (11H, m), 7.75 (1H, dt, J=1.8 Hz, 7.8 Hz), 7.87 (1H, d, J=8.7 Hz), 8.49 (1H, d, J=4.8 Hz), 9.94 (1H, s)

APCI-MS (m/z): 491 (M+H)⁺

Preparation 45

A solution of 4′-(trifluoromethyl)-1,1′-biphenyl-2-carbonyl chloride (11.5 g) in tetrahydrofuran (10 ml) was added to a mixture of 1-acetyl-2,3-dihydro-1H-indol-5-ylamine (7.1 g) and triethylamine (8.14 g) in tetrahydrofuran (150 ml) at ambient temperature. The mixture was stirred at ambient temperature for 3 hours. The reaction mixture was poured into a mixture of ethyl acetate and water. The organic layer was washed with 5% aqueous potassium carbonate solution and brine and dried over magnesium sulfate. The solvent was concentrated in vacuo and the precipitate was collected by filtration, washed with a mixture of ethyl acetate and diisopropyl ether to give N-(1-acetyl-2,3-dihydro-1H-indol-5-yl)-4′-(trifluoromethyl)-1,1′-biphenyl-2-carboxamide (15.6 g).

¹H-NMR (DMSO-d₆):δ 2.12 (3H, s), 3.08 (2H, t, J=8.44 Hz), 4.05 (2H, t, J=8.44 Hz), 7.22 (1H, d, J=8.62 Hz), 7.47-7.65 (8H, m), 7.76 (1H, d, J=8.28 Hz), 7.92 (1H, d, J=8.64 Hz), 10.26 (1H, s)

Preparation 46

A mixture of N-(1-acetyl-2,3-dihydro-1H-indol-5-yl)-4′-(trifluoromethyl)-1,1′-biphenyl-2-carboxamide (637 mg) and 6N hydrochloric acid (6 ml) in methanol (15 ml) and tetrahydrofuran (10 ml) was refluxed under stirring for 5 hours. The reaction mixture was poured into a mixture of ethyl acetate and water. The mixture was adjusted to pH 9.0 with 20% aqueous potassium carbonate solution and the organic layer was washed with brine and dried over magnesium sulfate. The solvent was concentrated in vacuo and the precipitate was collected by filtration to give N-(2,3-dihydro-1H-indol-5-yl)-4′-(trifluoromethyl)-1,1′-biphenyl-2-carboxamide (502 mg).

¹H-NMR (DMSO-d₆):δ 2.84 (2H, t, J=8.30 Hz), 3.37 (2H, t, J=8.30 Hz), 5.32 (1H, s), 6.39 (1H, d, J=8.26 Hz), 6.98 (1H, dd, J=2.08 Hz, 8.26 Hz), 7.19 (1H, s), 7.49-7.65 (6H, m), 7.76 (2H, d, J=8.26 Hz), 9.89 (1H, s)

### EXAMPLE 38

A mixture of N-(2,3-dihydro-1H-indol-5-yl)-4′-(trifluoromethyl)-1,1′-biphenyl-2-carboxamide (383 mg), 2-vinylpyridine (126 mg) and acetic acid (60 mg) in ethanol (10 ml) was refluxed under stirring for 8 hours. The reaction mixture was poured into a mixture of ethyl acetate and water. The mixture was adjusted to pH 9.0 with 20% aqueous potassium carbonate solution and the organic layer was washed with brine and dried over magnesium sulfate. The solvent was concentrated in vacuo and the residue was chromatographed on silica gel eluting with ethyl acetate:n-hexane (5:5-6:4 v/v). The eluted fractions containing the desired product were collected and evaporated in vacuo. The residue was recrystallized from ethyl acetate and diisopropyl ether to give N-{1-[2-(2-pyridinyl)ethyl]-2,3-dihydro-1H-indol-5-yl}-4′-(trifluoromethyl)-1,1′-biphenyl-2-carboxamide (260 mg).

¹H-NMR (DMSO-d₆):δ 2.81 (2H, t, J=7.98 Hz), 2.97 (2H, t, J=7.98 Hz), 3.19-3.42 (4H, m), 6.42 (1H, d, J=8.40 Hz), 7.18-7.20 (3H, m), 7.34 (1H, d, J=7.82 Hz), 7.36-7.78 (9H, m), 8.49-8.52 (1H, m), 9.95 (1H, s)

Preparation 47

A solution of 4′-methyl-1,1′-biphenyl-2-carboxylic acid (2.12 g) and 1-hydroxybenzotriazole hydrate (1.49 g) and 1-[3-(dimethylamino)propyl]-3-ethylcarbodiimide hydrochloride (2.10 g) in N,N-dimethylformamide (30 ml) was stirred at ambient temperature for an hour. To the above solution was added 1-acetyl-5-indolinamine (1.85 g), and the mixture was stirred at ambient temperature for 15 hours. The reaction mixture was poured into a mixture of ethyl acetate and water. The organic layer was washed with brine and dried over magnesium sulfate. The solvent was evaporated in vacuo and the residue was recrystallized from ethyl acetate and diisopropyl ether to give N-(1-acetyl-2,3-dihydro-1H-indol-5-yl)-4′-methyl-1,1′-biphenyl-2-carboxamide (3.40 g).

¹H-NMR (DMSO-d₆):δ 2.12 (3H, s), 2.28 (3H, s) 3.08 (2H, t, J=8.32 Hz), 4.04 (2H, t, J=8.32 Hz), 7.17 (2H, d, J=8.02 Hz), 7.21-7.58 (8H, m), 7.90 (1H, d, J=8.64 Hz), 10.16 (1H, s)

Preparation 48

A mixture of N-(1-acetyl-2,3-dihydro-1H-indol-5-yl)-4′-methyl-1,1′-biphenyl-2-carboxamide (3.324 g) and 6N hydrochloric acid (40 ml) in methanol (70 ml) and tetrahydrofuran (70 ml) was refluxed under stirring for 5 hours. The reaction mixture was concentrated in vacuo, and the precipitate was collected by filtration and dried to give N-(2,3-dihydro-1H-indol-5-yl)-4′-methyl-1,1′-biphenyl-2-carboxamide hydrochloride (3.03 g).

¹H-NMR (DMSO-d₆):δ 2.29 (3H, s), 3.15 (2H, t, J=7.68 Hz), 3.68 (2H, t, J=7.68 Hz), 7.17 (1H, d, J=8.00 Hz), 7.28-7.60 (8H, m), 7.69 (1H, s), 10.43 (1H, s), 11.08 (1H, br s)

### EXAMPLE 39

4′-Methyl-N-{1-[2-(2-pyridinyl)ethyl]-2,3-dihydro-1H-indol-5-yl}-1,1′-biphenyl-2-carboxamide

The title compound was obtained in the same manner as in Example 38.

¹H-NMR (DMSO-d₆):δ 2.29 (3H, s), 2.81 (2H, t, J=7.98 Hz), 2.96 (2H, t, J=7.98 Hz), 3.19-3.42 (4H, m), 6.42 (1H, d, J=8.38 Hz), 7.09-7.56 (12H, m), 7.65-7.74 (1H, m), 8.51 (1H, d, J=4.02 Hz), 9.85 (1H, s)

Preparation 49

N-(1-Acetyl-2,3-dihydro-1H-indol-5-yl)-4′-methoxy-1,1′-biphenyl-2-carboxamide

The title compound was obtained in the same manner as in Preparation 45.

¹H-NMR (DMSO-d₆):δ 2.12 (3H, s), 3.09 (2H, t, J=8.36 Hz), 3.78 (3H, s), 4.05 (2H, t, J=8.6 Hz), 6.93 (2H, d, J=8.66 Hz), 7.24 (1H, d, J=8.60 Hz), 7.37 (2H, d, J=8.66 Hz), 7.40-7.56 (5H, m), 7.90 (1H, d, J=8.66 Hz), 10.13 (1H, s)

Preparation 50

N-(2,3-Dihydro-1H-indol-5-yl)-4′-methoxy-1,1′-biphenyl-2-carboxamide

The title compound was obtained in the same manner as in Preparation 46.

¹H-NMR (DMSO-d₆):δ 2.84 (2H, t, J=8.24 Hz), 3.33-3.41 (2H, m), 3.84 (3H, s), 5.32 (1H, s), 6.39 (1H, d, J=8.27 Hz), 6.94 (2H, d, J=8.64 Hz), 7.02 (1H, dd, J=1.74 Hz, 8.27 Hz), 7.23 (1H, s), 7.36-7.54 (7H, m), 9.78 (1H, s)

### EXAMPLE 40

4′-Methoxy-N-{1-[2-(2-pyridinyl)ethyl]-2,3-dihydro-1H-indol-5-yl}-1,1′-biphenyl-2-carboxamide

The title compound was obtained in the same manner as in Example 38.

¹H-NMR (DMSO-d₆):δ 2.81 (2H, t, J=7.86 Hz), 2.96 (2H, t, J=7.86 Hz), 3.20-3.42 (4H, m), 6.42 (1H, d, J=8.38 Hz), 6.94 (2H, d, J=8.63 Hz), 7.10-7.53 (10H, m), 7.65-7.73 (1H, m), 8.50 (1H, d, J=4.25 Hz), 9.83 (1H, s)

### EXAMPLE 41

A mixture of N-(2,3-dihydro-1H-indol-5-yl)-4′-methoxy-1,1′-biphenyl-2-carboxamide (517 g), 2-pyridylacetic acid hydrochloride (313 g), 1-hydroxybenzotriazole hydrate (267 mg), 1-[3-(dimethylamino)propyl]-3-ethylcarbodiimide hydrochloride (378 mg) and triethylamine (400 mg) in dichloromethane (30 ml) was stirred at ambient temperature for 15 hours. The reaction mixture was poured into a mixture of dichloromethane and water and adjusted to pH 8.0 with aqueous potassium carbonate solution. The organic layer was washed with brine and dried over magnesium sulfate. The solvent was evaporated in vacuo. The residue was chromatographed on silica gel eluting with ethyl acetate:n-hexane (7:3-10:0 v/v). The eluted fractions containing the desired product were collected and evaporated in vacuo. The residue was recrystallized from a mixture of ethyl acetate and diisopropyl ether to give 4′-methoxy-N-[1-(2-pyridinylacetyl)-2,3-dihydro-1H-indol-5-yl]-1,1′-biphenyl-2-carboxamide (110 mg).

¹H-NMR (DMSO-d₆):δ 3.11 (2H, t, J=8.12 Hz), 3.74 (3H, s), 4.05 (2H, s), 4.18 (2H, t, J=8.12 Hz), 6.93 (2H, d, J=8.64 Hz), 6.93 (2H, d, J=8.64 Hz), 7.23-7.59 (10H, m), 7.71-7.70 (1H, m), 7.92 (1H, d, J=8.66 Hz), 8.49 (1H, d, J=4.84 Hz), 10.15 (1H, s)

Preparation 51

N-(1-Acetyl-2,3-dihydro-1H-indol-5-yl)-4′-chloro-1,1′-biphenyl-2-carboxamide

The title compound was obtained in the same manner as in Preparation 47.

¹H-NMR (DMSO-d₆):δ 2.13 (3H, s), 3.09 (2H, t, J=8.36 Hz), 4.06 (2H, t, J=8.36 Hz), 7.22 (1H, d, J=8.70 Hz), 7.33-7.70 (9H, m), 7.90 (1H, d, J=8.64 Hz), 10.19 (1H, s)

Preparation 52

4′-Chloro-N-(2,3-dihydro-1H-indol-5-yl)-1,1′-biphenyl-2-carboxamide

The title compound was obtained in the same manner as in Preparation 46.

¹H-NMR (DMSO-d₆):δ 2.85 (2H, t, J=8.30 Hz), 3.33-3.41 (2H, m), 5.32 (1H, s), 6.38 (1H, d, J=8.26 Hz), 7.01 (1H, dd, J=1.55 Hz, 8.26 Hz), 7.23 (1H, d, J=1.55 Hz), 7.41-7.64 (8H, m), 9.86 (1H, s)

### EXAMPLE 42

4′-Chloro-N-{1-[2-(2-pyridinyl)ethyl]-2,3-dihydro-1H-indol-5-yl}-1,1′-biphenyl-2-carboxamide

The title compound was obtained in the same manner as in Example 38.

¹H-NMR (DMSO-d₆):δ 2.82 (2H, t, J=7.99 Hz), 2.96 (2H, t, J=7.99 Hz), 3.20-3.42 (4H, m), 6.42 (1H, d, J=8.40 Hz), 7.03-7.23 (3H, m), 7.32-7.70 (8H, m), 7.65-7.73 (1H, m), 8.49-8.52 (1H, m), 9.90 (1H, s)

Preparation 53

N-(1-Acetyl-2,3-dihydro-1H-indol-5-yl)-3′-(trifluoromethyl)-1,1′-biphenyl-2-carboxamide

The title compound was obtained in the same manner as in Preparation 47.

¹H-NMR (DMSO-d₆):δ 2.12 (3H, s), 3.08 (2H, t, J=8.34 Hz), 4.06 (2H, t, J=8.34 Hz), 7.17 (1H, d, J=8.32 Hz), 7.42 (1H, ds), 7.56-7.75 (8H, m), 7.89 (1H, d, J=8.66 Hz), 10.22 (1H, s)

Preparation 54

N-(2,3-Dihydro-1H-indol-5-yl)-3′-(trifluoromethyl)-1,1′-biphenyl-2-carboxamide

The title compound was obtained in the same manner as in Preparation 46.

¹H-NMR (DMSO-d₆):δ 2.83 (2H, t, J=8.32 Hz), 3.33-3.62 (2H, m), 5.33 (1H, s), 6.38 (1H, d, J=8.24 Hz), 6.95 (1H, d, J=8.20 Hz), 7.16 (1H, s), 7.50-7.76 (8H, m), 9.88 (1H, s)

### EXAMPLE 43

N-{1-[2-(2-pyridinyl)ethyl]-2,3-dihydro-1H-indol-5-yl}-3′-(trifluoromethyl)-1,1′-biphenyl-2-carboxamide

The title compound was obtained in the same manner as in Example 38.

¹H-NMR (DMSO-d₆):δ 2.81 (2H, t, J=8.01 Hz), 2.96 (2H, t, J=8.01 Hz), 3.27-3.48 (4H, m), 6.40 (1H, d, J=8.47 Hz), 7.03 (1H, d, J=8.36 Hz), 7.20-7.24 (2H, m), 7.34 (1H, d, J=7.77 Hz), 7.50-7.76 (9H, m), 8.50 (1H, d, J=3.96 Hz), 9.93 (1H, s)

### EXAMPLE 44

N-[1-(2-Pyridinylacetyl)-2,3-dihydro-1H-indol-5-yl]-3′-(trifluoromethyl)-1,1′-biphenyl-2-carboxamide

The title compound was obtained in the same manner as in Example 41.

¹H-NMR (DMSO-d₆):δ 3.10 (2H, t, J=8.26 Hz), 3.99 (2H, s), 4.19 (2H, t, J=8.26 Hz), 7.17-7.30 (2H, m), 7.35 (1H, d, J=7.82 Hz), 7.44 (1H, s), 7.51-7.79 (9H, m), 7.91 (1H, d, J=8.66 Hz), 8.49 (1H, d, J=3.98 Hz), 10.25 (1H, s)

### EXAMPLE 45

4′-Methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.28 (3H, s), 3.15 (2H, t, J=8.34 Hz), 3.99 (2H, s), 4.19 (2H, t, J=8.34 Hz), 7.15-7.55 (12H, m), 7.71-7.76 (1H, m), 7.91 (1H, d, J=8.68 Hz), 8.49 (1H, d, J=4.02 Hz), 10.16 (1H, s)

### EXAMPLE 46

A solution of conc. hydrochloric acid (1 ml) in water (4 ml) was added to a mixture of 4′-methyl-N-[1-(2-pyridinylacetyl)-2,3-dihydro-1H-indol-5-yl]-1,1′-biphenyl-2-carboxamide (4.0 g) in ethanol (80 ml), and the mixture was heated at 75-80° C. The dissolved solution was filtrated and the filtrate was cooled at ambient temperature. The precipitate was collected by filtration to give crude 4′-methyl-N-[1-(2-pyridinylacetyl)-2,3-dihydro-1H-indol-5-yl]-1,1′-biphenyl-2-carboxamide hydrochloride (3.78 g). The crude product (3.78 g) was recrystallized from 85% aqueous ethanol to give pure 4′-methyl-N-[1-(2-pyridinylacetyl)-2,3-dihydro-1H-indol-5-yl]-1,1′-biphenyl-2-carboxamide hydrochloride (2.70 g).

¹H-NMR (DMSO-d₆):δ 2.26 (3H, s), 3.20 (2H, t, J=8.24 Hz), 4.25 (2H, t, J=8.24 Hz), 4.62 (2H, s), 7.17 (2H, d, J=8.00 Hz), 7.2-7.52 (9H, m), 7.86 (1H, d, J=8.70 Hz), 7.88-7.98 (1H, m), 8.46-8.51 (1H, m), 8.89 (1H, d, J=5.64 Hz), 10.23 (1H, s)

Elemental analysis calcd for C₂₉H₂₅N₃O₂.HCl: C; 71.99%, H; 5.41%, N: 8.68%, found: C; 71.64%, H; 5.66%, N; 8.48%

### EXAMPLE 47

4′-Chloro-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.12 (2H, t, J=8.30 Hz), 3.99 (2H, s), 4.19 (2H, t, J=8.30 Hz), 7.24-7.59 (12H, m), 7.72-7.80 (1H, m), 7.92 (1H, d, J=8.68 Hz), 8.49 (1H, d, J=3.96 Hz), 10.21 (1H, s)

### EXAMPLE 48

3′-Methoxy-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.15 (2H, t, J=8.22 Hz), 3.69 (3H, s), 4.05 (2H, s), 4.19 (2H, t, J=8.22 Hz), 6.99-7.01 (1H, m), 7.02 (2H, m), 7.23-7.59 (9H, m), 7.71-7.80 (1H, m), 7.92 (1H, d, J=8.68 Hz), 8.50 (1H, d, J=3.98 Hz), 10.20 (1H, s)

### EXAMPLE 49

3′-Chloro-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.11 (2H, t, J=8.18 Hz), 3.99 (2H, s), 4.19 (2H, t, J=8.18 Hz), 7.20-7.62 (12H, m), 7.71-7.80 (1H, m), 7.93 (1H, d, J=8.66 Hz), 8.49 (1H, d, J=4.86 Hz), 10.22 (1H, s)

Preparation 55

A mixture of 5-nitroindoline (1.64 g), 2-pyridinecarboxaldehyde (2.14 g) and sodium triacetoxyborohydride (6.36 g) in dichloromethane (40 ml) was stirred at ambient temperature for 2 hours. Water (20 ml) was added to the reaction mixture. The mixture was adjusted to pH 8.5 with 5% aqueous potassium carbonate solution and stirred for 30 minutes. The organic layer was washed with brine and dried over magnesium sulfate. The solvent was evaporated in vacuo and the residue was chromatographed on silica gel eluting with ethyl acetate:n-hexane (5:5-7:3 v/v). The eluted fractions containing the desired product were collected and evaporated in vacuo. The residue was recrystallized from ethyl acetate and diisopropyl ether to give 5-nitro-1-(2-pyridinylmethyl)indoline (0.737 g).

¹H-NMR (DMSO-d₆):δ 3.10 (2H, t, J=8.40 Hz), 3.76 (2H, t, J=8.40 Hz), 4.64 (2H, s), 6.56 (1H, d, J=8.88 Hz), 7.31-7.38 (1H, m), 7.75-7.84 (2H, m), 7.96 (2H, dd, J=2.66 Hz, 8.88 Hz), 8.52 (1H, m)

### EXAMPLE 50

A mixture of 5-nitro-1-(2-pyridinylmethyl)indoline (383 mg), iron powder (450 mg) and ammonium chloride (51 mg) in ethanol (30 ml) and water (6 ml) was refluxed under stirring for 2.5 hours. After removal of the insoluble material, the solvent was evaporated in vacuo and the residue was dissolved in ethyl acetate and water. The organic layer was washed with brine and dried over magnesium sulfate. The solvent was evaporated in vacuo and the residue was dissolved in tetrahydrofuran (20 ml) and triethylamine (606 mg). To the above solution was added a solution of 4′-(trifluoromethyl)-1,1′-biphenyl-2-carbonyl chloride (427 mg) in tetrahydrofuran (10 ml) at ambient temperature and the mixture was stirred for 2 hours. The reaction mixture was poured into a mixture of ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate:n-hexane (6:4 v/v). The eluted fractions containing the desired product were collected and evaporated in vacuo. The residue was recrystallized from ethyl acetate and diisopropyl ether to give N-[1-(2-pyridinylmethyl)-2,3-dihydro-1H-indol-5-yl]-4′-(trifluoromethyl)-1,1′-biphenyl-2-carboxamide (347 mg).

¹H-NMR (DMSO-d₆):δ 2.88 (2H, t, J=8.06 Hz), 3.35 (2H, t, J=8.06 Hz), 4.33 (2H, s), 6.46 (1H, d, J=8.60 Hz), 7.06 (1H, dd, J=1.82 Hz, 8.37 Hz), 7.38 (1H, d, J=7.78 Hz), 7.24-7.30 (3H, m), 7.49-7.79 (8H, m), 8.53 (2H, d, J=4.76 Hz), 9.98 (1H, s)

Preparation 56

A solution of tert-butyl 5-nitro-1-indolinecarboxylate (793 mg) in methanol (40 ml) and tetrahydrofuran (10 ml) was hydrogenated over 10% palladium on carbon (0.3 g) under an atmospheric pressure of hydrogen at ambient temperature under stirring for 4 hours. After removal of the catalyst, the solvent was evaporated in vacuo and the residue was dissolved in tetrahydrofuran (30 ml) and triethylamine (606 mg). To the above solution was added a solution of 4′-(trifluoromethyl)-1,1′-biphenyl-2-carbonyl chloride (854 mg) in tetrahydrofuran (10 ml) at ambient temperature and the mixture was stirred for an hour. The reaction mixture was poured into a mixture of ethyl acetate and water. The organic layer was washed with 5% aqueous potassium carbonate solution and brine and dried over magnesium sulfate. The solvent was evaporated in vacuo and the residue was recrystallized from a mixture of ethyl acetate and diisopropyl ether to give tert-butyl 5-({[4′-(trifluoromethyl)-1,1′-biphenyl-2-yl]carbonyl}amino)-1-indolinecarboxylate (1.28 g).

¹H-NMR (DMSO-d₆):δ 1.49 (9H, s), 3.00 (2H, t, J=8.44 Hz), 3.88 (2H, t, J=8.44 Hz), 7.22 (1H, d, J=6.90 Hz), 7.41 (1H, s), 7.48-7.65 (8H, m), 7.76 (1H, d, J=8.36 Hz), 10.21 (1H, s)

Preparation 57

A solution of tert-butyl 5-({[(4′-(trifluoromethyl)-1,1′-biphenyl-2-yl]carbonyl}amino)-1-indolinecarboxylate (1.18 g) in trifluoroacetic acid (8 ml) was stirred at ambient temperature for 2 hours. The reaction mixture was poured into a mixture of ethyl acetate and water and adjusted to pH 8.5 with 5% aqueous potassium carbonate solution. The organic layer was washed with brine and dried over magnesium sulfate. The solvent was evaporated in vacuo and the residue was recrystallized from a mixture of ethyl acetate and diisopropyl ether to give N-(2,3-dihydro-1H-indol-5-yl)-4′-(trifluoromethyl)-1,1′-biphenyl-2-carboxamide (0.855 g).

¹H-NMR (DMSO-d₆):δ 2.84 (2H, t, J=8.30 Hz), 3.37 (2H, t, J=8.30 Hz), 5.32 (1H, s), 6.39 (1H, d, J=8.26 Hz), 6.98 (1H, dd, J=2.08 Hz, 8.26 Hz), 7.19 (1H, s), 7.49-7.65 (6H, m), 7.76 (2H, d, J=8.26 Hz), 9.89 (1H, s)

### EXAMPLE 51

A mixture of N-(2,3-dihydro-1H-indol-5-yl)-4′-(trifluoromethyl)-1,1′-biphenyl-2-carboxamide (574 mg), 3-(2-pyridyl)propionic acid (238 mg), 1-hydroxybenzotriazole hydrate (223 mg) and 1-[3-(dimethylamino)propyl]-3-ethylcarbodiimide hydrochloride (316 mg) in N,N-dimethylformamide (10 ml) was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:n-hexane (8:2-10:0 v/v). The eluted fractions containing the desired product were collected and evaporated in vacuo. The residue was dissolved in a mixture of ethyl acetate, hydrochloric acid and dioxane and evaporated in vacuo. The residue was dissolved in tetrahydrofuran and diisopropyl ether and the precipitate was collected by filtration to give N-{1-[3-(2-pyridinyl)propanoyl]-2,3-dihydro-1H-indol-5-yl}-4′-(trifluoromethyl)-1,1′-biphenyl-2-carboxamide hydrochloride (500 mg).

¹H-NMR (DMSO-d₆):δ 3.12-3.15 (2H, m), 3.29-3.32 (2H, m), 3.57-3.63 (2H, m), 4.10 (2H, t, J=8.42 Hz), 7.23 (1H, d, J=8.52 Hz), 7.46-7.89 (11H, m), 7.99 (1H, d, J=8.02 Hz), 8.40-8.48 (1H, m), 8.77 (1H, d, J=50.10 Hz), 10.27 (1H, s)

Preparation 58

A mixture of methyl 4-chlorobenzoylacetate (6.38 g) and N,N-dimethylformamide dimethylacetal (7.15 g) in toluene (30 ml) was stirred at ambient temperature for 15 hours. The reaction mixture was evaporated in vacuo and the residue was dissolved in water and ethyl acetate. The organic layer was washed with brine and dried over magnesium sulfate. The solvent was evaporated in vacuo and the residue was dissolved in n-butanol (60 ml). To the above solution were added acetamidine hydrochloride. (3.4 g) and triethylamine (4.55 g) and the mixture was refluxed under stirring for 2 hours. The reaction mixture was evaporated in vacuo and the residue was dissolved in a mixture ethyl acetate and water. The organic layer was washed with 5% aqueous potassium carbonate solution and brine and dried over magnesium sulfate. The solvent was evaporated in vacuo and the residue was chromatographed on silica gel eluting with ethyl acetate:n-hexane (2:8 v/v). The eluted fractions containing the desired product were collected and evaporated in vacuo. The residue was triturated with diisopropyl ether to give methyl 4-(4-chlorophenyl)-2-methyl-5-pyrimidinecarboxylate (4.2 g).

¹H-NMR (DMSO-d₆):δ 2.73 (3H, s), 3.74 (3H, s), 7.54-7.64 (4H, m), 9.05 (1H, s)

Preparation 59

A mixture of methyl 4-(4-chlorophenyl)-2-methyl-5-pyrimidinecarboxylate (4.72 g) and 4N aqueous sodium hydroxide solution (6 ml) in methanol (30 ml) and tetrahydrofuran (15 ml) was stirred at 40-50° C. for 30 minutes. The reaction mixture was evaporated in vacuo and the residue was dissolved in water and ethyl acetate. The aqueous layer was adjusted to pH 2.0 with 10% hydrochloric acid and extracted with ethyl acetate and tetrahydrofuran. The extract was washed with brine and dried over magnesium sulfate. The solvent was concentrated in vacuo and the precipitate was collected by filtration to give 4-(4-chlorophenyl)-2-methyl-5-pyrimidinecarboxylic acid (3.44 g).

¹H-NMR (DMSO-d₆):δ 2.72 (3H, s), 7.54-7.66 (4H, m), 9.03 (1H, s), 13.57 (1H, br s)

### EXAMPLE 52

4-(4-Chlorophenyl)-2-methyl-N-[1-(2-pyridinylacetyl)-2,3-dihydro-1H-indol-5-yl]-5-pyrimidinecarboxamide

The title compound was obtained in the same manner as in Example 6.

¹H-NMR (DMSO-d₆):δ 2.74 (3H, s), 3.14 (2H, t, J=8.160 Hz), 4.00 (2H, s), 4.21 (2H, t, J=8.16 Hz), 7.53-7.58 (3H, m), 7.24-7.38 (3H, m), 7.72-7.79 (3H, m), 7.97 (1H, d, J=8.66 Hz), 8.46 (1H, d, J=4.78 Hz), 8.90 (1H, s), 10.53 (1H, s)

Preparation 60

Methyl 2-methyl-4-[4-(trifluoromethyl)phenyl]-5-pyrimidinecarboxylate

The title compound was obtained in the same manner as in Preparation 58.

¹H-NMR (DMSO-d₆):δ 2.68 (3H, s), 3.74 (3H, s), 7.77-7.89 (4H, m), 9.12 (1H, s)

Preparation 61

2-Methyl-4-[4-(trifluoromethyl)phenyl]-5-pyrimidinecarboxylic acid

The title compound was obtained in the same manner as in Preparation 59.

¹H-NMR (DMSO-d₆):δ 2.74 (3H, s), 7.75-7.92 (4H, m), 9.10 (1H, s), 13.60 (1H, br s)

### EXAMPLE 53

2-Methyl-N-[1-(2-pyridinylacetyl)-2,3-dihydro-1H-indol-5-yl]-4-[4-(trifluoromethyl)phenyl]-5-pyrimidinecarboxamide

The title compound was obtained in the same manner as in Example 6.

¹H-NMR (DMSO-d₆):δ 2.77 (3H, s), 3.14 (2H, t, J=8.30 Hz), 4.00 (2H, s), 4.21 (2H, t, J=8.30 Hz), 7.24-7.38 (3H, m), 7.53 (1H, s), 7.72-8.00 (6H, m), 8.50 (1H, d, J=2.28 Hz), 8.97 (1H, s), 10.59 (1H, s)

Preparation 62

Methyl 4-(4-chlorophenyl)-5-pyrimidinecarboxylate

The title compound was obtained in the same manner as in Preparation 58.

¹H-NMR (DMSO-d₆):δ 3.76 (3H, s), 7.57-7.68 (4H, m), 9.16 (1H, s), 9.40 (1H, s)

Preparation 63

4-(4-Chlorophenyl)-5-pyrimidinecarboxylic acid

The title compound was obtained in the same manner as in Preparation 59.

¹H-NMR (DMSO-d₆):δ 7.58 (2H, d, J=6.60 Hz), 7.69 (2H, d, J=6.60 Hz), 9.14 (1H, s), 9.36 (1H, s)

### EXAMPLE 54

4-(4-Chlorophenyl)-N-[1-(2-pyridinylacetyl)-2,3-dihydro-1H-indol-5-yl]-5-pyrimidinecarboxamide

The title compound was obtained in the same manner as in Example 6.

¹H-NMR (DMSO-d₆):δ 3.15 (2H, t, J=8.30 Hz), 4.00 (2H, s), 4.22 (2H, t, J=8.30 Hz), 7.25-7.38 (2H, m), 7.56-7.61 (3H, m), 7.72-7.82 (3H, m), 7.98 (1H, d, J=8.66 Hz), 8.50 (1H, d, J=3.98 Hz), 8.02 (1H, s), 9.23 (1H, s), 10.62 (1H, s)

Preparation 64

To a mixture of 4-(trifluoromethyl)benzoyl chloride (8.34 g) and ethyl 3-(dimethylamino)acrylate (6.0 g) in tetrahydrofuran (50 ml) was added triethylamine (4.24 g) under ice-cooling and the mixture was stirred at ambient temperature for 2 hours and 55-60° C. for 2.5 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate:n-hexane (5:5-7:3 v/v). The eluted fractions containing the desired product were collected and evaporated in vacuo. The residue was triturated with diisopropyl ether to give ethyl (2E)-3-(dimethylamino)-2-[4-(trifluoromethyl)benzoyl]-2-propenoate (10.63 g).

Preparation 65

To a mixture of ethyl (2E)-3-(dimethylamino)-2-[4-(trifluoromethyl)benzoyl]-2-propenoate (7.0 g), isobutyramidine hydrochloride (3.27 g) and triethylamine (3.36 g) in n-butanol (50 ml) was refluxed under stirring for 4 hours. The reaction mixture was evaporated in vacuo and the residue was dissolved in a mixture of ethyl acetate and water. The organic layer was washed with 5% aqueous potassium carbonate solution and brine and dried over magnesium sulfate. The solvent was evaporated in vacuo and the residue was chromatographed on silica gel eluting with n-hexane:ethyl acetate (8:2-3:7 v/v). The eluted fractions containing the desired product were collected and evaporated in vacuo to give ethyl 2-isopropyl-4-[4-(trifluoromethyl)phenyl]-5-pyrimidinecarboxylate (4.1 g).

¹H-NMR (DMSO-d₆):δ 1.07 (3H, t, J=7.12 Hz), 1.35 (6H, d, J=6.88 Hz), 3.20-3.40 (1H, m), 4.20 (2H, q, J=7.12 Hz), 7.80-8.20 (4H, m), 9.17 (1H, s)

Preparation 66

2-Isopropyl-4-[4-(trifluoromethyl)phenyl]-5-pyrimidinecarboxylic acid

The title compound was obtained in the same manner as in Preparation 59.

¹H-NMR (DMSO-d₆):δ 3.33 (6H, d, J=6.88 Hz), 3.15-3.36 (1H, m), 7.82-7.97 (4H, m), 9.18 (1H, s), 13.60 (1H, br s)

### EXAMPLE 55

2-Isopropyl-N-[1-(2-pyridinylacetyl)-2,3-dihydro-1H-indol-5-yl]-4-[4-(trifluoromethyl)phenyl]-5-pyrimidinecarboxamide

The title compound was obtained in the same manner as in Example 6.

¹H-NMR (DMSO-d₆):δ 1.36 (6H, d, J=6.88 Hz), 3.10-3.39 (1H, m), 3.15 (2H, t, J=8.30 Hz), 4.05 (2H, s), 4.21 (2H, t, J=8.30 Hz), 7.24-7.31 (2H, m), 7.37 (1H, d, J=7.81 Hz), 7.51 (1H, s), 7.28-8.00 (7H, m), 8.48-8.50 (1H, m), 9.03 (1H, s), 10.63 (1H, s)

Preparation 67

A mixture of (3E)-4-[4-(trifluoromethyl)phenyl]-3-buten-2-one (6.43 g) and ethyl 3-aminocrotonate (4.65 g) in n-butanol (40 ml) was refluxed under stirring for 25 hours. The reaction mixture was evaporated in vacuo and the residue was dissolved in ethyl acetate and water. The organic layer was washed with 5% aqueous potassium carbonate solution and brine and dried over magnesium sulfate. The solvent was evaporated in vacuo and the residue was dissolved in ethyl acetate (100 ml). Manganese dioxide (45 g) was added to the above solution and the resultant mixture was refluxed under stirring for 1.5 hours. After removal of the insoluble material, the solvent was evaporated in vacuo and the residue was chromatographed on silica gel eluting with ethyl acetate:n-hexane (3:7 v/v). The eluted fractions containing the desired product were collected and evaporated in vacuo. The residue was triturated with diisopropyl ether to give ethyl 2,6-dimethyl-4-[4-(trifluoromethyl)phenyl]nicotinate (5.88 g).

¹H-NMR (DMSO-d₆):δ 0.92 (3H, t, J=70.10 Hz), 2.53 (6H, s), 4.07 (2H, q, J=70.10 Hz), 7.24 (1H, s), 7.52 (2H, d, J=80.10 Hz), 7.79 (2H, d, J=8.10 Hz)

Preparation 68

A mixture of ethyl 2,6-dimethyl-4-[4-(trifluoromethyl)phenyl]nicotinate (4.72 g) and sodium hydroxide (1.17 g) in methanol (30 ml), dioxane (30 ml) and water (30 ml) was refluxed under stirring for 25 hours. The reaction mixture was evaporated in vacuo and the residue was dissolved in water and ethyl acetate. The aqueous layer was adjusted to pH 2.0 with 10% hydrochloric acid and extracted with ethyl acetate. The extract was washed with brine and dried over magnesium sulfate. The solvent was concentrated in vacuo and the precipitate was collected by filtration to give 2,6-dimethyl-4-[4-(trifluoromethyl)phenyl]nicotinic acid (1.48 g).

¹H-NMR (DMSO-d₆):δ 2.52 (6H, s), 7.20 (1H, s), 7.64 (2H, d, J=80.10 Hz), 7.86 (2H, d, J=80.10 Hz), 13.37 (1H, br s)

### EXAMPLE 56

A mixture of 2,6-dimethyl-4-[4-(trifluoromethyl)phenyl]nicotinic acid (591 mg) and N,N-dimethylformamide (1.2 ml) and thionyl chloride (286 mg) in dichloromethane (10 ml) was stirred under ice-cooling for an hour. The resultant mixture was added to a solution of 1-(2-pyridinylacetyl)-5-indolinamine (418 mg) and triethylamine (808 mg) in N,N-dimethylformamide (15 ml) under ice-cooling and the mixture was stirred at ambient temperature for 7 hours. The reaction mixture was poured into a mixture of ethyl acetate and water. The organic layer was washed with brine and dried over magnesium sulfate. The solvent was evaporated in vacuo and the residue was recrystallized from ethyl acetate and diisopropyl ether to give 2,6-dimethyl-N-[1-(2-pyridinylacetyl)-2,3-dihydro-1H-indol-5-yl]-4-[4-(trifluoromethyl)phenyl]nicotinamide (174 mg).

¹H-NMR (DMSO-d₆):δ 2.52 (6H, s), 3.11 (2H, t, J=8.14 Hz), 3.99 (2H, s), 4.18 (2H, t, J=8.14 Hz), 7.13-7.42 (4H, m), 7.62-7.93 (7H, m), 8.49 (1H, d, J=3.98 Hz), 10.35 (1H, s)

### EXAMPLE 57

A mixture of [2-(formylamino)-1,3-thiazol-4-yl]acetic acid (921 mg) and phosphorus pentachloride (1.56 g) in dichloromethane (30 ml) was stirred at ambient temperature for 2.5 hour. The resultant mixture was evaporated in vacuo and the residue was dissolved in tetrahydrofuran (30 ml). This solution was added to a solution of N-(2,3-dihydro-1H-indol-5-yl)-4′-(trifluoromethyl)-1,1′-biphenyl-2-carboxamide (1.54 g) and triethylamine (1.61 g) in tetrahydrofuran (80 ml) under ice-cooling and the mixture was stirred at ambient temperature for 3 hours. The reaction mixture was poured into a mixture of ethyl acetate and water. The organic layer was washed with brine and dried over magnesium sulfate. The solvent was evaporated in vacuo and the residue was chromatographed on silica gel eluting with ethyl acetate:n-hexane (5:5-9:1 v/v). The eluted fractions containing the desired product were collected and evaporated in vacuo. The residue was recrystallized from ethyl acetate and diisopropyl ether to give N-(1-{[2-(formylamino)-1,3-thiazol-4-yl]acetyl}-2,3-dihydro-1H-indol-5-yl)-4′-(trifluoromethyl)-1,1′-biphenyl-2-carboxamide (0.66 g).

¹H-NMR (DMSO-d₆):δ 3.11 (2H, t, J=8.22 Hz), 3.85 (2H, s), 4.17 (2H, t, J=8.22 Hz), 7.03 (1H, s), 7.23 (1H, d, J=8.66 Hz), 7.33-7.65 (7H, m), 7.76 (1H, d, J=8.28 Hz), 7.94 (1H, d, J=8.66 Hz), 10.28 (1H, s), 12.24 (1H, s)

### EXAMPLE 58

A mixture of N-(1-{[2-(formylamino)-1,3-thiazol-4-yl]acetyl}-2,3-dihydro-1H-indol-5-yl)-4′-(trifluoromethyl)-1,1′-biphenyl-2-carboxamide (0.66 g) and conc. hydrochloride (0.4 ml) in methanol (20 ml) was stirred at ambient temperature for 3 hours. The reaction mixture was poured into a mixture of ethyl acetate and water and adjusted to pH 8.5 with aqueous potassium carbonate solution. The organic layer was washed with brine and dried over magnesium sulfate. The solvent was evaporated in vacuo and the residue was recrystallized from ethyl acetate to give N-{1-[(2-amino-1,3-thiazol-4-yl)acetyl]-2,3-dihydro-1H-indol-5-yl}-4′-(trifluoromethyl)-1,1′-biphenyl-2-carboxamide (507 mg).

¹H-NMR (DMSO-d₆):δ 3.09 (2H, t, J=8.34 Hz), 3.60 (2H, s), 4.17 (2H, t, J=8.34 Hz), 6.31 (1H, s), 6.88 (2H, s), 7.21 (1H, d, J=8.64 Hz), 7.49-7.64 (7H, m), 7.76 (1H, d, J=8.28 Hz), 7.92 (1H, d, J=8.66 Hz), 10.26 (1H, s)

### EXAMPLE 59

N-{1-[(2-Formylamino-1,3-thiazol-4-yl)acetyl]-2,3-dihydro-1H-indol-5-yl}-4′-chloro-1,1′-biphenyl-2-carboxamide

The title compound was obtained in the same manner as in Example 57.

¹H-NMR (DMSO-d₆):δ 3.11 (2H, t, J=8.40 Hz), 3.84 (2H, s), 4.17 (2H, t, J=8.40 Hz), 7.02 (1H, s), 7.23 (1H, d, J=8.60 Hz), 7.44-7.59 (9H, m), 7.92 (1H, d, J=8.66 Hz), 8.46 (1H, s), 10.21 (1H, s), 12.22 (1H, s)

### EXAMPLE 60

N-{1-[(2-Amino-1,3-thiazol-4-yl)acetyl]-2,3-dihydro-1H-indol-5-yl}-4′-chloro-1,1′-biphenyl-2-carboxamide

The title compound was obtained in the same manner as in Example 58.

¹H-NMR (DMSO-d₆):δ 3.09 (2H, t, J=8.24 Hz), 3.60 (2H, s), 4.17 (2H, t, J=8.24 Hz), 6.31 (1H, s), 6.89 (2H, s), 7.23 (1H, d, J=8.56 Hz), 7.44-7.59 (9H, m), 7.93 (1H, d, J=8.66 Hz), 10.21 (1H, s)

### EXAMPLE 61

A mixture of [2-(formylamino)-1,3-thiazol-4-yl]acetic acid (368 mg) and N,N-dimethylformamide (1.2 ml) and thionyl chloride (286 mg) in dichloromethane (10 ml) was stirred under ice-cooling for an hour. The resultant mixture was added to a solution of N-(2,3-dihydro-1H-indol-5-yl)-4′-methoxy-1,1′-biphenyl-2-carboxamide (418 mg) and triethylamine (808 mg) in dichloromethane (15 ml) under ice-cooling and the mixture was stirred at ambient temperature for 5 hours. The reaction mixture was poured into a mixture of ethyl acetate and water. The precipitate was collected by filtration and dried to give N-{1-[(2-formylamino-1,3-thiazol-4-yl)acetyl]-2,3-dihydro-1H-indol-5-yl}-4′-methoxy-1,1′-biphenyl-2-carboxamide (406 mg).

¹H-NMR (DMSO-d₆):δ 3.09 (2H, t, J=8.40 Hz), 3.74 (3H, s), 3.84 (2H, s), 4.17 (2H, t, J=8.40 Hz), 6.93 (1H, d, J=8.76 Hz), 7.03 (1H, s), 7.24 (1H, d, J=8.62 Hz), 7.36 (2H, d, J=8.76 Hz), 7.40-7.56 (5H, m), 7.92 (1H, d, J=8.66 Hz), 8.46 (1H, s), 10.14 (1H, s), 12.23 (1H, s)

### EXAMPLE 62

N-{1-[(2-Amino-1,3-thiazol-4-yl)acetyl]-2,3-dihydro-1H-indol-5-yl}-4′-methoxy-1,1′-biphenyl-2-carboxamide

The title compound was obtained in the same manner as in Example 58.

¹H-NMR (DMSO-d₆):δ 3.09 (2H, t, J=8.14 Hz), 3.60 (2H, s), 3.74 (3H, s), 4.17 (2H, t, J=8.14 Hz), 6.31 (1H, s), 6.89 (2H, s), 6.93 (2H, d, J=8.72 Hz), 7.37 (2H, d, J=8.72 Hz), 7.38-7.56 (5H, m), 7.92 (1H, d, J=8.68 Hz), 10.14 (1H, s)

### EXAMPLE 63

N-{1-[(2-Formylamino-1,3-thiazol-4-yl)acetyl]-2,3-dihydro-1H-indol-5-yl}-4′-methyl-1,1′-biphenyl-2-carboxamide

The title compound was obtained in the same manner as in Example 61.

¹H-NMR (DMSO-d₆):δ 2.29 (3H, s), 3.11 (2H, t, J=8.16 Hz), 3.84 (2H, s), 4.17 (2H, t, J=8.16 Hz), 7.03 (1H, s), 7.17 (2H, d, J=8.12 Hz), 7.32 (2H, d, J=80.10 Hz), 7.15-7.59 (6H, m), 7.92 (1H, d, J=8.70 Hz), 8.46 (1H, s), 10.16 (1H, s), 12.23 (1H, s)

### EXAMPLE 64

N-{1-[(2-Amino-1,3-thiazol-4-yl)acetyl]-2,3-dihydro-1H-indol-5-yl}-4′-methyl-1,1′-biphenyl-2-carboxamide

The title compound was obtained in the same manner as in Example 58.

¹H-NMR (DMSO-d₆):δ 2.29 (2H, s), 3.09 (2H, t, J=8.30 Hz), 3.60 (2H, s), 4.17 (2H, t, J=8.30 Hz), 6.31 (1H, s), 6.92 (2H, s), 7.17 (2H, d, J=8.00 Hz), 7.19-7.90 (8H, m), 7.92 (1H, d, J=8.66 Hz), 10.15 (1H, s)

### EXAMPLE 65

A mixture of N-(2,3-dihydro-1H-indol-5-yl)-3′-(trifluoromethyl)-1,1′-biphenyl-2-carboxamide (330 mg), [2-(formylamino)-1,3-thiazol-4-yl]acetic acid (167 mg), 1-hydroxybenzotriazole hydrate (128 mg) and 1-[3-(dimethylamino)propyl]-3-ethylcarbodiimide hydrochloride (182 mg) in N,N-dimethylformamide (10 ml) was stirred at ambient temperature for 15 hours. The reaction mixture was poured into a mixture of ethyl acetate and water and the organic layer was washed with brine and dried over magnesium sulfate. The solvent was evaporated in vacuo and the residue was dissolved in methanol (10 ml) and conc. hydrochloric acid (1 ml). The solution was stirred at ambient temperature for 5 hours. The reaction mixture was poured into a mixture of ethyl acetate and water and adjusted to pH 8.5 with aqueous potassium carbonate solution. The organic layer was washed with brine and dried over magnesium sulfate. The solvent was evaporated in vacuo and the residue was chromatographed on silica gel eluting with ethyl acetate:n-hexane (8:2-10:0 v/v). The eluted fractions containing the desired product were collected and evaporated in vacuo. The residue was recrystallized from ethyl acetate and diisopropyl ether to give N-{1-[(2-amino-1,3-thiazol-4-yl)acetyl]-2,3-dihydro-1H-indol-5-yl}-3′-(trifluoromethyl)-1,1′-biphenyl-2-carboxamide (230 mg).

¹H-NMR (DMSO-d₆):δ 3.08 (2H, t, J=8.30 Hz), 3.84 (2H, s), 4.17 (2H, t, J=8.30 Hz), 6.32 (1H, s), 6.96 (2H, s), 7.18 (1H, dd, J=1.68 Hz, 8.68 Hz), 7.43 (1H, s), 7.49-7.74 (8H, m), 7.92 (1H, d, J=8.68 Hz), 10.23 (1H, s)

Preparation 69

N-(1-Acetyl-2,3-dihydro-1H-indol-5-yl)-5-methyl-4′-(trifluoromethyl)-1,1′-biphenyl-2-carboxamide

The title compound was obtained in the same manner as in Preparation 47.

¹H-NMR (DMSO-d₆):δ 1.99 (3H, s), 2.42 (3H, s), 3.08 (2H, t, J=8.38 Hz), 4.08 (2H, t, J=8.38 Hz), 7.19 (1H, dd, J=1.78 Hz, 8.68 Hz), 7.34 (2H, d, J=8.76 Hz), 7.45-7.63 (4H, m), 7.74 (2H, d, J=8.30 Hz), 7.90 (1H, d, J=8.66 Hz), 10.16 (1H, s)

Preparation 70

N-(2,3-Dihydro-1H-indol-5-yl)-5-methyl-4′-(trifluoromethyl)-1,1′-biphenyl-2-carboxamide

The title compound was obtained in the same manner as in Preparation 46.

¹H-NMR (DMSO-d₆):δ 2.41 (3H, s), 2.84 (2H, t, J=8.28 Hz), 3.41 (2H, t, J=8.28 Hz), 5.31 (1H, s), 6.38 (1H, d, J=8.26 Hz), 6.98 (1H, dd, J=2.00 Hz, 8.26 Hz), 7.18 (1H, d, J=2.00 Hz), 7.32 (2H, d, J=8.36 Hz), 7.48 (1H, d, J=7.60 Hz), 7.61 (2H, d, J=80.10 Hz), 7.74 (1H, d, J=80.10 Hz), 9.80 (1H, s)

### EXAMPLE 66

A mixture of N-(2,3-dihydro-1H-indol-5-yl)-5-methyl-4′-(trifluoromethyl)-1,1′-biphenyl-2-carboxamide hydrochloride (991 mg), [2-(formylamino)-1,3-thiazol-4-yl]acetic acid (483 mg), 1-hydroxybenzotriazole hydrate (372 mg) and 1-[3-(dimethylamino)propyl]-3-ethylcarbodiimide hydrochloride (526 mg) in N,N-dimethylformamide (20 ml) was stirred at ambient temperature for 15 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate:n-hexane (6:4-9:1 v/v). The eluted fractions containing the desired product were collected and evaporated in vacuo. The residue was triturated with ethyl acetate and diisopropyl ether to give N-(1-{[2-(formylamino)-1,3-thiazol-4-yl]acetyl}-2,3-dihydro-1H-indol-5-yl)-5-methyl-4′-(trifluoromethyl)-1 μl-biphenyl-2-carboxamide (1.29 g).

¹H-NMR (DMSO-d₆):δ 2.42 (3H, s), 3.10 (2H, t, J=8.28 Hz), 3.84 (2H, s), 4.17 (2H, t, J=8.28 Hz), 7.02 (1H, s), 7.21 (1H, dd, J=1.68 Hz, 8.68 Hz), 7.35 (2H, d, J=8.74 Hz), 7.47-7.63 (4H, m), 7.74 (2H, d, J=8.32 Hz), 7.92 (1H, d, J=8.78 Hz), 8.46 (1H, s), 10.17 (1H, s), 12.21 (1H, s)

### EXAMPLE 67

N-{1-[(2-Amino-1,3-thiazol-4-yl)acetyl]-2,3-dihydro-1H-indol-5-yl}-5-methyl-4′-(trifluoromethyl)-1,1′-biphenyl-2-carboxamide

The title compound was obtained in the same manner as in Example 58.

¹H-NMR (DMSO-d₆):δ 2.42 (3H, s), 3.08 (2H, t, J=8.30 Hz), 3.59 (2H, s), 4.17 (2H, t, J=8.30 Hz), 6.31 (1H, s), 6.89 (2H, s), 7.20 (1H, dd, J=1.64 Hz, 8.72 Hz), 7.34 (2H, d, J=8.68 Hz), 7.46-7.62 (4H, m), 7.72 (2H, d, J=8.34 Hz), 7.92 (1H, d, J=8.68 Hz), 10.17 (1H, s)

Preparation 71

2-Isopropyl-N-(1-acetyl-2,3-dihydro-1H-indol-5-yl)-4-[4-(trifluoromethyl)phenyl]-5-pyrimidinecarboxamide

The title compound was obtained in the same manner as in Preparation 47.

¹H-NMR (DMSO-d₆):δ 1.37 (6H, d, J=6.90 Hz), 3.12 (2H, t, J=8.34 Hz), 3.22-3.56 (1H, m), 4.07 (2H, t, J=8.34 Hz), 7.27 (1H, d, J=8.63 Hz), 7.51 (1H, s), 7.87 (2H, d, J=8.46 Hz), 7.95-8.00 (3H, m), 9.02 (1H, s), 10.61 (1H, s)

Preparation 72

2-Isopropyl-N-(2,3-dihydro-1H-indol-5-yl)-4-[4-(trifluoromethyl)phenyl]-5-pyrimidinecarboxamide

The title compound was obtained in the same manner as in Preparation 46.

¹H-NMR (DMSO-d₆):δ 1.36 (6H, d, J=6.91 Hz), 2.89 (2H, t, J=8.24 Hz), 3.20-3.64 (3H, m), 5.73 (1H, br s), 6.46 (1H, d, J=8.28 Hz), 7.07 (1H, dd, J=1.50 Hz, 8.26 Hz), 7.28 (1H, d, J=1.50 Hz), 7.88 (2H, d, J=8.28 Hz), 7.97 (2H, d, J=8.28 Hz), 8.94 (1H, s), 10.28 (1H, s)

### EXAMPLE 68

2-Isopropyl-N-{1-[(2-formylamino-1,3-thiazol-4-yl)acetyl]-2,3-dihydro-1H-indol-5-yl}-4-[4-(trifluoromethyl)phenyl]-5-pyrimidinecarboxamide

The title compound was obtained in the same manner as in Example 57.

¹H-NMR (DMSO-d₆):δ 1.37 (6H, d, J=6.88 Hz), 3.14 (2H, t, J=8.30 Hz), 3.18-3.35 (1H, m), 3.86 (2H, s), 4.19 (2H, t, J=8.30 Hz), 7.03 (1H, s), 7.26 (1H, d, J=7.14 Hz), 7.87 (2H, d, J=8.46 Hz), 7.94-8.00 (3H, m), 8.46 (1H, s), 9.01 (1H, s), 10.21 (1H, s), 12.21 (1H, s)

### EXAMPLE 69

2-Isopropyl-N-{1-[(2-amino-1,3-thiazol-4-yl)acetyl]-2,3-dihydro-1H-indol-5-yl}-4-[4-(trifluoromethyl)phenyl]-5-pyrimidinecarboxamide

The title compound was obtained in the same manner as in Example 58.

¹H-NMR (DMSO-d₆):δ 1.36 (6H, d, J=6.86 Hz), 3.12 (2H, t, J=8.26 Hz), 3.21-3.33 (1H, m), 3.66 (2H, s), 4.19 (2H, t, J=8.26 Hz), 6.31 (1H, s), 6.56 (2H, s), 7.26 (1H, d, J=8.70 Hz), 7.53 (1H, s), 7.85-8.00 (5H, m), 8.46 (1H, s), 9.01 (1H, s), 10.60 (1H, s)

### EXAMPLE 70

To a mixture of 4′-ethoxy-1,1′-biphenyl-2-carboxylic acid (300 mg) and N,N-dimethylformamide (0.0048 ml) in toluene (3.0 ml) was added thionyl chloride (0.180 ml) dropwise under a nitrogen atmosphere and the solution was stirred for 2 hours at 100° C. The resultant mixture was cooled to ambient temperature, and then the solvent was evaporated in vacuo. The excess thionyl chloride was removed as the toluene azeotrope twice. The residue was dissolved in N,N-dimethylformamide (6.3 ml) and the solution was cooled to 5° C. on an ice bath under a nitrogen atmosphere. 1-(2-Pyridinylacetyl)-5-indolinamine (314 mg) was added portionwise to the above solution at 5° C., and then triethylamine (0.207 ml) was added dropwise. After the mixture was stirred at ambient temperature for 18 hours, the reaction mixture was poured into a mixture of ethyl acetate and tetrahydrofuran (1:1 v/v). The solution was washed with water three times and brine, dried over magnesium sulfate, and evaporated in vacuo. The crystallization of the residue was induced by scratching the flask. The resulting crystals were washed with diethyl ether-ethyl acetate (1:1) and methanol, and dried in vacuo to give 4′-ethoxy-N-[1-(2-pyridinylacetyl)-2,3-dihydro-1H-indol-5-yl]-1,1′-biphenyl-2-carboxamide (248 mg).

¹H-NMR (DMSO-d₆):δ 1.30 (3H, t, J=6.9 Hz), 3.07-3.15 (2H, m), 3.99 (1H, s), 4.00 (2H, q, J=6.9 Hz), 4.15-4.23 (2H, m), 6.91 (2H, d, J=8.6 Hz), 7.21-7.49 (10H, m), 7.76 (2H, td, J=7.6 Hz, 1.7 Hz), 7.91 (1H, d, J=8.7 Hz), 8.49 (1H, d, J=4.1 Hz), 10.12 (1H, s)

APCI-MS (m/z): 478 (M+H)⁺

### EXAMPLE 71

4′-(Benzyloxy)-N-[1-(2-pyridinylacetyl)-2,3-dihydro-1H-indol-5-yl]-1,1′-biphenyl-2-carboxamide

The title compound was obtained in the same manner as in Example 70.

¹H-NMR (DMSO-d₆):δ 3.07-3.16 (2H, m), 3.99 (1H, s), 4.15-4.23 (2H, m), 5.08 (2H, s), 7.01 (2H, d, J=8.7 Hz), 7.21-7.56 (15H, m), 7.76 (1H, dt, J_{d}=1.8 Hz, Jₜ=7.6 Hz), 7.92 (1H, d, J=8.7 Hz), 8.49 (1H, d, J=4.1 Hz), 10.15 (1H, s)

MS(m/z): 540 (M⁺+1)

Preparation 73

To a mixture of 4′-(trifluoromethyl)-1,1′-biphenyl-2-carboxylic acid (8.53 g) and N,N-dimethylformamide (0.124 ml) in toluene (85 ml) was added thionyl chloride (4.66 ml) dropwise under a nitrogen atmosphere and the solution was stirred for 2 hours at 100° C. The resultant mixture was cooled to ambient temperature, and then the solvent was evaporated in vacuo. The excess thionyl chloride was removed as the toluene azeotrope twice. The residue was dissolved in N,N-dimethylformamide (50 ml) and the solution was cooled to 5° C. on an ice bath under a nitrogen atmosphere. 4-Fluoro-3-nitroaniline (5.0 g) was added portionwise to the above solution at 5° C., and then triethylamine (8.93 ml) was added dropwise. After the solution was stirred at 5° C. for 30 minutes, the reaction mixture was diluted with ethyl acetate (200 ml). The solution was washed with water, 5% aq. KHSO₄, saturated aq. NaHCO₃ and brine, dried over magnesium sulfate, and then evaporated in vacuo. The resultant solid was washed with diethyl ether to give N-(4-fluoro-3-nitrophenyl)-4′-(trifluoromethyl)-1,1′-biphenyl-2-carboxamide (11.32 g).

¹H-NMR (DMSO-d₆):δ 7.53-7.78 (10H, m), 8.43-8.48 (1H, m), 10.84 (1H, s)

APCI-MS (m/z): 405 (M+H)⁺

Preparation 74

To a solution of N-(4-fluoro-3-nitrophenyl)-4′-(trifluoromethyl)-1,1′-biphenyl-2-carboxamide (300 mg) and triethylamine (0.362 ml) dissolved in N,N-dimethylformamide (6.0 ml) was added 2-pyridinylmethanamine (0.191 ml) under a nitrogen atmosphere and the mixture was stirred for 2 days. The resultant orange solution was poured into water and extracted with ethyl acetate. The extract was washed with water three times and brine, dried over magnesium sulfate, and evaporated in vacuo to give an orange solid. The solid was washed with diisopropyl ether to give N-{3-nitro-4-[(2-pyridinylmethyl)amino]phenyl}-4′-(trifluoromethyl)-1,1′-biphenyl-2-carboxamide (343 mg) as an orange solid.

¹H-NMR (DMSO-d₆):δ 4.70 (2H, d, J=5.4 Hz), 6.96 (1H, d, J=9.3 Hz), 7.28-7.40 (2H, m), 8.45 (1H, d, J=2.4 Hz), 8.57-8.59 (1H, m), 8.94 (1H, d, J=5.4 Hz), 10.38 (1H, s)

Preparation 75

To a solution of N-{3-nitro-4-[(2-pyridinylmethyl)amino]phenyl}-4′-(trifluoromethyl)-1,1′-biphenyl-2-carboxamide (330 mg) in methanol (6.6 ml) was added 10% palladium on carbon (50% wet, 66 mg). The mixture was stirred vigorously, and hydrogen gas was bubbled through the mixture for 1 hour. The catalyst was removed by filtration, washed with dichloromethane-methanol (9:1), and then the filtrate was evaporated in vacuo. The residue was chromatographed on silica gel eluting with dichloromethane:methanol (from dichloromethane only to 20:1). The eluate was concentrated in vacuo to give a yellow solid. The solid was washed with ethyl acetate, and then with diisopropyl ether to give N-{3-amino-4-[(2-pyridinylmethyl)amino]phenyl}-4′-(trifluoromethyl)-1,1′-biphenyl-2-carboxamide (172 mg).

¹H-NMR (DMSO-d₆):δ 4.34 (2H, d, J=5.7 Hz), 4.64 (2H, br s), 5.16 (1H, t, J=5.8 Hz), 6.17 (1H, d, J=8.5 Hz), 6.48 (1H, dd, J=2.1 Hz, 8.4 Hz), 6.91 (1H, d, J=2.1 Hz), 7.20-7.2-6 (1H, m), 7.34 (1H, d, J=7.8 Hz), 7.45-7.77 (9H, m), 8.50-8.53 (1H, m), 9.82 (1H, s)

APCI-MS (m/z): 463 (M+H)⁺

### EXAMPLE 72

A solution of N-{3-amino-4-[(2-pyridinylmethyl)amino]phenyl}-4′-(trifluoromethyl)-1,1′-biphenyl-2-carboxamide (155 mg) dissolved in 98% formic acid (3.1 ml) was refluxed for 1 hour. The reaction mixture was cooled to 5° C. on an ice bath and the pH of the reaction mixture was adjusted to 8.0 with 28% aq. NH₃. The solution was extracted with ethyl acetate and the extract was washed with water and brine, dried over magnesium sulfate, and evaporated in vacuo. The resultant solid was washed with ethyl acetate to give N-[1-(2-pyridinylmethyl)-1H-benzimidazol-5-yl]-4′-(trifluoromethyl)-1,1′-biphenyl-2-carboxamide (109 mg) as a white solid.

¹H-NMR (DMSO-d₆):δ 5.55 (2H, s), 7.22-7.39 (4H, m), 7.50-7.81 (9H, m), 7.89 (1H, d, J=1.6 Hz), 8.32 (1H, s), 8.51 (1H, d, J=4.8 Hz), 10.30 (1H, s)

APCI-MS (m/z): 473 (M+H)⁺

Preparation 76

N-(3-Nitro-4-{[2-(2-pyridinyl)ethyl]amino}phenyl)-4′-(trifluoromethyl)-1,1′-biphenyl-2-carboxamide

The title compound was obtained in the same manner as in Preparation 74.

¹H-NMR (DMSO-d₆):δ 3.06-3.23 (2H, m), 3.66-3.76 (2H, m), 7.09 (1H, d, J=9.4 Hz), 7.25 (1H, dd, J=4.9 Hz, 7.5 Hz), 7.35 (1H, d, J=7.8 Hz), 7.50-7.78 (10H, m), 8.29 (1H, t, J=5.5 Hz), 8.39 (1H, d, J=2.5 Hz), 8.53 (1H, d, J=4.0 Hz), 10.36 (1H, s)

APCI-MS (m/z): 507 (M⁺+H)+

Preparation 77

N-(3-Amino-4-{[2-(2-pyridinyl)ethyl]amino}phenyl)-4′-(trifluoromethyl)-1,1′-biphenyl-2-carboxamide

The title compound was obtained in the same manner as in Preparation 75.

¹H-NMR (DMSO-d₆):δ 4.34 (2H, d, J=5.7 Hz), 4.64 (2H, br s), 5.16 (1H, t, J=5.8 Hz), 6.39 (1H, d, J=5.8 Hz), 6.62 (1H, dd, J=2.2 Hz, 8.4 Hz), 6.88 (1H, d, J=2.2 Hz), 7.19-7.256 (1H, m), 7.32 (1H, d, J=7.8 Hz), 7.46-7.77 (9H, m), 8.51 (1H, d, J=4.7 Hz), 9.84 (1H, s)

APCI-MS (m/z): 477 (M+H)⁺

### EXAMPLE 73

A solution of N-(3-amino-4-{[2-(2-pyridinyl)ethyl]-amino}phenyl)-4′-(trifluoromethyl)-1,1′-biphenyl-2-carboxamide (231 mg) dissolved in 98% formic acid (4.6 ml) was refluxed for 1 hour. The reaction mixture was cooled to 5° C. on an ice bath and the pH of the reaction mixture was adjusted to 8.0 with 28% aq. NH₃. The solution was extracted with ethyl acetate and the extract was washed with water and brine, dried over magnesium sulfate, and evaporated in vacuo. To an ice cooled solution of the residue in ethyl acetate (5.0 ml) was added 4N HCl in dioxane (0.30 ml) dropwise at 5° C. under a nitrogen atmosphere. After the mixture was stirred at ambient temperature for 30 minutes, the resulting pink precipitate was collected by filtration and dried in vacuo to give N-{1-[2-(2-pyridinyl)ethyl]-1H-benzimidazol-5-yl}-4′-(trifluoromethyl)-1,1′-biphenyl-2-carboxamide dihydrochloride (201 mg).

¹H-NMR (DMSO-d₆):δ 3.63 (2H, t, J=7.0 Hz), 4.94 (2H, t, J=7.0 Hz), 7.53-7.78 (11H, m), 7.96 (1H, d, J=9.0 Hz), 8.22 (1H, t, J=7.7 Hz), 8.30 (1H, d, J=1.4 Hz), 8.70 (1H, d, J=4.7 Hz), 9.60 (1H, s), 10.82 (1H, s)

APCI-MS (m/z): 487 (M+H)⁺

Preparation 78

N-(3-Nitro-4-{[3-(2-pyridinyl)propyl]amino}phenyl)-4′-(trifluoromethyl)-1,1′-biphenyl-2-carboxamide

The title compound was obtained in the same manner as in Preparation 74.

¹H-NMR (DMSO-d₆):δ 1.94-2.09 (2H, m), 2.79-2.86 (2H, m), 3.34-3.44 (2H, m), 7.03 (1H, d, J=9.4 Hz), 7.16-7.23 (1H, m), 7.27 (1H, d, J=7.8 Hz), 7.50-7.79 (10H, m), 8.11-8.18 (1H, m), 8.39 (1H, d, J=2.5 Hz), 8.46-8.48 (1H, m), 10.35 (1H, s)

APCI-MS (m/z): 521 (M+H)⁺

Preparation 79

N-(3-Amino-4-{[3-(2-pyridinyl)propyl]amino}phenyl)-41-(trifluoromethyl)-1,1′-biphenyl-2-carboxamide

The title compound was obtained in the same manner as in Preparation 75.

¹H-NMR (DMSO-d₆):δ 1.88-2.02 (2H, m), 2.80-2.88 (2H, m), 2.95-3.04 (2H, m), 4.28-4.32 (1H, m), 4.56 (2H, br s), 6.26 (1H, d, J=8.5 Hz), 6.58 (1H, dd, J=2.2 Hz, 8.3 Hz), 6.85 (1H, d, J=2.2 Hz), 7.16-7.23 (1H, m), 7.27 (1H, d, J=7.8 Hz), 7.46-7.77 (9H, m), 8.48 (1H, d, J=4.0 Hz), 9.81 (1H, s)

APCI-MS (m/z): 491 (M+H)⁺

### EXAMPLE 74

N-{1-[3-(2-Pyridinyl)propyl]-1H-benzimidazol-5-yl}-4′-(trifluoromethyl)-1,1′-biphenyl-2-carboxamide

The title compound was obtained in the same manner as in Example 72.

¹H-NMR (DMSO-d₆):δ 2.12-2.27 (2H, m), 2.66-2.74 (2H, m), 4.27 (2H, t, J=6.9 Hz), 7.17-7.25 (2H, m), 7.33 (1H, dd, J=8.7 Hz, 1.7 Hz), 7.46-7.78 (10H, m), 7.89 (1H, d, J=1.6 Hz), 8.20 (1H, s), 8.47 (1H, d, J=4.8 Hz), 10.30 (1H, s)

APCI-MS (m/z): 501 (M+H)⁺

Preparation 80

A solution of 7-nitro-3-oxo-3,4-dihydro-2H-1,4-benzoxazine (1.94 g) in methanol (50 ml) and tetrahydrofuran (40 ml) was hydrogenated over 10% palladium on carbon (0.6 g) under an atmospheric pressure of hydrogen at ambient temperature under stirring for 8 hours. After removal of the catalyst, the solvent was evaporated in vacuo and the residue was dissolved in tetrahydrofuran (60 ml) and triethylamine (2.02 g). To the above solution was added a solution of 4′-(trifluoromethyl)-1,1′-biphenyl-2-carbonyl chloride (2.85 g) in tetrahydrofuran (10 ml) at ambient temperature and the mixture was stirred for 5 hours. The reaction mixture was poured into a mixture of ethyl acetate and water. The organic layer was washed with 5% aqueous potassium carbonate solution and brine and dried over magnesium sulfate. The solvent was concentrated in vacuo and the precipitate was collected by filtration to give N-(3-oxo-3,4-dihydro-2H-1,4-benzoxazin-7-yl)-4′-(trifluoromethyl)-1,1′-biphenyl-2-carboxamide (3.357 g).

¹H-NMR (DMSO-d₆):δ 4.52 (2H, s), 6.78 (1H, d, J=8.46 Hz), 7.09 (1H, dd, J=1.98 Hz, 8.46 Hz), 7.22 (1H, d, J=1.98 Hz), 7.48-7.64 (6H, m), 7.76 (2H, d, J=8.34 Hz), 10.27 (1H, s), 10.64 (1H, s)

Preparation 81

A solution of N-(3-oxo-3,4-dihydro-2H-1,4-benzoxazin-7-yl)-4′-(trifluoromethyl)-1,1′-biphenyl-2-carboxamide (2.89 g) in tetrahydrofuran (50 ml) was added portionwise to a mixture of lithium aluminum hydride (0.53 g) in tetrahydrofuran (50 ml) at 55-60° C. under stirring. The mixture was stirred at 55-60° C. under an atmospheric pressure of nitrogen for 1.5 hours. A mixture of ethyl acetate and water was added to the reaction mixture under ice-cooling and the mixture was extracted with ethyl acetate. The extract was washed with brine and dried over magnesium sulfate. The solvent was evaporated in vacuo and the residue was recrystallized from ethyl acetate and diisopropyl ether to give N-(3,4-dihydro-2H-1,4-benzoxazin-7-yl)-4′-(trifluoromethyl)-1,1′-biphenyl-2-carboxamide (2.09 g).

¹H-NMR (DMSO-d₆):δ 3.22 (2H, br s), 4.06-4.10 (2H, m), 5.56 (1H, s), 6.44 (1H, d, J=8.45 Hz), 6.79 (1H, dd, J=2.25 Hz, 8.45 Hz), 6.91 (1H, d, J=2.25 Hz), 7.45-7.64 (6H, m), 7.76 (2H, d, J=8.36 Hz), 9.94 (1H, s)

### EXAMPLE 75

A mixture of N-(3,4-dihydro-2H-1,4-benzoxazin-7-yl)-4′-(trifluoromethyl)-1,1′-biphenyl-2-carboxamide (996 mg) and 2-vinylpyridine (315 mg) and acetic acid (150 mg) in 2-methoxyethanol (4 ml) was stirred at 155-160° C. for 6 hours. The reaction mixture was poured into a mixture of ethyl acetate and water. The mixture was adjusted to pH 9.0 with 20% aqueous potassium carbonate solution and the organic layer was washed with brine and dried over magnesium sulfate. The solvent was concentrated in vacuo and the residue was chromatographed on silica gel eluting with ethyl acetate:n-hexane (7:3 v/v). The eluted fractions containing the desired product were collected and evaporated in vacuo. The residue was recrystallized from ethyl acetate and diisopropyl ether to give N-{4-[2-(2-pyridinyl)ethyl]-3,4-dihydro-2H-1,4-benzoxazin-7-yl}-4′-(trifluoromethyl)-1,1′-biphenyl-2-carboxamide (0.76 g).

¹H-NMR (DMSO-d₆):δ 2.94 (2H, t, J=7.80 Hz), 3.20-3.24 (2H, m), 3.58 (2H, t, J=7.80 Hz), 4.18-4.12 (2H, m), 6.67 (1H, d, J=8.48 Hz), 6.90-6.97 (2H, m), 7.19-7.25 (1H, m), 7.21 (1H, d, J=7.76 Hz), 7.47-7.70 (7H, m), 7.77 (2H, d, J=8.38 Hz), 8.51 (1H, d, J=4.18 Hz), 10.01 (1H, s)

APCI-MS (m/z): 503 (M+)

### EXAMPLE 76

A mixture of N-(3,4-dihydro-2H-1,4-benzoxazin-7-yl)-4′-(trifluoromethyl)-1,1′-biphenyl-2-carboxamide (797 mg), 2-pyridylacetic acid hydrochloride (417 mg), 1-hydroxybenzotriazole hydrate (356 mg) and 1-[3-(dimethylamino)propyl]-3-ethylcarbodiimide (411 mg) in N,N-dimethylformamide (15 ml) was stirred at ambient temperature for 15 hours. The reaction mixture was poured into a mixture of ethyl acetate and water and adjusted to pH 8.0 with aqueous potassium carbonate solution. The organic layer was washed with brine and dried over magnesium sulfate. The solvent was evaporated in vacuo and the residue was chromatographed on silica gel eluting with ethyl acetate:n-hexane (6:4-9:1 v/v). The eluted fractions containing the desired product were collected and evaporated in vacuo. The residue was recrystallized from a mixture of ethyl acetate and diisopropyl ether to give N-[4-(2-pyridinylacetyl)-3,4-dihydro-2H-1,4-benzoxazin-7-yl]-4′-(trifluoromethyl)-1,1′-biphenyl-2-carboxamide (120 mg).

¹H-NMR (DMSO-d₆):δ 3.89-3.91 (2H, m), 4.11 (2H, s), 4.22-4.24 (2H, m), 6.98 (1H, d, J=8.88 Hz), 7.20-7.31 (3H, m), 7.49-7.71 (8H, m), 7.76 (2H, d, J=8.14 Hz), 8.48 (1H, d, J=4.42 Hz), 10.34 (1H, s)

### EXAMPLE 77

A mixture of N-(3,4-dihydro-2H-1,4-benzoxazin-7-yl)-4′-(trifluoromethyl)-1,1′-biphenyl-2-carboxamide (598 mg), 2-pyridinecarboxaldehyde (321 mg) and sodium triacetoxyborohydride (954 mg) in dichloromethane (30 ml) was stirred at ambient temperature for 15 hours. Water (20 ml) was added to the reaction mixture. The mixture was adjusted to pH 8.5 with 5% aqueous potassium carbonate solution and stirred for 30 minutes. The organic layer was washed with brine and dried over magnesium sulfate. The solvent was evaporated in vacuo and the residue was chromatographed on silica gel eluting with ethyl acetate:n-hexane (7:3 v/v). The eluted fractions containing the desired product were collected and evaporated in vacuo. The residue was recrystallized from ethyl acetate and diisopropyl ether to give N-[4-(2-pyridinylmethyl)-3,4-dihydro-2H-1,4-benzoxazin-7-yl]-4′-(trifluoromethyl)-1,1′-biphenyl-2-carboxamide (446 mg).

¹H-NMR (DMSO-d₆):δ 3.45-3.49 (2H, m), 4.19-4.23 (2H, m), 4.51 (2H, s), 6.48 (1H, d, J=8.74 Hz), 6.79 (1H, dd, J=2.31 Hz, 8.70 Hz), 6.98 (1H, d, J=2.28 Hz), 7.23-7.32 (2H, m), 7.46-7.70 (7H, m), 7.75 (2H, d, J=7.97 Hz), 8.53 (2H, d, J=4.23 Hz), 10.00 (1H, s)

APCI-MS (m/z): 489 (M+)

Preparation 82

A mixture of ethyl 3-(2-chloro-5-nitrophenyl)acrylate (2.56 g), 2-(2-aminoethyl)pyridine (1.83 g) and triethylamine (2.02 g) in N,N-dimethylimidazole (10 ml) was stirred at 85-90° C. for 4 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate:n-hexane (6:4 v/v). The eluted fractions containing the desired product were collected and evaporated in vacuo to give ethyl 3-(5-nitro-2-{[2-(2-pyridinyl)ethyl]amino}phenyl)acrylate (3.8 g) as a yellow oil.

¹H-NMR (DMSO-d₆):δ 1.28 (3H, t, J=7.10 Hz), 3.06 (2H, t, J=7.58 Hz), 3.52-3.69 (2H, m), 4.25 (2H, q, J=70.10 Hz), 6.57 (1H, d, J=15.60 Hz), 6.84 (1H, d, J=9.37 Hz), 7.17-7.34 (1H, m), 7.46-7.52 (1H, m), 7.64-7.77 (1H, m), 8.03-8.28 (1H, m), 8.42 (1H, d, J=2.67 Hz), 8.54 (1H, d, J=4.16 Hz)

Preparation 83

A solution of ethyl 3-(5-nitro-2-{[2-(2-pyridinyl)ethyl]amino}phenyl)acrylate (3.80 g) in methanol (50 ml) was hydrogenated over 10% palladium on carbon (0.3 g) under an atmospheric pressure of hydrogen at ambient temperature under stirring for 4 hours. After removal of the catalyst, the solvent was evaporated in vacuo. The residue was dissolved in ethanol (40 ml) and the solution was refluxed under stirring for 3 hours. The reaction mixture was evaporated in vacuo and the residue was chromatographed on silica gel eluting with ethyl acetate:n-hexane (7:3-10:0 v/v). The eluted fractions containing the desired product were collected and evaporated in vacuo to give 6-amino-1-[2-(2-pyridinyl)ethyl]-3,4-dihydro-2(1H)-quinolinone (1.10 g).

¹H-NMR (DMSO-d₆):δ 2.39-2.43 (2H, m), 2.57-2.63 (2H, m), 3.28-3.03 (2H, m), 4.05-4.18 (2H, m), 5.17 (2H, s), 6.45-6.55 (2H, m), 6.88 (1H, d, J=8.25 Hz), 7.19-7.29 (2H, m), 7.65-7.73 (1H, m), 8.51 (1H, d, J=4.55 Hz)

### EXAMPLE 78

A solution of 4′-(trifluoromethyl)-1,1′-biphenyl-2-carbonyl chloride (1.17 g) in tetrahydrofuran (10 ml) was added to a mixture of 6-amino-1-[2-(2-pyridinyl)ethyl]-3,4-dihydro-2(1H)-quinolinone (1.10 g) and triethylamine (0.83 g) in tetrahydrofuran (40 ml) at ambient temperature and the mixture was stirred for 5 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate:n-hexane (7:3-9:1 v/v). The eluted fractions containing the desired product were collected and evaporated in vacuo. The residue was triturated in diisopropyl ether to give N-{2-oxo-1-[2-(2-pyridinyl)ethyl]-1,2,3,4-tetrahydro-6-quinolinyl}-4′-(trifluoromethyl)-1,1′-biphenyl-2-carboxamide (1.45 g).

¹H-NMR (DMSO-d₆):δ 2.48-2.51 (2H, m), 2.72-2.79 (2H, m), 2.93-3.00 (2H, m), 4.16-4.23 (2H, m), 7.12 (1H, d, J=8.78 Hz), 7.15-7.76 (11H, m), 7.78 (2H, d, J=8.34 Hz), 8.501 (1H, d, J=4.46 Hz), 10.34 (1H, s)

### EXAMPLE 79

A solution of N-{2-oxo-1-[2-(2-pyridinyl)ethyl]-1,2,3,4-tetrahydro-6-quinolinyl}-4′-(trifluoromethyl)-1,1′-biphenyl-2-carboxamide (1.17 g) in tetrahydrofuran (0.40 ml) was added dropwise to a mixture of lithium aluminum hydride (173 mg) in tetrahydrofuran (30 ml) at 55-60° C. under stirring. The mixture was stirred at 55-60° C. under an atmospheric pressure of nitrogen for 1.5 hours. A mixture of ethyl acetate and water was added to the reaction mixture under ice-cooling and the mixture was extracted with ethyl acetate. The extract was washed with brine and dried over magnesium sulfate. The solvent was evaporated in vacuo and the residue was chromatographed on silica gel eluting with ethyl acetate:n-hexane (7:3-9:1 v/v). The eluted fractions containing the desired product were collected and evaporated in vacuo. The residue was recrystallized from ethyl acetate and diisopropyl ether to give N-{1-[2-(2-pyridinyl)ethyl]-1,2,3,4-tetrahydro-6-quinolinyl}-4′-(trifluoromethyl)-1,1′-biphenyl-2-carboxamide (628 mg).

¹H-NMR (DMSO-d₆):δ 1.75-1.81 (2H, m), 2.56-2.62 (2H, m), 2.92 (2H, t, J=7.70 Hz), 3.18 (2H, m), 3.56 (2H, t, J=7.70 Hz), 6.58 (1H, d, J=8.59 Hz), 7.07 (1H, s), 7.12-7.30 (3H, m), 7.46-7.73 (7H, m), 7.77 (2H, d, J=8.31 Hz), 8.52 (1H, d, J=4.08 Hz), 9.91 (1H, s)

Preparation 84

A solution of ethyl chlorooxoacetate (410 mg) in tetrahydrofuran (5 ml) was added to a mixture of N-(3-nitro-4-{[2-(2-pyridinyl)ethyl]amino}phenyl)-4′-(trifluoromethyl)-1,1′-biphenyl-2-carboxamide (1.013 g) and triethylamine (404 mg) in tetrahydrofuran (40 ml) and the resultant mixture was stirred at ambient temperature for 6 hours. The reaction mixture was poured into a mixture of ethyl acetate and water. The organic layer was washed with brine and dried over magnesium sulfate. The solvent was evaporated in vacuo to give ethyl {[2-nitro-4-({[4′-(trifluoromethyl)-1,1′-biphenyl-2-yl]carbonyl}amino)phenyl][2-(2-pyridinyl)ethyl]amino}(oxo)acetate (1.23 g).

¹H-NMR (DMSO-d₆):δ 0.85 (2H, t, J=7.09 Hz), 2.99-3.07 (2H, m), 3.91 (2H, q, J=7.09 Hz), 4.25-4.38 (2H, m), 7.17-7.42 (2H, m), 7.41-7.91 (2H, m), 7.59-7.92 (9H, m), 8.40-8.48 (2H, m), 10.98 (1H, s)

### EXAMPLE 80

A mixture of ethyl {[2-nitro-4-({[4′-(trifluoromethyl)-1,1′-biphenyl-2-yl]carbonyl}amino)phenyl][2-(2-pyridinyl)ethyl]amino}(oxo)acetate (1.21 g), iron powder (600 mg) and ammonium chloride (68 mg) in ethanol (40 ml) and water (8 ml) was refluxed under stirring for 3 hours. After removal of the insoluble material, the solvent was evaporated in vacuo and the residue was dissolved in ethyl acetate and water. The organic layer was washed with brine and dried over magnesium sulfate. The solvent was evaporated in vacuo and the residue was recrystallized from ethyl acetate and diisopropyl ether to give N-{2,3-dioxo-1-[2-(2-pyridinyl)ethyl]-1,2,3,4-tetrahydro-6-quinoxalinyl}-4′-(trifluoromethyl)-1,1′-biphenyl-2-carboxamide (714 mg).

¹H-NMR (DMSO-d₆):δ 3.05 (2H, t, J=7.98 Hz), 4.42 (2H, t, J=7.98 Hz), 7.21-7.36 (4H, m), 7.50-7.79 (10H, m), 8.52 (1H, d, J=4.72 Hz), 10.55 (1H, s), 11.99 (1H, s)

### EXAMPLE 81

N-{1-[2-(2-Pyridinyl)ethyl]-1,2,3,4-tetrahydro-6-quinoxalinyl}-4′-(trifluoromethyl)-1,1′-biphenyl-2-carboxamide

The title compound was obtained in the same manner as in Example 79.

¹H-NMR (DMSO-d₆):δ 2.93 (2H, t, J=7.91 Hz), 3.17 (4H, br s), 3.51 (2H, t, J=7.91 Hz), 5.21 (1H, s), 6.45 (1H, d, J=8.60 Hz), 6.57 (1H, dd, J=2.11 Hz, 8.52 Hz), 6.79 (1H, d, J=2.48 Hz), 7.18-7.24 (1H, m), 7.30 (1H, d, J=7.75 Hz), 7.46-7.70 (4H, m), 7.76 (2H, d, J=8.40 Hz), 8.52 (1H, d, J=4.05 Hz), 9.88 (1H, s)

Preparation 85

A mixture of N-(4-fluoro-3-nitrophenyl)-4′-(trifluoromethyl)-1,1′-biphenyl-2-carboxamide (4.04 g) and glycine ethyl ester hydrochloride (2.1 g) and triethylamine (3.03 g) in N,N-dimethylformamide (30 ml) was stirred at 85-90° C. for 4 hours. The reaction mixture was poured into a mixture of ethyl acetate and water and the organic layer was washed with brine and dried over magnesium sulfate. The solvent was evaporated in vacuo and the residue was chromatographed on silica gel eluting with ethyl acetate:n-hexane (1:1 v/v). The eluted fractions containing the desired product were collected and evaporated in vacuo. The residue was recrystallized from aqueous ethanol to give ethyl [2-nitro-4-({[4′-(trifluoromethyl)-1,1′-biphenyl-2-yl]carbonyl}amino)anilino]acetate (3.98 g).

¹H-NMR (DMSO-d₆):δ 1.22 (3H, t, J=7.12 Hz), 4.62 (2H, q, J=7.12 Hz), 4.25 (2H, d, J=5.76 Hz), 6.91 (1H, d, J=9.30 Hz), 7.51-8.09 (9H, m), 8.31 (1H, t, J=5.76 Hz), 8.43 (1H, d, J=2.38 Hz), 10.40 (1H, s)

Preparation 86

A mixture of ethyl [2-nitro-4-({[4′-(trifluoromethyl)-1,1′-biphenyl-2-yl]carbonyl}amino)anilino]acetate (3.9 g), iron powder (2.4 g) and ammonium chloride (272 mg) in ethanol (80 ml) and water (16 ml) was refluxed under stirring for 3 hours. After removal of the insoluble material, the solvent was evaporated in vacuo and the residue was dissolved in ethyl acetate and water. The organic layer was washed with brine and dried over magnesium sulfate. The solvent was evaporated in vacuo and the residue was chromatographed on silica gel eluting with ethyl acetate:n-hexane (6:4-8:2 v/v). The eluted fractions containing the desired product were collected and concentrated in vacuo. The precipitate was collected by filtration to give N-(3-oxo-1,2,3,4-tetrahydro-6-quinoxalinyl)-4′-(trifluoromethyl)-1,1′-biphenyl-2-carboxamide (1.62 g).

¹H-NMR (DMSO-d₆):δ 3.66 (2H, s), 5.81 (1H, s), 6.55 (1H, d, J=8.40 Hz), 6.81 (1H, dd, J=2.16 Hz, 8.40 Hz), 7.15 (1H, d, J=2.16 Hz), 7.46-7.65 (6H, m), 7.86 (2H, d, J=8.38 Hz), 10.08 (1H, s), 10.22 (1H, s)

### EXAMPLE 82

A mixture of N-(3-oxo-1,2,3,4-tetrahydro-6-quinoxalinyl)-4′-(trifluoromethyl)-1,1′-biphenyl-2-carboxamide (620 mg), 2-pyridylacetic acid hydrochloride (313 mg), 1-hydroxybenzotriazole hydrate (255 mg) and 1-[3-(dimethylamino)propyl]-3-ethylcarbodiimide (294 mg) in tetrahydrofuran (20 ml) was stirred at ambient temperature for 15 hours. The reaction mixture was poured into a mixture of ethyl acetate and water and adjusted to pH 8.0 with aqueous potassium carbonate solution. The organic layer was washed with brine and dried over magnesium sulfate. The solvent was evaporated in vacuo and the residue was chromatographed on silica gel eluting with ethyl acetate:n-hexane (8:2-10:0 v/v). The eluted fractions containing the desired product were collected and evaporated in vacuo. The residue was recrystallized from a mixture of ethyl acetate and diisopropyl ether to give N-[3-oxo-1-(2-pyridinylacetyl)-1,2,3,4-tetrahydro-6-quinoxalinyl]-4′-(trifluoromethyl)-1,1′-biphenyl-2-carboxamide (114 mg).

¹H-NMR (DMSO-d₆):δ 4.21 (2H, m), 4.34 (2H, s), 7.08-7.12 (1H, m), 7.32-7.39 (2H, m), 7.51-7.87 (10H, m), 8.51 (1H, d, J=4.34 Hz), 10.54 (1H, s), 10.68 (1H, s)

APCI-MS (m/z): 531 (M+H)+

### EXAMPLE 83

A mixture of N-(3-oxo-1,2,3,4-tetrahydro-6-quinoxalinyl)-4′-(trifluoromethyl)-1,1′-biphenyl-2-carboxamide (890 mg) and 2-vinylpyridine (318 mg) and acetic acid (130 mg) in ethanol (15 ml) was refluxed under stirring for 6 hours. The reaction mixture was poured into a mixture of ethyl acetate and water. The mixture was adjusted to pH 9.0 with 20% aqueous potassium carbonate solution and the organic layer was washed with brine and dried over magnesium sulfate. The solvent was evaporated in vacuo and the residue was chromatographed on silica gel eluting with ethyl acetate:n-hexane (7:3-10:0 v/v). The eluted fractions containing the desired product were collected and evaporated in vacuo. The residue was triturated with diisopropyl ether to give N-{3-oxo-1-[2-(2-pyridinyl)ethyl]-1,2,3,4-tetrahydro-6-quinoxalinyl}-4′-(trifluoromethyl)-1,1′-biphenyl-2-carboxamide (110 mg).

¹H-NMR (DMSO-d₆):δ 3.00 (2H, t, J=8.22 Hz), 3.55 (2H, t, J=8.22 Hz), 3.71 (2H, s), 6.73 (1H, d, J=8.74 Hz), 6.94-6.96 (1H, m), 7.23-7.36 (3H, m), 7.52-7.78 (9H, m), 8.53 (1H, d, J=3.98 Hz), 10.17 (1H, s), 10.38 (1H, s)

### EXAMPLE 84

A mixture of N-(3-amino-4-{[2-(2-pyridinyl)ethyl]-amino}phenyl)-4′-(trifluoromethyl)-1,1′-biphenyl-2-carboxamide (953 mg) and cyanogen bromide (233 mg) in ethanol (30 ml) and tetrahydrofuran (15 ml) was stirred at ambient temperature for 8 hours. The reaction mixture was poured into a mixture of ethyl acetate and water and adjusted to pH 8.5 with aqueous potassium carbonate solution. The organic layer was washed with brine and dried over magnesium sulfate. The solvent was evaporated in vacuo and the residue was chromatographed on silica gel eluting with chloroform:methanol (96:4-92:8 v/v). The eluted fractions containing the desired product were collected and evaporated in vacuo. The residue was recrystallized from a mixture of ethyl acetate and diisopropyl ether to give N-{2-amino-1-[2-(2-pyridinyl)ethyl]-1H-benzimidazol-5-yl}-4′-(trifluoromethyl)-1,1′-bipenyl-2-carboxamide (0.58 g).

¹H-NMR (DMSO-d₆):δ 3.08 (2H, t, J=7.16 Hz), 4.31 (2H, t, J=7.16 Hz), 6.42 (2H, s), 6.84-6.96 (2H, m), 7.20-7.35 (2H, m), 7.48 (1H, s), 7.52-7.78 (9H, m), 8.53 (1H, d, J=4.58 Hz), 10.05 (1H, s)

### EXAMPLE 85

A mixture of N-(3,4-dihydro-2H-1,4-benzoxazin-7-yl)-4′-(trifluoromethyl)-1,1′-biphenyl-2-carboxamide (797 mg), [2-(formylamino)-1,3-thiazol-4-yl]acetic acid (386 mg), 1-hydroxybenzotriazole hydrate (298 mg) and 1-[3-(dimethylamino)propyl]-3-ethylcarbodiimide hydrochloride (422 mg) in N,N-dimethylformamide (15 ml) was stirred at ambient temperature for 15 hours. The reaction mixture was poured into a mixture of ethyl acetate and water and the organic layer was washed with brine and dried over magnesium sulfate. The solvent was evaporated in vacuo and the residue was dissolved in methanol (10 ml) and conc. hydrochloric acid (1 ml). The mixture was stirred at ambient temperature for 5 hours. The reaction mixture was poured into a mixture of ethyl acetate and water and adjusted to pH 8.5 with aqueous potassium carbonate solution. The organic layer was washed with brine and dried over magnesium sulfate. The solvent was evaporated in vacuo and the residue was chromatographed on silica gel eluting with ethyl acetate:n-hexane (8:2-10:0 v/v). The eluted fractions containing the desired product were collected and evaporated in vacuo. The residue was recrystallized from a mixture of ethyl acetate and diisopropyl ether to give N-{4-[(2-amino-1,3-thiazol-4-yl)acetyl]-3,4-dihydro-2H-1,4-benzoxazin-7-yl}-4′-(trifluoromethyl)-1,1′-biphenyl-2-carboxamide (320 mg).

¹H-NMR (DMSO-d₆):δ 3.88-3.98 (2H, m), 3.71 (2H, s), 4.21-4.28 (2H, m), 6.29-6.33 (2H, m), 6.96-7.00 (4H, m), 7.20 (1H, d, J=2.06 Hz), 7.45-7.75 (5H, m), 7.76 (1H, d, J=8.32 Hz), 10.33 (1H, s)

Preparation 87

A mixture of 5-methyl-4′-(trifluoromethyl)-1,1′-biphenyl-2-carboxylic acid (7.0 g), sodium hydrogencarbonate (3.15 g) and methyl iodide (2.33 ml) in N,N-dimethylformamide (47 ml) was stirred at ambient temperature for 30 hours. The reaction mixture was poured into water and the mixture was extracted with ethyl acetate. The organic layer was washed with water, dried over magnesium sulfate and evaporated in vacuo to give methyl 5-methyl-4′-(trifluoromethyl)-1,1′-biphenyl-2-carboxylate (7.27 g).

¹H-NMR (DMSO-d₆):δ 2.41 (3H, s), 3.59 (3H, s), 7.27 (1H, d, J=0.9 Hz), 7.37 (1H, dd, J=0.9 Hz, 8.0 Hz), 7.49 (2H, d, J=8.0 Hz), 7.72-7.80 (3H, m)

Preparation 88

A mixture of methyl 5-methyl-4′-(trifluoromethyl)-1,1′-biphenyl-2-carboxylate (7.2 g), N-bromosuccinimide (4.8 g) and 2,2′-azobis(4-methoxy-2,4-dimethylvaleronitrile) (0.15 g) in benzene (108 ml) was stirred at 50° C. for 4 hours. The reaction mixture was poured into ethyl acetate and the mixture was washed with saturated aqueous sodium hydrogencarbonate and water. The organic layer was dried over magnesium sulfate and evaporated in vacuo to give methyl 5-(bromomethyl)-4′-(trifluoromethyl)-1,1′-biphenyl-2-carboxylate (9.55 g) as a crude oil.

¹H-NMR (DMSO-d₆):δ 3.62 (3H, s), 4.80 (2H, s), 7.47-7.88 (7H, m)

Preparation 89

To a solution of methyl 5-(bromomethyl)-4′-(trifluoromethyl)-1,1′-biphenyl-2-carboxylate (2.0 g) in tetrahydrofuran (30 ml) was added 2M dimethylamine in tetrahydrofuran (4.0 ml) and the mixture was stirred at ambient temperature for 3 hours. The reaction mixture was poured into water and the mixture was adjusted to pH 9 with 20% aqueous potassium carbonate solution. The mixture was extracted with ethyl acetate. The organic layer was washed with water, dried over magnesium sulfate and evaporated in vacuo. The residue was purified by column chromatography on silica gel eluting with chloroform:methanol (19:1 v/v). The eluted fractions containing the desired product were collected and evaporated in vacuo to give methyl 5-[(dimethylamino)methyl]-4′-(trifluoromethyl)-1,1′-biphenyl-2-carboxylate (1.21 g).

¹H-NMR (DMSO-d₆):δ 2.17 (6H, s), 3.50 (2H, s), 3.61 (3H, s), 7.35 (1H, d, J=1.3 Hz), 7.44-7.54 (3H, m), 7.77 (2H, d, J=8.1 Hz), 7.82 (1H, d, J=8.0 Hz)

Preparation 90

A mixture of methyl 5-[(dimethylamino)methyl]-4′-(trifluoromethyl)-1,1′-biphenyl-2-carboxylate (1.1 g) and sodium hydroxide (0.33 g) in a mixture of water (3.3 ml) and ethanol (5.5 ml) was stirred under reflux for 5 hours. The solvent was removed by concentration. The residue was dissolved in water and the solution was adjusted to pH 7 with 6N hydrochloric acid. The mixture was extracted with a mixture of ethyl acetate and tetrahydrofuran. The organic layer was washed with brine, dried over magnesium sulfate and evaporated in vacuo. The residue was triturated with diisopropyl ether to give 5-[(dimethylamino)methyl]-4′-(trifluoromethyl)-1,1′-biphenyl-2-carboxylic acid (0.88 g).

¹H-NMR (DMSO-d₆):δ 2.29 (6H, s), 3.66 (2H, s), 7.36 (1H, d, J=1.4 Hz), 7.48 (1H, dd, J=1.4 Hz, 8.0 Hz), 7.54 (2H, d, J=8.0 Hz), 7.77 (2H, d, J=8.0 Hz), 7.81 (1H, d, J=8.0 Hz)

APCI-MS (m/z): 324 (M+H)⁺

### EXAMPLE 86

5-[(Dimethylamino)methyl]-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 2.20 (6H, s), 3.11 (2H, t, J=8.4 Hz), 3.51 (2H, s), 3.99 (2H, s), 4.19 (2H, t, J=8.4 Hz), 7.17-7.51 (6H, m), 7.58 (1H, d, J=7.6 Hz), 7.61 (2H, d, J=8.1 Hz), 7.70-7.81 (3H, m), 7.91 (1H, d, J=8.7 Hz), 8.49 (1H, dd, J=0.9 Hz, 4.9 Hz), 10.25 (1H, s)

APCI-MS (m/z): 559 (M+H)⁺

Preparation 91

To a solution of methyl 5-(bromomethyl)-4′-(trifluoromethyl)-1,1′-biphenyl-2-carboxylate (1.5 g) in methanol (10 ml) was added 28% methanolic sodium methoxide (2.3 ml) and the mixture was stirred at ambient temperature for 3 hours. To the reaction mixture was added water (5.0 ml) and the mixture was stirred under reflux for 5 hours. The solvent was removed by concentration. The residue was dissolved in water and the solution was adjusted to pH 2 with 6N hydrochloric acid. The mixture was extracted with ethyl acetate. The organic layer was washed with water, dried over magnesium sulfate and evaporated in vacuo. The residue was triturated with a mixture of hexane and diisopropyl ether (3:1 v/v) to give 5-(methoxymethyl)-4′-(trifluoromethyl)-1,1′-biphenyl-2-carboxylic acid (0.52 g).

¹H-NMR (DMSO-d₆):δ 3.32 (3H, s), 4.52 (2H, s), 7.32 (1H, s, J=1.6 Hz), 7.46 (1H, dd, J=1.6 Hz, 8.0 Hz), 7.54 (2H, d, J=8.0 Hz), 7.77 (2H, d, J=8.0 Hz), 7.83 (1H, d, J=8.0 Hz), 12.82 (1H, s)

### EXAMPLE 87

5-(Methoxymethyl)-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 3.11 (2H, d, J=8.2 Hz), 3.34 (3H, s), 3.99 (2H, s), 4.19 (2H, d, J=8.2 Hz), 4.54 (2H, s), 7.18-7.31 (2H, m), 7.35 (1H, d, J=7.8 Hz), 7.42-7.53 (3H, m), 7.57-7.68 (3H, m), 7.72-7.82 (3H, m), 7.92 (1H, d, J=8.7 Hz), 8.49 (1H, dd, J=0.9 Hz, 4.9 Hz), 10.26 (1H, s)

APCI-MS (m/z): 546 (M+H)⁺

Preparation 92

A mixture of methyl 5-(bromomethyl)-4′-(trifluoromethyl)-1,1′-biphenyl-2-carboxylate (2.0 g) and potassium acetate (1.1 g) in N,N-dimethylformamide (16 ml) was stirred at ambient temperature for 3 hours. The reaction mixture was poured into water and the mixture was extracted with ethyl acetate. The organic layer was washed with water, dried over magnesium sulfate and evaporated in vacuo. The residue was purified by column chromatography on silica gel eluting with n-hexane:ethyl acetate (17:3 v/v). The eluted fractions containing the desired product were collected and evaporated in vacuo to give methyl 5-[(acetyloxy)methyl]-4′-(trifluoromethyl)-1,1′-biphenyl-2-carboxylate (1.24 g).

¹H-NMR (DMSO-d₆):δ 2.10 (3H, s), 3.61 (3H, s), 5.19 (2H, s), 7.45 (1H, d, J=1.3 Hz), 7.52 (2H, d, J=8.1 Hz), 7.54 (1H, dd, J=1.3 Hz, 8.0 Hz), 7.79 (2H, d, J=8.1 Hz), 7.86 (1H, d, J=8.0 Hz)

Preparation 93

A mixture of methyl 5-[(acetyloxy)methyl]-4′-(trifluoromethyl)-1,1′-biphenyl-2-carboxylate (1.1 g) and sodium hydroxide (0.38 g) in a mixture of water (3.8 ml) and ethanol (5.5 ml) was stirred under reflux for 5 hours. The solvent was removed by concentration. The residue was dissolved in water and the solution was adjusted to pH 2 with 6N hydrochloric acid. The isolated precipitate was collected by filtration to give 5-(hydroxymethyl)-4′-(trifluoromethyl)-1,1′-biphenyl-2-carboxylic acid (0.91 g).

¹H-NMR (DMSO-d₆):δ 4.60 (2H, s), 5.38 (1H, s), 7.33 (1H, d, J=1.3 Hz), 7.46 (1H, dd, J=1.3 Hz, 8.0 Hz), 7.53 (2H, d, J=8.0 Hz), 7.77 (2H, d, J=8.0 Hz), 7.82 (1H, d, J=8.0 Hz), 12.76 (1H, s)

Preparation 94

To a mixture of 5-(hydroxymethyl)-4′-(trifluoromethyl)-1,1′-biphenyl-2-carboxylic acid (0.88 g) and triethylamine (0.91 ml) in tetrahydrofuran (9.0 ml) was added acetyl chloride (0.47 ml) under ice-cooling and the mixture was stirred at ambient temperature for 3 hours. The reaction mixture was poured into water and the mixture was adjusted to pH 2 with 6N hydrochloric acid. The mixture was extracted with ethyl acetate. The organic layer was washed with water, dried over magnesium sulfate and evaporated in vacuo. The residue was purified by column chromatography on silica gel eluting with n-hexane:ethyl acetate (1:3 v/v). The eluted-fractions containing the desired product were collected and evaporated in vacuo to give 5-[(acetyloxy)methyl]-4′-(trifluoromethyl)-1,1′-biphenyl-2-carboxylic acid (0.62 g).

¹H-NMR (DMSO-d₆):δ 2.09 (3H, s), 5.17 (2H, s), 7.40 (1H, d, J=1.4 Hz), 7.50 (1H, dd, J=1.4 Hz, 7.9 Hz), 7.54 (2H, d, J=8.2 Hz), 7.78 (2H, d, J=8.2 Hz), 7.84 (1H, d, J=7.9 Hz), 12.91 (1H, s)

### EXAMPLE 88

[6-({[1-(2-Pyridinylacetyl)-2,3-dihydro-1H-indol-5-yl]amino}carbonyl)-4′-(trifluoromethyl)-1,1′-biphenyl-3-yl]methyl acetate

The title compound was obtained in the same manner as in Example 6.

¹H-NMR (DMSO-d₆):δ 2.09 (3H, s), 3.11 (2H, t, J=8.3 Hz), 3.99 (2H, s), 4.19 (2H, t, J=8.3 Hz), 5.19 (2H, s), 7.16-7.32 (2H, m), 7.35 (1H, d, J=7.8 Hz), 7.46-7.67 (6H, m), 7.70-7.80 (3H, m), 7.91 (1H, d, J=8.7 Hz), 8.46-8.52 (1H, m), 10.25 (1H, s)

### EXAMPLE 89

A mixture of [6-({[1-(2-pyridinylacetyl)-2,3-dihydro-1H-indol-5-yl]amino}carbonyl)-4′-(trifluoromethyl)-1,1′-biphenyl-3-yl]methyl acetate (0.24 g) and 1N aqueous sodium hydroxide (0.54 ml) in a mixture of methanol (2.4 ml) and tetrahydrofuran (2.4 ml) was stirred at ambient temperature for 3 hours. The reaction mixture was poured into water and the mixture was extracted with ethyl acetate. The organic layer was washed with water, dried over magnesium sulfate and evaporated in vacuo. The residue was crystallized from ethyl acetate to give 5-(hydroxymethyl)-N-[1-(2-pyridinylacetyl)-2,3-dihydro-1H-indol-5-yl]-4′-(trifluoromethyl)-1,1′-biphenyl-2-carboxamide (0.16 g).

¹H-NMR (DMSO-d₆):δ 3.11 (2H, t, J=8.3 Hz), 3.99 (2H, s), 4.19 (2H, t, J=8.3 Hz), 4.62 (2H, d, J=5.7 Hz), 5.37 (1H, t, J=5.7 Hz), 7.17-7.32 (2H, m), 7.35 (1H, d, J=7.8 Hz), 7.41-7.53 (3H, m), 7.55-7.68 (3H, m), 7.70-7.82 (3H, m), 7.92 (1H, d, J=8.7 Hz), 8.47-8.53 (1H, m), 10.21 (1H, s)

APCI-MS (m/z): 532 (M+H)⁺

Preparation 95

To a stirred mixture of methyl 5-nitro-2-{[(trifluoromethyl)sulfonyl]oxy}benzoate (21.0 g), lithium chloride (8.1 g) and tetrakis(triphenylphosphine)palladium(0) (3.7 g) in toluene (210 ml) was added a solution of sodium carbonate (17.6 g) in water (70 ml) and followed by addition of 4-tolylboronic acid (9.5 g). The mixture was stirred at 100° C. for 6 hours. The reaction mixture was poured into a mixture of ethyl acetate and water. The separated organic layer was washed with water and evaporated in vacuo. The residue was triturated with a mixture of hexane and diisopropyl ether (2:3 v/v) to give methyl 4′-methyl-4-nitro-1,1′-biphenyl-2-carboxylate (13.25 g).

¹H-NMR (DMSO-d₆):δ 2.37 (3H, s), 3.69 (3H, s), 7.28 (4H, s), 7.72 (1H, d, J=8.5 Hz), 8.41 (1H, dd, J=2.5 Hz, 8.5 Hz), 8.50 (1H, d, J=2.5 Hz)

Preparation 96

4′-Methyl-4-nitro-1,1′-biphenyl-2-carboxylic acid

The title compound was obtained in the same manner as in Preparation 93.

¹H-NMR (DMSO-d₆):δ 2.37 (3H, s), 7.29 (4H, s), 7.67 (1H, d, J=8.5 Hz), 8.37 (1H, dd, J=2.5 Hz, 8.5 Hz), 8.46 (1H, d, J=2.5 Hz), 13.33 (1H, br s)

Preparation 97

4-(Dimethylamino)-4′-methyl-1,1′-biphenyl-2-carboxylic acid

The title compound was obtained in the same manner as in Preparation 32.

¹H-NMR (DMSO-d₆):δ 2.31 (3H, s), 2.94 (6H, s), 6.88 (1H, dd, J=2.7 Hz, 8.5 Hz), 6.94 (1H, d, J=2.7 Hz), 7.15 (4H, s), 7.17 (1H, d, J=8.5 Hz), 12.59 (1H, s)

### EXAMPLE 90

4-(Dimethylamino)-4′-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.26 (3H, s), 2.96 (6H, s), 3.11 (2H, t, J=8.4 Hz), 3.99 (2H, s), 4.19 (2H, t, J=8.4 Hz), 6.79 (1H, d, J=2.6 Hz), 6.88 (1H, dd, J=2.6 Hz, 8.6 Hz), 7.10 (2H, d, J=8.0 Hz), 7.20-7.31 (5H, m), 7.35 (1H, d, J=7.7 Hz), 7.52 (1H, s), 7.76 (1H, dt, J=1.8 Hz, 7.7 Hz), 7.91 (1H, d, J=8.6 Hz), 8.46-8.52 (1H, m), 10.10 (1H, s)

negative APCI-MS (m/z): 489 (M−H)⁻

### EXAMPLE 91

4-Methyl-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 2.42 (3H, s), 3.11 (2H, t, J=8.3 Hz), 3.99 (2H, s), 4.19 (2H, t, J=8.3 Hz), 7.17-7.54 (7H, m), 7.60 (2H, d, J=8.1 Hz), 7.70-7.82 (3H, m), 7.92 (1H, d, J=8.7 Hz), 8.49 (1H, dd, J=0.9 Hz, 4.8 Hz), 10.26 (1H, s)

APCI-MS (m/z): 516 (M+H)⁺

### EXAMPLE 92

4,4′-Dimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.28 (3H, s), 2.39 (3H, s), 3.11 (2H, t, J=8.3 Hz), 3.99 (2H, s), 4.19 (2H, t, J=8.3 Hz), 7.15 (2H, d, J=8.0 Hz), 7.20-7.39 (8H, m), 7.52 (1H, d, J=1.5 Hz), 7.76 (1H, dt, J=1.9 Hz, 7.6 Hz), 7.91 (1H, d, J=8.7 Hz), 8.46-8.52 (1H, m), 10.15 (1H, s)

APCI-MS (m/z): 462 (M+H)⁺

### EXAMPLE 93

4′-Chloro-4-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.40 (3H, s), 3.11 (2H, t, J=8.3 Hz), 3.99 (2H, s), 4.19 (2H, t, J=8.3 Hz), 7.02-7.45 (10H, m), 7.51 (1H, s), 7.75 (1H, dt, J=1.9 Hz, 7.6 Hz), 7.93 (1H, d, J=8.7 Hz), 8.46-8.52 (1H, m), 10.21 (1H, s)

APCI-MS (m/z): 482 (M+H)⁺

### EXAMPLE 94

4-Methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.40 (3H, s), 3.10 (2H, t, J=8.4 Hz), 3.98 (2H, s), 4.18 (2H, t, J=8.4 Hz), 7.21 (1H, dd, J=1.5 Hz, 8.8 Hz), 7.24-7.30 (2H, m), 7.32-7.38 (6H, m), 7.39-7.42 (2H, m), 7.49 (1H, s), 7.75 (1H, dt, J=1.8 Hz, 7.7 Hz), 7.90 (1H, d, J=8.8 Hz), 8.49 (1H, dd, J=0.8 Hz, 4.0 Hz), 10.12 (1H, S)

ESI-MS(m/z): 448 (M+H)⁺, 470 (M+Na)⁺

### EXAMPLE 95

5-Methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.40 (3H, s), 3.10 (2H, t, J=8.4 Hz), 3.98 (2H, s), 4.18 (2H, t, J=8.4 Hz), 7.19 (1H, d, J=7.3 Hz), 7.24-7.31 (4H, m), 7.32-7.38 (3H, m), 7.39-7.49 (3H, m), 7.50 (1H, s), 7.75 (1H, dt, J=1.8 Hz, 7.7 Hz), 7.89 (1H, d, J=8.7 Hz), 8.49 (1H, dd, J=0.8 Hz, 4.8 Hz), 10.02 (1H, s)

ESI-MS(m/z): 448 (M+H)⁺, 470 (M+Na)⁺

### EXAMPLE 96

4′-Fluoro-4-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.40 (3H, s), 3.11 (2H, t, J=8.4 Hz), 3.99 (2H, s), 4.19 (2H, t, J=8.4 Hz), 7.13-7.46 (10H, m), 7.51 (1H, s), 7.76 (1H, dt, J=1.8 Hz, 7.7 Hz), 7.91 (1H, d, J=8.7 Hz), 8.49 (1H, dd, J=0.9 Hz, 5.0 Hz), 10.16 (1H, s)

ESI-MS(m/z): 466 (M+H)⁺, 488 (M+Na)⁺

Preparation 98

4,5-Dimethyl-4′-(trifluoromethyl)-1,1′-biphenyl-2-carboxylic acid

The title compound was obtained in the same manner as in Preparation 15.

¹H-NMR (DMSO-d₆):δ 2.30 (6H, s), 7.17 (1H, s), 7.49 (2H, d, J=8.0 Hz), 7.64 (1H, s), 7.73 (2H, d, J=8.0 Hz), 12.62 (1H, s)

### EXAMPLE 97

4,5-Dimethyl-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 2.33 (6H, s), 3.10 (2H, t, J=8.3 Hz), 3.99 (2H, s), 4.19 (2H, t, J=8.3 Hz), 7.18-7.47 (5H, m), 7.50 (1H, s), 7.58 (2H, d, J=8.2 Hz), 7.68-7.81 (3H, m), 7.92 (1H, d, J=8.7 Hz), 8.46-8.51 (1H, m), 10.17 (1H, s)

negative ESI-MS(m/z): 528 (M−H)⁻

Preparation 99

4,4′,5-Trimethyl-1,1′-biphenyl-2-carboxylic acid

The title compound was obtained in the same manner as in Preparation 15.

¹H-NMR (DMSO-d₆):δ 2.27 (6H, s), 2.33 (3H, s), 7.11 (1H, s), 7.17 (4H, s), 7.50 (1H, s), 12.48 (1H, s)

### EXAMPLE 98

4,4′,5-Trimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.27 (3H, s), 2.30 (6H, s), 3.10 (2H, t, J=8.3 Hz), 3.98 (2H, s), 4.18 (2H, t, J=8.3 Hz), 7.10-7.38 (9H, m), 7.52 (1H, s), 7.75 (1H, dt, J=1.8 Hz, 7.6 Hz), 7.91 (1H, d, J=8.7 Hz), 8.46-8.51 (1H, m), 10.06 (1H, s)

negative ESI-MS(m/z): 474 (M−H)⁻

Preparation 100

Palladium(II)acetate (48 mg) was added to a mixture of 2-iodo-4,5-dimethoxybenzoic acid (6.6 g), 4-(trifluoromethyl)phenylboronic acid (5.3 g) and sodium carbonate (6.8 g) in water (90 ml) and the mixture was stirred at 60° C. for 4 hours. The reaction mixture was adjusted to pH 7 with 6N hydrochloric acid and the mixture was extracted with ethyl acetate. The organic layer was washed with water, dried over magnesium sulfate and evaporated in vacuo. The residue was triturated with diisopropyl ether to give 4,5-dimethoxy-4′-(trifluoromethyl)-1,1′-biphenyl-2-carboxylic acid (2.62 g).

¹H-NMR (DMSO-d₆):δ 3.85 (6H, s), 6.90 (1H, s), 7.42 (1H, s), 7.52 (2H, d, J=8.0 Hz), 7.73 (2H, d, J=8.0 Hz)

negative ESI-MS(m/z): 325 (M−H)⁻

### EXAMPLE 99

4,5-Dimethoxy-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 3.10 (2H, t, J=8.2 Hz), 3.87 (6H, s), 3.99 (2H, s), 4.19 (2H, t, J=8.2 Hz), 7.04 (1H, s), 7.15-7.32 (3H, m), 7.35 (1H, d, J=7.7 Hz), 7.46 (1H, s), 7.61 (2H, d, J=8.2 Hz), 7.67-7.80 (3H, m), 7.91 (1H, d, J=8.7 Hz), 8.46-8.51 (1H, m), 10.08 (1H, s)

negative ESI-MS(m/z): 560 (M−H)⁻

Preparation 101

4,5-Dimethoxy-4′-methyl-1,1′-biphenyl-2-carboxylic acid

The title compound was obtained in the same manner as in Preparation 100.

¹H-NMR (DMSO-d₆):δ 2.33 (3H, s), 3.82 (6H., s), 7.16 (2H, d, J=8.8 Hz), 7.20 (2H, d, J=8.8 Hz), 7.31 (1H, s), 12.41 (1H, br s)

negative ESI-MS(m/z): 271 (M−H)⁻

### EXAMPLE 100

4,5-Dimethoxy-4′-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.28 (3H, s), 3.11 (2H, t, J=8.2 Hz), 3.84 (6H, s), 3.99 (2H, s), 4.19 (2H, t, J=8.2 Hz), 6.95 (1H, s), 7.07-7.39 (8H, m), 7.48 (1H, s), 7.76 (1H, dt, J=1.8 Hz, 7.7 Hz), 7.90 (1H, d, J=8.7 Hz), 8.49 (1H, d, J=4.9 Hz), 9.95 (1H, s)

negative ESI-MS(m/z): 506 (M−H)⁻

### EXAMPLE 101

4′-Chloro-5-methyl-N-[1-(2-pyridinylacetyl)-2,3-dihydro-1H-indol-5-yl]-1,1′-biphenyl-2-carboxamide hydrochloride

The title compound was obtained in the same manner as in Example 12.

¹H-NMR (DMSO-d₆):δ 2.40 (3H, s), 3.19 (2H, t, J=8.2 Hz), 4.25 (2H, t, J=8.2 Hz), 4.47 (2H, s), 7.23-7.34 (3H, m), 7.42 (4H, s), 7.47 (1H, d, J=7.6 Hz), 7.54 (1H, s), 7.86 (1H, d, J=8.7 Hz), 7.89-8.02 (2H, m), 8.52 (1H, dt, J=1.2 Hz, 7.8 Hz), 8.86-8.92 (1H, m), 10.20 (1H, s)

Preparation 102

N-(1H-Indol-4-yl)-4′-(trifluoromethyl)-1,1′-biphenyl-2-carboxamide

The title compound was obtained from 1H-indol-4-amine and 4′-(trifluoromethyl)-1,1′-biphenyl-2-carbonyl chloride in the same manner as in Example 1.

¹H-NMR (DMSO-d₆):δ 6.25-6.30 (1H, m), 6.99 (1H, t, J=7.8 Hz), 7.12-7.20 (2H, m), 7.38 (1H, d, J=7.4 Hz), 7.50-7.80 (8H, m), 10.00 (1H, s), 11.05 (1H, s)

Preparation 103

N-(2,3-Dihydro-1H-indol-4-yl)-4′-(trifluoromethyl)-1,1′-biphenyl-2-carboxamide

The title compound was obtained in the same manner as in Preparation 10.

¹H-NMR (DMSO-d₆):δ 2.49 (2H, t, J=8.4 Hz), 3.28 (2H, t, J=8.4 Hz), 5.44 (1H, s), 6.27 (1H, d, J=7.6 Hz), 6.66 (1H, d, J=7.6 Hz), 6.83 (1H, t, J=7.6 Hz), 7.48-7.69 (6H, m), 7.79 (2H, d, J=8.2 Hz), 9.61 (1H, s)

APCI-MS (m/z): 383 (M+H)⁺

### EXAMPLE 102

N-[1-(2-Pyridinylacetyl)-2,3-dihydro-1H-indol-4-yl]-4′-(trifluoromethyl)-1,1′-biphenyl-2-carboxamide

The title compound was obtained from N-(2,3-dihydro-1H-indol-4-yl)-4′-(trifluoromethyl)-1,1′-biphenyl-2-carboxamide and 2-pyridinylacetic acid hydrochloride in the same manner as in Example 6.

¹H-NMR (DMSO-d₆):δ 2.76 (2H, t, J=8.3 Hz), 3.99 (2H, s), 4.12 (2H, t, J=8.3 Hz), 7.05-7.17 (2H, m), 7.22-7.32 (1H, m), 7.35 (1H, d, J=7.8 Hz), 7.50-7.90 (10H, m), 8.46-8.52 (1H, m), 9.87 (1H, s)

APCI-MS (m/z): 502 (M+H)⁺

### EXAMPLE 103

N-{1-[3-(2-Pyridinyl)propanoyl]-2,3-dihydro-1H-indol-4-yl}-4′-(trifluoromethyl)-1,1′-biphenyl-2-carboxamide

The title compound was obtained from N-(2,3-dihydro-1H-indol-4-yl)-4′-(trifluoromethyl)-1,1′-biphenyl-2-carboxamide and 3-(2-pyridinyl)propanoic acid in the same manner as in Example 6.

¹H-NMR (DMSO-d₆):δ 2.77 (2H, t, J=8.2 Hz), 2.89 (2H, t, J=7.0 Hz), 3.05 (2H, t, J=7.0 Hz), 4.05 (2H, t, J=8.2 Hz), 7.05-7.15 (2H, m), 7.19 (1H, dd, J=5.8 Hz, 7.3 Hz), 7.34 (1H, d, J=7.8 Hz), 7.48-7.94 (10H, m), 8.44-8.51 (1H, m), 9.88 (1H, s)

negative APCI-MS (m/z): 514 (M−H)⁻

### EXAMPLE 104

To a solution of [2-(2,5-dimethyl-1H-pyrrol-1-yl)-4-pyrimidinyl]acetic acid (666 mg), N-(2,3-dihydro-1H-indol-5-yl)-4′-(trifluoromethyl)-1,1′-biphenyl-2-carboxamide hydrochloride (1.009 g) and 1-hydroxybenzotriazole (391 mg) in N,N-dimethylformamide (20 ml) was added 1-[3-(dimethylamino)propyl]-3-ethylcarbodiimide hydrochloride (WSC.HCl) (554 mg), followed by addition of triethylamine (0.84 ml) at ambient temperature. The reaction mixture was stirred at 50° C. for 12 hours and concentrated in vacuo. The residue was dissolved in ethyl acetate and water, and the solution was extracted with ethyl acetate. The organic layer was washed with water and brine, dried over magnesium sulfate, filtered, and concentrated in vacuo. The residue was purified by column chromatography on silica gel eluting with n-hexane:ethyl acetate (1:3 v/v) to give N-(1-{[2-(2,5-dimethyl-1H-pyrrol-1-yl)-4-pyrimidinyl]acetyl}-2,3-dihydro-1H-indol-5-yl)-4′-(trifluoromethyl)-1,1′-biphenyl-2-carboxamide (488 mg) as a yellow oil.

¹H-NMR (DMSO-d₆):δ 2.21 (6H, s), 3.12 (2H, t, J=8.6 Hz), 4.17 (2H, t, J=8.6 Hz), 5.80 (2H, s), 7.20-7.77 (11H, m), 7.91 (1H, d, J=8.9 Hz), 8.82 (1H, d, J=4.9 Hz), 10.28 (1H, s) ESI-MS(m/z): 596 (M+H)+

### EXAMPLE 105

To a solution of N-(1-{[2-(2,5-dimethyl-1H-pyrrol-1-yl)-4-pyrimidinyl]acetyl}-2,3-dihydro-1H-indol-5-yl)-4′-(trifluoromethyl)-1,1′-biphenyl-2-carboxamide (488 mg) in ethanol (8 ml) and water (2 ml) was added hydroxylamine hydrochloride (569 mg), followed by addition of triethylamine (0.35 ml) at ambient temperature. The reaction mixture was heated to 100° C. and stirred for 15 hours. The reaction mixture was cooled to ambient temperature and concentrated in vacuo. The residue was dissolved in ethyl acetate and water, and extracted. The organic layer was washed with water and brine, dried over magnesium sulfate, filtered, and concentrated in vacuo. The residue was purified by column chromatography on silica gel eluting with n-hexane:ethyl acetate (1:3 v/v) to give N-{1-[(2-amino-4-pyrimidinyl)acetyl]-2,3-dihydro-1H-indol-5-yl}-4′-(trifluoromethyl)-1,1′-biphenyl-2-carboxamide (34 mg) as a pale brown solid.

¹H-NMR (DMSO-d₆):δ 3.13 (2H, t, J=7.7 Hz), 4.15 (2H, t, J=8.4 Hz), 6.52 (1H, d, J=4.9 Hz), 6.55 (2H, s), 7.21 (1H, d, J=8.6 Hz), 7.48-7.77 (9H, m), 8.15 (1H, d, J=4.9 Hz), 10.28 (1H, s)

ESI-MS(m/z): 518 (M+H)⁺

### EXAMPLE 106

To a solution of N-(2,3-dihydro-1H-indol-5-yl)-4′-(trifluoromethyl)-1,1′-biphenyl-2-carboxamide hydrochloride (0.164 g) and 2-vinylpyrazine (50 mg) in methoxyethanol (4 ml) was added acetic acid (20 μl). The reaction mixture was refluxed for 2 days, cooled to ambient temperature, and extracted with ethyl acetate. The organic layer was washed with brine, dried over magnesium sulfate, filtered and concentrated in vacuo. The residue was purified by column chromatography on silica gel eluting with n-hexane:ethyl acetate (1:2 v/v) to give N-{1-[2-(2-pyrazinyl)ethyl]-2,3-dihydro-1H-indol-5-yl}-4′-(trifluoromethyl)-1,1′-biphenyl-2-carboxamide(79 mg) as a pale orange solid.

¹H-NMR (DMSO-d₆):δ 2.87 (2H, t, J=8.2 Hz), 3.04 (2H, t, J=7.4 Hz), 3.35 (2H, t, J=8.4 Hz), 3.46 (2H, t, J=7.4 Hz), 6.31 (1H, d, J=8.2 Hz), 6.68 (1H, dd, J=8.2 Hz, 2.0 Hz), 6.94 (2H, d, J=9.9 Hz), 7.38-7.75 (9H, m), 8.39 (1H, d, J=2.6 Hz), 8.45 (1H, d, J=1.6 Hz), 8.49 (1H, d, J=1.6 Hz)

ESI-MS(m/z): 489 (M+H)⁺

### EXAMPLE 107

N-[1-(3-Pyridinylacetyl)-2,3-dihydro-1H-indol-5-yl]-4′-(trifluoromethyl)-1,1′-biphenyl-2-carboxamide

The title compound was obtained from N-(2,3-dihydro-1H-indol-4-yl)-4′-(trifluoromethyl)-1,1′-biphenyl-2-carboxamide and 3-pyridinylacetic acid hydrochloride in the same manner as in Example 6 as white crystals.

¹H-NMR (DMSO-d₆):δ 3.14 (2H, t, J=8.3 Hz), 3.88 (2H, s), 4.20 (2H, d, J=8.3 Hz), 7.20 (1H, d, J=8.9 Hz), 7.35 (1H, dd, J=7.7 Hz, 1.9 Hz), 7.5-7.8 (7H, m), 7.90 (1H, d, J=8.7 Hz), 8.4-8.5 (2H, m) 10.27 (1H, s)

APCI-MS (m/z): 502 (M+H)⁺

### EXAMPLE 108

N-[1-(4-pyridinylacetyl)-2,3-dihydro-1H-indol-5-yl]-4′-(trifluoromethyl)-1,1′-biphenyl-2-carboxamide

The title compound was obtained from N-(2,3-dihydro-1H-indol-4-yl)-4′-(trifluoromethyl)-1,1′-biphenyl-2-carboxamide and 4-pyridinylacetic acid hydrochloride in the same manner as in Example 6 as white crystals.

¹H-NMR (DMSO-d₆):δ 3.17 (2H, t, J=8.5 Hz), 3.89 (2H, s), 4.17 (2H, d, J=8.5 Hz), 7.21 (1H, d, J=8.6 Hz), 7.30 (1H, d, J=5.9 Hz), 7.5-7.7 (7H, m), 7.75 (1H, d, J=8.3 Hz), 7.90 (1H, d, J=8.6 Hz), 8.50 (1H, d, J=6.0 Hz), 10.27 (1H, s)

APCI-MS (m/z): 502 (M+H)⁺

Preparation 104

To a solution of ethyl N-methylidyneglycinate (5.66 g) and triethylamine (6.07 g) in tetrahydrofuran (60 ml) was added dropwise 4-(trifluoromethyl)benzoyl chloride (10.4 g) at 5° C. The mixture was gradually warmed to ambient temperature and stirred at ambient temperature for 72 hours. The insoluble materials were filtered off and the filtrate was evaporated in vacuo. To the residue was added a mixture of ethyl acetate and water. The separated organic layer was washed with water and brine, dried over magnesium sulfate, and evaporated to dryness. The residue was purified by column chromatography on silica gel eluting with n-hexane:ethyl acetate (1:1 v/v) to give ethyl 5-[4-(trifluoromethyl)phenyl]-1,3-oxazole-4-carboxylate (9.72 g) as white crystals.

¹H-NMR (DMSO-d₆):δ 1.28 (3H, t, J=8.3 Hz), 4.31 (2H, q, J=8.3 Hz), 7.91 (2H, d, J=8.1 Hz), 8.19 (2H, d, J=8.1 Hz), 8.67 (1H, s)

APCI-MS (m/z): 286 (M+H)⁺

Preparation 105

To a solution of ethyl 5-[4-(trifluoromethyl)phenyl]-1,3-oxazole-4-carboxylate (9.70 g) in a mixture of tetrahydrofuran (30 ml) and methanol (30 ml) was added 5N aqueous sodium hydroxide solution (13.6 ml) at ambient temperature. The mixture was warmed to 50° C. and stirred at 50° C. for 3 hours. The reaction mixture was cooled to 5° C. and poured into a mixture of ethyl acetate and water. The separated organic layer was washed with water and brine, dried over magnesium sulfate and evaporated in vacuo. The residue was crystallized from ethyl acetate and collected by filtration, washed with ethyl acetate and dried in vacuo to give 5-[4-(trifluoromethyl)phenyl]-1,3-oxazole-4-carboxylic acid (7.88 g) as white crystals.

¹H-NMR (DMSO-d₆):δ 7.90 (2H, d, J=8.1 Hz), 8.21 (2H, d, J=8.1 Hz), 8.63 (1H, s)

negative ESI-MS(m/z): 256 (M−H)⁻

### EXAMPLE 109

N-[1-(2-Pyridinylacetyl)-2,3-dihydro-1H-indol-5-yl]-5-[4-(trifluoromethyl)phenyl]-1,3-oxazole-4-carboxamide

The title compound was obtained in the same manner as in Example 6 as white crystals.

¹H-NMR (DMSO-d₆):δ 3.17 (2H, t, J=8.3 Hz), 4.01 (2H, s), 4.23 (2H, t, J=8.3 Hz), 7.28 (1H, dd, J=7.5 Hz, 4.9 Hz), 7.37 (1H, d, J=7.8 Hz), 7.55 (1H, d, J=8.7 Hz), 7.7-7.85 (2H, m), 7.90 (2H, d, J=8.7 Hz), 7.99 (1H, d, J=8.7 Hz), 8.40 (2H, d, J=8.4 Hz), 8.50 (1H, d, J=4.0 Hz), 8.78 (1H, s), 10.26 (1H, s)

APCI-MS (m/z): 493 (M+H)⁺

Preparation 106

To a suspension of 3-bromo-2-thiophenecarbaldehyde (1.91 g), 4-(trifluoromethyl)phenylboronic acid (2.47 g) and potassium carbonate (3.46 g) in water (15 ml) were added palladium(II)acetate (112 mg) and tetra-n-butylammonium bromide (3.23 g) at ambient temperature and the mixture was stirred vigorously at ambient temperature for 3 hours. To the residue was added toluene (20 ml) and vigorous stirring was continued for 16 hours. The mixture was poured into a mixture of ethyl acetate and water, and activated carbon (5 g) was added thereto. The mixture was stirred for 20 minutes and the activated carbon was filtered off. The filtrate was separated and the organic layer was washed with water and brine, dried over magnesium sulfate and evaporated in vacuo. The residue was purified by flash chromatography eluting with n-hexane:ethyl acetate (10:1 v/v) to give crude 3-[4-(trifluoromethyl)phenyl]-2-thiophenecarbaldehyde (2.06 g) as a brown oil. The crude product was used for the next step without further purification.

Preparation 107

To a solution of 3-[4-(trifluoromethyl)phenyl]-2-thiophenecarbaldehyde (2.06 g) in acetone (20 ml) and tert-butanol (20 ml) was added 2-pentene (5.66 g) at ambient temperature and the mixture was cooled to 10° C. To this mixture was added dropwise a solution of sodium chlorite (2.18 g) in water (20 ml) and the mixture was warmed to ambient temperature and stirred vigorously for 4 hours. The mixture was poured into a mixture of ethyl acetate and ice-water and adjusted to pH 2 with 6N hydrochloric acid. The separated organic layer was washed with water and brine, dried over magnesium sulfate and evaporated in vacuo. The residue was purified by column chromatography on silica gel eluting with ethyl acetate to give 3-[4-(trifluoromethyl)phenyl]-2-thiophenecarboxylic acid (1.93 g) as a pale brown solid.

¹H-NMR (DMSO-d₆):δ 7.6-7.8 (4H, m), 7.85-8.0 (2H, m), 13.03 (1H, br)

negative ESI-MS(m/z): 271 (M−H)⁻

### EXAMPLE 110

N-[1-(2-Pyridinylacetyl)-2,3-dihydro-1H-indol-5-yl]-3-[4-(trifluoromethyl)phenyl]-2-thiophenecarboxamide

The title compound was obtained in the same manner as in Example 6 as white crystals.

¹H-NMR (DMSO-d₆):δ 3.13 (2H, t, J=8.4 Hz), 4.00 (2H, s), 4.20 (2H, d, J=8.4 Hz), 7.2-7.4 (4H, m), 7.50 (1H, br s), 7.65-8.0 (4H, m), 8.49 (1H, d, J=4.0 Hz), 10.20 (1H, s)

ESI-MS(m/z): 530 (M+Na)⁺, 508 (M+H)⁺

### EXAMPLE 111

To a solution of N-(2,3-dihydro-1H-indol-5-yl)-4′-(trifluoromethyl)-1,1′-biphenyl-2-carboxamide (757 mg) in 2-methoxyethanol (10 ml) was added 4-vinylpyridine (231 mg) at ambient temperature and the mixture was refluxed for 16 hours. The mixture was cooled to ambient temperature and purified by column chromatography on silica gel eluting with ethyl acetate to give N-{1-[2-(4-pyridinyl)ethyl]-2,3-dihydro-1H-indol-5-yl}-4′-(trifluoromethyl)-1,1′-biphenyl-2-carboxamide (635 mg) as white crystals.

¹H-NMR (DMSO-d₆):δ 2.8-2.95 (4H, m), 3.3-3.45 (4H, m), 6.46 (1H, d, J=8.4 Hz), 7.09 (1H, d, J=8.4 Hz), 7.20 (1H, s), 7.35-7.4 (2H, m), 7.45-7.6 (4H, m)

ESI-MS(m/z): 510 (M+Na)⁺, 488 (M+H)⁺

### EXAMPLE 112

N-{1-[(E)-2-(2-Pyridinyl)ethenyl]-2,3-dihydro-1H-indol-5-yl}-4′-(trifluoromethyl)-1,1′-biphenyl-2-carboxamide

The title compound was obtained from N-(2,3-dihydro-1H-indol-5-yl)-4′-(trifluoromethyl)-1,1′-biphenyl-2-carboxamide and 2-ethynylpyridine in the same manner as in Example 111 as a yellow powder.

¹H-NMR (DMSO-d₆):δ 3.13 (2H, t, J=8.6 Hz), 3.82 (2H, t, J=8.6 Hz), 5.56 (1H, d, J=13.4 Hz), 6.85-7.05 (2H, m), 7.36 (1H, br s), 7.45-7.7 (7H, m), 7.76 (2H, d, J=8.2 Hz), 8.05 (1H, d, J=13.4 Hz), 8.34 (1H, d, J=3.8 Hz), 10.15 (1H, s)

APCI-MS (m/z): 486 (M+H)⁺

Preparation 108

To a suspension of methyl(triphenyl)phosphonium bromide (3.47 g) in tetrahydrofuran (30 ml) was added potassium tert-butoxide (1.09 g) at 5° C. under a nitrogen atmosphere and the mixture was stirred at ambient temperature for an hour. To the resulting solution was added dropwise a solution of 1,3-thiazole-2-carbaldehyde (1.0 g) in tetrahydrofuran (10 ml) at 5° C. and the mixture was stirred at ambient temperature for 19 hours. The mixture was poured into a mixture of ethyl acetate and water. The separated organic layer was washed with brine, dried over magnesium sulfate and evaporated in vacuo. The residue was purified by flash column chromatography on silica gel eluting with n-hexane:ethyl acetate (10:1 v/v) to give 2-vinyl-1,3-thiazole (352 mg) as a colorless oil.

¹H-NMR (DMSO-d₆):δ 5.58 (1H, d, J=10.9 Hz), 6.05 (1H, d, J=17.5 Hz), 6.98 (1H, dd, J=17.5 Hz, 10.9 Hz), 7.69 (1H, d, J=3.2 Hz), 7.83 (1H, d, J=3.2 Hz)

### EXAMPLE 113

To a solution of N-(2,3-dihydro-1H-indol-5-yl)-4′-(trifluoromethyl)-1,1′-biphenyl-2-carboxamide (1.01 g) in 2-methoxyethanol (10 ml) was added 2-vinyl-1,3-thiazole (323 mg) and methanesulfonic acid (254 mg) at ambient temperature and the mixture was refluxed for 20 hours. The mixture was cooled to ambient temperature and purified by column chromatography on silica gel eluting with n-hexane:ethyl acetate (1:2 v/v). The obtained compound was purified again by preparative HPLC to give N-{1-[2-(1,3-thiazol-2-yl)ethyl]-2,3-dihydro-1H-indol-5-yl}-4′-(trifluoromethyl)-1,1′-biphenyl-2-carboxamide (470 mg) as yellow crystals.

¹H-NMR (DMSO-d₆):δ 2.84 (2H, d, J=8.2 Hz), 3.2-3.5 (6H, m), 6.42 (1H, d, J=8.4 Hz), 7.08 (1H, d, J=8.4 Hz), 7.21 (1H, s), 7.45-7.8 (10H, m), 9.97 (1H, s)

ESI-MS(m/z): 516 (M+Na)⁺, 494 (M+H)⁺

Preparation 109

N-(2-Methyl-1H-indol-5-yl)-4′-(trifluoromethyl)-1,1′-biphenyl-2-carboxamide

The title compound was obtained from 2-methyl-1H-indol-5-amine and 4′-(trifluoromethyl)-1,1′-biphenyl-2-carbonyl chloride in the same manner as in Example 1 as light brown crystals.

¹H-NMR (DMSO-d₆):δ 2.35 (3H, s), 6.03 (1H, s), 7.02 (1H, d, J=8.7 Hz), 7.13 (1H, d, J=8.7 Hz), 7.5-7.8 (9H, m), 10.04 (1H, s), 10.80 (1H, br s)

APCI-MS (m/z): 395 (M+H)⁺

Preparation 110

N-(2-Methyl-2,3-dihydro-1H-indol-5-yl)-4′-(trifluoromethyl)-1,1′-biphenyl-2-carboxamide

The title compound was obtained in the same manner as in Preparation 10 as a light brown solid.

¹H-NMR (DMSO-d₆):δ 1.15 (3H, d, J=6.1 Hz), 2.35-2.5 (1H, m), 2.95-3.1 (1H, m), 5.42 (1H, d, J=2.0 Hz), 6.33 (1H, d, J=8.2 Hz), 6.96 (1H, dd, J=8.2 Hz, 2.0 Hz), 7.13 (1H, d, J=2.0 Hz), 7.45-7.55 (4H, m), 7.62 (2H, d, J=8.3 Hz), 7.76 (2H, d, J=8.3 Hz), 9.88 (1H, s)

APCI-MS (m/z): 397 (M+H)⁺

### EXAMPLE 114

N-[2-Methyl-1-(2-pyridinylacetyl)-2,3-dihydro-1H-indol-5-yl]-4′-(trifluoromethyl)-1,1′-biphenyl-2-carboxamide

The title compound was obtained from N-(2-methyl-2,3-dihydro-1H-indol-5-yl)-4′-(trifluoromethyl)-1,1′-biphenyl-2-carboxamide and 2-pyridinylacetic acid hydrochloride in the same manner as in Preparation 5 as white crystals.

¹H-NMR (DMSO-d₆): 1.27 (3H, d, J=6.1 Hz), 2.63 (1H, d, J=15.9 Hz), 3.2-3.4 (1H, m), 3.9-4.2 (2H, m), 4.7-4.9 (1H, m), 7.2-7.3 (2H, m), 7.38 (1H, d, J=7.8 Hz), 7.45-7.95 (1H, m), 8.49 (1H, d, J=4.1 Hz), 10.30 (1H, s)

APCI-MS (m/z): 516 (M+H)⁺

### EXAMPLE 115

N-{2-Methyl-1-[2-(2-pyridinyl)ethyl]-2,3-dihydro-1H-indol-5-yl}-4′-(trifluoromethyl)-1,1′-biphenyl-2-carboxamide

The title compound was obtained from N-(2-methyl-2,3-dihydro-1H-indol-5-yl)-4′-(trifluoromethyl)-1,1′-biphenyl-2-carboxamide and 2-vinylpyridine in the same manner as in Preparation 1 as light brown crystals.

¹H-NMR (DMSO-d₆):δ 1.17 (3H, d, J=6.1 Hz), 2.35-2.5 (1H, m), 2.8-3.1 (4H, m), 3.3-3.7 (3H, m), 6.33 (1H, d, J=8.4 Hz), 7.05-7.3 (3H, m), 7.30 (1H, d, J=7.8 Hz), 7.5-7.8 (9H, m), 8.51 (1H, d, J=3.9 Hz), 9.94 (1H, s)

APCI-MS (m/z): 502 (M+H)⁺

Preparation 111

N-(1H-Indol-5-yl)-4′-methyl-1,1′-biphenyl-2-carboxamide

The title compound was obtained from 5-indolamine and 4′-methyl-1,1′-biphenyl-2-carbonyl chloride in the same manner as in Example 1 as brown crystals.

¹H-NMR (DMSO-d₆):δ 2.28 (3H, s), 6.35 (2H, br s), 7.1-7.6 (11H, m), 7.81 (1H, d, J=1.5 Hz), 9.99 (1H, d, J=1.5 Hz), 10.97 (1H, s)

APCI-MS (m/z): 327 (M+H)⁺

Preparation 112

N-(2,3-Dihydro-1H-indol-5-yl)-4′-methyl-1,1′-biphenyl-2-carboxamide

The title compound was obtained in the same manner as in Preparation 10 as a light brown solid.

¹H-NMR (DMSO-d₆):δ 2.30 (3H, s), 2.84 (2H, t, J=8.5 Hz), 3.37 (2H, t, J=8.5 Hz), 5.31 (1H, br s), 6.38 (1H, d, J=8.3 Hz), 7.0-7.6 (10H, m), 9.78 (1H, s)

ESI-MS(m/z): 351 (M+Na)⁺, 329 (M+H)⁺

### EXAMPLE 116

N-(1-{[6-(2,5-Dimethyl-1H-pyrrol-1-yl)-2-pyridinyl]acetyl}-2,3-dihydro-1H-indol-5-yl)-4′-methyl-1,1′-biphenyl-2-carboxamide

The title compound was obtained from N-(2,3-dihydro-1H-indol-5-yl)-4′-methyl-1,1′-biphenyl-2-carboxamide and [6-(2,5-dimethyl-1H-pyrrol-1-yl)-2-pyridinyl]acetic acid in the same manner as in Preparation 5 as a light brown solid.

¹H-NMR (DMSO-d₆):δ 2.02 (6H, s), 2.29 (3H, s), 3.10 (2H, t, J=8.5 Hz), 4.05 (2H, s), 4.16 (2H, t, J=8.5 Hz), 5.77 (2H, s), 7.15-7.6 (14H, m), 7.85-8.0 (2H, m), 10.16 (1H, s)

APCI-MS (m/z): 541 (M+H)⁺

### EXAMPLE 117

N-{1-[(6-Amino-2-pyridinyl)acetyl]-2,3-dihydro-1H-indol-5-yl}-4′-methyl-1,1′-biphenyl-2-carboxamide

The title compound was obtained in the same manner as in Example 9 as white crystals.

¹H-NMR (DMSO-d₆):δ 2.29 (3H, s), 3.09 (2H, t, J=7.9 Hz), 3.69 (2H, s), 4.17 (2H, t, J=7.9 Hz), 5.85 (2H, br s), 6.29 (1H, d, J=7.7 Hz), 6.42 (1H, d, J=6.8 Hz), 7.17 (1H, d, J=8.2 Hz), 7.26 (1H, d, J=4.3 Hz), 7.33 (2H, d, J=8.2 Hz) 7.4-7.6 (7H, m), 7.91 (2H, d, J=8.7 Hz), 10.15 (1H, s)

APCI-MS (m/z): 463 (M+H)⁺

Preparation 113

1-{[6-(2,5-Dimethyl-1H-pyrrol-1-yl)-2-pyridinyl]acetyl}-5-nitroindoline

The title compound was obtained from 5-nitroindoline and [6-(2,5-dimethyl-1H-pyrrol-1-yl)-2-pyridinyl]acetic acid in the same manner as in Example 8 as light yellow crystals.

¹H-NMR (DMSO-d₆):δ 2.02 (6H, s), 3.25 (2H, t, J=8.6 Hz), 4.16 (2H, s), 4.30 (2H, t, J=8.6 Hz), 5.77 (2H, s), 7.31 (1H, d, J=8.6 Hz), 7.31 (1H, d, J=8.6 Hz), 7.98 (1H, dd, J=8.6 Hz, 8.6 Hz), 8.00-8.15 (3H, m)

APCI-MS (m/z): 377 (M+H)⁺

Preparation 114

1-{[6-(2,5-Dimethyl-1H-pyrrol-1-yl)-2-pyridinyl]acetyl}-5-indolinamine

The title compound was obtained in the same manner as in Preparation 6 as light yellow crystals.

¹H-NMR (DMSO-d₆):δ 2.22 (6H, s), 2.99 (2H, t, J=8.4 Hz), 3.98 (2H, s), 4.08 (2H, t, J=8.4 Hz), 4.84 (2H, br s), 5.77 (2H, s), 6.32 (1H, dd, J=8.5 Hz, 2.2 Hz), 6.45 (1H, d, J=2.2 Hz), 7.27 (1H, d, J=7.7 Hz), 7.39 (1H, d, J=7.3 Hz), 7.73 (1H, d, J=8.5 Hz), 7.94 (1H, dd, J=7.7 Hz, 7.3 Hz)

ESI-MS(m/z): 369 (M+Na)⁺, 347 (M+H)⁺

### EXAMPLE 118

N-(1-{[6-(2,5-Dimethyl-1H-pyrrol-1-yl)-2-pyridinyl]acetyl}-2,3-dihydro-1H-indol-5-yl)-41,5-dimethyl-1,1′-biphenyl-2-carboxamide

The title compound was obtained from 1-{[6-(2,5-dimethyl-1H-pyrrol-1-yl)-2-pyridinyl]acetyl}-5-indolinamine and 4′,5-dimethyl-1,1′-biphenyl-2-carboxylic acid in the same manner as in Preparation 5 as a light brown solid.

¹H-NMR (DMSO-d₆):δ 2.02 (6H, s), 2.28 (3H, s), 2.39 (3H, s), 3.09 (2H, t, J=8.2 Hz), 3.98 (2H, s), 4.16 (2H, t, J=8.2 Hz), 5.77 (2H, s), 7.15-7.55 (11H, m), 7.9-8.05 (2H, m), 10.06 (1H, s)

APCI-MS (m/z): 555 (M+H)⁺

### EXAMPLE 119

N-{1-[(6-Amino-2-pyridinyl)acetyl]-2,3-dihydro-1H-indol-5-yl}-4′,5-dimethyl-1,1′-biphenyl-2-carboxamide

The title compound was obtained in the same manner as in Example 9 as white crystals.

¹H-NMR (DMSO-d₆):δ 2.28 (3H, s), 2.39 (3H, s), 3.09 (2H, t, J=8.1 Hz), 3.69 (2H, s), 4.17 (2H, t, J=8.1 Hz), 5.84 (2H, br s), 6.30 (1H, d, J=7.9 Hz), 6.42 (1H, d, J=6.9 Hz), 7.1-7.5 (11H, m), 7.91 (1H, d, J=8.6 Hz), 10.04 (1H, s)

APCI-MS (m/z): 477 (M+H)⁺

Preparation 115

N-(1-Acetyl-2,3-dihydro-1H-indol-5-yl)-4′-nitro-1,1′-biphenyl-2-carboxamide

The title compound was obtained in the same manner as in Preparation 47.

¹H-NMR (DMSO-d₆):δ 2.12 (3H, s), 3.08 (2H, t, J=8.32 Hz), 4.04 (2H, t, J=8.32 Hz), 7.23 (1H, d, J=8.60 Hz), 7.50-7.70 (7H, m), 7.92 (1H, d, J=8.64 Hz), 8.25 (2H, d, J=8.68 Hz), 10.31 (1H, s)

Preparation 116

N-(2,3-Dihydro-1H-indol-5-yl)-4′-nitro-1,1′-biphenyl-2-carboxamide

The title compound was obtained in the same manner as in Preparation 46.

¹H-NMR (DMSO-d₆):δ 2.84 (2H, t, J=8.28 Hz), 3.33-3.37 (2H, m), 5.35 (1H, s), 6.39 (1H, d, J=8.24 Hz), 7.01 (1H, dd, J=1.44 Hz, 8.24 Hz), 4.23 (1H, d, J=1.44 Hz), 7.50-7.71 (6H, m), 8.25 (2H, dd, J=1.70 Hz, 7.04 Hz), 9.97 (1H, s)

### EXAMPLE 120

4′-Nitro-N-[1-(2-pyridinylacetyl)-2,3-dihydro-1H-indol-5-yl]-1,1′-biphenyl-2-carboxamide

The title compound was obtained in the same manner as in Example 41.

¹H-NMR (DMSO-d₆):δ 3.11 (2H, t, J=8.30 Hz), 3.99 (2H, s), 4.19 (2H, t, J=8.30 Hz), 7.26-7.37 (3H, m), 7.51-7.76 (8H, m), 8.25 (2H, d, J=8.78 Hz), 7.92 (1H, d, J=8.68 Hz), 8.48 (1H, d, J=4.98 Hz), 10.34 (1H, s)

### EXAMPLE 121

A mixture of 4′-nitro-N-[1-(2-pyridinylacetyl)-2,3-dihydro-1H-indol-5-yl]-1,1′-biphenyl-2-carboxamide (480 mg) in methanol (25 ml) and tetrahydrofuran (25 ml) was hydrogenated over 10% palladium-carbon (200 mg) under an atmospheric pressure of hydrogen at ambient temperature for 8 hours. After removal of the catalyst, the solvent was evaporated in vacuo and the residue was crystallized from acetone and diisopropyl ether to give 4′-amino-N-[1-(2-pyridinylacetyl)-2,3-dihydro-1H-indol-5-yl]-1,1′-biphenyl-2-carboxamide (305 mg).

¹H-NMR (DMSO-d₆):δ 3.12 (2H, t, J=8.12 Hz), 3.99 (2H, s), 4.19 (2H, t, J=8.12 Hz), 5.14 (2H, s), 6.52 (2H, d, J=8.42 Hz), 7.13 (2H, d, J=8.42 Hz), 7.23-7.76 (8H, m), 7.75-7.76 (1H, m), 7.91 (1H, d, J=8.66 Hz), 8.50 (1H, d, J=5.00 Hz), 10.03 (1H, s)

### EXAMPLE 122

A mixture of 4′-amino-N-[1-(2-pyridinylacetyl)-2,3-dihydro-1H-indol-5-yl]-1,1′-biphenyl-2-carboxamide (170 mg) and acetic anhydride (116 mg) in ethyl acetate (20 ml) was refluxed under stirring for 5 hours. The reaction mixture was poured into a mixture of ethyl acetate and water, and adjusted to pH 8.0 with aqueous potassium carbonate solution. The organic layer was washed with brine and dried over magnesium sulfate. The solvent was evaporated in vacuo and the residue was recrystallized from ethyl acetate and diisopropyl ether to give 4′-acetylamino-N-[1-(2-pyridinylacetyl)-2,3-dihydro-1H-indol-5-yl]-1,1′-biphenyl-2-carboxamide (140 mg).

¹H-NMR (DMSO-d₆):δ 2.03 (3H, s), 3.11 (2H, t, J=8.00 Hz), 3.99 (2H, s), 4.18 (2H, t, J=8.00 Hz), 7.23-7.59 (12H, m), 7.75-7.76 (1H, m), 7.91 (1H, d, J=8.68 Hz), 8.47-8.50 (1H, m), 10.00 (1H, s), 10.15 (1H, s)

### EXAMPLE 123

4′-(Methylthio)-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.46 (3H, s), 3.11 (2H, t, J=8.30 Hz), 3.99 (2H, s), 4.19 (2H, t, J=8.30 Hz), 7.23-7.56 (12H, m), 7.45-7.76 (1H, m), 7.92 (1H, d, J=8.64 Hz), 8.49 (1H, d, J=5.00 Hz), 10.21 (1H, s)

negative EPI-MS(m/z): 478 (M−H)⁻

### EXAMPLE 124

4′-(Isopropylthio)-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.26 (6H, d, J=6.62 Hz), 3.10 (2H, t, J=8.32 Hz), 3.45-3.55 (1H, m), 3.99 (2H, s), 4.18 (2H, t, J=8.32 Hz), 7.17-7.57 (12H, m), 7.75-7.76 (1H, m), 7.90 (1H, d, J=8.64 Hz), 8.50 (1H, d, J=5.00 Hz), 10.10 (1H, s)

negative EPI-MS(m/z): 506 (M−H)⁻

### EXAMPLE 125

4′-Acetyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.57 (3H, s), 3.10 (2H, t, J=8.18 Hz), 3.98 (2H, s), 4.18 (2H, t, J=8.18 Hz), 7.23-7.30 (2H, m), 7.35 (1H, d, J=7.88 Hz), 7.52-7.63 (7H, m), 7.75-7.76 (1H, m), 7.91-7.98 (3H, m), 8.48-8.50 (1H, m), 10.30 (1H, s)

APCI-MS (m/z): 476 (M+H)⁺

### EXAMPLE 126

A mixture of 4′-acetyl-N-[1-(2-pyridinylacetyl)-2,3-dihydro-1H-indol-5-yl]-1,1′-biphenyl-2-carboxamide (476 mg) and sodium borohydride (76 mg) in methanol (10 ml) and tetrahydrofuran (10 ml) was stirred at ambient temperature for 3 hours. The reaction mixture was evaporated in vacuo and the residue was dissolved in a mixture of ethyl acetate and water. The organic layer was washed with brine and dried over magnesium sulfate. The solvent was concentrated in vacuo and the precipitate was collected by filtration to give 4′-(1-hydroxyethyl)-N-[1-(2-pyridinylacetyl)-2,3-dihydro-1H-indol-5-yl]-1,1′-biphenyl-2-carboxamide (384 mg).

¹H-NMR (DMSO-d₆):δ 1.31 (3H, d, J=6.42 Hz), 3.11 (2H, t, J=8.28 Hz), 3.99 (2H, s), 4.18 (2H, t, J=8.28 Hz), 4.65-4.74 (1H, m), 5.16 (1H, d, J=4.28 Hz), 7.21-7.55 (12H, m), 7.75-7.76 (1H, m), 7.92 (1H, d, J=8.66 Hz), 8.49 (1H, d, J=5.00 Hz), 10.19 (1H, s)

EPI-MS(m/z): 478 (M+H)⁺

Preparation 117

A mixture of methyl 4′-acetyl-1,1′-biphenyl-2-carboxylate (5.09 g), methyl(triphenyl)phosphonium bromide (10.7 g) and potassium tert-butoxide (3.82 g) in tetrahydrofuran (100 ml) was refluxed under stirring for 3 hours. The reaction mixture was poured into a mixture of ethyl acetate and water, and adjusted to pH 8.0 with aqueous potassium carbonate solution. The organic layer was washed with brine and dried over magnesium sulfate. The solvent was evaporated in vacuo and the residue was chromatographed on silica gel eluting with n-hexane:ethyl acetate (9:1 v/v). The fraction was evaporated in vacuo to give methyl 4′-isopropenyl-1,1′-biphenyl-2-carboxylate (2.84 g).

¹H-NMR (DMSO-d₆):δ 2.15 (3H, s), 3.62 (3H, s), 5.14 (1H, s), 5.50 (1H, s), 7.28 (2H, d, J=6.69 Hz), 7.43-7.63 (5H, m), 7.74 (1H, d, J=7.62 Hz)

Preparation 118

A mixture of methyl 4′-isopropenyl-1,1′-biphenyl-2-carboxylate (1.3 g) and sodium hydroxide (412 mg) in methanol (15 ml) and water (15 ml) was refluxed under stirring for 2 hours. The reaction mixture was evaporated in vacuo and the residue was dissolved in a mixture of water and ethyl acetate. The aqueous layer was adjusted to pH 2.0 with 10% hydrochloric acid and extracted with ethyl acetate. The extract was washed with brine and dried over magnesium sulfate. The solvent was concentrated in vacuo and the precipitate was collected by filtration to give 4′-isopropenyl-1,1′-biphenyl-2-carboxylic acid (930 mg).

¹H-NMR (DMSO-d₆):δ 2.15 (3H, s), 5.12 (1H, s), 5.49 (1H, s), 7.30-7.58 (7H, m), 7.73 (1H, d, J=7.56 Hz), 12.78 (1H, s)

### EXAMPLE 127

4′-Isopropenyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.10 (3H, s), 3.11 (2H, t, J=8.28 Hz), 3.99 (2H, s), 4.18 (2H, t, J=8.28 Hz), 5.10 (1H, s), 5.45 (1H, s), 7.23-7.57 (12H, m), 7.75-7.76 (1H, m), 7.92 (1H, d, J=8.72 Hz), 8.50 (1H, d, J=5.02 Hz), 10.23 (1H, s)

### EXAMPLE 128

A mixture of 4′-isopropenyl-N-[1-(2-pyridinylacetyl)-2,3-dihydro-1H-indol-5-yl]-1,1′-biphenyl-2-carboxamide (500 mg) and 10% hydrochloric acid-methanol solution (20 ml) in chloroform (20 ml) was stirred at ambient temperature for 1 day. The reaction mixture was evaporated in vacuo and the residue was dissolved in a mixture of ethyl acetate and water, and adjusted to pH 8.0 with aqueous potassium carbonate solution. The organic layer was washed with brine and dried over magnesium sulfate. The solvent was evaporated in vacuo and the residue was chromatographed on silica gel eluting with ethyl acetate and ethyl acetate:methanol (9:1 v/v). The fraction was evaporated in vacuo and the residue was recrystallized from ethyl acetate and diisopropyl ether to give 4′-(1-hydroxy-1-methylethyl)-N-[1-(2-pyridinylacetyl)-2,3-dihydro-1H-indol-5-yl]-1,1′-biphenyl-2-carboxamide (132 mg).

¹H-NMR (DMSO-d₆):δ 1.42 (6H, s), 3.11 (2H, t, J=8.12 Hz), 3.99 (2H, s), 4.18 (2H, t, J=8.12 Hz), 5.02 (1H, s), 7.18-7.55 (12H, m), 7.71-7.76 (1H, m), 7.92 (1H, d, J=8.66 Hz), 8.49 (1H, d, J=5.00 Hz), 10.16 (1H, s)

ESI-MS(m/z): 492 (M+H)⁺

Preparation 119

A mixture of methyl 4′-isopropenyl-1,1′-biphenyl-2-carboxylate (1.55 g) in methanol (50 ml) was hydrogenated over 10% palladium-carbon (400 mg) under an atmospheric pressure of hydrogen at ambient temperature for 10 hours. After removal of the catalyst, the solvent was evaporated in vacuo to give methyl 4′-isopropyl-1,1′-biphenyl-2-carboxylate (1.27 g).

¹H-NMR (DMSO-d₆):δ 1.23 (6H, d, J=6.94 Hz), 2.86-3.00 (1H, m), 3.60 (3H, s), 7.19-7.31 (4H, m), 7.41-7.73 (4H, m)

Preparation 120

4′-Isopropyl-1,1′-biphenyl-2-carboxylic acid

The title compound was obtained in the same manner as in Preparation 118.

¹H-NMR (DMSO-d₆):δ 1.23 (6H, d, J=6.94 Hz), 2.86-2.99 (1H, m), 7.23-7.27 (4H, m), 7.35-7.58 (3H, m), 7.69 (1H, dd, J=1.19 Hz, 7.52 Hz), 12.74 (1H, br s)

### EXAMPLE 129

4′-Isopropyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.15 (6H, d, J=6.72 Hz), 2.81-2.91 (1H, m), 3.11 (2H, t, J=8.14 Hz), 3.99 (2H, s), 4.05 (2H, t, J=8.14 Hz), 7.17-7.55 (12H, m), 7.75-7.76 (1H, m), 7.91 (1H, d, J=8.68 Hz) 8.50 (1H, d, J=5.00 Hz), 10.14 (1H, s)

negative EPI-MS(m/z): 474 (M−H)⁻

### EXAMPLE 130

A mixture of 4′-acetyl-N-[1-(2-pyridinylacetyl)-2,3-dihydro-1H-indol-5-yl]-1,1-biphenyl-2-carboxamide (476 mg), benzylamine (215 mg) and sodium triacetoxyborohydride (636 mg) in dichloromethane (20 ml) was stirred at ambient temperature for 15 hours. Water (20 ml) was added to a resultant mixture. The mixture was adjusted to pH 8.5 with 5% aqueous potassium carbonate solution and stirred for 30 minutes. The organic layer was washed with brine and dried over magnesium sulfate. The solvent was evaporated in vacuo and the residue was chromatographed on silica gel eluting with chloroform:methanol (97:3 v/v). The fraction was evaporated in vacuo and the residue was recrystallized from ethyl acetate and diisopropyl ether to give 4′-[1-(benzylamino)ethyl]-N-[1-(2-pyridinylacetyl)-2,3-dihydro-1H-indol-5-yl]-1,1′-biphenyl-2-carboxamide (130 mg).

¹H-NMR (DMSO-d₆):δ 1.24 (3H, d, J=6.50 Hz), 3.01 (2H, t, J=8.20 Hz), 3.23-3.38 (2H, m), 3.62-3.68 (2H, m), 3.95 (2H, s), 4.13 (2H, t, J=8.20 Hz), 7.18-7.57 (17H, m), 7.71-7.76 (1H, m), 7.87 (1H, d, J=8.68 Hz), 8.49 (1H, d, J=5.00 Hz), 9.97 (1H, s)

APCI-MS (m/z): 567 (M+H)⁺

### EXAMPLE 131

A mixture of 4′-[1-(benzylamino)ethyl]-N-[1-(2-pyridinylacetyl)-2,3-dihydro-1H-indol-5-yl]-1,1′-biphenyl-2-carboxamide (320 mg) in methanol (30 ml) was hydrogenated over 10% palladium-carbon (200 mg) under an atmospheric pressure of hydrogen at ambient temperature for 10 hours. After removal of the catalyst, the solvent was evaporated in vacuo and the residue was chromatographed on silica gel eluting with chloroform:methanol (97:3 v/v). The fraction was evaporated in vacuo to give 4′-(1-aminoethyl)-N-[1-(2-pyridinylacetyl)-2,3-dihydro-1H-indol-5-yl]-1,1′-biphenyl-2-carboxamide (105 mg).

¹H-NMR (DMSO-d₆):δ 1.24 (3H, d, J=6.50 Hz), 3.01 (2H, t, J=8.38 Hz), 3.61-3.71 (1H, m), 3.95 (2H, s), 4.09 (2H, t, J=8.38 Hz), 6.55-7.53 (13H, m), 7.75-7.86 (1H, m), 7.89 (1H, d, J=8.70 Hz), 8.45-8.50 (1H, m), 9.96 (1H, s)

Preparation 121

A mixture of 4′-methylsulfonyl-1,1′-biphenyl-2-carboxylic acid (552 mg), thionyl chloride (357 mg) and N,N-dimethylformamide (4.4 mg) in toluene (50 ml) was stirred at 80° C. for 3 hours. The rection mixture was evaporated in vacuo and the residue was dissolved in toluene (30 ml). The solution was evaporated in vacuo to give 4′-methylsulfonyl-1,1′-biphenyl-2-carbonyl chloride (590 mg).

Preparation 122

N-(1-Acetyl-2,3-dihydro-1H-indol-5-yl)-4′-(methylsulfonyl)-1,1′-biphenyl-2-carboxamide

The title compound was obtained in the same manner as in Preparation 47.

¹H-NMR (DMSO-d₆):δ 2.12 (3H, s), 3.09 (2H, t, J=8.20 Hz), 3.23 (3H, s), 4.05 (2H, t, J=8.20 Hz), 7.22 (1H, dd, J=1.58 Hz, 8.63 Hz), 7.44-7.69 (7H, m), 7.90-7.96 (2H, m), 10.29 (1H, s)

Preparation 123

N-(2,3-Dihydro-1H-indol-5-yl)-4′-(methylsulfonyl)-1,1′-biphenyl-2-carboxamide

The title compound was obtained in the same manner as in Preparation 46.

¹H-NMR (DMSO-d₆):δ 2.85 (2H, t, J=8.29 Hz), 3.24 (3H, s), 3.24-3.37 (2H, m), 5.35 (1H, s), 6.39 (1H, d, J=8.29 Hz), 6.99 (1H, dd, J=1.92 Hz, 8.29 Hz), 7.20 (1H, s), 7.44-7.69 (6H, m), 7.94 (2H, d, J=8.37 Hz), 9.93 (1H, s)

### EXAMPLE 132

4′-(Methylsulfonyl)-N-[1-(2-pyridinylacetyl)-2,3-dihydro-1H-indol-5-yl]-1,1′-biphenyl-2-carboxamide

The title compound was obtained in the same manner as in Example 41.

¹H-NMR (DMSO-d₆):δ 3.11 (2H, t, J=8.26 Hz), 3.23 (3H, s), 4.01 (2H, s), 4.19 (2H, t, J=8.26 Hz), 7.21-7.33 (2H, m), 7.51 (1H, d, J=7.84 Hz), 7.49-7.76 (8H, m), 7.93 (2H, d, J=1.40 Hz, 8.50 Hz), 8.48-8.51 (1H, m), 10.30 (1H, s)

EPI-MS(m/z): 512 (M+H)⁺

Preparation 124

5-Methyl-N-(3-oxo-3,4-dihydro-2H-1,4-benzoxazin-7-yl)-4′-(trifluoromethyl)-1,1′-biphenyl-2-carboxamide

The title compound was obtained in the same manner as in Preparation 80.

¹H-NMR (DMSO-d₆):δ 2.42 (3H, s), 4.53 (2H, s), 6.78 (1H, d, J=8.44 Hz), 7.08 (1H, dd, J=2.06 Hz, 8.44 Hz), 7.21 (1H, d, J=2.06 Hz), 7.33-7.37 (2H, m), 7.52 (1H, d, J=7.60 Hz), 7.60 (2H, d, J=8.28 Hz), 7.75 (2H, d, J=8.28 Hz), 10.21 (1H, s), 10.63 (1H, s)

Preparation 125

N-(3,4-Dihydro-2H-1,4-benzoxazin-7-yl)-5-methyl-4′-(trifluoromethyl)-1,1′-biphenyl-2-carboxamide

The title compound was obtained in the same manner as in Preparation 81.

¹H-NMR (DMSO-d₆):δ 2.41 (3H, s), 3.20-3.23 (2H, m), 4.06-4.08 (2H, m), 5.53 (1H, s), 6.43 (1H, d, J=8.48 Hz), 6.77 (1H, dd, J=2.24 Hz, 8.48 Hz), 6.90 (1H, d, J=2.24 Hz), 7.30-7.33 (2H, m), 7.47 (1H, d, J=7.72 Hz), 7.61 (2H, d, J=8.28 Hz), 7.74 (2H, d, J=8.28 Hz), 9.83 (1H, s)

### EXAMPLE 133

5-Methyl-N-{4-[2-(2-pyridinyl)ethyl]-3,4-dihydro-2H-1,4-benzoxazin-7-yl}-4′-(trifluoromethyl)-1,1′-biphenyl-2-carboxamide

The title compound was obtained in the same manner as in Example 75.

¹H-NMR (DMSO-d₆):δ 2.41 (3H, s), 2.90-2.98 (2H, m), 3.22-3.24 (2H, m), 3.55-3.62 (2H, m), 4.05-4.10 (2H, m), 6.67 (1H, d, J=8.58 Hz), 6.91-6.99 (2H, m), 7.22-7.35 (2H, m), 7.49 (1H, d, J=7.60 Hz), 7.58-7.78 (5H, m), 8.51 (1H, d, J=5.00 Hz), 9.96 (1H, s)

APCI-MS (m/z): 518 (M+H)⁺

### EXAMPLE 134

5-Methyl-N-[4-(2-pyridinylacetyl)-3,4-dihydro-2H-1,4-benzoxazin-7-yl]-4′-(trifluoromethyl)-1,1′-biphenyl-2-carboxamide

The title compound was obtained in the same manner as in Example 76.

¹H-NMR (DMSO-d₆):δ 2.42 (3H, s), 3.88-3.90 (2H, m), 4.11 (2H, s), 4.21-4.26 (2H, m), 6.97 (1H, dd, J=1.86 Hz, 8.89 Hz), 7.30-7.37 (5H, m), 7.50-7.77 (7H, m), 8.49 (1H, d, J=4.18 Hz), 10.26 (1H, s)

APCI-MS (m/z): 532 (M+H)⁺

### EXAMPLE 135

A mixture of N-(3,4-dihydro-2H-1,4-benzoxazin-7-yl)-4′-(trifluoromethyl)-1,1′-biphenyl-2-carboxamide (1.53 g), [6-(2,5-dimethyl-1H-pyrrol-1-yl)-2-pyridinyl]acetic acid (929 mg), 1-hydroxybenzotriazole hydrate (618 mg) and 1-[3-(dimethylamino)propyl]-3-ethylcarbodiimide hydrochloride (770 mg) in N,N-dimethylformamide(20 ml) was stirred at ambient temperature for 15 hours. The reaction mixture was poured into a mixture of ethyl acetate and water, and the organic layer was washed with brine and dried over magnesium sulfate. The solvent was evaporated in vacuo and the residue was chromatographed on silica gel eluting with ethyl acetate:n-hexane (6:4 v/v). The fraction was evaporated in vacuo to give N-(4-{[6-(2,5-dimethyl-1H-pyrrol-1-yl)-2-pyridinyl]acetyl}-3,4-dihydro-2H-1,4-benzoxazin-7-yl)-4′-(trifluoromethyl)-1,1′-biphenyl-2-carboxamide (0.95 g).

¹H-NMR (DMSO-d₆):δ 1.28 (6H, s), 3.86-3.88 (2H, m), 4.17-4.20 (2H, m), 5.78 (1H, s), 6.99 (1H, d, J=8.92 Hz), 7.20-7.97 (13H, m), 10.35 (1H, s)

### EXAMPLE 136

A mixture of N-(4-{[6-(2,5-dimethyl-1H-pyrrol-1-yl)-2-pyridinyl]acetyl}-3,4-dihydro-2H-1,4-benzoxazin-7-yl)-4′-(trifluoromethyl)-1,1′-biphenyl-2-carboxamide (0.95 g), hydroxylamine hydrochloride (1.08 g) and triethylamine (315 mg) in ethanol (40 ml) and water (10 ml) was refluxed under stirring for 10 hours. The reaction mixture was evaporated in vacuo and the residue was dissolved in a mixture of ethyl acetate and water. The aqueous layer was adjusted to pH 8.0 with aqueous potassium carbonate solution and extracted with ethyl acetate. The extract was washed with brine and dried over magnesium sulfate. The solvent was evaporated in vacuo and the residue was chromatographed on silica gel eluting with ethyl acetate and ethyl acetate:methanol (9:1 v/v). The fraction was evaporated in vacuo to give N-{4-[(6-amino-2-pyridinyl)acetyl]-3,4-dihydro-2H-1,4-benzoxazin-7-yl}-4′-(trifluoromethyl)-1,1′-biphenyl-2-carboxamide (100 mg).

¹H-NMR (DMSO-d₆):δ 3.82 (2H, s), 3.97-3.98 (2H, m), 4.21-4.23 (2H, m), 5.87 (2H, s), 6.29 (1H, d, J=8.12 Hz), 6.40 (1H, d, J=7.14 Hz), 6.98 (1H, d, J=8.86 Hz), 7.20 (1H, d, J=2.14 Hz), 7.26-7.34 (1H, m), 7.48-7.64 (7H, m), 7.76 (2H, d, J=8.32 Hz), 10.34 (1H, s)

ESI-MS(m/z): 533 (M+H)⁺

### EXAMPLE 137

N-(4-{[6-(2,5-Dimethyl-1H-pyrrol-1-yl)-2-pyridinyl]acetyl}-3,4-dihydro-2H-1,4-benzoxazin-7-yl)-5-methyl-4′-(trifluoromethyl)-1,1′-biphenyl-2-carboxamide

The title compound was obtained in the same manner as in Example 135.

¹H-NMR (DMSO-d₆):δ 2.02 (6H, s), 2.42 (3H, s), 3.86-3.88 (2H, m), 4.16-4.19 (4H, m), 5.78 (2H, s), 6.90-6.91 (1H, m), 7.20-7.62 (11H, m), 7.90-7.93 (2H, m), 10.26 (1H, s)

### EXAMPLE 138

N-{4-[(6-Amino-2-pyridinyl)acetyl]-3,4-dihydro-2H-1,4-benzoxazin-7-yl}-5-methyl-4′-(trifluoromethyl)-1,1′-biphenyl-2-carboxamide

The title compound was obtained in the same manner as in Example 136.

¹H-NMR (DMSO-d₆):δ 2.43 (3H, s), 3.63 (2H, s), 3.81-3.87 (2H, m), 4.20-4.22 (2H, m), 5.87 (2H, s), 6.29 (1H, d, J=8.14 Hz), 6.39 (1H, d, J=7.18 Hz), 6.98 (1H, d, J=8.84 Hz), 7.20 (1H, d, J=2.02 Hz), 7.26-7.37 (3H, m), 7.50-7.62 (4H, m), 7.75 (2H, d, J=8.26 Hz), 10.25 (1H, s)

negative EPI-MS(m/z): 545 (M−H)⁻

Preparation 126

A mixture of 5-methyl-4′-(trifluoromethyl)-1,1′-biphenyl-2-carboxylic acid (1.4 g), thionyl chloride (892 mg) and N,N-dimethylformamide (11 mg) in toluene (14 ml) was stirred at 55-60° C. for 3 hours. The reaction mixture was evaporated in vacuo and the residue was dissolved in toluene (30 ml). The solution was evaporated in vacuo to give 5-methyl-4′-(trifluoromethyl)-1,1′-biphenyl-2-carbonyl chloride (1.5 g).

Preparation 127

N-(4-Fluoro-3-nitrophenyl)-5-methyl-4′-(trifluoromethyl)-1,1′-biphenyl-2-carboxamide

The title compound was obtained in the same manner as in Preparation 73.

¹H-NMR (DMSO-d₆):δ 2.44 (3H, s), 7.39-7.87 (9H, m), 8.46 (1H, dd, J=2.58 Hz, 6.92 Hz), 10.77 (1H, s)

Preparation 128

5-Methyl-N-(3-nitro-4-{[2-(2-pyridinyl)ethyl]amino}phenyl)-4′-(trifluoromethyl)-1,1′-biphenyl-2-carboxamide

The title compound was obtained in the same manner as in Preparation 74.

¹H-NMR (DMSO-d₆):δ 2.43 (3H, s), 3.10 (2H, t, J=6.66 Hz), 3.67-3.76 (2H, m), 7.09 (1H, d, J=9.38 Hz), 7.24-7.38 (4H, m), 7.55-7.78 (7H, m), 8.30 (1H, m), 8.42 (1H, d, J=2.44 Hz), 8.53-8.55 (1H, m), 10.31 (1H, s)

Preparation 129

Ethyl {[4-({[5-methyl-4′-(trifluoromethyl)-1,1′-biphenyl-2-yl]carbonyl}amino)-2-nitrophenyl][2-(2-pyridinyl)ethyl]amino}(oxo)acetate

The title compound was obtained in the same manner as in Preparation 84.

¹H-NMR (DMSO-d₆):δ 0.85 (3H, t, J=7.10 Hz), 2.43 (3H, s), 3.07-3.10 (2H, m), 3.67-3.72 (2H, m), 3.97 (2H, q, J=7.10 Hz), 7.11-7.43 (7H, m), 7.54-7.77 (7H, m), 8.39-8.52 (3H, m), 10.91 (1H, s)

### EXAMPLE 139

N-{2,3-Dioxo-1-[2-(2-pyridinyl)ethyl]-1,2,3,4-tetrahydro-6-quinoxalinyl}-5-methyl-4′-(trifluoromethyl)-1,1′-biphenyl-2-carboxamide

The title compound was obtained in the same manner as in Example 80.

¹H-NMR (DMSO-d₆):δ 2.43 (3H, s), 3.03-3.10 (2H, m), 4.34-4.46 (2H, m), 7.26-7.39 (6H, m), 7.54-7.78 (7H, m), 8.53 (1H, d, J=4.86 Hz), 10.48 (1H, s), 12.00 (1H, s)

### EXAMPLE 140

5-Methyl-N-{1-[2-(2-pyridinyl)ethyl]-1,2,3,4-tetrahydro-6-quinoxalinyl}-4′-(trifluoromethyl)-1,1′-biphenyl-2-carboxamide

The title compound was obtained in the same manner as in Example 79.

¹H-NMR (DMSO-d₆):δ 2.41 (3H, s), 2.93 (2H, m), 3.18 (4H, br s), 3.34 (2H, m), 5.51 (1H, m), 6.50-6.56 (2H, m), 6.78 (1H, br s), 7.21-7.29 (4H, m), 7.46 (1H, d, J=7.60 Hz), 7.48-7.77 (5H, m), 8.51 (1H, d, J=5.00 Hz), 9.78 (1H, s)

ESI-MS(m/z): 517 (M+H)⁺

Preparation 130

N-(3-Amino-4-{[2-(2-pyridinyl)ethyl]amino}phenyl)-5-methyl-4′-(trifluoromethyl)-1,1′-biphenyl-2-carboxamide

The title compound was obtained in the same manner as in Preparation 75.

¹H-NMR (DMSO-d₆):δ 2.41 (3H, s), 2.97-3.05 (2H, m), 3.34 (2H, m), 4.44-4.47 (3H, m), 6.39 (1H, d, J=8.46 Hz), 6.62 (1H, dd, J=2.14 Hz, 8.46 Hz), 6.87 (1H, d, J=2.14 Hz), 7.21-7.24 (4H, m), 7.31 (1H, d, J=8.56 Hz), 7.44-7.76 (5H, m), 8.50 (1H, d, J=5.00 Hz), 9.75 (1H, s)

### EXAMPLE 141

5-Methyl-N-{1-[2-(2-pyridinyl)ethyl]-1H-benzimidazol-5-yl}-4′-(trifluoromethyl)-1,1′-biphenyl-2-carboxamide

The title compound was obtained in the same manner as in Example 73.

¹H-NMR (DMSO-d₆):δ 2.43 (3H, s), 3.26 (2H, t, J=6.88 Hz), 4.61 (2H, t, J=6.86 Hz), 7.13-7.47 (6H, m), 7.55-7.86 (6H, m), 8.00 (1H, s), 8.17 (1H, s), 8.51 (1H, d, J=4.88 Hz), 10.22 (1H, S)

ESI-MS(m/z): 501 (M+H)⁺

### EXAMPLE 142

A solution of N-(3-amino-4-{[2-(2-pyridinyl)ethyl]amino}phenyl)-5-methyl-4′-(trifluoromethyl)-1,1′-biphenyl-2-carboxamide (295 mg) and acetic anhydride (307 mg) in ethyl acetate (10 ml) was refluxed under stirring for 3 hours. The reaction mixture was evaporated in vacuo and the residue was dissolved in ethanol (10 ml) and conc. hydrochloric acid (2 ml). The resultant solution was refluxed under stirring for 2.5 hours. The reaction mixture was poured into a mixture of ethyl acetate and water, and adjusted to pH 8.0 with aqueous potassium carbonate solution. The organic layer was washed with brine and dried over magnesium sulfate. The solvent was evaporated in vacuo and the residue was chromatographed on silica gel eluting with chloroform:methanol (97:3 v/v). The fraction was evaporated in vacuo and the residue was recrystallized from ethyl acetate and diisopropyl ether to give 5-methyl-N-{2-methyl-1-[2-(2-pyridinyl)ethyl]-1H-benzimidazol-5-yl}-4′-(trifluoromethyl)-1,1′-biphenyl-2-carboxamide (84 mg).

¹H-NMR (DMSO-d₆):δ 2.26 (3H, s), 2.43 (3H, s), 3.16 (2H, t, J=6.88 Hz), 4.50 (2H, t, J=6.86 Hz), 7.06 (1H, d, J=7.80 Hz), 7.19-7.38 (5H, m), 7.53-7.77 (7H, m), 8.51 (1H, d, J=5.00 Hz), 10.16 (1H, s) ESI-MS(m/z): 515 (M+H)+

Preparation 131

N-(1-Acetyl-2,3-dihydro-1H-indol-5-yl)-4-methoxy-4′-methyl-1,1′-biphenyl-2-carboxamide

The title compound was obtained in the same manner as in Preparation 47.

¹H-NMR (DMSO-d₆):δ 2.12 (3H, s), 2.17 (3H, s), 3.08 (2H, t, J=8.40 Hz), 3.84 (3H, s), 4.02 (2H, t, J=8.40 Hz), 7.06-7.15 (4H, m), 7.23-7.56 (4H, m), 7.48 (1H, s), 7.91 (1H, d, J=8.64 Hz), 10.13 (1H, s)

Preparation 132

N-(2,3-Dihydro-1H-indol-5-yl)-4-methoxy-4′-methyl-1,1-biphenyl-2-carboxamide

The title compound was obtained in the same manner as in Preparation 46.

¹H-NMR (DMSO-d₆):δ 2.28 (3H, s), 2.85 (2H, t, J=8.25 Hz), 3.27-3.41 (2H, m), 3.67 (3H, s), 5.38 (1H, br s), 6.40 (1H, d, J=8.22 Hz), 7.02-7.16 (5H, m), 7.24-7.35 (4H, m), 9.79 (1H, s)

### EXAMPLE 143

A mixture of N-(2,3-dihydro-1H-indol-5-yl)-4-methoxy-4′-methyl-1,1′-biphenyl-2-carboxamide (896 mg), [2-(formylamino)-1,3-thiazol-4-yl]acetic acid (483 mg), 1-hydroxybenzotriazole hydrate (421 mg) and 1-[3-(dimethylamino)propyl]-3-ethylcarbodiimide hydrochloride (525 mg) in N,N-dimethylformamide(10 ml) was stirred at ambient temperature for 15 hours. The reaction mixture was poured into a mixture of ethyl acetate and water and the organic layer was washed with brine and dried over magnesium sulfate. The solvent was evaporated in vacuo and the residue was dissolved in methanol (10 ml) and conc. hydrochloric acid (1.5 ml). The solution was stirred at ambient temperature for 5 hours. The resultant solution was poured into a mixture of ethyl acetate and water, and adjusted to pH 8.0 with aqueous potassium carbonate solution. The organic layer was washed with brine and dried over magnesium sulfate. The solvent was evaporated in vacuo and the residue was chromatographed on silica gel eluting with ethyl acetate:methanol (95:5 v/v). The fraction was evaporated in vacuo and the residue was recrystallized with ethyl acetate and diisopropyl ether to give N-{1-[(2-amino-1,3-thiazol-4-yl)acetyl]-2,3-dihydro-1H-indol-5-yl}-4-methoxy-4′-(trifluoromethyl)-1,1′-biphenyl-2-carboxamide (437 mg).

¹H-NMR (DMSO-d₆):δ 2.27 (3H, s), 3.09 (2H, t, J=8.08 Hz), 3.78 (2H, s), 3.84 (3H, s), 4.16 (2H, t, J=8.08 Hz), 6.31 (1H, s), 6.89 (2H, s), 7.07-7.37 (8H, m), 7.50 (1H, s), 7.93 (1H, d, J=8.66 Hz), 10.14 (1H, s)

Preparation 133

A solution of 4-bromo-3-oxobutanoyl bromide (6.74 g) in dichloromethane (10 ml) was added portionwise to a mixture of N-(2,3-dihydro-1H-indol-5-yl)-4′-methyl-1,1′-biphenyl-2-carboxamide hydrochloride (9.12 g) and triethylamine (7.59 g) in tetrahydrofuran (120 ml) at 5-20° C. under stirring, and the resultant mixture was stirred at ambient temperature for 3 hours. The reaction mixture was poured into a mixture of ethyl acetate and water. The organic layer was washed with brine and dried over magnesium sulfate. The solvent was evaporated in vacuo and the residue was chromatographed on silica gel eluting with n-hexane:ethyl acetate (6:4 v/v). The fraction was evaporated in vacuo to give N-[1-(4-bromo-3-oxobutanoyl)-2,3-dihydro-1H-indol-5-yl]-4′-methyl-1,1′-biphenyl-2-carboxamide (2.5 g).

### EXAMPLE 144

A mixture of N-[1-(4-bromo-3-oxobutanoyl)-2,3-dihydro-1H-indol-5-yl]-4′-methyl-1,1′-biphenyl-2-carboxamide (1.23 g) and amino{[amino(imino)methyl]amino}-thioxomethane (444 mg) in ethanol (20 ml) was refluxed under stirring for 4 hours. The reaction mixture was poured into a mixture of ethyl acetate and water, and adjusted to pH 8.5 with aqueous potassium carbonate solution. The organic layer was washed with brine and dried over magnesium sulfate. The solvent was evaporated in vacuo and the residue was chromatographed on silica gel eluting with chloroform:methanol (90:10 v/v). The fraction was concentrated in vacuo and the precipitate was collected by filtration to give N-{1-[(2-{[amino(imino)methyl]amino}-1,3-thiazol-4-yl)acetyl]-2,3-dihydro-1H-indol-5-yl}-4′-methyl-1,1′-biphenyl-2-carboxamide (690 mg).

¹H-NMR (DMSO-d₆):δ 2.29 (3H, s), 3.16 (2H, t, J=8.30 Hz), 3.68 (2H, s), 4.16 (2H, t, J=8.30 Hz), 6.41 (1H, s), 6.82 (2H, s), 7.17 (2H, d, J=8.06 Hz), 7.19-7.57 (9H, m), 7.93 (1H, d, J=8.66 Hz), 10.17 (1H, s)

### EXAMPLE 145

4′-Methyl-N-(1-{[2-(methylamino)-1,3-thiazol-4-yl]acetyl}-2,3-dihydro-1H-indol-5-yl)-1,1′-biphenyl-2-carboxamide

The title compound was obtained in the same manner as in Example 144 from N-[1-(4-bromo-3-oxobutanoyl)-2,3-dihydro-1H-indol-5-yl]-4′-methyl-1,1′-biphenyl-2-carboxamide and N-methylthiourea.

¹H-NMR (DMSO-d₆):δ 2.28 (3H, s), 2.77 (3H, d, J=4.68 Hz), 3.09 (2H, t, J=8.22 Hz), 3.63 (2H, s), 4.20 (2H, t, J=8.22 Hz), 6.37 (1H, s), 7.15-7.55 (11H, m), 7.91 (1H, d, J=8.64 Hz), 10.16 (1H, s)

negative EPI-MS(m/Z): 481 (M−H)⁻

### EXAMPLE 146

A solution of 4′-methyl-N-(1-{[2-(methylamino)-1,3-thiazol-4-yl]acetyl}-2,3-dihydro-1H-indol-5-yl)-1,1′-biphenyl-2-carboxamide (535 mg) in tetrahydrofuran (30 ml) was added dropwise to a mixture of lithium aluminum hydride (126 mg) in tetrahydrofuran (20 ml) at 55-60° C. under stirring. The mixture was stirred at 55-60° C. under an atmospheric pressure of nitrogen for 1.5 hours. A mixture of ethyl acetate and water was added to the reaction mixture under ice-cooling and extracted with ethyl acetate. The extract was washed with brine and dried over magnesium sulfate. The solvent was evaporated in vacuo and the residue was chromatographed on silica gel eluting with ethyl acetate:methanol (98:2 v/v). The fraction was evaporated in vacuo and the residue was recrystallized from ethyl acetate and diisopropyl ether to give 4′-methyl-N-(1-{2-[2-(methylamino)-1,3-thiazol-4-yl]ethyl}-2,3-dihydro-1H-indol-5-yl)-1,1′-biphenyl-2-carboxamide (43 mg).

¹H-NMR (DMSO-d₆):δ 2.30 (3H, s), 2.64-2.71 (2H, m), 2.78-2.86 (5H, m), 3.22-3.34 (4H, m), 6.29 (1H, s), 6.40 (1H, d, J=8.42 Hz), 7.09-7.23 (4H, m), 7.32-7.55 (5H, m), 9.85 (1H, s)

negative EPI-MS(m/z): 467 (M−H)⁻

### EXAMPLE 147

To a solution of N-{1-[(6-amino-2-pyridinyl)acetyl]-2,3-dihydro-1H-indol-5-yl}-4′-(trifluoromethyl)-1,1′-biphenyl-2-carboxamide (2.58 g) in methanol (30 ml) was added dropwise 10% hydrogen chloride in methanol (5.5 ml) at 5° C. To the solution was added dropwise diisopropyl ether (60 ml) at 5° C. and the resultant precipitate was collected by filtration and washed with methanol:diisopropyl ether (1:2 v/v) (60 ml) to give N-{1-[(6-amino-2-pyridinyl)acetyl]-2,3-dihydro-1H-indol-5-yl}-4′-(trifluoromethyl)-1,1′-biphenyl-2-carboxamide hydrochloride (2.42 g) as white crystals.

¹H-NMR (DMSO-d₆):δ 3.17 (2H, t, J=8.4 Hz), 4.09 (2H, s), 4.19 (2H, t, J=8.4 Hz), 6.78 (1H, d, J=7.0 Hz), 6.92 (1H, d, J=8.5 Hz), 7.3-7.4 (1H, m), 7.5-8.0 (1H, m), 10.33 (1H, s), 13.91 (1H, br)

### EXAMPLE 148

N-{1-[(6-Amino-2-pyridinyl)acetyl]-2,3-dihydro-1H-indol-5-yl}-4′-methyl-1,1′-biphenyl-2-carboxamide hydrochloride

The title compound was obtained in the same manner as in Example 147 as white crystals.

¹H-NMR (DMSO-d₆):δ 2.29 (3H, s), 3.18 (2H, t, J=8.3 Hz), 4.09 (2H, s), 4.19 (2H, t, J=8.3 Hz), 6.78 (1H, d, J=7.1 Hz), 6.93 (1H, d, J=8.8 Hz), 7.2-7.6 (11H, m), 7.85-8.05 (3H, m), 10.22 (1H, s), 14.08 (1H, br)

### EXAMPLE 149

To a solution of N-{1-[(6-amino-2-pyridinyl)acetyl]-2,3-dihydro-1H-indol-5-yl}-4′-(trifluoromethyl)-1,1′-biphenyl-2-carboxamide (1.55 g) in acetonitrile (20 ml) and methanol (20 ml) was added a solution of sodium hydrogencarbonate (605 mg) in water (10 ml), followed by dropwise addition of a solution of OXONE (1.84 g) in water (10 ml) at ambient temperature. The resulting suspension was stirred at ambient temperature for 6 hours and quenched with 10% aqueous sodium thiosulfate solution (20 ml). The mixture was evaporated in vacuo and the residue was extracted with chloroform:methanol (20:1 v/v). The organic layer was washed with brine, dried over magnesium sulfate, and evaporated in vacuo. The residue was purified by column chromatography on silica gel and recrystallized from tetrahydrofuran:methanol (1:1 v/v) to give N-{1-[(6-amino-1-oxido-2-pyridinyl)acetyl]-2,3-dihydro-1H-indol-5-yl}-4-′-(trifluoromethyl)-1,1′-biphenyl-2-carboxamide (545 mg) as white crystals.

¹H-NMR (DMSO-d₆):δ 3.14 (2H, t, J=8.4 Hz), 3.92 (2H, s), 4.28 (2H, t, J=8.4 Hz), 6.6-6.8 (4H, m), 7.0-7.25 (2H, m), 7.45-7.9 (10H, m), 10.26 (1H, s)

ESI-MS(m/z): 555 (M+Na)⁺, 533 (M+H)⁺

### EXAMPLE 150

N-{1-[(6-amino-1-oxido-2-pyridinyl)acetyl]-2,3-dihydro-1H-indol-5-yl}-4′-methyl-1,1′-biphenyl-2-carboxamide

The title compound was obtained in the same manner as in Example 149 as white crystals.

¹H-NMR (DMSO-d₆):δ 2.29 (3H, s), 3.11 (2H, t, J=8.4 Hz), 3.92 (2H, s), 4.29 (2H, t, J=8.4 Hz), 6.6-6.8 (4H, m), 7.0-7.6 (1H, m), 7.85 (1H, d, J=8.7 Hz), 10.15 (1H, s)

ESI-MS(m/z): 501 (M+Na)⁺, 479 (M+H)⁺

### EXAMPLE 151

To a solution of 1-{([6-(2,5-dimethyl-1H-pyrrol-1-yl)-2-pyridinyl]acetyl}-5-indolinamine (693 mg), 5-methyl-4′-(trifluoromethyl)-1,1′-biphenyl-2-carboxylic acid (617 mg) and 1-hydroxybenzotriazole hydrate (337 mg) in N,N-dimethylformamide (20 ml) was added 1-[3-(dimethylamino)propyl]-3-ethylcarbodiimide (342 mg) at ambient temperature and the mixture was stirred at ambient temperature for 18 hours. The reaction mixture was poured into a mixture of ethyl acetate and water. The separated organic layer was washed with water and brine, dried over magnesium sulfate and evaporated in vacuo. The residue was purified by column chromatogaraphy eluting with ethyl acetate on silica gel to give N-(1-{[6-(2,5-dimethyl-1H-pyrrol-1-yl)-2-pyridinyl]acetyl}-2,3-dihydro-1H-indol-5-yl)-5-methyl-4′-(trifluoromethyl)-1,1′-biphenyl-2-carboxamide (722 mg) as a white solid.

¹H-NMR (DMSO-d₆):δ 2.04 (6H, s), 2.42 (3H, s), 3.09 (2H, t, J=8.3 Hz), 4.05 (2H, s), 4.16 (2H, t, J=8.3 Hz), 5.77 (2H, s), 7.2-7.55 (7H, m), 7.60 (2H, d, J=8.3 Hz), 7.74 (2H, d, J=8.3 Hz), 7.85-8.0 (2H, m), 10.17 (1H, s)

ESI-MS(m/z): 609 (M+H)⁺

### EXAMPLE 152

N-{1-[(6-Amino-2-pyridinyl)acetyl]-2,3-dihydro-1H-indol-5-yl}-5-methyl-4′-(trifluoromethyl)-1,1′-biphenyl-2-carboxamide

The title compound was obtained in the same manner as in Example 9 as white crystals.

¹H-NMR (DMSO-d₆):δ 2.42 (3H, s), 3.08 (2H, t, J=8.2 Hz), 3.69 (2H, s), 4.17 (2H, t, J=8.2 Hz), 5.84 (2H, br s), 6.29 (1H, d, J=8.0 Hz), 6.42 (1H, d, J=6.9 Hz), 7.15-7.55 (6H, m), 7.60 (2H, d, J=8.3 Hz), 7.74 (2H, d, J=8.3 Hz), 7.92 (1H, d, J=8.7 Hz), 10.17 (1H, s)

ESI-MS(m/z): 531 (M+H)⁺

### EXAMPLE 153

4′-Chloro-N-(1-{[6-(2,5-dimethyl-1H-pyrrol-1-yl)-2-pyridinyl]acetyl}-2,3-dihydro-1H-indol-5-yl)-5-methyl-1,1′-biphenyl-2-carboxamide

The title compound was obtained in the same manner as in Example 151 as a white solid.

¹H-NMR (DMSO-d₆):δ 2.02 (6H, s), 2.40 (3H, s), 3.10 (2H, t, J=8.4 Hz), 4.05 (2H, s), 4.16 (2H, t, J=8.4 Hz), 5.77 (2H, s), 7.2-7.6 (11H, m), 7.9-8.05 (2H, m), 10.12 (1H, s)

ESI-MS(m/z): 577, 575 (M+H)⁺

### EXAMPLE 154

N-{1-[(6-Amino-2-pyridinyl)acetyl]-2,3-dihydro-1H-indol-5-yl}-4′-chloro-5-methyl-1,1′-biphenyl-2-carboxamide

The title compound was obtained in the same manner as in Example 9 as white crystals.

¹H-NMR (DMSO-d₆):δ 2.40 (3H, s), 3.09 (2H, t, J=8.2 Hz), 3.69 (2H, s), 4.17 (2H, t, J=8.2 Hz), 5.84 (2H, br s), 6.30 (1H, d, J=7.8 Hz), 6.42 (1H, d, J=6.8 Hz), 7.2-7.5 (10H, m), 7.92 (1H, d, J=8.7 Hz), 10.10 (1H, s) ESI-MS(m/z): 499, 497 (M+H)⁺

### EXAMPLE 155

To a solution of 4′-ethyl-1,1′-biphenyl-2-carboxylic acid (1.22 g) in toluene (20 ml) were added thionyl chloride (892 mg) and N,N-dimethylformamide (5 drops) and the mixture was stirred at 50° C. for 3 hours. The mixture was evaporated in vacuo and the residue was dissolved in dichloromethane (10 ml). This solution was added dropwise to a solution of 1-{[6-(2,5-dimethyl-1H-pyrrol-1-yl)-2-pyridinyl]acetyl}-5-indolinamine (1.732 g) in dichloromethane (30 ml) at 5° C. and the mixture was stirred at ambient temperature for 6 hours. Water was added and the separated organic layer was washed with brine, dried over magnesium sulfate, and evaporated in vacuo. The residue was purified by column chromatography on silica gel to give N-(1-{[6-(2,5-dimethyl-1H-pyrrol-1-yl)-2-pyridinyl]acetyl}-2,3-dihydro-1H-indol-5-yl)-4′-ethyl-1,1′-biphenyl-2-carboxamide (1.916 g) as a white solid.

¹H-NMR (DMSO-d₆):δ 1.17 (3H, t, J=7.6 Hz), 2.02 (6H, s), 2.65 (2H, q, J=7.6 Hz), 3.09 (2H, t, J=8.3 Hz), 4.05 (2H, s), 4.16 (2H, t, J=8.3 Hz), 5.77 (2H, s), 7.15-7.55 (12H, m), 7.85-8.0 (2H, m), 10.15 (1H, s)

negative ESI-MS(m/z): 553 (M−H)⁻

### EXAMPLE 156

N-{1-[(6-Amino-2-pyridinyl)acetyl]-2,3-dihydro-1H-indol-5-yl}-4′-ethyl-1,1′-biphenyl-2-carboxamide

The title compound was obtained in the same manner as in Example 9 as white crystals.

¹H-NMR (DMSO-d₆):δ 1.17 (3H, t, J=7.6 Hz), 2.59 (2H, q, J=7.6 Hz), 3.09 (2H, t, J=8.4 Hz), 3.69 (2H, s), 4.17 (2H, t, J=8.4 Hz), 5.84 (2H, br s), 6.30 (1H, d, J=8.2 Hz), 6.42 (1H, d, J=7.2 Hz), 7.20 (2H, d, J=8.2 Hz), 7.3-7.55 (9H, m), 7.92 (1H, d, J=8.7 Hz), 10.12 (1H, s)

ESI-MS(m/z): 499 (M+Na)⁺, 477 (M+H)⁺

### EXAMPLE 157

N-(1-{[6-(2,5-Dimethyl-1H-pyrrol-1-yl)-2-pyridinyl]acetyl}-2,3-dihydro-1H-indol-5-yl)-4′-methoxy-1,1′-biphenyl-2-carboxamide

The title compound was obtained in the same manner as in Example 151 as a light brown solid.

¹H-NMR (DMSO-d₆):δ 2.02 (6H, s), 3.01 (2H, t, J=8.1 Hz), 3.74 (3H, s), 4.05 (2H, s), 4.16 (2H, t, J=8.1 Hz), 5.77 (2H, s), 6.93 (2H, d, J=8.7 Hz), 7.25-7.55 (9H, m), 7.85-8.0 (2H, m), 10.14 (1H, s)

ESI-MS(m/z): 557 (M+H)⁺

### EXAMPLE 158

N-{1-[(6-Amino-2-pyridinyl)acetyl]-2,3-dihydro-1H-indol-5-yl}-4′-methoxy-1,1′-biphenyl-2-carboxamide

The title compound was obtained in the same manner as in Example 9 as white crystals.

¹H-NMR (DMSO-d₆):δ 3.09 (2H, t, J=8.3 Hz), 3.69 (2H, s), 3.74 (3H, s), 4.17 (2H, t, J=8.3 Hz), 5.85 (2H, br s), 6.30 (1H, d, J=8.2 Hz), 6.42 (1H, d, J=7.2 Hz), 6.93 (2H, d, J=8.7 Hz), 7.2-7.6 (9H, m), 7.92 (1H, d, J=8.7 Hz), 10.13 (1H, s)

ESI-MS(m/z): 479 (M+H)⁺

Preparation 134

To a solution of 1-{[6-(2,5-dimethyl-1H-pyrrol-1-yl)-2-pyridinyl]acetyl}-5-indolinamine (3.464 g) in dichloromethane (80 ml) was added a solution of 2-bromobenzoyl chloride (2.19 g) in dichloromethane (20 ml) at 5° C. and the mixture was stirred at ambient temperature for 18 hours. Water was added and the separated organic layer was washed with brine, dried over magnesium sulfate, and evaporated in vacuo. The residue was purified by column chromatography on silica gel to give 2-bromo-N-(1-{[6-(2,5-dimethyl-1H-pyrrol-1-yl)-2-pyridinyl]acetyl}-2,3-dihydro-1H-indol-5-yl)benzamide (4.29 g) as white crystals.

¹H-NMR (DMSO-d₆):δ 2.06 (6H, s), 3.15 (2H, t, J=8.4 Hz), 4.11 (2H, s), 4.19 (2H, t, J=8.4 Hz), 5.78 (2H, s), 7.29 (1H, d, J=7.7 Hz), 7.35-7.55 (4H, m), 7.65-7.75 (2H, m), 7.9-8.058 (2H, m), 10.40 (1H, s)

ESI-MS(m/z): 531, 529 (M+H)⁺

### EXAMPLE 159

To a solution of 2-bromo-N-(1-{[6-(2,5-dimethyl-1H-pyrrol-1-yl)-2-pyridinyl]acetyl}-2,3-dihydro-1H-indol-5-yl)benzamide (1.06 g) in N,N-dimethylformamide (40 ml) were added 5-chloro-2-thienylboronic acid (422 mg), triethylamine (607 mg) and tetrakis(triphenylphosphine)palladium(0) (116 mg) at ambient temperature and the mixture was stirred at 100° C. under a nitrogen atmosphere for 16 hours. The reaction mixture was poured into a mixture of ethyl acetate and water and the separated organic layer was washed with brine, dried over magnesium sulfate, and evaporated in vacuo. The residue was purified by column chromatography on silica gel to give 2-(5-chloro-2-thienyl)-N-(1-{[6-(2,5-dimethyl-1H-pyrrol-1-yl)-2-pyridinyl]acetyl}-2,3-dihydro-1H-indol-5-yl)benzamide (863 mg) as a yellowish white powder.

¹H-NMR (DMSO-d₆):δ 2.03 (6H, s), 3.14 (2H, t, J=8.0 Hz), 4.08 (2H, s), 4.18 (2H, t, J=8.0 Hz), 5.78 (2H, s), 7.09 (2H, s), 7.25-7.6 (8H, m), 7.9-8.05 (2H, m), 10.43 (1H, s)

ESI-MS(m/z): 569, 567 (M+H)⁺

### EXAMPLE 160

N-{1-[(6-Amino-2-pyridinyl)acetyl]-2,3-dihydro-1H-indol-5-yl}-2-(5-chloro-2-thienyl)benzamide

The title compound was obtained in the same manner as in Example 9 as white crystals.

¹H-NMR (DMSO-d₆):δ 3.13 (2H, t, J=8.4 Hz), 3.70 (2H, s), 4.19 (2H, t, J=8.4 Hz), 5.85 (2H, br s), 6.31 (1H, d, J=8.2 Hz), 6.43 (1H, d, J=7.2 Hz), 7.09 (2H, s), 7.31 (1H, dd, J=8.2 Hz, 7.2 Hz), 7.45-7.6 (6H, m), 7.97 (1H, d, J=8.7 Hz), 10.42 (1H, s)

ESI-MS(m/z): 491, 489 (M+H)⁺

### EXAMPLE 161

N-(1-{[6-(2,5-Dimethyl-1H-pyrrol-1-yl)-2-pyridinyl]acetyl}-2,3-dihydro-1H-indol-5-yl)-2-(4-fluorophenyl)-1-cyclohexene-1-carboxamide

The title compound was obtained in the same manner as in Example 151 as a white solid.

¹H-NMR (DMSO-d₆):δ 1.6-1.8 (4H, m), 2.01 (6H, s), 2.3-2.45 (4H, m), 3.05 (2H, t, J=8.3 Hz), 4.02 (2H, s), 4.12 (2H, t, J=8.3 Hz), 5.77 (2H, s), 7.0-7.15 (3H, m), 7.25-7.4 (5H, m), 7.83 (1H, d, J=8.7 Hz), 7.94 (1H, dd, J=7.7 Hz, 7.7 Hz), 9.48 (1H, s)

ESI-MS(m/z): 571 (M+Na)⁺, 549 (M+H)⁺

### EXAMPLE 162

N-{1-[(6-Amino-2-pyridinyl)acetyl]-2,3-dihydro-1H-indol-5-yl}-2-(4-fluorophenyl)-1-cyclohexene-1-carboxamide

The title compound was obtained in the same manner as in Example 9 as white crystals.

¹H-NMR (DMSO-d₆):δ 1.6-1.8 (4H, m), 2.25-2.45 (4H, m), 3.04 (2H, t, J=8.3 Hz), 3.67 (2H, s), 4.13 (2H, t, J=8.3 Hz), 5.85 (2H, br s), 6.29 (1H, d, J=8.0 Hz), 6.90 (1H, d, J=7.0 Hz), 7.0-7.2 (3H, m), 7.25-7.4 (4H, m), 7.84 (1H, d, J=8.6 Hz), 9.47 (1H, s)

negative ESI-MS(m/z): 469 (M−H)⁻

### EXAMPLE 163

2-(4-Chlorophenyl)-N-(1-{[6-(2,5-dimethyl-1H-pyrrol-1-yl)-2-pyridinyl]acetyl}-2,3-dihydro-1H-indol-5-yl)-1-cyclohexene-1-carboxamide

The title compound was obtained in the same manner as in Example 151 as a white solid.

¹H-NMR (DMSO-d₆):δ 1.6-1.8 (4H, m), 2.01 (6H, s), 2.3-2.5 (4H, m), 3.06 (2H, t, J=8.3 Hz), 4.03 (2H, s), 4.13 (2H, t, J=8.3 Hz), 5.77 (2H, s), 7.05 (1H, dd, J=8.7 Hz, 2.0 Hz), 7.25-7.45 (7H, m), 7.84 (1H, d, J=8.7 Hz), 7.85-7.95 (1H, m), 9.53 (1H, s)

negative ESI-MS(m/z): 563 (M−H)⁻

### EXAMPLE 164

N-{1-[(6-Amino-2-pyridinyl)acetyl]-2,3-dihydro-1H-indol-5-yl}-2-(4-chlorophenyl)-1-cyclohexene-1-carboxamide

The title compound was obtained in the same manner as in Example 9 as white crystals.

¹H-NMR (DMSO-d₆):δ 1.6-1.8 (4H, m), 2.25-2.4 (4H, m), 3.05 (2H, t, J=8.3 Hz), 3.67 (2H, s), 4.14 (2H, t, J=8.3 Hz), 5.85 (2H, br s), 6.29 (1H, d, J=8.0 Hz), 6.40 (1H, d, J=7.0 Hz), 7.04 (1H, dd, J=8.5 Hz, 1.8 Hz), 7.25-7.4 (6H, m), 7.85 (1H, d, J=8.5 Hz), 9.52 (1H, s)

negative ESI-MS(m/z): 485 (M−H)⁻

### EXAMPLE 165

N-(1-{[6-(2,5-Dimethyl-1H-pyrrol-1-yl)-2-pyridinyl]acetyl}-2,3-dihydro-1H-indol-5-yl)-2-(4-methylphenyl)-1-cyclohexene-1-carboxamide

The title compound was obtained in the same manner as in Example 151 as a white solid.

¹H-NMR (DMSO-d₆):δ 1.6-1.8 (4H, m), 2.21 (3H, s), 2.25-2.4 (4H, m), 3.05 (2H, d, J=8.4 Hz), 4.02 (2H, s), 4.12 (2H, d, J=8.4 Hz), 5.77 (2H, s), 7.04 (2H, d, J=8.1 Hz), 7.17 (2H, d, J=8.1 Hz), 7.28 (1H, d, J=7.7 Hz), 7.35-7.45 (2H, m), 7.82 (1H, d, J=8.7 Hz), 7.94 (1H, dd, J=7.7 Hz, 7.7 Hz), 9.44 (1H, s)

ESI-MS(m/z): 567 (M+Na)⁺, 545 (M+H)⁺

### EXAMPLE 166

N-{1-[(6-Amino-2-pyridinyl)acetyl]-2,3-dihydro-1H-indol-5-yl}-2-(4-methylphenyl)-1-cyclohexene-1-carboxamide

The title compound was obtained in the same manner as in Example 9 as white crystals.

¹H-NMR (DMSO-d₆):δ 1.6-1.8 (4H, m), 2.21 (3H, s), 2.25-2.4 (4H, m), 3.04 (2H, t, J=8.3 Hz), 3.67 (2H, s), 4.13 (2H, t, J=8.3 Hz), 5.85 (2H, br s), 6.29 (1H, d, J=8.1 Hz), 6.40 (1H, d, J=7.0 Hz), 7.03 (2H, d, J=8.1 Hz), 7.05 (1H, s), 7.17 (2H, d, J=8.1 Hz), 7.25-7.35 (2H, m), 7.84 (1H, d, J=8.6 Hz), 9.43 (1H, s)

ESI-MS(m/z): 489 (M+Na)⁺, 467 (M+H)⁺

### EXAMPLE 167

N-(1-{[6-(2,5-Dimethyl-1H-pyrrol-1-yl)-2-pyridinyl]acetyl}-2,3-dihydro-1H-indol-5-yl)-2-(4-methoxyphenyl)-1-cyclohexene-1-carboxamide

The title compound was obtained in the same manner as in Example 151 as a white solid.

¹H-NMR (DMSO-d₆):δ 1.6-1.8 (4H, m), 2.01 (6H, s), 2.25-2.4 (4H, m), 3.05 (2H, t, J=8.3 Hz), 3.67 (3H, s), 4.01 (2H, s), 4.13 (2H, t, J=8.3 Hz), 5.77 (2H, s), 6.80 (2H, d, J=8.8 Hz), 7.05 (1H, dd, J=8.7 Hz, 1.8 Hz), 7.21 (2H, d, J=8.7 Hz), 7.28 (1H, d, J=7.9 Hz), 7.39 (2H, d, J=7.5 Hz), 7.83 (1H, d, J=8.7 Hz), 7.94 (1H, dd, J=7.8 Hz, 7.8 Hz), 9.43 (1H, s)

ESI-MS(m/z): 583 (M+Na)⁺, 561 (M+H)⁺

### EXAMPLE 168

N-{1-[(6-Amino-2-pyridinyl)acetyl]-2,3-dihydro-1H-indol-5-yl}-2-(4-methoxyphenyl)-1-cyclohexene-1-carboxamide

The title compound was obtained in the same manner as in Example 9 as white crystals.

¹H-NMR (DMSO-d₆):δ 1.6-1.8 (4H, m), 2.25-2.4 (4H, m), 3.04 (2H, t, J=8.5 Hz), 3.67 (3H, s), 4.13 (2H, t, J=8.5 Hz), 5.84 (2H, br s), 6.29 (1H, d, J=8.2 Hz), 6.40 (1H, d, J=7.4 Hz), 6.79 (2H, d, J=8.8 Hz), 7.00 (1H, dd, J=7.4 Hz, 2.1 Hz), 7.20 (2H, d, J=8.8 Hz), 7.28 (1H, d, J=7.4 Hz), 7.34 (1H, d, J=2.1 Hz), 7.84 (1H, d, J=8.7 Hz), 9.40 (1H, s)

negative ESI-MS(m/z): 481 (M−H)⁻

### EXAMPLE 169

N-(1-{[6-(2,5-Dimethyl-1H-pyrrol-1-yl)-2-pyridinyl]acetyl}-2,3-dihydro-1H-indol-5-yl)-2-[4-(trifluoromethyl)phenyl]-1-cyclohexene-1-carboxamide

The title compound was obtained in the same manner as in Example 151 as a white solid.

¹H-NMR (DMSO-d₆):δ 1.6-1.8 (4H, m), 2.01 (6H, s), 2.25-2.4 (4H, m), 3.04 (2H, t, J=8.4 Hz), 4.02 (2H, s), 4.12 (2H, t, J=8.4 Hz), 5.76 (2H, s), 7.00 (1H, dd, J=8.6 Hz, 1.8 Hz), 7.28 (2H, d, J=7.8 Hz), 7.38 (1H, d, J=7.5 Hz), 7.47 (2H, d, J=8.2 Hz), 7.62 (2H, d, J=8.2 Hz), 7.85 (1H, d, J=8.6 Hz), 7.94 (1H, dd, J=8.6 Hz, 7.5 Hz), 9.56 (1H, s)

ESI-MS(m/z): 621 (M+Na)⁺, 599 (M+H)⁺

### EXAMPLE 170

N-{1-[(6-Amino-2-pyridinyl)acetyl]-2,3-dihydro-1H-indol-5-yl}-2-[4-(trifluoromethyl)phenyl]-1-cyclohexene-1-carboxamide

The title compound was obtained in the same manner as in Example 9 as white crystals.

¹H-NMR (DMSO-d₆): 1.65-1.85 (4H, m), 2.3-2.5 (4H, m), 3.03 (2H, t, J=8.1 Hz), 3.67 (2H, s), 4.13 (2H, t, J=8.1 Hz), 5.85 (2H, br s), 6.29 (1H, d, J=8.0 Hz), 6.40 (1H, d, J=7.2 Hz), 6.99 (1H, dd, J=8.7 Hz, 1.8 Hz), 7.30 (1H, dd, J=8.0 Hz, 7.2 Hz), 7.31 (1H, d, J=1.8 Hz), 7.47 (2H, d, J=8.3 Hz), 7.62 (2H, d, J=8.3 Hz), 7.84 (1H, d, J=8.7 Hz), 9.56 (1H, s)

ESI-MS(m/z): 543 (M+Na)⁺, 521 (M+H)⁺

### EXAMPLE 171

N-(1-{[6-(2,5-Dimethyl-1H-pyrrol-1-yl)-2-pyridinyl]acetyl}-2,3-dihydro-1H-indol-5-yl)-4′-(methylthio)-1,1′-biphenyl-2-carboxamide

The title compound was obtained in the same manner as in Example 151 as a white solid.

¹H-NMR (DMSO-d₆):δ 2.05 (6H, s), 2.46 (3H, s), 3.10 (2H, t, J=8.3 Hz), 4.05 (2H, s), 4.16 (2H, t, J=8.3 Hz), 5.78 (2H, s), 7.2-7.6 (12H, m), 7.9-8.05 (2H, m), 10.21 (1H, s)

negative ESI-MS(m/z): 571 (M−H)⁻

### EXAMPLE 172

N-{1-[(6-Amino-2-pyridinyl)acetyl]-2,3-dihydro-1H-indol-5-yl}-4′-(methylthio)-1,1′-biphenyl-2-carboxamide

The title compound was obtained in the same manner as in Example 9 as white crystals.

¹H-NMR (DMSO-d₆):δ 2.46 (3H, s), 3.10 (2H, t, J=8.3 Hz), 3.69 (2H, s), 4.17 (2H, t, J=8.3 Hz), 5.86 (2H, br s), 6.30 (1H, d, J=8.1 Hz), 6.42 (1H, d, J=7.5 Hz), 7.2-7.6 (11H, m), 7.93 (1H, d, J=8.6 Hz), 10.19 (1H, s)

ESI-MS(m/z): 517 (M+Na)⁺, 495 (M+H)⁺

### EXAMPLE 173

To a solution of 4′-hydroxy-1,1′-biphenyl-2-carboxylic acid (643 mg) in dichloromethane (30 ml) was added triethylamine (911 mg), followed by dropwise addition of methanesulfonyl chloride (860 mg) at 5° C. and the mixture was stirred at 5° C. for 3 hours. Water was added and the separated organic layer was washed with brine, dried over magnesium sulfate and concentrated in vacuo to give a dimesylate compound. A solution of the obtained dimesylate compound and 1-(2-pyridinylacetyl)-5-indolinamine (684 mg) in N,N-dimethylformamide (30 ml) was added triethylamine (911 mg) and the mixture was stirred at 80° C. for 16 hours under a nitrogen atmosphere. The reaction mixture was poured into a mixture of ethyl acetate and water, and the separated organic layer was washed with water and brine, dried over magnesium sulfate, and evaporated in vacuo. The residue was purified by column chromatography on silica gel and recrystallized from ethyl acetate to give 2′-({([1-(2-pyridinylacetyl)-2,3-dihydro-1H-indol-5-yl]amino}carbonyl)-1,1′-biphenyl-4-yl methanesulfonate (779 mg) as white crystals.

¹H-NMR (DMSO-d₆):δ 3.11 (2H, t, J=8.4 Hz), 3.36 (3H, s), 3.99 (2H, s), 4.19 (2H, t, J=8.4 Hz), 7.19 (1H, dd, J=8.7 Hz, 1.6 Hz), 7.27 (1H, dd, J=8.7 Hz, 4.9 Hz), 7.35-7.6 (10H, m), 7.75 (1H, ddd, J=8.7 Hz, 7.7 Hz, 1.8 Hz), 7.90 (1H, d, J=8.7 Hz), 8.5-8.55 (1H, m), 10.14 (1H, s)

ESI-MS(m/z): 550 (M+Na)⁺, 528 (M+H)⁺

Preparation 135

2-Bromo-N-[1-(2-pyridinylacetyl)-2,3-dihydro-1H-indol-5-yl]benzamide

The title compound was obtained in the same manner as in Preparation 134 as white crystals.

¹H-NMR (DMSO-d₆):δ 3.01 (2H, t, J=8.2 Hz), 3.98 (2H, s), 4.23 (2H, t, J=8.2 Hz), 7.2-7.55 (6H, m), 7.65-7.8 (3H, m), 7.98 (1H, d, J=8.7 Hz), 8.51 (1H, d, J=4.8 Hz), 10.40 (1H, s)

ESI-MS(m/z): 438, 436 (M+H)⁺

### EXAMPLE 174

2-(5-Chloro-2-thienyl)-N-[1-(2-pyridinylacetyl)-2,3-dihydro-1H-indol-5-yl]benzamide

The title compound was obtained in the same manner as in Example 159 as white crystals.

¹H-NMR (DMSO-d₆):δ 3.11 (2H, t, J=8.7 Hz), 4.00 (2H, s), 4.21 (2H, t, J=8.7 Hz), 7.07 (1H, s), 7.2-7.4 (3H, m), 7.45-7.6 (6H, m), 7.7-7.85 (1H, m), 7.96 (1H, d, J=8.6 Hz), 8.49 (1H, d, J=4.8 Hz), 10.43 (1H, s)

negative ESI-MS(m/z): 472 (M−H)⁻

Preparation 136

To a suspension of 2-iodobenzoic acid (2.48 g) in water (50 ml) were added 1,1′-biphenyl-4-ylboronic acid (2.57 g), palladium acetate (112 mg) and sodium carbonate (3.18 g) at ambient temperature and the mixture was stirred at 100° C. for 16 hours. Ethyl acetate (80 ml) and active charcoal (2 g) were added and the mixture was stirred at ambient temperature for 30 minutes. The active charcoal was removed by filtration with celite and the filtrate was adjusted to pH 2 with 6N HCl. The separated organic layer was washed with brine, dried over magnesium sulfate and evaporated in vacuo. The residue was purified by column chromatography on silica gel and triturated with diisopropyl ether to give 1,1′:4′,1″-terphenyl-2-carboxylic acid (1.40 g) as white crystals.

¹H-NMR (DMSO-d₆):δ 7.2-7.3 (1H, m), 7.35-7.5 (6H, m), 7.55-7.8 (5H, m), 8.0-8.05 (1H, m)

negative ESI-MS(m/z): 273 (M−H)⁻

### EXAMPLE 175

N-{1-[2-(2-Pyridinyl)acetyl]-2,3-dihydro-1H-indol-5-yl}-1,1′:4′,1″-terphenyl-2-carboxamide

The title compound was obtained in the same manner as in Example 151 as a white solid.

¹H-NMR (DMSO-d₆):δ 3.10 (2H, t, J=8.69 Hz), 6.98 (2H, s), 4.18 (2H, t, J=8.6 Hz), 7.2-7.95 (19H, m), 8.48 (1H, d, J=4.4 Hz), 10.23 (1H, s)

negative ESI-MS(m/z): 508 (M−H)⁻

### EXAMPLE 176

4′-Fluoro-N-[1-(2-pyridinylacetyl)-2,3-dihydro-1H-indol-5-yl]-1,1′-biphenyl-2-carboxamide

The title compound was obtained in the same manner as in Example 151 as a white solid.

¹H-NMR (DMSO-d₆):δ 3.11 (2H, t, J=8.5 Hz), 3.99 (2H, s), 4.19 (2H, t, J=8.5 Hz), 7.15-7.6 (12H, m), 7.7-7.85 (1H, m), 7.91 (1H, d, J=8.7 Hz), 8.45-8.55 (1H, m), 10.18 (1H, s)

ESI-MS(m/z): 474 (M+Na)⁺, 452 (M+H)⁺

### EXAMPLE 177

2-(4-Fluorophenyl)-N-[1-(2-pyridinylacetyl)-2,3-dihydro-1H-indol-5-yl]-1-cyclohexene-1-carboxamide

The title compound was obtained in the same manner as in Example 151 as a white solid.

¹H-NMR (DMSO-d₆):δ 1.6-1.8 (4H, m), 2.25-2.45 (4H, m), 3.06 (2H, t, J=8.2 Hz), 3.96 (2H, s), 4.15 (2H, t, J=8.2 Hz), 7.0-7.4 (8H, m), 7.7-7.95 (2H, m), 8.45-8.55 (1H, m), 9.49 (1H, s)

negative ESI-MS(m/z): 454 (M−H)⁻

Preparation 137

To a solution of 4-methyl-2-pyrimidinamine (10.0 g) in toluene (200 ml) were added 2,5-hexanedione (11.5 g) and p-toluenesulfonic acid hydrate (1.74 g) at ambient temperature and the mixture was refluxed for 20 hours. The reaction mixture was concentrated to ca. 50 ml and purified by column chromatography on silica gel to give 2-(2,5-dimethyl-1H-pyrrol-1-yl)-4-methylpyrimidine (14.10 g) as a red oil.

¹H-NMR (DMSO-d₆):δ 2.23 (6H, s), 2.52 (3H, s), 5.81 (2H, s), 7.35 (1H, d, J=5.1 Hz), 8.73 (1H, d, J=5.1 Hz)

ESI-MS(m/z): 210 (M+Na)⁺, 188 (M+H)⁺

Preparation 138

To a 1 mol/L solution of sodium bis(trimethylsilyl)amide in tetrahydrofuran (82.2 ml) was added dropwise a solution of 2-(2,5-dimethyl-1H-pyrrol-1-yl)-4-methylpyrimidine (14.0 g) in tetrahydrofuran (100 ml) at 5° C. under a nitrogen atmosphere and the mixture was stirred at 5° C. for 1.5 hours. To the mixture was added carefully crashed Dry Ice (ca. 10 g) and the mixture was stirred at ambient temperature for 30 minutes. The reaction mixture was poured into a mixture of ethyl acetate and water, and adjusted to pH 2 with 6N HCl. The separated organic layer was washed with water and brine, dried over magnesium sulfate, and evaporated in vacuo. The residue was purified by column chromatography on silica gel and triturated with diisopropyl ether to give [2-(2,5-dimethyl-1H-pyrrol-1-yl)-4-pyrimidinyl]acetic acid (8.86 g) as light brown crystals.

¹H-NMR (DMSO-d₆):δ 2.23 (6H, s), 3.85 (2H, s), 5.82 (2H, s), 7.43 (1H, d, J=5.1 Hz), 8.83 (1H, d, J=5.1 Hz), 12.72 (1H, br)

Preparation 139

1-{[2-(2,5-Dimethyl-1H-pyrrol-1-yl)-4-pyrimidinyl]acetyl}-5-nitroindoline

The title compound was obtained in the same manner as in Example 8 as light yellow crystals.

¹H-NMR (DMSO-d₆):δ 2.21 (6H, s), 3.27 (2H, t, J=8.5 Hz), 4.21 (2H, s), 4.31 (2H, t, J=8.5 Hz), 5.80 (2H, s), 7.47 (1H, d, J=5.1 Hz), 8.1-8.2 (3H, m), 8.85 (1H, d, J=5.1 Hz)

ESI-MS(m/z): 400 (M+Na)⁺, 378 (M+H)⁺

Preparation 140

1-{[2-(2,5-Dimethyl-1H-pyrrol-1-yl)-4-pyrimidinyl]acetyl}-5-indolinamine

The title compound was obtained in the same manner as in Preparation 6 as light yellow crystals.

¹H-NMR (DMSO-d₆):δ 3.02 (2H, t, J=8.2 Hz), 4.04 (2H, s), 4.10 (2H, t, J=8.2 Hz), 4.88 (2H, br s), 5.80 (2H, s), 6.33 (1H, dd, J=8.5 Hz, 1.8 Hz), 6.46 (1H, d, J=1.8 Hz), 7.43 (1H, d, J=5.1 Hz), 7.73 (1H, d, J=8.5 Hz), 8.81 (1H, d, J=5.1 Hz)

ESI-MS(m/z): 370 (M+Na)+, 348 (M+H)+

### EXAMPLE 178

N-(1-{[2-(2,5-Dimethyl-1H-pyrrol-1-yl)-4-pyrimidinyl]acetyl}-2,3-dihydro-1H-indol-5-yl)-4′-ethyl-1,1′-biphenyl-2-carboxamide

The title compound was obtained in the same manner as in Example 151 as a light brown solid.

¹H-NMR (DMSO-d₆):δ 1.13 (3H, t, J=7.5 Hz), 2.21 (6H, s), 2.59 (2H, q, J=7.5 Hz), 3.12 (2H, t, J=8.4 Hz), 4.11 (2H, s), 4.18 (2H, t, J=8.4 Hz), 5.80 (2H, s), 7.20 (2H, d, J=8.1 Hz), 7.15-7.25 (1H, m), 7.35 (2H, d, J=8.1 Hz), 7.4-7.6 (6H, m), 7.90 (1H, d, J=8.7 Hz), 8.83 (1H, d, J=5.1 Hz), 10.17 (1H, s)

ESI-MS(m/z): 578 (M+Na)⁺, 556 (M+H)⁺

### EXAMPLE 179

N-{1-[(2-Amino-4-pyrimidinyl)acetyl]-2,3-dihydro-1H-indol-5-yl}-4′-ethyl-1,1′-biphenyl-2-carboxamide

The title compound was obtained in the same manner as in Example 9 as white crystals.

¹H-NMR (DMSO-d₆):δ 1.17 (3H, t, J=7.3 Hz), 2.59 (2H, q, J=7.3 Hz), 3.11 (2H, t, J=8.3 Hz), 3.74 (2H, s), 4.15 (2H, t, J=8.3 Hz), 6.5-6.6 (2H, m), 7.20 (2H, t, J=8.1 Hz), 7.35 (2H, t, J=8.1 Hz), 7.4-7.6 (7H, m), 7.90 (1H, d, J=8.6 Hz), 8.15 (1H, d, J=5.0 Hz), 10.16 (1H, s)

ESI-MS(m/z): 476 (M+Na)⁺, 454 (M+H)⁺

### EXAMPLE 180

N-(1-{[2-(2,5-Dimethyl-1H-pyrrol-1-yl)-4-pyrimidinyl]acetyl}-2,3-dihydro-1H-indol-5-yl)-2-(4-fluorophenyl)-1-cyclohexene-1-carboxamide

The title compound was obtained in the same manner as in Example 151 as a light brown solid.

¹H-NMR (DMSO-d₆):δ 1.6-1.8 (4H, m), 2.20 (6H, s), 2.25-2.4 (4H, m), 3.08 (2H, t, J=7.6 Hz), 4.08 (2H, s), 4.14 (2H, t, J=7.6 Hz), 5.80 (2H, s), 6.95-7.15 (3H, m), 7.2-7.35 (3H, m), 7.43 (1H, d, J=5.0 Hz), 7.83 (1H, d, J=8.7 Hz), 8.82 (1H, d, J=5.0 Hz), 9.50 (1H, s)

### EXAMPLE 181

N-{1-[(2-Amino-4-pyrimidinyl)acetyl]-2,3-dihydro-1H-indol-5-yl}-2-(4-fluorophenyl)-1-cyclohexene-1-carboxamide

The title compound was obtained in the same manner as in Example 9 as white crystals.

¹H-NMR (DMSO-d₆):δ 1.65-1.9 (4H, m), 2.3-2.5 (4H, m), 3.06 (2H, t, J=8.5 Hz), 3.71 (2H, s), 4.12 (2H, t, J=8.5 Hz), 6.51 (1H, d, J=5.0 Hz), 6.56 (2H, br s), 7.0-7.15 (3H, m), 7.25-7.4 (3H, m), 7.83 (1H, d, J=8.7 Hz), 8.14 (1H, d, J=5.0 Hz), 9.49 (1H, s)

negative ESI-MS(m/z): 470 (M−H)⁻

Preparation 141

A mixture of 4,6-dimethylindoline (1.47 g) and methyl 2-pyridinylacetate (2.27 g) was stirred at 150° C. for 20 hours. After cooling to ambient temperature, the mixture was purified by column chromatography on silica gel to give 4,6-dimethyl-1-(2-pyridinylacetyl)indoline (1.79 g) as a yellow oil.

ESI-MS(m/z): 289 (M+Na)⁺, 267 (M+H)⁺

Preparation 142

To a solution of 4,6-dimethyl-1-(2-pyridinylacetyl)indoline (1.78 g) in acetic acid (40 ml) was added dropwise nitric acid (fuming, d=1.50, 2.4 ml) at 10° C. and the mixture was stirred at ambient temperature for 3 hours. Ice-water (40 ml) was added and the mixture was adjusted to pH 8 with 50% aqueous potassium carbonate solution. The mixture was extracted with ethyl acetate and the separated organic layer was washed with brine, dried over magnesium sulfate, and evaporated in vacuo. The residue was purified by column chromatography on silica gel to give 4,6-dimethyl-5-nitro-1-(2-pyridinylacetyl)indoline (1.23 g) as a light brown solid.

¹H-NMR (DMSO-d₆):δ 2.13 (3H, s), 2.21 (3H, s), 3.12 (2H, d, J=8.4 Hz), 4.05 (2H, s), 4.28 (2H, t, J=8.4 Hz), 7.25-7.4 (2H, m), 7.7-7.8 (1H, m), 7.92 (1H, s), 8.50 (1H, d, J=4.1 Hz)

ESI-MS (m/z): 334 (M+Na)⁺, 312 (M+H)⁺

Preparation 143

To a mixture of 4,6-dimethyl-5-nitro-1-(2-pyridinylacetyl)indoline (1.22 g), iron chloride (32 mg) and active charcoal (2 g) in ethanol (40 ml) was added dropwise hydrazine hydrate (785 mg) at 80° C. The mixture was stirred at 80° C. for 3 hours and the active charcoal was removed by filtration with celite. The filtrate was evaporated in vacuo and the residue was purified by column chromatography on silica gel to give 4,6-dimethyl-1-(2-pyridinylacetyl)-5-indolinamine (862 mg) as a light brown solid.

¹H-NMR (DMSO-d₆):δ 1.98 (3H, s), 2.03 (3H, s), 2.98 (2H, d, J=8.2 Hz), 3.92 (2H, s), 4.11 (2H, d, J=8.2 Hz), 7.2-7.8 (4H, m), 8.45-8.55 (1H, m)

ESI-MS(m/z): 304 (M+Na)⁺, 282 (M+H)⁺

### EXAMPLE 182

To a solution of 4,6-dimethyl-1-(2-pyridinylacetyl)-5-indolinamine (282 mg) were added triethylamine (152 mg) and 4′-(trifluoromethyl)-1,1′-biphenyl-2-carbonyl chloride (342 mg) and the mixture was stirred at 50° C. for 6 hours. The reaction mixture was extracted with ethyl acetate and the separated organic layer was washed with water and brine, dried over magnesium sulfate, and evaporated in vacuo. The residue was purified by column chromatography on silica gel and recrystallized from ethyl acetate to give N-[4,6-dimethyl-1-(2-pyridinylacetyl)-2,3-dihydro-1H-indol-5-yl]-4′-(trifluoromethyl)-1,1′-biphenyl-2-carboxamide (345 mg) as white crystals.

¹H-NMR (DMSO-d₆):δ 1.87 (3H, s), 1.96 (3H, s), 3.01 (2H, t, J=8.2 Hz), 3.99 (2H, s), 4.19 (2H, t, J=8.2 Hz), 7.2-7.8 (12H, m), 8.48 (1H, d, J=4.1 Hz), 9.63 (1H, s) ESI-MS(m/z): 552 (M+Na)⁺, 530 (M+H)+

Preparation 144

1-Acetyl-4,6-dimethyl-5-nitroindoline

The title compound was obtained in the same manner as in Preparation 142 as a light brown solid.

¹H-NMR (DMSO-d₆):δ 2.12 (3H, s), 2.18 (3H, s), 2.22 (3H, s), 3.05 (2H, t, J=8.4 Hz), 4.19 (2H, t, J=8.4 Hz), 7.90 (1H, s)

ESI-MS(m/z): 257 (M+Na)⁺, 235 (M+H)⁺

Preparation 145

1-Acetyl-4,6-dimethyl-5-indolinamine

The title compound was obtained in the same manner as in Preparation 143 as a light brown solid.

¹H-NMR (DMSO-d₆):δ 1.98 (3H, s), 2.33 (3H, s), 2.34 (3H, s), 2.97 (2H, t, J=8.2 Hz), 3.98 (2H, t, J=8.2 Hz), 4.29 (2H, br s), 7.61 (1H, s)

ESI-MS(m/z): 227 (M+Na)⁺, 205 (M+H)⁺

Preparation 146

N-(1-Acetyl-4,6-dimethyl-2,3-dihydro-1H-indol-5-yl)-4′-(trifluoromethyl)-1,1′-biphenyl-2-carboxamide

The title compound was obtained in the same manner as in Example 182 as a light brown solid.

¹H-NMR (DMSO-d₆):δ 1.86 (3H, s), 1.98 (3H, s), 2.18 (3H, s), 2.9-3.1 (2H, m), 4.0-4.2 (2H, m), 7.55-7.8 (9H, m), 9.61 (1H, s)

ESI-MS(m/z): 475 (M+Na)⁺

Preparation 147

To a suspension of N-(1-acetyl-4,6-dimethyl-2,3-dihydro-1H-indol-5-yl)-4′-(trifluoromethyl)-1,1′-biphenyl-2-carboxamide (1.20 g) was added 6N HCl and the mixture was refluxed for 6 hours. The resulting solution was evaporated to dryness and to the residue were added ethyl acetate, tetrahydrofuran and water. The mixture was adjusted to pH 8 with 50% aqueous potassium carbonate solution and the separated organic layer was washed with brine, dried over magnesium sulfate, and evaporated in vacuo. The residue was purified by column chromatography on silica gel to give N-(4,6-dimethyl-2,3-dihydro-1H-indol-5-yl)-4′-(trifluoromethyl)-1,1′-biphenyl-2-carboxamide (828 mg) as a brown solid.

¹H-NMR (DMSO-d₆):δ 1.81 (3H, s), 1.86 (3H, s), 2.77 (2H, t, J=8.6 Hz), 3.37 (2H, t, J=8.6 Hz), 5.31 (1H, br s), 6.16 (1H, s), 7.4-7.7 (6H, m), 7.77 (2H, d, J=8.2 Hz), 9.33 (1H, s)

ESI-MS(m/z): 433 (M+Na)⁺, 411 (M+H)⁺

### EXAMPLE 183

To a solution of N-(4,6-dimethyl-2,3-dihydro-1H-indol-5-yl)-4′-(trifluoromethyl)-1,1′-biphenyl-2-carboxamide (800 mg) were added 2-vinylpyridine (225 mg) and methanesulfonic acid (187 mg) in 2-methoxyethanol (5 ml) and the mixture was stirred at 160° C. for 16 hours. The reaction mixture was cooled to ambient temperature and purified by column chromatography on silica gel and preparative HPLC to give N-{4,6-dimethyl-1-[2-(2-pyridinyl)ethyl]-2,3-dihydro-1H-indol-5-yl}-4′-(trifluoromethyl)-1,1′-biphenyl-2-carboxamide (313 mg) as a light brown solid.

ESI-MS(m/z): 538 (M+Na)⁺, 516 (M+H)⁺

Preparation 148

To a solution diisopropylamine (1.11 g) in tetrahydrofuran (80 ml) was added dropwise n-butyllithium (1.54 mol/L hexane solution) (7.2 ml) at −60° C. under a nitrogen atmosphere and the mixture was stirred at −60° C. for 15 minutes. To this solution was added dropwise a solution of tert-butyl 3-methyl-1,2,4-thiadiazol-5-ylcarbamate (2.153 g) in tetrahydrofuran (30 ml) at −60° C. and the mixture was stirred at −60° C. for 1.5 hours. To the mixture was added carefully crashed Dry Ice (ca. 5 g) and the mixture was warmed to ambient temperature. The reaction mixture was poured into a mixture of ethyl acetate and water, and adjusted to pH 2 with 6N HCl. The separated organic layer was washed with water and brine, dried over magnesium sulfate, and evaporated in vacuo. The residue was purified by column chromatography on silica gel and triturated with diisopropyl ether to give {5-[(tert-butoxycarbonyl)amino]-1,2,4-thiadiazol-3-yl}acetic acid (1.74 g) as light brown crystals.

¹H-NMR (DMSO-d₆):δ 1.50 (9H, s), 3.73 (2H, s), 12.30 (1H, br), 12.55 (1H, br)

negative APCI-MS (m/z): 258 (M−H)⁻

Preparation 149

tert-Butyl 3-[2-(5-nitro-2,3-dihydro-1H-indol-1-yl)-2-oxoethyl]-1,2,4-thiadiazol-5-ylcarbamate

The title compound was obtained in the same manner as in Example 8 as light yellow crystals.

¹H-NMR (DMSO-d₆):δ 1.50 (9H, s), 3.25 (2H, t, J=8.4 Hz), 4.11 (2H, s), 4.27 (2H, t, J=8.4 Hz), 8.1-8.2 (3H, m), 12.33 (1H, br s)

negative APCI-MS (m/z): 404 (M−H)⁻

Preparation 150

1-{[2-(2,5-Dimethyl-1H-pyrrol-1-yl)-4-pyrimidinyl]acetyl}-5-indolinamine

The title compound was obtained in the same manner as in Preparation 6 as light yellow crystals.

¹H-NMR (DMSO-d₆):δ 1.50 (9H, s), 3.00 (2H, t, J=8.4 Hz), 3.93 (2H, s), 4.05 (2H, t, J=8.4 Hz), 4.85 (2H, br s), 6.33 (1H, dd, J=8.5 Hz, 2.3 Hz), 6.45 (1H, d, J=2.3 Hz), 7.71 (1H, d, J=8.5 Hz), 12.28 (1H, br s)

negative APCI-MS (m/z): 374 (M−H)⁻

### EXAMPLE 184

tert-Butyl 3-{2-oxo-2-[5-({[4′-(trifluoromethyl)-1,1′-biphenyl-2-yl]carbonyl}amino)-2,3-dihydro-1H-indol-1-yl]ethyl}-1,2,4-thiadiazol-5-ylcarbamate

The title compound was obtained in the same manner as in Example 1 as a light brown solid.

¹H-NMR (DMSO-d₆):δ 1.49 (9H, s), 3.10 (2H, t, J=8.7 Hz), 4.00 (2H, s), 4.13 (2H, t, J=8.7 Hz), 7.21 (1H, dd, J=8.7 Hz, 2.0 Hz), 7.55-7.7 (7H, m), 7.75 (2H, d, J=8.3 Hz), 7.89 (1H, d, J=8.6 Hz), 10.27 (1H, s), 12.27 (1H, br s)

ESI-MS(m/z): 646 (M+Na)⁺

### EXAMPLE 185

To a solution of tert-butyl 3-{2-oxo-2-[5-({[4′-(trifluoromethyl)-1,1′-biphenyl-2-yl]carbonyl}amino)-2,3-dihydro-1H-indol-1-yl]ethyl}-1,2,4-thiadiazol-5-ylcarbamate (1.28 g) in dichloromethane (10 ml) was added trifluoroacetic acid (1.87 ml) and the mixture was stirred at ambient temperature for 20 hours. The reaction mixture was evaporated to dryness and to the residue were added ethyl acetate and water. The mixture was adjusted to pH 8 with 50% aqueous potassium carbonate solution and the separated organic layer was washed with brine, dried over magnesium sulfate and evaporated in vacuo. The residue was purified by column chromatography on silica gel and recrystallized with ethyl acetate to give N-{1-[(5-amino-1,2,4-thiadiazol-3-yl)acetyl]-2,3-dihydro-1H-indol-5-yl}-4′-(trifluoromethyl)-1,1′-biphenyl-2-carboxamide (658 mg) as white crystals.

¹H-NMR (DMSO-d₆):δ 3.09 (2H, t, J=8.2 Hz), 3.79 (2H, s), 4.13 (2H, t, J=8.2 Hz), 7.15-7.3 (1H, m), 7.45-8.0 (13H, m), 10.27 (1H, s)

ESI-MS (m/z): 524 (M+H)⁺

Preparation 151

2-(2,5-Dimethyl-1H-pyrrol-1-yl)-5-methyl-1,3-thiazole

The title compound was obtained in the same manner as in Preparation 137 as a red oil.

¹H-NMR (DMSO-d₆):δ 2.10 (6H, s), 2.49 (3H, s), 5.84 (2H, s), 7.51 (1H, s)

ESI-MS(m/z): 215 (M+Na)⁺, 193 (M+H)⁺

Preparation 152

{5-[(tert-Butoxycarbonyl)amino]-1,2,4-thiadiazol-3-yl}acetic acid

The title compound was obtained in the same manner as in Preparation 148 as light brown crystals.

¹H-NMR (DMSO-d₆):δ 2.11 (6H, s), 3.98 (2H, s), 5.86 (2H, s), 7.62 (1H, s), 12.85 (1H, br)

negative ESI-MS(m/z): 235 (M−H)⁻

### EXAMPLE 186

N-(1-{[2-(2,5-Dimethyl-1H-pyrrol-1-yl)-1,3-thiazol-5-yl]acetyl}-2,3-dihydro-1H-indol-5-yl)-4′-methyl-1,1′-biphenyl-2-carboxamide

The title compound was obtained in the same manner as in Example 151 as a white solid.

¹H-NMR (DMSO-d₆):δ 2.13 (6H, s), 2.29 (3H, s), 3.16 (2H, t, J=8.3 Hz), 4.21 (2H, t, J=8.3 Hz), 4.24 (2H, s), 5.86 (2H, s), 7.15-7.65 (10H, m), 7.94 (2H, d, J=8.7 Hz), 10.18 (1H, s)

### EXAMPLE 187

N-{1-[(2-Amino-1,3-thiazol-5-yl)acetyl]-2,3-dihydro-1H-indol-5-yl}-4′-methyl-1,1′-biphenyl-2-carboxamide

The title compound was obtained in the same manner as in Example 9 as white crystals.

¹H-NMR (DMSO-d₆):δ 2.29 (3H, s), 3.16 (2H, t, J=8.5 Hz), 3.83 (2H, s), 4.14 (2H, t, J=8.5 Hz), 6.80 (1H, s), 7.0-7.6 (10H, m), 7.90 (1H, d, J=8.5 Hz), 10.16 (1H, s)

ESI-MS(m/z): 491 (M+Na)⁺, 469 (M+H)⁺

Preparation 153

To a solution of phenyl 2-pyridinylcarbamate (4.28 g) and 5-nitroindoline (2.95 g) in N,N-dimethylformamide (50 ml) was added triethylamine (4.05 g) and the mixture was stirred at 150° C. for 20 hours under a nitrogen atmosphere. The reaction mixture was poured into a mixture of ethyl acetate and water and the separated organic layer was washed with water and brine, dried over magnesium sulfate, and evaporated in vacuo. The residue was purified by column chromatography on silica gel and triturated with diisopropyl ether to give 5-nitro-N-(2-pyridinyl)-1-indolinecarboxamide (3.37 g) as yellow crystals.

ESI-MS(m/z): 285 (M+H)⁺

Preparation 154

To a suspension of 5-nitro-N-(2-pyridinyl)-1-indolinecarboxamide (3.37 g) in methanol (100 ml) were added ammonium formate (2.24 g) and 10% palladium-carbon (50% wet) (3.4 g). The mixture was warmed carefully to 60° C. and stirred at 60° C. for 5 hours. Palladium-carbon was removed by filtration and the filtrate was evaporated in vacuo. The residue was purified by column chromatography on silica gel and triturated with diisopropyl ether to give 5-amino-N-(2-pyridinyl)-1-indolinecarboxamide (2.45 g) as light brown crystals.

¹H-NMR (DMSO-d₆):δ 3.02 (2H, t, J=8.4 Hz), 4.10 (2H, t, J=8.4 Hz), 4.75 (2H, br s), 6.3-6.4 (1H, m), 6.45-6.5 (1H, m), 6.95-7.05 (1H, m), 7.57 (1H, d, J=8.4 Hz), 7.65-7.75 (1H, m), 7.90 (1H, d, J=8.4 Hz), 8.2-8.3 (1H, m), 8.76 (1H, s) ESI-MS(m/z): 255 (M+H)+

### EXAMPLE 188

N-(2-Pyridinyl)-5-({[4′-(trifluoromethyl)-1,1′-biphenyl-2-yl]carbonyl}amino)-1-indolinecarboxamide

The title compound was obtained in the same manner as in Example 1 as a light brown solid.

¹H-NMR (DMSO-d₆):δ 3.11 (2H, t, J=8.5 Hz), 4.18 (2H, t, J=8.5 Hz), 7.0-7.1 (1H, m), 7.2-7.35 (1H, m), 7.4-7.9 (12H, m), 8.29 (1H, d, J=4.8 Hz), 8.98 (1H, s), 10.22 (1H, s) ESI-MS(m/z): 525 (M+Na)⁺, 503 (M+H)+

### EXAMPLE 189

5-{[(4′-Methyl-1,1′-biphenyl-2-yl)carbonyl]amino}-N-(2-pyridinyl)-1-indolinecarboxamide

The title compound was obtained in the same manner as in Example 1 as a light brown solid.

¹H-NMR (DMSO-d₆):δ 2.29 (3H, s), 3.12 (2H, t, J=8.4 Hz), 4.18 (2H, t, J=8.4 Hz), 7.0-7.1 (1H, m), 7.15-7.6 (10H, m), 7.7-7.8 (2H, m), 7.91 (1H, d, J=8.3 Hz), 8.25-8.30 (1H, m), 8.98 (1H, s), 10.11 (1H, s)

ESI-MS(m/z): 471 (M+Na)⁺, 449 (M+H)⁺

Preparation 155

6-(2,5-Dimethyl-1H-pyrrol-1-yl)-2-pyridinamine

The title compound was obtained in the same manner as in Preparation 137 as a light brown solid.

¹H-NMR (DMSO-d₆):δ 2.03 (6H, s), 5.71 (2H, s), 6.12 (2H, br s), 6.38 (1H, d, J=7.4 Hz), 6.44 (1H, d, J=8.1 Hz), 7.51 (1H, dd, J=8.1 Hz, 7.4 Hz)

ESI-MS(m/z): 210 (M+Na)⁺, 188 (M+H)⁺

Preparation 156

To a solution of 6-(2,5-dimethyl-1H-pyrrol-1-yl)-2-pyridinamine (1.87 g) and triethylamine (1.21 g) in dichloromethane was added dropwise phenyl chloroformate (1.72 g) at ambient temperature and the mixture was stirred at ambient temperature for 6 hours. Water was added and the separated organic layer was washed with brine, dried over magnesium sulfate, and evaporated in vacuo. The residue was purified by flash chromatography on silica gel and triturated with diisopropyl ether to give phenyl 6-(2,5-dimethyl-1H-pyrrol-1-yl)-2-pyridinylcarbamate (1.57 g) as a light brown solid.

¹H-NMR (DMSO-d₆):δ 2.03 (6H, s), 5.84 (2H, s), 7.15-7.5 (6H, m), 7.57 (1H, d, J=7.5 Hz), 7.98 (1H, d, J=7.4 Hz), 8.24 (1H, dd, J=7.5 Hz, 7.4 Hz)

### EXAMPLE 190

N-[6-(2,5-Dimethyl-1H-pyrrol-1-yl)-2-pyridinyl]-5-({[4′-(trifluoromethyl)-1,1′-biphenyl-2-yl]carbonyl}amino)-1-indolinecarboxamide

The title compound was obtained in the same manner as in Preparation 153 as a yellow solid.

¹H-NMR (DMSO-d₆):δ 2.07 (6H, s), 3.10 (2H, t, J=8.6 Hz), 4.18 (2H, t, J=8.6 Hz), 5.78 (2H, s), 7.0-7.1 (1H, m), 7.2-7.3 (1H, m), 7.45-7.6 (6H, m), 7.63 (2H, d, J=8.6 Hz), 7.76 (2H, d, J=8.6 Hz), 7.9-8.0 (2H, m), 9.20 (1H, s), 10.23 (1H, s)

ESI-MS(m/z): 618 (M+Na)⁺, 596 (M+H)⁺

### EXAMPLE 191

N-(6-Amino-2-pyridinyl)-5-({[4′-(trifluoromethyl)-1,1′-biphenyl-2-yl]carbonyl}amino)-1-indolinecarboxamide

The title compound was obtained in the same manner as in Example 9 as white crystals.

¹H-NMR (DMSO-d₆):δ 3.09 (2H, t, J=8.6 Hz), 4.12 (2H, t, J=8.6 Hz), 5.68 (2H, br s), 6.12 (1H, d, J=7.9 Hz), 7.07 (1H, d, J=7.5 Hz), 7.15-7.2 (1H, m), 7.3-7.35 (1H, m), 7.43 (1H, s), 7.5-7.65 (7H, m), 7.7-7.8 (3H, m), 8.16 (1H, s), 10.18 (1H, s)

ESI-MS(m/z): 540 (M+Na)⁺, 518 (M+H)⁺

### EXAMPLE 192

To a solution of N-(3-amino-4-{[2-(2-pyridinyl)ethyl]amino}phenyl)-4′-(trifluoromethyl)-1,1′-biphenyl-2-carboxamide (477 mg) in tetrahydrofuran (15 ml) was added dropwise a solution of tert-butyl nitrite (206 mg) in tetrahydrofuran (5 ml) at 50° C. and the mixture was stirred at 50° C. for 30 minutes. The reaction mixture was evaporated in vacuo and the residue was purified by column chromatography on silica gel and triturated with diisopropyl ether to give N-{1-[2-(2-pyridinyl)ethyl]-1H-1,2,3-benzotriazol-5-yl}-4′-(trifluoromethyl)-1,1′-biphenyl-2-carboxamide (250 mg) as a light brown solid.

¹H-NMR (DMSO-d₆):δ 3.39 (2H, t, J=7.3 Hz), 5.05 (2H, t, J=7.3 Hz), 7.15-7.25 (2H, m), 7.4-7.8 (11H, m), 8.24 (1H, s), 8.47 (1H, d, J=4.0 Hz), 10.54 (1H, s)

APCI-MS (m/z): 488 (M+H)⁺

### EXAMPLE 193

To a solution of N-(3-amino-4-{[2-(2-pyridinyl)ethyl]amino}phenyl)-4′-(trifluoromethyl)-1,1′-biphenyl-2-carboxamide (477 mg) in tetrahydrofuran (30 ml) was added 1-(1H-imidazol-1-ylcarbonyl)-1H-imidazole (487 mg) and the mixture was refluxed for 4 hours. The reaction mixture was evaporated in vacuo and the residue was purified by column chromatography on silica gel and triturated with diisopropyl ether to give N-{2-oxo-1-[2-(2-pyridinyl)ethyl]-2,3-dihydro-1H-benzimidazol-5-yl}-4′-(trifluoromethyl)-1,1′-biphenyl-2-carboxamide (156 mg) as a red powder.

¹H-NMR (DMSO-d₆):δ 3.06 (2H, t, J=7.3 Hz), 4.10 (2H, t, J=7.3 Hz), 6.86 (1H, d, J=8.4 Hz), 6.99 (1H, dd, J=8.4 Hz, 1.7 Hz), 7.2-7.3 (2H, m), 7.39 (1H, d, J=1.7 Hz), 7.5-7.8 (9H, m), 8.48 (1H, d, J=4.9 Hz), 10.23 (1H, s), 10.74 (1H, s)

APCI-MS (m/z): 503 (M+H)⁺

### EXAMPLE 194

2-[4-(Dimethylamino)phenyl]-N-(1-{[6-(2,5-dimethyl-1H-pyrrol-1-yl)-2-pyridinyl]acetyl}-2,3-dihydro-1H-indol-5-yl)-1-cyclohexene-1-carboxamide

The title compound was obtained in the same manner as in Example 151 as a light brown solid.

¹H-NMR (DMSO-d₆):δ 1.6-1.8 (4H, m), 2.01 (6H, s), 2.3-2.55 (4H, m), 2.82 (6H, s), 3.05 (2H, t, J=8.3 Hz), 4.02 (2H, s), 4.13 (2H, t, J=8.3 Hz), 5.76 (2H, s), 6.58 (2H, d, J=8.9 Hz), 7.07 (1H, d, J=7.2 Hz), 7.13 (2H, d, J=8.9 Hz), 7.28 (1H, d, J=7.8 Hz), 7.37 (1H, s), 7.39 (1H, d, J=7.2 Hz), 7.83 (1H, d, J=8.7 Hz), 7.94 (1H, dd, J=8.7 Hz, 7.8 Hz), 9.66 (1H, s)

ESI-MS(m/z): 596 (M+Na)⁺, 574 (M+H)⁺

### EXAMPLE 195

N-{1-[(6-Amino-2-pyridinyl)acetyl]-2,3-dihydro-1H-indol-5-yl}-2-[4-(dimethylamino)phenyl]-1-cyclohexene-1-carboxamide

The title compound was obtained in the same manner as in Example 9 as white crystals.

¹H-NMR (DMSO-d₆):δ 1.6-1.8 (4H, m), 2.2-2.35 (4H, m), 2.82 (6H, s), 3.05 (2H, t, J=8.4 Hz), 3.67 (2H, s), 4.13 (2H, t, J=8.4 Hz), 5.84 (2H, br s), 6.29 (1H, d, J=8.1 Hz), 6.40 (1H, d, J=7.0 Hz), 6.58 (2H, d, J=8.9 Hz), 7.07 (1H, d, J=8.7 Hz), 7.13 (2H, d, J=8.9 Hz), 7.30 (1H, dd, J=8.1 Hz, 7.0 Hz), 7.84 (1H, d, J=8.7 Hz), 9.35 (1H, s)

ESI-MS(m/z): 518 (M+Na)⁺, 496 (M+H)⁺

Preparation 157

To a solution of (1-trityl-1H-1,2,4-triazol-3-yl)methanol (1.71 g) and triethylamine (607 mg) in dichloromethane (40 ml) was added methanesulfonyl chloride (630 mg) at 5° C. and the mixture was stirred at ambient temperature for 3 hours. Water was added and the separated organic layer was washed with brine, dried over magnesium sulfate, and evaporated in vacuo. The residue was purified by column chromatography on silica gel and triturated with diisopropyl ether to give (1-trityl-1H-1,2,4-triazol-3-yl)methyl methanesulfonate (1.72 g) as a white solid.

¹H-NMR (DMSO-d₆):δ 3.04 (3H, s), 4.50 (2H, s), 6.95-7.1 (5H, m), 7.35-7.5 (10H, m), 8.15 (1H, s)

ESI-MS(m/z): 442 (M+Na)⁺

### EXAMPLE 196

To a solution of N-(2,3-dihydro-1H-indol-5-yl)-4′-(trifluoromethyl)-1,1′-biphenyl-2-carboxamide (1.30 g) and (1-trityl-1H-1,2,4-triazol-3-yl)methyl methanesulfonate (1.71 g) in N,N,-dimethylformamide (40 ml) was added triethylamine (688 mg) and the mixture was stirred at 50° C. for 20 hours. The reaction mixture was poured into a mixture of ethyl acetate and water and the separated organic layer was washed with water and brine, dried over magnesium sulfate, and evaporated in vacuo. The residue was purified by column chromatography on silica gel and triturated with diisopropyl ether to give 4′-(trifluoromethyl)-N-{1-[(1-trityl-1H-1,2,4-triazol-3-yl)methyl]-2,3-dihydro-1H-indol-5-yl}-1,1′-biphenyl-2-carboxamide (1.49 g) as a brown solid.

¹H-NMR (DMSO-d₆):δ 2.79 (2H, t, J=8.3 Hz), 3.40 (2H, t, J=8.3 Hz), 4.29 (2H, s), 6.49 (1H, d, J=8.4 Hz), 6.95-7.75 (25H, m), 8.01 (1H, s), 10.01 (1H, s)

ESI-MS(m/z): 728 (M+Na)⁺

### EXAMPLE 197

To a suspension of 4′-(trifluoromethyl)-N-{1-[(1-trityl-1H-1,2,4-triazol-3-yl)methyl]-2,3-dihydro-1H-indol-5-yl}-1,1′-biphenyl-2-carboxamide (1.48 g) in methanol (15 ml) was added conc. HCl (1.75 ml) and the mixture was stirred at ambient temperature for 24 hours. The reaction mixture was poured into a mixture of ethyl acetate and water and the separated organic layer was washed with water and brine, dried over magnesium sulfate, and evaporated in vacuo. The residue was purified by column chromatography on silica gel and triturated with diisopropyl ether to give N-[1-(1H-1,2,4-triazol-3-ylmethyl)-2,3-dihydro-1H-indol-5-yl]-4′-(trifluoromethyl)-1,1′-biphenyl-2-carboxamide (215 mg) as a light brown solid.

¹H-NMR (DMSO-d₆):δ 2.83 (2H, t, J=8.3 Hz), 3.32 (2H, t, J=8.3 Hz), 4.30 (2H, s), 6.52 (1H, d, J=8.4 Hz), 7.08 (1H, d, J=8.4 Hz), 7.21 (1H, s), 7.5-7.8 (8H, m), 8.1-8.3 (1H, br), 9.98 (1H, s), 13.88 (1H, br)

ESI-MS(m/z): 486 (M+Na)⁺, 464 (M+H)⁺

### EXAMPLE 198

2-(4-Ethylphenyl)-N-[1-(2-pyridinylacetyl)-2,3-dihydro-1H-indol-5-yl]-1-cyclohexene-1-carboxamide

The title compound was obtained in the same manner as in Example 8 as white crystals.

¹H-NMR (DMSO-d₆):δ 1.11 (3H, t, J=7.6 Hz), 1.6-1.8 (4H, m), 2.25-2.4 (4H, m), 2.53 (2H, q, J=7.6 Hz), 3.06 (2H, t, J=8.3 Hz), 3.96 (2H, s), 4.15 (2H, t, J=8.3 Hz), 6.9-7.35 (7H, m), 7.7-7.9 (2H, m), 8.49 (1H, d, J=5.0 Hz), 9.37 (1H, s)

ESI-MS(m/z):488 (M+Na)⁺, 466 (M+H)⁺

### EXAMPLE 199

N-(1-{[6-(2,5-Dimethyl-1H-pyrrol-1-yl)-2-pyridinyl]acetyl}-2,3-dihydro-1H-indol-5-yl)-2-(4-ethylphenyl)-1-cyclohexene-1-carboxamide

The title compound was obtained in the same manner as in Example 8 as a light brown solid.

¹H-NMR (DMSO-d₆):δ 1.11 (3H, t, J=7.6 Hz), 1.6-1.8 (4H, m), 2.01 (6H, s), 2.3-2.45 (4H, m), 2.54 (2H, q, J=7.6 Hz), 3.04 (2H, t, J=7.8 Hz), 4.02 (2H, s), 4.12 (2H, t, J=7.8 Hz), 5.76 (2H, s), 6.9-7.4 (8H, m), 7.8-8.0 (2H, m), 9.38 (1H, s)

negative ESI-MS(m/z): 557 (M−H)⁻

### EXAMPLE 200

N-{1-[(6-Amino-2-pyridinyl)acetyl]-2,3-dihydro-1H-indol-5-yl}-2-(4-ethylphenyl)-1-cyclohexene-1-carboxamide

The title compound was obtained in the same manner as in Example 9 as white crystals.

¹H-NMR (DMSO-d₆):δ 1.11 (3H, t, J=7.6 Hz), 1.6-1.8 (4H, m), 2.25-2.4 (4H, m), 2.51 (2H, q, J=7.6 Hz), 3.12 (2H, t, J=8.4 Hz), 4.04 (2H, s), 4.15 (2H, t, J=8.4 Hz), 6.75 (1H, d, J=7.1 Hz), 6.87 (1H, d, J=8.7 Hz), 7.04 (1H, dd, J=8.7 Hz, 1.6 Hz), 7.07 (2H, d, J=8.1 Hz), 7.20 (2H, d, J=8.1 Hz), 7.35 (1H, d, J=1.6 Hz), 7.78 (2H, br s), 7.75-7.9 (2H, m), 9.45 (1H, s)

ESI-MS(m/z): 503 (M+Na)⁺, 481 (M+H)⁺

Preparation 158

5,6-Dimethyl-4′-(trifluoromethyl)-1,1′-biphenyl-2-carboxylic acid

The title compound was obtained in the same manner as in Preparation 15.

¹H-NMR (DMSO-d₆):δ 1.89 (3H, s), 2.33 (3H, s), 7.28-7.38 (3H, m), 7.60 (1H, d, J=7.8 Hz), 7.75 (2H, d, J=8.1 Hz), 12.42 (1H, s)

### EXAMPLE 201

5,6-Dimethyl-N-[1-(2-pyridinylacetyl)-2,3-dihydro-1H-indol-5-yl]-4′-(trifluoromethyl)-1,1′-biphenyl-2-carboxamide

The title compound was obtained in the same manner as in Example 6.

¹H-NMR (DMSO-d₆):δ 1.96 (3H, s), 2.34 (3H, s), 3.06 (2H, t, J=8.3 Hz), 3.97 (2H, s), 4.16 (2H, t, J=8.3 Hz), 7.09 (1H, dd, J=1.4 Hz, 8.7 Hz), 7.21-7.30 (1H, m), 7.30-7.39 (4H, m), 7.40-7.48 (2H, m), 7.68-7.81 (3H, m), 7.87 (1H, d, J=8.7 Hz), 8.45-8.52 (1H, m), 9.99 (1H, s)

negative ESI-MS(m/z): 528 (M−H)⁻

Preparation 159

Methyl 4′,5,6-trimethyl-1,1′-biphenyl-2-carboxylate

The title compound was obtained in the same manner as in Preparation 15.

¹H-NMR (DMSO-d₆):δ 1.91 (3H, s), 2.30 (3H, s), 2.34 (3H, s), 6.99 (2H, d, J=8.0 Hz), 7.18 (2H, d, J=8.0 Hz), 7.23 (1H, d, J=7.8 Hz), 7.45 (1H, d, J=7.8 Hz), 12.23 (1H, s)

### EXAMPLE 202

4′,5,6-Trimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.97 (3H, s), 2.28 (3H, s), 2.32 (3H, s), 3.06 (2H, t, J=8.4 Hz), 3.97 (2H, s), 4.16 (2H, t, J=8.4 Hz), 7.05-7.21 (5H, m), 7.21-7.42 (5H, m), 7.75 (1H, dt, J=1.9 Hz, 7.6 Hz), 7.86 (1H, d, J=8.7 Hz), 8.45-8.52 (1H, m), 9.83 (1H, s)

ESI-MS(m/z): 476 (M+H)⁺, 498 (M+Na)⁺

Preparation 160

5,6-Dimethyl-1,1′-biphenyl-2-carboxylic acid

The title compound was obtained in the same manner as in Preparation 15.

¹H-NMR (DMSO-d₆):δ 1.91 (3H, s), 2.31 (3H, s), 7.05-7.15 (2H, m), 7.25 (1H, d, J=7.9 Hz), 7.29-7.45 (3H, m), 7.48 (1H, d, J=7.9 Hz), 12.27 (1H, s)

### EXAMPLE 203

5,6-Dimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.97 (3H, s), 2.33 (3H, s), 3.06 (2H, t, J=8.3 Hz), 3.97 (2H, s), 4.15 (2H, t, J=8.3 Hz), 7.08 (1H, d, J=8.7 Hz), 7.17-7.43 (10H, m), 7.75 (1H, dt, J=1.6 Hz, 7.6 Hz), 7.84 (1H, d, J=8.7 Hz), 8.45-8.53 (1H, m), 9.82 (1H, s)

ESI-MS(m/z): 462 (M+H)⁺, 484 (M+Na)⁺

Preparation 161

4′-Fluoro-5,6-dimethyl-1,1′-biphenyl-2-carboxylic acid

The title compound was obtained in the same manner as in Preparation 15.

¹H-NMR (DMSO-d₆):δ 1.91 (3H, s), 2.31 (3H, s), 7.10-7.23 (4H, m), 7.26 (1H, d, J=7.8 Hz), 7.51 (1H, d, J=7.8 Hz), 12.32 (1H, s)

### EXAMPLE 204

4′-Fluoro-5,6-dimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.97 (3H, s), 2.33 (3H, s), 3.07 (2H, t, J=8.3 Hz), 3.97 (2H, s), 4.18 (2H, t, J=8.3 Hz), 7.07-7.44 (10H, m), 7.75 (1H, dt, J=1.8 Hz, 7.6 Hz), 7.86 (1H, d, J=8.7 Hz), 8.46-8.53 (1H, m), 9.89 (1H, s)

ESI-MS(m/z): 480 (M+H)⁺, 502 (M+Na)⁺

Preparation 162

4′-Chloro-5,6-dimethyl-1,1′-biphenyl-2-carboxylic acid

The title compound was obtained in the same manner as in Preparation 15.

¹H-NMR (DMSO-d₆):δ 1.90 (3H, s), 2.32 (3H, s), 7.09-7.15 (2H, m), 7.28 (1H, d, J=7.9 Hz), 7.40-7.47 (2H, m), 7.53 (1H, d, J=7.9 Hz), 12.35 (1H, s)

### EXAMPLE 205

4′-Chloro-5,6-dimethyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.97 (3H, s), 2.33 (3H, s), 3.07 (2H, t, J=8.3 Hz), 3.97 (2H, s), 4.16 (2H, d, J=8.3 Hz), 7.07-7.17 (1H, m), 7.18-7.46 (9H, m), 7.70-7.80 (1H, m), 7.87 (1H, d, J=8.6 Hz), 8.46-8.53 (1H, m), 9.94 (1H, s)

### EXAMPLE 206

N-[1-(2-Pyridinylacetyl)-2,3-dihydro-1H-indol-5-yl]-2-[4-(trifluoromethyl)phenyl]-1-cyclohexene-1-carboxamide

The title compound was obtained in the same manner as in Example 6.

¹H-NMR (DMSO-d₆):δ 1.64-1.84 (4H, m), 2.33-2.48 (4H, m), 3.05 (2H, t, J=8.4 Hz), 3.96 (2H, s), 4.15 (2H, t, J=8.4 Hz), 6.96-7.06 (1H, m), 7.22-7.38 (3H, m), 7.47 (2H, d, J=8.2 Hz), 7.62 (2H, d, J=8.2 Hz), 7.75 (1H, dt, J=1.8 Hz, 7.7 Hz), 7.83 (1H, d, J=8.7 Hz), 8.45-8.52 (1H, m), 9.56 (1H, s)

negative ESI-MS(m/z): 504 (M−H)⁻

### EXAMPLE 207

2-(4-Methylphenyl)-N-[1-(2-pyridinylacetyl)-2,3-dihydro-1H-indol-5-yl]-1-cyclohexene-1-carboxamide

The title compound was obtained in the same manner as in Example 6.

¹H-NMR (DMSO-d₆):δ 1.60-1.81 (4H, m), 2.20 (3H, s), 2.28-2.42 (4H, m), 3.06 (2H, t, J=8.3 Hz), 3.96 (2H, s), 4.15 (2H, t, J=8.3 Hz), 7.00-7.09 (3H, m), 7.17 (2H, d, J=8.1 Hz), 7.22-7.40 (3H, m), 7.75 (1H, dt, J=1.8 Hz, 7.7 Hz), 7.83 (1H, d, J=8.7 Hz), 8.44-8.52 (1H, m), 9.44 (1H, s)

negative ESI-MS(m/z): 450 (M−H)⁻

### EXAMPLE 208

2-(4-Chlorophenyl)-N-[1-(2-pyridinylacetyl)-2,3-dihydro-1H-indol-5-yl]-1-cyclohexene-1-carboxamide

The title compound was obtained in the same manner as in Example 6.

¹H-NMR (DMSO-d₆):δ 1.62-1.80 (4H, m), 2.39-2.43 (4H, m), 3.07 (2H, t, J=8.3 Hz), 3.97 (2H, s), 4.16 (2H, t, J=8.3 Hz), 7.05 (1H, dd, J=1.8 Hz, 8.6 Hz), 7.22-7.38 (7H, m), 7.75 (1H, dt, J=1.8 Hz, 7.6 Hz), 7.84 (1H, d, J=8.6 Hz), 8.46-8.52 (1H, m), 9.53 (1H, s)

negative ESI-MS(m/z): 470 (M−H)⁻

### EXAMPLE 209

2-(4-Methoxyphenyl)-N-[1-(2-pyridinylacetyl)-2,3-dihydro-1H-indol-5-yl]-1-cyclohexene-1-carboxamide

The title compound was obtained in the same manner as in Example 6.

¹H-NMR (DMSO-d₆):δ 1.60-1.80 (4H, m), 2.27-2.40 (4H, m), 3.06 (2H, t, J=8.3 Hz), 3.67 (3H, s), 3.96 (2H, s), 4.15 (2H, t, J=8.3 Hz), 6.80 (2H, d, J=8.7 Hz), 7.01-7.09 (1H, m), 7.17-7.40 (5H, m), 7.75 (1H, dt, J=1.9 Hz, 7.6 Hz), 7.84 (1H, d, J=8.7 Hz), 8.45-8.52 (1H, m), 9.42 (1H, s)

negative ESI-MS (m/z): 466 (M−H)⁻

Preparation 163

2-[4-(Dimethylamino)phenyl]-1-cyclohexene-1-carboxylic acid

The title compound was obtained in the same manner as in Preparation 15.

¹H-NMR (DMSO-d₆):δ 1.53-1.75 (4H, m), 2.20-2.37 (4H, m), 2.87 (6H, s), 6.57-6.68 (2H, m), 6.98-7.07 (2H, m), 11.84 (1H, s)

negative ESI-MS(m/z): 244 (M−H)⁻

### EXAMPLE 210

2-[4-(Dimethylamino)phenyl]-N-[1-(2-pyridinylacetyl)-2,3-dihydro-1H-indol-5-yl]-1-cyclohexene-1-carboxamide

The title compound was obtained in the same manner as in Example 6.

¹H-NMR (DMSO-d₆):δ 1.58-1.80 (4H, m), 2.26-2.40 (4H, m), 2.81 (6H, s), 3.06 (2H, t, J=8.3 Hz), 3.96 (2H, s), 4.14 (2H, t, J=8.3 Hz), 6.58 (2H, d, J=8.7 Hz), 7.04-7.19 (3H, m), 7.21-7.42 (3H, m), 7.74 (1H, dt, J=1.8 Hz, 7.6 Hz), 7.84 (1H, d, J=8.7 Hz), 8.44-8.52 (1H, m), 9.38 (1H, s)

negative ESI-MS(m/z): 479 (M−H)⁻

### EXAMPLE 211

4′-Iodo-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.11 (2H, t, J=8.3 Hz), 3.99 (2H, s), 4.19 (2H, t, J=8.3 Hz), 7.17-7.64 (10H, m), 7.68-7.82 (3H, m), 7.94 (1H, d, J=8.7 Hz), 8.46-8.56 (1H, m), 10.24 (1H, s)

ESI-MS(m/z): 560 (M+H)⁺, 582 (M+Na)⁺

### EXAMPLE 212

To a mixture of 4′-iodo-N-[1-(2-pyridinylacetyl)-2,3-dihydro-1H-indol-5-yl]-1,1′-biphenyl-2-carboxamide (3.2 g), potassium carbonate (3.2 g) and palladium(II)acetate (0.26 g) in a mixture of N,N-dimethylformamide (48 ml) and water (16 ml) was introduced carbon monoxide for 1 hour. The reaction mixture was stirred for 20 hours at ambient temperature under carbon monoxide atmosphere. The reaction mixture was poured into water and the mixture was adjusted to pH 5 with 6N hydrochloric acid. The mixture was extracted with a mixture of ethyl acetate and tetrahydrofuran, and the catalyst was filtered off. The extract layer was washed with water, dried over magnesium sulfate and evaporated in vacuo. The residue was crystallized from ethyl acetate to give 2′-({[1-(2-pyridinylacetyl)-2,3-dihydro-1H-indol-5-yl]amino}carbonyl)-1,1′-biphenyl-4-carboxylic acid (2.22 g).

¹H-NMR (DMSO-d₆):δ 3.11 (2H, t, J=8.3 Hz), 3.99 (2H, s), 4.18 (2H, t, J=8.3 Hz), 7.18-7.31 (2H, m), 7.35 (1H, d, J=7.9 Hz), 7.46-7.67 (7H, m), 7.71-7.82 (1H, m), 7.87-7.98 (3H, m), 8.49 (1H, dd, J=0.9 Hz, 4.1 Hz), 10.24 (1H, s), 12.97 (1H, s)

negative ESI-MS(m/z): 476 (M−H)⁻

### EXAMPLE 213

1-[3-(Dimethylamino)propyl]-3-ethylcarbodiimide (0.39 g) was added to a mixture of 2′-({[1-(2-pyridinylacetyl)-2,3-dihydro-1H-indol-5-yl]amino}carbonyl)-1,1′-biphenyl-4-carboxylic acid (1.0 g), methanol (0.85 ml) and 1-hydroxybenzotriazole (0.34 g) in N,N-dimethylformamide (15 ml) under ice-cooling and the mixture was stirred at ambient temperature for 20 hours. The reaction mixture was poured into a mixture of ethyl acetate and water under stirring. The resulting precipitate was collected by filtration to give methyl 2′-({[1-(2-pyridinylacetyl)-2,3-dihydro-1H-indol-5-yl]amino}carbonyl)-1,1′-biphenyl-4-carboxylate (0.73 g).

¹H-NMR (DMSO-d₆):δ 3.10 (2H, t, J=8.3 Hz), 3.84 (3H, s), 3.99 (2H, s), 4.18 (2H, t, J=8.3 Hz), 7.16-7.31 (2H, m), 7.35 (1H, d, J=7.8 Hz), 7.46-7.67 (7H, m), 7.70-7.81 (1H, m), 7.87-8.00 (3H, m), 8.46-8.54 (1H, m), 10.23 (1H, s)

ESI-MS(m/z): 492 (M+H)⁺, 514 (M+Na)⁺

### EXAMPLE 214

Isopropyl 2′-({[1-(2-pyridinylacetyl)-2,3-dihydro-1H-indol-5-yl]amino}carbonyl)-1,1′-biphenyl-4-carboxylate

The title compound was obtained in the same manner as in Example 213.

¹H-NMR (DMSO-d₆):δ 1.31 (6H, d, J=6.2 Hz), 3.11 (2H, t, J=8.3 Hz), 3.99 (2H, s), 4.19 (2H, t, J=8.3 Hz), 5.03-5.23 (1H, m), 7.02-7.32 (2H, m), 7.35 (1H, d, J=7.8 Hz), 7.46-7.66 (7H, m), 7.71-7.81 (1H, m), 7.87-7.97 (3H, m), 8.46-8.53 (1H, m), 10.27 (1H, s)

ESI-MS(m/z): 520 (M+H)⁺, 542 (M+Na)⁺

### EXAMPLE 215

A mixture of 2′-({[1-(2-pyridinylacetyl)-2,3-dihydro-1H-indol-5-yl]amino}carbonyl)-1,1′-biphenyl-4-carboxylic acid (0.4 g), potassium carbonate (0.15 g) and chloromethyl pivalate (0.12 ml) in N,N-dimethylformamide (4 ml) was stirred at ambient temperature for 20 hours. The reaction mixture was poured into water and the mixture was extracted with ethyl acetate. The extract layer was washed with water, dried over magnesium sulfate and evaporated in vacuo. The residue was crystallized from ethyl acetate to give pivaloyloxymethyl 2′-({[1-(2-pyridinylacetyl)-2,3-dihydro-1H-indol-5-yl]amino}carbonyl)-1,1′-biphenyl-4-carboxylate (0.21 g).

¹H-NMR (DMSO-d₆):δ 1.14 (9H, s), 3.10 (2H, t, J=8.3 Hz), 3.99 (2H, s), 4.18 (2H, t, J=8.3 Hz), 5.95 (2H, s), 7.16-7.32 (2H, m), 7.35 (1H, d, J=7.8 Hz), 7.47-7.67 (7H, m), 7.71-7.82 (1H, m), 7.87-8.02 (3H, m), 8.45-8.54 (1H, m), 10.25 (1H, s)

ESI-MS(m/z): 592 (M+H)⁺, 614 (M+Na)⁺

### EXAMPLE 216

1-[3-(Dimethylamino)propyl]-3-ethylcarbodiimide (0.16 g) was added to a solution of 2′-({[1-(2-pyridinylacetyl)-2,3-dihydro-1H-indol-5-yl]amino}carbonyl)-1,1′-biphenyl-4-carboxylic acid (0.4 g), 4-(dimethylamino)pyridine (5 mg), dimethylamine (0.17 ml) and 1-hydroxybenzotriazole (0.14 g) in N,N-dimethylformamide (4 ml) under ice-cooling and the mixture was stirred at ambient temperature for 20 hours. The reaction mixture was poured into a mixture of ethyl acetate and water under stirring. The resulting precipitate was collected by filtration to give N⁴′,N⁴′-diethyl-N²-[1-(2-pyridinylacetyl)-2,3-dihydro-1H-indol-5-yl]-1,1′-biphenyl-2,4′-dicarboxamide (0.20 g).

¹H-NMR (DMSO-d₆):δ 0.80-1.25 (6H, m), 2.92-3.52 (4H, m), 3.10 (2H, t, J=8.3 Hz), 3.98 (2H, s), 4.18 (2H, t, J=8.3 Hz), 7.16 (1H, d, J=8.7 Hz), 7.21-7.41 (4H, m), 7.41-7.65 (7H, m), 7.69-7.82 (1H, m), 7.89 (1H, d, J=8.6 Hz), 8.46-8.54 (1H, m), 10.07 (1H, s)

ESI-MS(m/z): 533 (M+H)⁺, 555 (M+Na)⁺

Preparation 164

4′-(Dimethylamino)-5-methyl-1,1′-biphenyl-2-carboxylic acid

The title compound was obtained in the same manner as in Preparation 15.

¹H-NMR (DMSO-d₆):δ 2.36 (3H, s), 2.92 (6H, s), 6.73 (2H, d, J=8.7 Hz), 7.12-7.20 (4H, m), 7.54 (1H, d, J=8.3 Hz), 12.50 (1H, s)

negative ESI-MS(m/z): 254 (M−H)⁻

### EXAMPLE 217

4′-(Dimethylamino)-5-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.38 (3H, s), 2.87 (6H, s), 3.10 (2H, t, J=8.2 Hz), 3.98 (2H, s), 4.18 (2H, t, J=8.2 Hz), 6.69 (2H, d, J=8.7 Hz), 7.13-7.40 (8H, m), 7.53 (1H, s), 7.75 (1H, dt, J=1.7 Hz, 7.6 Hz), 7.91 (1H, d, J=8.7 Hz), 8.46-8.54 (1H, m), 10.03 (1H, s)

negative ESI-MS(m/z): 489 (M−H)⁻

Preparation 165

5-Chloro-4′-(dimethylamino)-1,1′-biphenyl-2-carboxylic acid

The title compound was obtained in the same manner as in Preparation 15.

¹H-NMR (DMSO-d₆):δ 2.94 (6H, s), 6.75 (2H, d, J=8.8 Hz), 7.20 (2H, d, J=8.8 Hz), 7.37-7.44 (2H, m), 7.95 (1H, d, J=8.5 Hz), 12.86 (1H, s)

negative ESI-MS(m/z): 274 (M−H)⁻

### EXAMPLE 218

5-Chloro-4′-(dimethylamino)-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.89 (6H, s), 3.11 (2H, t, J=8.3 Hz), 3.99 (2H, s), 4.19 (2H, t, J=8.3 Hz), 6.70 (2H, d, J=8.8 Hz), 7.21-7.54 (9H, m), 7.76 (1H, dt, J=1.8 Hz, 7.6 Hz), 7.92 (1H, d, J=8.7 Hz), 8.46-8.52 (1H, m), 10.18 (1H, s)

Preparation 166

4′-(Dimethylamino)-4-methyl-1,1′-biphenyl-2-carboxylic acid

The title compound was obtained in the same manner as in Preparation 15.

¹H-NMR (DMSO-d₆):δ 2.34 (3H, s), 2.92 (6H, s), 6.70-6.80 (2H, m), 7.11-7.20 (2H, m), 7.22 (1H, d, J=7.9 Hz), 7.31 (1H, dd, J=1.6 Hz, 7.9 Hz), 7.42 (1H, d, J=1.6 Hz), 12.60 (1H, s)

negative ESI-MS(m/z): 254 (M−H)⁻

### EXAMPLE 219

4′-(Dimethylamino)-4-methyl-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 2.36 (3H, s), 2.87 (6H, s), 3.11 (2H, t, J=8.3 Hz), 3.99 (2H, s), 4.19 (2H, t, J=8.3 Hz), 6.69 (2H, d, J=8.8 Hz), 7.20-7.38 (8H, m), 7.54 (1H, s), 7.75 (1H, dt, J=1.8 Hz, 7.6 Hz), 7.92 (1H, d, J=8.7 Hz), 8.46-8.53 (1H, m), 10.12 (1H, s)

negative ESI-MS(m/z): 489 (M−H)⁻

Preparation 167

A mixture of 4′-iodo-1,1′-biphenyl-2-carboxylic acid (2.5 g), sodium hydrogencarbonate (0.97 g) and methyl iodide (0.72 ml) in N,N-dimethylformamide (25 ml) was stirred at ambient temperature for 20 hours. The reaction mixture was poured into water and the mixture was extracted with ethyl acetate. The extract layer was washed with water, dried over magnesium sulfate and evaporated in vacuo to give methyl 4′-iodo-1,1′-biphenyl-2-carboxylate (2.44 g).

¹H-NMR (DMSO-d₆):δ 3.62 (3H, s), 7.04-7.14 (2H, m), 7.38-7.46 (1H, m), 7.46-7.56 (1H, m), 7.57-7.69 (1H, m), 7.73-7.83 (3H, m)

ESI-MS(m/z): 339 (M+H)⁺, 361 (M+Na)⁺

Preparation 168

A mixture of methyl 4′-iodo-1,1′-biphenyl-2-carboxylate (1.0 g), piperidine (0.29 ml), cesium carbonate (1.9 g), rac-2,2′-bis(diphenylphosphino)-1,1′-binaphthyl (0.18 g) and palladium(II)acetate (66 mg) in toluene (20 ml) was stirred at 90° C. for 20 hours. The reaction mixture was poured into water and the mixture was extracted with ethyl acetate. The extract layer was washed with water, dried over magnesium sulfate and evaporated in vacuo. To the residue was added a solution of sodium hydroxide (0.3 g) in a mixture water (3 ml) and ethanol (9 ml), and the mixture was stirred under reflux for 18 hours. The solvent was evaporated. The residue was dissolved in water and the solution was adjusted to pH 6 with 6N hydrochloric acid. The mixture was extracted with ethyl acetate. The extract layer was washed with brine, dried over magnesium sulfate and evaporated in vacuo. The residue was purified by column chromatography on silica gel using a mixture of hexane and ethyl acetate (1:1 v/v) as an eluent. The eluted fractions containing the desired product were collected and evaporated in vacuo to give 4′-(1-piperidinyl)-1,1′-biphenyl-2-carboxylic acid (0.24 g).

¹H-NMR (DMSO-d₆):δ 1.48-1.70 (6H, m), 3.10-3.27 (4H, m), 6.94 (2H, d, J=8.7 Hz), 7.18 (2H, d, J=8.7 Hz), 7.30-7.43 (2H, m), 7.47-7.67 (2H, m), 12.70 (1H, s)

negative ESI-MS(m/z): 280 (M−H)⁻

### EXAMPLE 220

4′-(1-Piperidinyl)-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 1.43-1.67 (6H, m), 3.04-3.23 (6H, m), 3.99 (2H, s), 4.19 (2H, t, J=8.4 Hz), 6.90 (2H, d, J=8.7 Hz), 7.20-7.56 (10H, m), 7.76 (1H, dt, J=1.8 Hz, 7.7 Hz), 7.91 (1H, d, J=8.6 Hz), 8.46-8.53 (1H, m), 10.10 (1H, s)

ESI-MS(m/z): 517 (M+H)⁺, 539 (M+Na)⁺

Preparation 169

4′-(4-Morpholinyl)-1,1′-biphenyl-2-carboxylic acid

The title compound was obtained in the same manner as in Preparation 168.

¹H-NMR (DMSO-d₆):δ 3.15 (4H, t, J=4.8 Hz), 3.75 (4H, t, J=4.8 Hz), 6.97 (2H, d, J=8.8 Hz), 7.22 (2H, d, J=8.8 Hz), 7.32-7.42 (2H, m), 7.47-7.57 (1H, m), 7.64 (1H, dd, J=1.4 Hz, 7.5 Hz), 12.70 (1H, s)

negative ESI-MS(m/z): 282 (M−H)⁻

### EXAMPLE 221

4′-(4-Morpholinyl)-N-[1-(2-pyridinylacetyl)-2,3-dihydro-1H-indol-5-yl]-1,1′-biphenyl-2-carboxamide

The title compound was obtained in the same manner as in Example 6.

¹H-NMR (DMSO-d₆):δ 3.02-3.21 (6H, m), 3.64-3.80 (4H, m), 3.99 (2H, s), 4.19 (2H, t, J=8.3 Hz), 6.93 (2H, d, J=8.7 Hz), 7.21-7.58 (10H, m), 7.76 (1H, dt, J=1.8 Hz, 7.6 Hz), 7.92 (1H, d, J=8.7 Hz), 8.46-8.55 (1H, m), 10.14 (1H, s)

ESI-MS(m/z): 519 (M+H)⁺, 541 (M+Na)⁺

Preparation 170

A mixture of methyl 4′-iodo-1,1′-biphenyl-2-carboxylate (1.8 g), thiomorpholine (0.55 ml), cesium carbonate (3.5 g), rac-2,2′-bis(diphenylphosphino)-1,1′-binaphthyl (0.33 g) and palladium(II)acetate (0.12 g) in toluene (36 ml) was stirred at 90° C. for 20 hours. The reaction mixture was poured into water and the mixture was extracted with ethyl acetate. The extract layer was washed with water, dried over magnesium sulfate and evaporated in vacuo. The residue was purified by column chromatography on silica gel using a mixture of hexane and ethyl acetate (9:1 v/v) as an eluent. The eluted fractions containing the desired product were collected and evaporated in vacuo to give methyl 4′-(4-thiomorpholinyl)-1,1′-biphenyl-2-carboxylate (1.02 g).

¹H-NMR (DMSO-d₆):δ 2.63-2.72 (4H, m), 3.54-3.66 (4H, m), 3.62 (3H, s), 6.92-7.01 (2H, m), 7.12-7.20 (2H, m), 7.36-7.46 (2H, m), 7.55 (1H, dd, J=1.5 Hz, 7.0 Hz), 7.62-7.68 (1H, m)

ESI-MS(m/z): 314 (M+H)⁺, 336 (M+Na)⁺

Preparation 171

A mixture of methyl 4′-(4-thiomorpholinyl)-1,1′-biphenyl-2-carboxylate (1.0 g) and sodium hydroxide (0.32 g) in a mixture of water (3 ml) and ethanol (10 ml) was stirred under reflux for 18 hours. The solvent was evaporated. The residue was dissolved in water and the solution was adjusted to pH 6 with 6N hydrochloric acid. The mixture was extracted with ethyl acetate. The extract layer was washed with brine, dried over magnesium sulfate and evaporated in vacuo. The residue was triturated with a mixture of hexane and diisopropyl ether to give 4′-(4-thiomorpholinyl)-1,1′-biphenyl-2-carboxylic acid (0.87 g).

¹H-NMR (DMSO-d₆):δ 2.60-2.73 (4H, m), 3.51-3.65 (4H, m), 6.95 (2H, d, J=8.7 Hz), 7.21 (2H, d, J=8.7 Hz), 7.31-7.43 (2H, m), 7.47-7.57 (1H, m), 7.60-7.67 (1H, m), 12.70 (1H, s)

negative ESI-MS(m/z): 298 (M−H)⁻

### EXAMPLE 222

N-[1-(2-Pyridinylacetyl)-2,3-dihydro-1H-indol-5-yl]-4′-(4-thiomorpholinyl)-1,1′-biphenyl-2-carboxamide

The title compound was obtained in the same manner as in Example 6.

¹H-NMR (DMSO-d₆):δ 2.55-2.70 (4H, m), 3.11 (2H, t, J=8.3 Hz), 3.48-3.64 (4H, m), 3.99 (2H, s), 4.18 (2H, t, J=8.3 Hz), 6.90 (2H, d, J=8.7 Hz), 7.21-7.58 (10H, m), 7.75 (1H, dt, J=1.7 Hz, 7.6 Hz), 7.92 (1H, d, J=8.7 Hz), 8.46-8.53 (1H, m), 10.10 (1H, s)

ESI-MS(m/z): 535 (M+H)⁺, 557 (M+Na)⁺

### EXAMPLE 223

N-[1-(2-Pyridinylacetyl)-2,3-dihydro-1H-indol-5-yl]-2-(1H-pyrrol-1-yl)benzamide

The title compound was obtained in the same manner as in Example 6.

¹H-NMR (DMSO-d₆):δ 3.12 (2H, t, J=8.3 Hz), 3.99 (2H, s), 4.19 (2H, t, J=8.3 Hz), 6.12-6.21 (2H, m), 6.95-7.04 (2H, m), 7.20-7.64 (8H, m), 7.71-7.82 (1H, m), 7.94 (1H, d, J=8.7 Hz), 8.46-8.54 (1H, m), 10.25 (1H, s)

ESI-MS(m/z): 423 (M+H)⁺, 445 (M+Na)⁺

Preparation 172

To a suspension of sodium hydride (60% oil dispersion) (5.16 g) in N,N-dimethylformamide (160 ml) was added dropwise a solution of methyl 2-oxocycloheptanecarboxylate (20.0 g) at 10° C. under a nitrogen atmosphere and the mixture was warmed to ambient temperature and stirred for an hour. To this mixture was added dropwise 1,1,2,2,3,3,4,4,4-nonafluoro-1-butanesulfonyl fluoride (39.0 g) at ambient temperature and the mixture was warmed to 35° C. and stirred at 35° C. for 20 hours. The reaction mixture was poured into a mixture of ethyl acetate and ice water and adjusted to pH ca.2 with 6N hydrochloric acid. The separated organic layer was washed with water and brine, dried over magnesium sulfate and evaporated in vacuo. The residue was purified by column chromatography on silica gel eluting with hexane:toluene (1:1 v/v) to give methyl 2-{[(nonafluorobutyl)sulfonyl]oxy}-1-cycloheptene-1-carboxylate (29.82 g) as a colorless oil.

¹H-NMR (DMSO-d₆):δ 1.6-1.9 (6H, m), 2.6-2.9 (4H, m), 3.70 (3H, s)

ESI-MS(m/z): 475 (M+Na)⁺

Preparation 173

To a suspension of zinc chloride (17.91 g) in tetrahydrofuran (200 ml) was added dropwise a 1 mol/L solution of tolylmagnesium bromide in tetrahydrofuran (98.6 ml) at 0° C. under a nitrogen atmosphere and the mixture was stirred at 0° C. for 30 minutes. To this suspension were added bis(dibenzylideneacetone)palladium (1.13 g) and 1,1′-bis(diphenylphosphino)ferrocene (1.09 g), followed by dropwise addition of methyl 2-{[(nonafluorobutyl)sulfonyl]oxy}-1-cycloheptene-1-carboxylate (29.72 g) in tetrahydrofuran (90 ml). The mixture was refluxed for 16 hours under a nitrogen atmosphere. The reaction mixture was poured into a mixture of ethyl acetate and ice water and adjusted to pH ca.2 with 6N hydrochloric acid. The separated organic layer was washed with water and brine, dried over magnesium sulfate and evaporated in vacuo. The residue was purified by column chromatography on silica gel eluting with hexane:toluene (1:3 v/v) to give methyl 2-(4-methylphenyl)-1-cycloheptene-1-carboxylate (13.77 g) as a colorless oil.

¹H-NMR (DMSO-d₆):δ 1.6-1.9 (6H, m), 2.28 (3H, s), 2.5-2.5 (4H, m) 3.70 (3H, s), 6.95-7.0 (2H, m), 7.1-7.15 (2H, m)

ESI-MS(m/z): 267 (M+Na)⁺

Preparation 174

To a solution of methyl 2-(4-methylphenyl)-1-cycloheptene-1-carboxylate (13.76 g) in ethanol (130 ml) was added 5N aqueous sodium hydroxide solution (22.6 ml) at ambient temperature and the mixture was refluxed for 4 hours. The reaction mixture was cooled to 5° C. and ice-water (60 ml) was added. The mixture was adjusted to ca.7 with 6N hydrochloric acid and concentrated in vacuo. To the residue was added a mixture of ethyl acetate and water and the mixture was adjusted to pH ca.2 with 6N hydrochloric acid. The separated organic layer was washed with water and brine, dried over magnesium sulfate and evaporated in vacuo. The residue was triturated with hexane to give 2-(4-methylphenyl)-1-cycloheptene-1-carboxylic acid (3.58 g) as white crystals.

¹H-NMR (DMSO-d₆):δ 1.45-1.6 (4H, m), 1.7-1.9 (2H, m), 2.27 (3H, s), 2.4-2.55 (4H, m), 7.0-7.15 (4H, m), 11.90 (1H, br s)

ESI-MS(m/z): 253 (M+Na)⁺

### EXAMPLE 224

2-(4-Methylphenyl)-N-[1-(2-pyridinylacetyl)-2,3-dihydro-1H-indol-5-yl]-1-cycloheptene-1-carboxamide

The title compound was obtained in the same manner as in Example 151 as white crystals.

¹H-NMR (DMSO-d₆):δ 1.6-1.9 (6H, m), 2.21 (3H, s), 2.4-2.5 (4H, m), 2.85 (2H, t, J=7.7 Hz), 3.99 (2H, t, J=7.7 Hz), 7.0-7.3 (8H, m), 7.37 (2H, d, J=8.7 Hz), 7.6-7.7 (1H, m), 8.25 (1H, s), 8.45 (1H, d, J=3.9 Hz), 9.42 (1H, s)

ESI-MS(m/z): 488 (M+Na)⁺, 466 (M+H)⁺

### EXAMPLE 225

4′-(Dimethylamino)-N-(1-{[6-(2,5-dimethyl-1H-pyrrol-1-yl)-2-pyridinyl]acetyl}-2,3-dihydro-1H-indol-5-yl)-1,1′-biphenyl-2-carboxamide

The title compound was obtained in the same manner as in Example 151 as white crystals.

¹H-NMR (DMSO-d₆):δ 2.03 (6H, s), 2.88 (6H, s), 3.10 (2H, t, J=8.1 Hz), 4.05 (2H, s), 4.17 (2H, t, J=8.1 Hz), 5.77 (2H, s), 6.70 (2H, d, J=8.8 Hz), 7.27 (2H, d, J=8.8 Hz), 7.2-7.55 (8H, m), 7.85-8.0 (2H, m), 10.13 (1H, s)

ESI-MS(m/z): 592 (M+Na)⁺, 570 (M+H)⁺

### EXAMPLE 226

N-{1-[(6-Amino-2-pyridinyl)acetyl]-2,3-dihydro-1H-indol-5-yl}-4′-(dimethylamino)-1,1′-biphenyl-2-carboxamide

The title compound was obtained in the same manner as in Example 9 as white crystals.

¹H-NMR (DMSO-d₆):δ 2.88 (6H, s), 3.10 (2H, t, J=8.3 Hz), 3.69 (2H, s), 4.17 (2H, t, J=8.3 Hz), 5.85 (2H, br s), 6.30 (1H, d, J=8.2 Hz), 6.42 (1H, d, J=7.2 Hz), 6.70 (2H, d, J=8.8 Hz), 7.2-7.6 (11H, m), 7.92 (1H, d, J=8.7 Hz), 10.11 (1H, s)

ESI-MS(m/z): 514 (M+Na)⁺, 492 (M+H)⁺

### EXAMPLE 227

A solution of 4-pyrimidinylacetic acid (0.141 g), N-(2,3-dihydro-1H-indol-5-yl)-4′-methyl-1,1′-biphenyl-2-carboxamide hydrochloride (0.298 g) and benzotriazol-1-yloxytripyrrolidinophosphonium hexafluorophosphate (PyBOP) (0.637 g) in N,N-dimethylformamide (5 ml) was cooled to 5° C. and diisopropylethylamine (0.9 ml) was dropwise added to the solution. The reaction mixture was stirred at ambient temperature for 15 hours and concentrated in vacuo. The residue was dissolved in ethyl acetate and water, and extracted with ethyl acetate. The organic layer was washed with water and brine, dried over magnesium sulfate and concentrated in vacuo. The residue was purified by column chromatography on silica gel by eluting with hexane:ethyl acetate (3:7 v/v) to give 4′-methyl-N-[1-(4-pyrimidinylacetyl)-2,3-dihydro-1H-indol-5-yl]-1,1′-biphenyl-2-carboxamide (0.132 g) as a yellow foam.

¹H-NMR (DMSO-d₆):δ 2.29 (3H, s), 3.13 (2H, t, J=8.4 Hz), 4.04 (2H, s), 4.19 (2H, t, J=8.4 Hz), 7.16-7.55 (11H, m), 7.89 (1H, d, J=8.9 Hz), 8.74 (1H, d, J=5.4 Hz), 9.11 (1H, d, J=1.3 Hz), 10.17 (1H, s)

ESI-MS(m/z): 449 (M+H)⁺

### EXAMPLE 228

A solution of 4-pyrimidinylacetic acid (0.154 g), N-(2,3-dihydro-1H-indol-5-yl)-4′-(trifluoromethyl)-1,1′-biphenyl-2-carboxamide hydrochloride (0.374 g) and benzotriazol-1-yloxytripyrrolidinophosphonium hexafluorophosphate (PyBOP) (0.580 g) in N,N-dimethylformamide (5 ml) was cooled to 5° C. and diisopropylethylamine (0.8 ml) was dropwise added to the solution. The reaction mixture was stirred at ambient temperature for 15 hours and concentrated in vacuo. The residue was dissolved in ethyl acetate and water, and extracted with ethyl acetate. The organic layer was washed with water and brine, dried over magnesium sulfate and concentrated in vacuo. The residue was purified by column chromatography on silica gel by eluting with hexane:ethyl acetate (3:7 v/v) to give N-[1-(4-pyrimidinylacetyl)-2,3-dihydro-1H-indol-5-yl]-4′-(trifluoromethyl)-1,1′-biphenyl-2-carboxamide (0.143 g) as a yellow foam.

¹H-NMR (DMSO-d₆):δ 3.13 (2H, t, J=8.4 Hz), 4.05 (2H, s), 4.19 (2H, t, J=8.4 Hz), 7.21 (1H, d, J=8.9 Hz), 7.50-7.77 (10H, m), 7.90 (1H, d, J=8.9 Hz), 8.74 (1H, d, J=5.3 Hz), 9.11 (1H, d, J=1.3 Hz), 10.28 (1H, s)

ESI-MS(m/z): 438 (M+H)⁺

### EXAMPLE 229

To a solution of N-(2,3-dihydro-1H-indol-5-yl)-4′-(trifluoromethyl)-1,1′-biphenyl-2-carboxamide hydrochloride (226 mg), (2-methyl-1,3-thiazol-4-yl)acetic acid (85 mg) and 1-hydroxybenzotriazole (99 mg) in N,N-dimethylformamide (15 ml) was added 1-[3-(dimethylamino)propyl]-3-ethylcarbodiimide hydrochloride (WSC.HCl) (124 mg), followed by addition of triethylamine (142 mg) at ambient temperature. The reaction mixture was stirred at 45° C. for 16 hours and concentrated in vacuo. The residue was dissolved in ethyl acetate and water, and extracted with ethyl acetate. The organic layer was washed with brine, dried over magnesium sulfate and concentrated in vacuo. The residue was purified by column chromatography on silica gel by eluting with hexane:ethyl acetate (1:3 v/v) to give an orange oil. The obtained oil was recrystallized from ethyl acetate-diisopropyl ether to give N-{1-[(2-methyl-1,3-thiazol-4-yl)acetyl]-2,3-dihydro-1H-indol-5-yl}-4′-(trifluoromethyl)-1,1′-biphenyl-2-carboxamide (93 mg) as colorless crystals.

¹H-NMR (DMSO-d₆):δ 2.62 (3H, s), 3.11 (2H, t, J=8.3 Hz), 3.89 (2H, s), 4.19 (2H, t, J=8.3 Hz), 7.22 (1H, d, J=8.6 Hz), 7.27 (1H, s), 7.48-7.64 (7H, m), 7.76 (2H, d, J=8.3 Hz), 7.92 (1H, d, J=8.9 Hz), 10.26 (1H, s).

ESI-MS(m/z): 522 (M+H)⁺

### EXAMPLE 230

N-[1-(1,3-Thiazol-4-ylacetyl)-2,3-dihydro-1H-indol-5-yl]-4′-(trifluoromethyl)-1,1′-biphenyl-2-carboxamide

The title compound was obtained in the same manner as in Example 229 as colorless crystals.

¹H-NMR (DMSO-d₆):δ 3.11 (2H, t, J=8.3 Hz), 4.01 (2H, s), 4.21 (2H, t, J=8.3 Hz), 7.21 (1H, d, J=8.9 Hz), 7.49-7.77 (10H, m), 7.92 (1H, d, J=8.9 Hz), 10.26 (1H, s).

ESI-MS(m/z): 508 (M+H)⁺

### EXAMPLE 231

4′-Methyl-N-{1-[(2-methyl-1,3-thiazol-4-yl)acetyl]-2,3-dihydro-1H-indol-5-yl}-1,1′-biphenyl-2-carboxamide

The title compound was obtained in the same manner as in Example 229 as faintly orange crystals.

¹H-NMR (DMSO-d₆):δ 2.29 (3H, s), 2.62 (3H, s), 3.11 (2H, t, J=8.3 Hz), 3.89 (2H, s), 4.19 (2H, t, J=8.3 Hz), 7.16-7.57 (11H, m), 7.92 (1H, d, J=8.9 Hz), 10.15 (1H, s).

ESI-MS(m/z): 490 (M+Na)⁺, 468 (M+H)⁺

### EXAMPLE 232

4′-Methyl-N-[1-(1,3-thiazol-4-ylacetyl)-2,3-dihydro-1H-indol-5-yl]-1,1′-biphenyl-2-carboxamide

The title compound was obtained in the same manner as in Example 229 as faintly orange crystals.

¹H-NMR (DMSO-d₆):δ 2.29 (3H, s), 3.12 (2H, t, J=8.3 Hz), 4.01 (2H, s), 4.21 (2H, t, J=8.3 Hz), 7.16-7.57 (11H, m), 7.91 (1H, d, J=8.6 Hz), 9.05 (1H, s), 10.16 (1H, s).

ESI-MS(m/z): 476 (M+Na)⁺, 454 (M+H)⁺

### EXAMPLE 233

To a solution of [2-(2,5-dimethyl-1H-pyrrol-1-yl)-4-pyrimidinyl]acetic acid (0.396 g), N-(2,3-dihydro-1H-indol-5-yl)-4′-methyl-1,1′-biphenyl-2-carboxamide hydrochloride (0.521 g) and 1-hydroxybenzotriazole (0.232 g) in N,N-dimethylformamide (10 ml) was added 1-[3-(dimethylamino)propyl]-3-ethylcarbodiimide hydrochloride (WSC.HCl) (0.33 g), followed by addition of triethylamine (0.3 ml) at ambient temperature. The reaction mixture was stirred at 50° C. for 12 hours and concentrated in vacuo. The residue was dissolved in ethyl acetate and water, and extracted with ethyl acetate. The organic layer was washed with water and brine, dried over magnesium sulfate and concentrated in vacuo. The residue was purified by column chromatography on silica gel by eluting with hexane:ethyl acetate (1:3 v/v) to give N-(1-{[2-(2,5-dimethyl-1H-pyrrol-1-yl)-4-pyrimidinyl]acetyl}-2,3-dihydro-1H-indol-5-yl)-4′-methyl-1,1′-biphenyl-2-carboxamide (0.35 g) as a yellow oil.

¹H-NMR (DMSO-d₆):δ 2.21 (6H, s), 2.28 (3H, s), 3.12 (2H, t, J=8.3 Hz), 4.17 (2H, t, J=8.3 Hz), 5.80 (2H, s), 7.15-7.54 (11H, m), 7.90 (1H, d, J=8.6 Hz), 8.82 (1H, d, J=5.3 Hz), 10.19 (1H, s)

### EXAMPLE 234

To a solution of N-(1-{[2-(2,5-dimethyl-1H-pyrrol-1-yl)-4-pyrimidinyl]acetyl}-2,3-dihydro-1H-indol-5-yl)-4′-methyl-1,1′-biphenyl-2-carboxamide in ethanol and water was added hydroxylamine hydrochloride, followed by addition of triethylamine at ambient temperature. The reaction mixture was heated to 100° C. and stirrred for 15 hours. The reaction mixture was cooled to ambient temperature and concentrated in vacuo. The residue was dissolved in ethyl acetate and water, and extracted with ethyl acetate. The organic layer was washed with water and brine, dried over magnesium sulfate and concentrated in vacuo. The residue was purified by column chromatography on silica gel by eluting with hexane:ethyl acetate (1:3 v/v) to give N-{1-[(2-amino-4-pyrimidinyl)acetyl]-2,3-dihydro-1H-indol-5-yl}-4′-methyl-1,1′-biphenyl-2-carboxamide (42 mg) as a pale brown solid.

¹H-NMR (DMSO-d₆):δ 2.29 (3H, s), 3.11 (2H, t, J=7.9 Hz), 3.74 (2H, s), 4.15 (2H, t, J=7.9 Hz), 6.51-6.55 (3H, m), 7.15-7.53 (10H, m), 7.90 (1H, d, J=8.6 Hz), 8.15 (1H, d, J=4.9 Hz), 10.16 (1H, s)

ESI-MS(m/z): 464 (M+H)⁺

### EXAMPLE 235

4′-Trifluoromethyl-N-[1-(2-pyrazinylacetyl)-2,3-dihydro-1H-indol-5-yl]-1,1′-biphenyl-2-carboxamide

The title compound was obtained in the same manner as in Example 229 as a beige powder.

¹H-NMR (DMSO-d₆):δ 3.14 (2H, t, J=8 Hz), 4.10 (2H, s), 4.23 (2H, t, J=8 Hz), 7.19-7.90 (11H, m), 8.53-8.63 (3H, m), 10.27 (1H, s).

EI-MS (m/z): 502 (M+H)⁺

### EXAMPLE 236

4′-Methyl-N-[1-(1,3-thiazol-4-ylacetyl)-2,3-dihydro-1H-indol-5-yl]-1,1′-biphenyl-2-carboxamide

The title compound was obtained in the same manner as in Example 229 as a brown powder.

¹H-NMR (DMSO-d₆):δ 2.29 (3H, s), 3.15 (2H, t, J=8 Hz), 4.10 (2H, s), 4.23 (2H, t, J=8 Hz), 7.16-7.90 (11H, m), 8.53-8.64 (3H, m), 10.17 (1H, s).

EI-MS (m/z): 448 (M+H)⁺

This application is based on application No. PR 4722 filed in Australia on Apr. 30, 2001, and application No. PR 9937 filed in Australia on Jan. 11, 2002, the content of which is incorporated hereinto by reference.

1. A compound of formula (I): 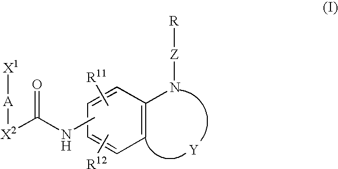
wherein
X¹ is 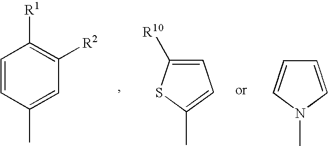
wherein R¹, R² and R¹⁰ are independently hydrogen or a suitable substituent;
R¹¹ and R¹² are independently hydrogen or a suitable substituent;
R is unsaturated 5 to 6-membered heteromonocyclic group, which is optionally substituted by one or more suitable substituent(s);
A is direct bond or —NH—;
X² is monocyclic arylene, unsaturated 5 to 6-membered heteromonocyclic group or cycloalkenylene, each of which is optionally substituted by one or more suitable substituent(s);
Y is bivalent group selected from the group consisting of ethylene, trimethylene and vinylene, wherein CH₂ is optionally replaced by NH or O, and CH is optionally replaced by N, and said bivalent group is optionally substituted by one or more suitable substituent(s);
and
Z is —(CH₂)ₙ—, —CO—(CH₂)ₘ—, —CH═CH— or —CO—NH—, wherein n is 1, 2 or 3 and m is 1 or 2, or a salt thereof.

2. The compound of claim 1 wherein
R¹ is hydrogen, lower alkyl, lower alkenyl, lower alkoxy, aryl, aryloxy, halogen, trihalo(lower)alkyl, di(lower)alkylamino(lower)alkyl and lower alkanoyloxy(lower)alkyl; and
Y is bivalent group selected from the group consisting of ethylene, trimethylene and vinylene, wherein CH₂ is optionally replaced by NH or O, and CH is optionally replaced by N, and said bivalent group is optionally substituted by one or more substituent(s) selected from the group consisting of lower alkyl, oxo and amino,
or a salt thereof.

3. The compound of claim 2 wherein
R is pyridinyl, pyrimidinyl, pyrazinyl, thiazolyl, thiadiazolyl or triazolyl, each of which is optionally substituted by lower alkyl, optionally protected amino, lower alkylamino, aryl(lower)alkyl, guanidino or oxido; and
X² is bivalent group selected from 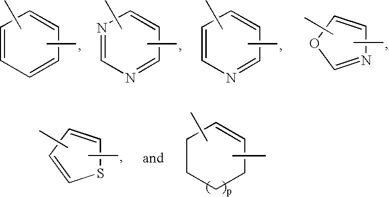
wherein p is 0, 1 or 2,
said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl,
or a salt thereof.

4. The compound of claim 3 wherein
R is
trihalo(lower)alkoxy, nitro, optionally protected amino, lower alkylamino, di(lower)alkylamino, cyclic amino group, lower alkylthio, lower alkylsulfonyl, lower alkylsulfonyloxy, hydroxy(lower)alkyl, optionally protected amino(lower)alkyl, lower alkanoyl, optionally protected carboxy or N,N-di(lower)alkylcarbamoyl;
R² is hydrogen, lower alkyl, lower alkoxy, halogen or trihalo(lower)alkyl;
R¹⁰ is hydrogen or halogen;
R¹¹ and R¹² are independently hydrogen or lower alkyl;
R is unsaturated 5-membered heteromonocyclic group containing 1 or 2 nitrogen atom(s) and a sulfur atom, unsaturated 5-membered heteromonocyclic group containing 1 or 3 nitrogen atom(s), or unsaturated 6-membered heteromonocyclic group containing 1 or 2 nitrogen atom(s), each of said heteromonocyclic groups is optionally substituted by one or more substituent(s) selected from the group consisting of lower alkyl, optionally protected amino, lower alkylamino, aryl(lower)alkyl, guanidino and oxido;
X² is bivalent group selected from the group consisting of phenylene,
cycloalkenylene,
unsaturated 5-membered heteromonocyclic group containing 1 or 2 hetero atom(s) selected from the group consisting of nitrogen, oxygen and sulfur atoms, and
unsaturated 6-membered heteromonocyclic group containing 1 or 2 nitrogen atom(s),
said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N 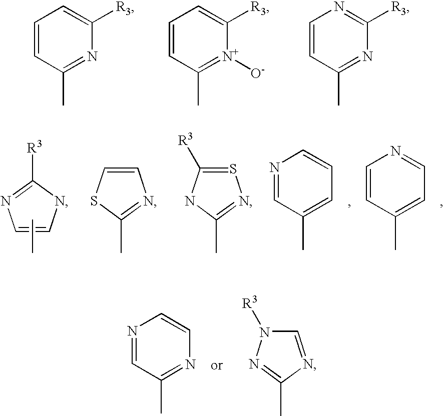
wherein R³ is hydrogen, lower alkyl, optionally protected amino, lower alkylamino, trityl or guanidino;
X² is 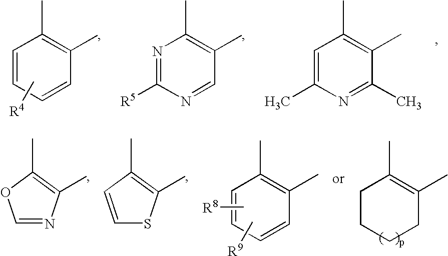
wherein R⁴ is hydrogen, lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl or lower alkanoyloxy(lower)alkyl;
R⁵ is hydrogen or lower alkyl;
R⁸ and R⁹ are independently lower alkyl or lower alkoxy; and
p is 0, 1 or 2; and
Y is 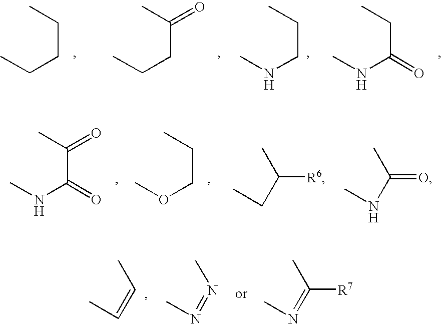
wherein R⁶ is hydrogen or lower alkyl; and
R⁷ is hydrogen, lower alkyl or amino,
or a salt thereof.

5. The compound of claim 4 wherein
R¹ is hydrogen, methyl, ethyl, isopropyl, isopropenyl, methoxy, ethoxy, phenyl, phenoxy, chloro, fluoro, trifluoromethyl, trifluoromethoxy, nitro, amino, dimethylamino, piperidino, 4-morpholinyl, 4-thiomorpholinyl, 1,1-dioxothiomorpholin-4-yl, methylthio, isopropylthio, methylsulfonyl, methylsulfonyloxy, 1-hydroxyethyl, 1-hydroxy-1-methylethyl, 1-aminoethyl, 1-(benzylamino)ethyl, acetyl, acetylamino, carboxy, methoxycarbonyl, isopropoxycarbonyl, pivaloyloxymethoxycarbonyl or N,N-diethylcarbamoyl;
R² is hydrogen, methyl, methoxy, chloro or trifluoromethyl;
R¹⁰ is chloro;
R¹¹ and R¹² are independently hydrogen or methyl;
A is direct bond;
Z is —CH₂CH₂—, —CO—CH₂—, —CH═CH— or —CO—NH—;
R³ is hydrogen, methyl, amino, methylamino, formylamino, tert-butoxycarbonylamino, 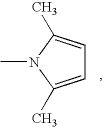
trityl or guanidino;
R⁴ is hydrogen, methyl, methoxy, chloro, nitro, amino, dimethylamino, hydroxymethyl, methoxymethyl, N,N-dimethylaminomethyl or acetyloxymethyl;
R⁵ is hydrogen, methyl or isopropyl;
R⁶ is hydrogen or methyl;
R⁷ is hydrogen, methyl or amino; and
R⁸ and R⁹ are independently methyl or methoxy,
or a salt thereof.

6. A compound of formula (II): 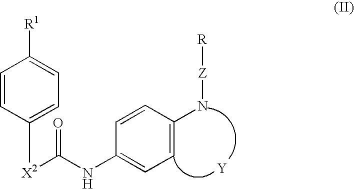
wherein
R¹ is hydrogen, methyl, ethyl, isopropyl, isopropenyl, methoxy, ethoxy, phenyl, phenoxy, chloro, fluoro, trifluoromethyl, trifluoromethoxy, nitro, amino, dimethylamino, piperidino, 4-morpholinyl, 4-thiomorpholinyl, 1,1-dioxothiomorpholin-4-yl, methylthio, isopropylthio, methylsulfonyl, methylsulfonyloxy, 1-hydroxyethyl, 1-hydroxy-1-methylethyl, 1-aminoethyl, 1-(benzylamino)ethyl, acetyl, acetylamino, carboxy, methoxycarbonyl, isopropoxycarbonyl, pivaloyloxymethoxycarbonyl or N,N-diethylcarbamoyl;
R is 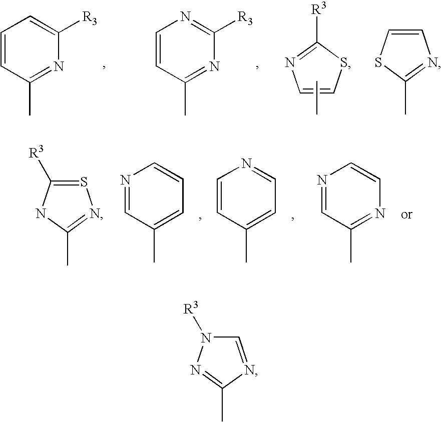
wherein R³ is hydrogen, methyl, amino, methylamino, formylamino, tert-butoxycarbonylamino, 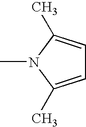
or trityl;
X² is 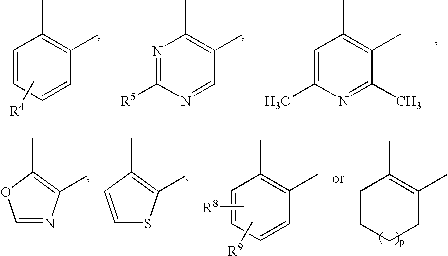
wherein R⁴ is hydrogen, methyl, methoxy, chloro, nitro, amino, dimethylamino, hydroxymethyl, methoxymethyl, N,N-dimethylaminomethyl or acetyloxymethyl;
R⁵ is hydrogen, methyl or isopropyl;
R⁸ and R⁹ are independently methyl or methoxy; and
p is 0, 1 or 2;
Y is 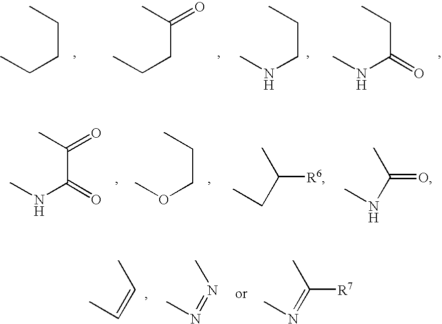
wherein R⁶ is hydrogen or methyl; and
R⁷ is hydrogen, methyl or amino; and
Z is —CH₂CH₂—, —CO—CH₂— or —CH═CH—,
or a salt thereof.

7. A compound of formula (III): 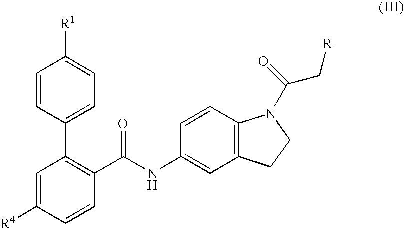
wherein
R¹ is hydrogen, lower alkyl, halogen, trihalo(lower)alkyl or di(lower)alkylamino;
R is 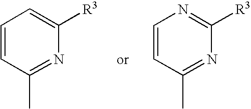
wherein R³ is hydrogen or amino; and
R⁴ is hydrogen or lower alkyl;
or a salt thereof.

8. The compound of claim 7, which is selected from the group consisting of
N-[1-(2-pyridinylacetyl)-2,3-dihydro-1H-indol-5-yl]-4′-(trifluoromethyl)-1,1′-biphenyl-2-carboxamide,
4′-ethyl-N-[1-(2-pyridinylacetyl)-2,3-dihydro-1H-indol-5-yl]-1,1′-biphenyl-2-carboxamide,
N-{1-[(6-amino-2-pyridinyl)acetyl]-2,3-dihydro-1H-indol-5-yl}-4′-(trifluoromethyl)-1,1′-biphenyl-2-carboxamide,
4′,5-dimethyl-N-[1-(2-pyridinylacetyl)-2,3-dihydro-1H-indol-5-yl]-1,1′-biphenyl-2-carboxamide,
4′-chloro-5-methyl-N-[1-(2-pyridinylacetyl)-2,3-dihydro-1H-indol-5-yl]-1,1′-biphenyl-2-carboxamide,
4′-(dimethylamino)-N-[1-(2-pyridinylacetyl)-2,3-dihydro-1H-indol-5-yl]-1,1′-biphenyl-2-carboxamide,
N-{1-[(6-amino-2-pyridinyl)acetyl]-2,3-dihydro-1H-indol-5-yl}-4′-methyl-1,1′-biphenyl-2-carboxamide,
N-{1-[(6-amino-2-pyridinyl)acetyl]-2,3-dihydro-1H-indol-5-yl}-4′-ethyl-1,1′-biphenyl-2-carboxamide,
N-{1-[(2-amino-4-pyrimidinyl)acetyl]-2,3-dihydro-1H-indol-5-yl}-4′-ethyl-1,1′-biphenyl-2-carboxamide, and
N-{1-[(2-amino-4-pyrimidinyl)acetyl]-2,3-dihydro-1H-indol-5-yl}-4′-methyl-1,1′-biphenyl-2-carboxamide, or a salt thereof.

9. The compound of claim 1 wherein
X¹ is 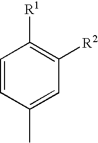
R¹ and R² are independently hydrogen or a suitable substituent;
X² is monocyclic arylene or unsaturated 5 or 6-membered heteromonocyclic group, each of which is optionally substituted by one or more suitable substituent(s);
and
Z is —(CH₂)ₙ—, —CO—(CH₂)ₘ— or —CH═CH—, wherein n is 1, 2 or 3 and m is 1 or 2,
or a salt thereof.

10. The compound of claim 9 wherein
R¹ is hydrogen, lower alkyl, lower alkoxy, aryloxy, halogen, trihalo(lower)alkyl, trihalo(lower)alkoxy, nitro, optionally protected amino, lower alkylamino or di(lower)alkylamino;
R² is hydrogen, lower alkyl, lower alkoxy, halogen or trihalo(lower)alkyl;
R is unsaturated 5-membered heteromonocyclic group containing 1 or 2 nitrogen atom(s) and a sulfur atom, or
unsaturated 6-membered heteromonocyclic group containing 1 or 2 nitrogen atom(s),
each of said heteromonocyclic groups is optionally substituted by one or more substituent(s) selected from the group consisting of lower alkyl, optionally protected amino and lower alkylamino;
X² is bivalent group selected from the group consisting of phenylene,
unsaturated 5-membered heteromonocyclic group containing 1 or 2 hetero atom(s) selected from the group consisting of nitrogen, oxygen and sulfur atoms, or
unsaturated 6-membered heteromonocyclic group containing 1 or 2 nitrogen atom(s),
said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl; and
Y is bivalent group selected from the group consisting of ethylene, trimethylene and vinylene, wherein CH₂ is optionally replaced by NH or O, and CH is optionally replaced by N, and said bivalent group is optionally substituted by one or more substituent(s) selected from the group consisting of lower alkyl, oxo and amino,
or a salt thereof.

11. The compound of claim 10 wherein
R is pyridinyl, pyrimidinyl, pyrazinyl, thiazolyl or thiadiazolyl, each of which is optionally substituted by lower alkyl, optionally protected amino or lower alkylamino; and
X² is bivalent group selected from 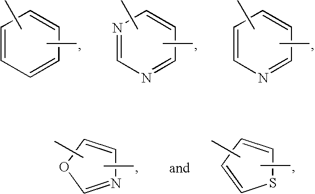
said bivalent group is optionally substituted by one or more substituent(s) selected from the group consisting of lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl and lower alkanoyloxy(lower)alkyl,
or a salt thereof.

12. The compound of claim 11 wherein
R is 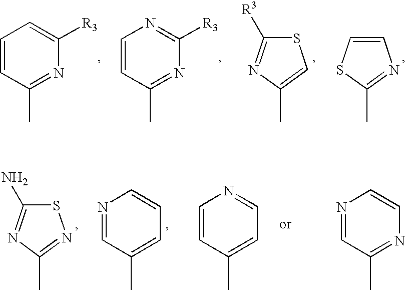
wherein R³ is hydrogen, lower alkyl, optionally protected amino or lower alkylamino;
X² is 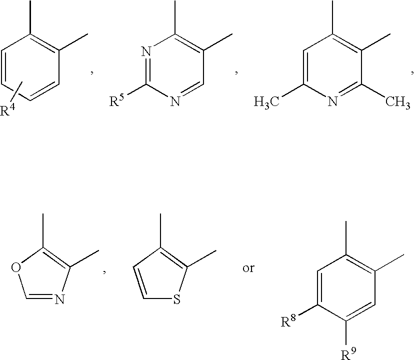
wherein R⁴ is hydrogen, lower alkyl, lower alkoxy, halogen, nitro, optionally protected amino, lower alkylamino, di(lower)alkylamino, hydroxy(lower)alkyl, lower alkoxy(lower)alkyl, amino(lower)alkyl, N-lower alkylamino(lower)alkyl, N,N-di(lower)alkylamino(lower)alkyl or lower alkanoyloxy(lower)alkyl;
R⁵ is hydrogen or lower alkyl; and
R⁸ and R⁹ are independently lower alkyl or lower alkoxy; and
Y is 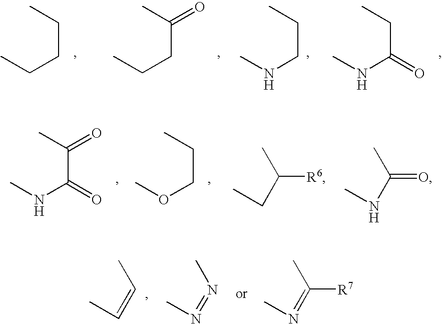
wherein R⁶ is hydrogen or lower alkyl; and
R⁷ is hydrogen or amino,
or a salt thereof.

13. The compound of claim 12 wherein
R¹ is hydrogen, methyl, ethyl, methoxy, ethoxy, phenoxy, chloro, fluoro, trifluoromethyl, trifluoromethoxy, nitro, amino or dimethylamino;
R² is hydrogen, methyl, methoxy, chloro or trifluoromethyl;
A is direct bond;
Z is —CH₂CH₂—, —CO—CH₂— or —CH═CH—;
R³ is hydrogen, methyl, amino, methylamino, formylamino, tert-butoxycarbonylamino or 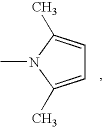
R⁴ is hydrogen, methyl, methoxy, chloro, nitro, amino, dimethylamino, hydroxymethyl, methoxymethyl, N,N-dimethylaminomethyl or acetyloxymethyl;
R⁵ is hydrogen, methyl or isopropyl;
R⁶ is hydrogen or methyl; and
R⁸ and R⁹ are independently methyl or methoxy,
or a salt thereof.

14. The compound of claim 1 wherein
X¹ is 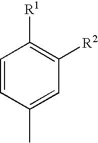
R¹ and R² are independently hydrogen or a suitable substituent;
X² is monocyclic arylene or unsaturated 5 or 6-membered heteromonocyclic group, each of which is optionally substituted by one or more suitable substituent(s);
and
Z is —(CH)ₙ— or —CO—(CH₂)ₘ—, wherein n is 1, 2 or 3 and m is 1 or 2,
or a salt thereof.

15. The compound of claim 14 wherein
R¹ is hydrogen, lower alkyl, lower alkoxy, aryloxy, halogen, trihalo(lower)alkyl, trihalo(lower)alkoxy, nitro, optionally protected amino, lower alkylamino or di(lower)alkylamino;
R² is hydrogen, lower alkyl, lower alkoxy, halogen or trihalo(lower)alkyl;
R is 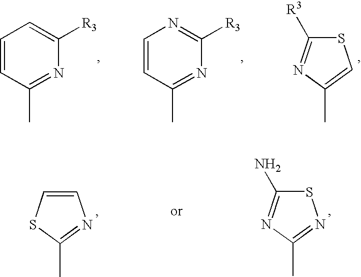
wherein R³ is hydrogen, lower alkyl, optionally protected amino, or lower alkylamino;
X² is 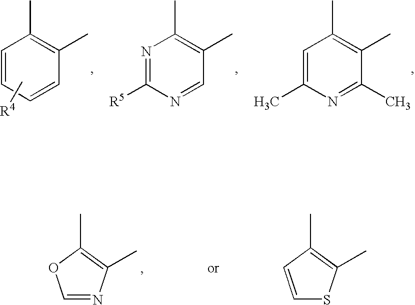
wherein R⁴ is hydrogen, lower alkyl, lower alkoxy, halogen, nitro, optionally protected amino, (lower)alkylamino, di(lower)alkylamino, hydroxy(lower)alkyl, lower alkoxy(lower)alkyl, amino(lower)alkyl, N-lower alkylamino(lower)alkyl, or N,N-di(lower)alkylamino(lower)alkyl; and
R⁵ is hydrogen or lower alkyl; and
Y is 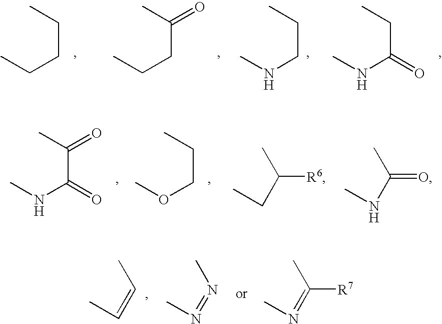
wherein R⁶ is hydrogen or lower alkyl; and
R⁷ is hydrogen or amino,
or a salt thereof.

16. The compound of claim 15 wherein
R¹ is hydrogen, methyl, ethyl, methoxy, ethoxy, phenoxy, chloro, fluoro, trifluoromethyl, trifluoromethoxy, nitro, amino or dimethylamino;
R² is hydrogen, methyl, methoxy, chloro or trifluoromethyl;
R³ is hydrogen, methyl, amino, methylamino, formylamino, tert-butoxycarbonylamino or 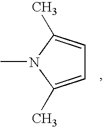
R⁴ is hydrogen, methyl, methoxy, chloro, nitro, amino, dimethylamino, hydroxymethyl, methoxymethyl or N,N-dimethylaminomethyl;
R⁵ is hydrogen, methyl or isopropyl; and
R⁶ is hydrogen or methyl,
or a salt thereof.

17. The compound of claim 16 above wherein A is direct bond and Z is —CH₂CH₂— or —CO—CH₂—, or a salt thereof.

18. The compound of claim 1 or a pharmaceutically acceptable salt thereof for use as a medicament.

19. A pharmaceutical composition comprising a compound of claim 1 or a pharmaceutically acceptable salt thereof in admixture with a pharmaceutically acceptable carrier.

20. Use of a compound of claim 1 or a pharmaceutically acceptable salt thereof for preparing a medicament as an apolipoprotein B (Apo B) secretion inhibitor.

21. Use of a compound of claim 1 or a pharmaceutically acceptable salt thereof for preparing a medicament for the prophylaxis or treatment of a disease or condition resulting from elevated circulating levels of Apo B.

22. Use of a compound of claim 1 or a pharmaceutically acceptable salt thereof for preparing a medicament for the prophylaxis or treatment of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis or Syndrome X.

23. A method for inhibiting or decreasing Apo B secretion in a mammal, which comprises administering an Apo B secretion inhibiting or decreasing amount of a compound of claim 1 or a pharmaceutically acceptable salt thereof to the mammal.

24. A method for preventing or treating a disease or condition resulting from elevated circulating levels of Apo B in a mammal, which comprises administering an effective amount of a compound of claim 1 or a pharmaceutically acceptable salt thereof to the mammal.

25. The method of claim 24 wherein the disease or condition resulting from the elevated circulating levels of Apo B is selected from the group consisting of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis and Syndrome X.

26. An Apo B secretion inhibitor, which comprises a compound of claim 1 or a pharmaceutically acceptable salt thereof.

27. A medicament for the prophylaxis or treatment of a disease or condition resulting from elevated circulating levels of Apo B, which comprises a compound of claim 1 or a pharmaceutically acceptable salt thereof.

28. A medicament for the prophylaxis or treatment of hyperlipemia, hyperlipidemia, hyperlipoproteinemia, hypoalphalipoproteinemia, hypercholesterolemia, hypertriglyceridemia, atherosclerosis, pancreatitis, non-insulin dependent diabetes mellitus (NIDDM), obesity, coronary heart diseases, myocardial infarction, stroke, restenosis or Syndrome X, which comprises a compound of claim 1 or a pharmaceutically acceptable salt thereof.
